(19) United States
(12) Patent Application Publication
(10) Pub. No.: US 2024/0092885 A1
(43) Pub. Date: Mar. 21, 2024

(54) VECTOR CONSTRUCTS FOR DELIVERY OF NUCLEIC ACIDS ENCODING THERAPEUTIC ANTI-TNF ANTIBODIES AND METHODS OF USING THE SAME

(71) Applicant: Kriya Therapeutics, Inc., Palo Alto, CA (US)

(72) Inventors: Brian FURMANSKI, Morrisville, NC (US); Bruce SCHNEPP, Morrisville, NC (US); Nachi GUPTA, Morrisville, NC (US); Shankar RAMASWAMY, Morrisville, NC (US); Weiran SHEN, Morrisville, NC (US)

(21) Appl. No.: 18/262,880

(22) PCT Filed: Jan. 26, 2022
(86) PCT No.: PCT/US2022/013935

§ 371 Date: Jul. 25, 2023

Related U.S. Application Data
(60) Provisional application No. 63/141,916, filed on Jan. 26, 2021.

(51) Int. Cl. C07K 16/24; C12N 15/86

(52) U.S. Cl. CPC C07K 16/241; C12N 15/86; A61K 2039/5256; C07K 2317/76; C07K 2319/50; C12N 2750/14143; C12N 2830/50; C12N 2840/203

(57) ABSTRACT

The present disclosure provides gene therapy expression constructs comprising a nucleic acid encoding a therapeutic anti-TNFalpha antibody (e.g., a monoclonal antibody) or an antigen binding fragment thereof, delivery vectors (e.g., viral vectors) comprising the same, compositions comprising the same, and methods of using the same (e.g., to treat ocular diseases or disorders). Some aspects of the disclosure are directed to a recombinant adeno-viral vector (rAAV) delivery comprising an AAV particle (e.g., AAV2) and a nucleic acid encoding a therapeutic anti-TNFalpha antibody (e.g., a monoclonal antibody) or an antigen binding fragment thereof.

### CROSS REFERENCE TO RELATED APPLICATIONS

The present application claims the priority benefit of U.S. Provisional Application No. 63/141,916 filed Jan. 26, 2021, which is hereby incorporated by reference in its entirety.

### REFERENCE TO SEQUENCE LISTING SUBMITTED ELECTRONICALLY

The content of the electronically submitted sequence listing in ASCII text file (Name: 4525_030PC01_Seqlisting_ST25.txt; Size: 370,287 bytes; and Date of Creation: Jan. 26, 2022) filed with the application is incorporated herein by reference in its entirety.

### FIELD OF DISCLOSURE

The present disclosure pertains to the medical field, including gene therapy constructs encoding anti-TNFα antibodies or antigen-binding fragments thereof, compositions comprising vectors (e.g., viral vectors) suitable for delivery of nucleic acids encoding therapeutic anti-TNFα antibodies or antigen-binding fragments thereof, and methods of using the same. Certain aspects of the disclosure are directed to adeno-associated virus vector (AAV) delivery of nucleic acids encoding anti-TNFα antibodies (e.g., monoclonal antibodies) or antigen-binding fragments thereof to a subject in need thereof.

### BACKGROUND

Noninfectious uveitis is a group of disorders characterized by intraocular inflammation at different levels of the eye, and is a leading cause of irreversible blindness in the working-age population of the developed world. Current standard of care for noninfectious uveitis includes the administration of corticosteroids as first-line agents, followed by oral and systemic immunosuppressants. Corticosteroid treatment is suboptimal because of the risk of increased intraocular pressure leading to glaucoma and cataracts, as well as systemic side effects. Further, corticosteroids often fail to achieve long-term resolution of inflammation. Approved oral and systemic immunosuppressants similarly have significant systemic side effects, and may require frequent blood monitoring, and are contraindicated for patients with liver dysfunction or pregnancy.

Immunosuppressive drugs are drugs that inhibit or prevent activity of the immune system. TNF inhibitors can suppress the physiologic response to tumor necrosis factor (TNF), which is part of the inflammatory response. Inhibition of TNF (e.g., TNFα) can be achieved with monoclonal antibodies such adalimumab (sold under the brand name Humira among others). Adalimumab was the first fully human monoclonal antibody approved by the U.S. Food and Drug Administration (FDA). In the U.S., adalimumab is indicated for the treatment of diseases such as rheumatoid arthritis, juvenile idiopathic arthritis, psoriatic arthritis, ankylosing spondylitis, adult crohn's disease, pediatric crohn's disease, ulcerative colitis, plaque psoriasis, hidradenitis suppurativa, and uveitis. Adalimumab is administered by subcutaneous injection. For most indications, the maintenance treatment requires regular injections, e.g., every other week.

Adalimumab has been approved for the treatment of non-infectious intermediate, posterior, and panuveitis. Adult patients receive an initial dose of 80 mg via subcutaneous injection (SQ), followed by 40 mg SQ given every other week starting one week after the initial injection. Adalimumab treatment must continue for as long as the disease persists. Chronic systemic treatment with adalimumab can lead to serious side effects including serious infections.

Gene therapy focuses on the utilization of the therapeutic delivery of nucleic acids into a patient's cells as a drug to treat disease. Advances in the field of gene therapy have been achieved using viruses to deliver therapeutic genetic material. Although a variety of physical and chemical methods have been developed for introducing exogenous DNA into eukaryotic cells, viruses have generally been shown to be more efficient for this purpose. Several DNA-containing viruses such as parvoviruses, adenoviruses, herpesviruses and poxviruses, and RNA-containing viruses, such as retroviruses, have been used to develop eukaryotic cloning and expression vectors. Adeno-associated virus (AAV) vectors are considered to be safe for the delivery of genes in humans in vivo. Some challenges with the viral vectors include low efficiency, DNA packaging capacity, and a lack of target cell specificity.

### BRIEF SUMMARY

Certain aspects of the disclosure are directed to gene therapy compositions comprising expression constructs for sustained intraocular expression of adalimumab. Sustained intraocular expression of adalimumab following a single intravitreal injection has the potential to improve patient compliance leading to improved treatment outcomes, and reduce systemic side effects.

Certain aspects of the disclosure are directed to a recombinant adeno-associated virus (rAAV) particle comprising a capsid and a vector genome, the vector genome comprising an inverted terminal repeat (ITR) and an antibody expression cassette, wherein the antibody expression cassette comprises (a) a promoter, (b) a first leader sequence operably linked to a nucleic acid sequence encoding a heavy chain variable region (VH) of an anti-tumor necrosis factor alpha (anti-TNFalpha) antibody or an antigen-binding fragment thereof, (c) a linker sequence comprising a proteolytic cleavage site, and (d) a second leader sequence operably linked to a nucleic acid sequence encoding a light chain variable region (VL) of an anti-TNFalpha antibody or an antigen-binding fragment thereof, optionally, wherein the AAV capsid serotype is AAV2 or a modified version thereof.

In some aspects, the nucleic acid sequence encoding the VH of the anti-TNFalpha antibody or an antigen-binding fragment thereof comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 7, 8, 9, 102, 143, or 147; and the nucleic acid sequence encoding the VL of the anti-TNFalpha antibody or an antigen-binding fragment thereof comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 11, 12, 13, 103, 145, or 149.

Certain aspects of the disclosure are directed to a vector comprising an antibody expression cassette comprising: (a) a promoter; (b) a first leader sequence; (c) a nucleic acid sequence encoding a heavy chain variable region (VH) of an anti-tumor necrosis factor alpha (anti-TNFalpha) antibody or an antigen-binding fragment thereof comprising a nucleotide sequence at least 85%, 86%, 87%, 88%, 89%, 90%, 91%, 92%, 93%, 94%, 95%, 96%, 97%, 98%, 99%, or 100% identical to SEQ ID NO: 7, 8, 9, 102, 143, or 147; (d) a linker sequence; (e) a second leader sequence; (f) a nucleic acid sequence encoding a light chain variable region (VL) of an anti-TNFalpha antibody or an antigen-binding fragment thereof comprising a nucleotide sequence at least 85%, 86%, 87%, 88%, 89%, 90%, 91%, 92%, 93%, 94%, 95%, 96%, 97%, 98%, 99%, or 100% identical to SEQ ID NO: 11, 12, 13, 103, 145, or 149.

In some aspects, the nucleic acid sequence encoding the VH comprises a nucleotide sequence at least 90%, 91%, 92%, 93%, 94%, 95%, 96%, 97%, 98%, 99%, or 100% identical to SEQ ID NO: 7, 8, 9, 102, 143, or 147.

In some aspects, the nucleic acid sequence encoding the VL comprises a nucleotide sequence at least 90%, 91%, 92%, 93%, 94%, 95%, 96%, 97%, 98%, 99%, or 100% identical to SEQ ID NO: 11, 12, 13, 103, 145, or 149.

In some aspects, the nucleic acid sequence encoding the having chain (HC) comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 17, 18, 19, 110, 142, 146, 150, or 151.

In some aspects, the nucleic acid sequence encoding the light chain (LC) comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 21, 22, 23, 111, 144, or 148.

In some aspects, the linker sequence comprises a proteolytic cleavage site comprising a furin cleavage site, a 2A cleavage site, or a combination thereof. In some aspects, the proteolytic cleavage site comprises a furin cleavage site and a 2A cleavage site.

In some aspects, the furin cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 26.

In some aspects, the 2A cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 28.

In some aspects, the first leader sequence is an IL-10 leader sequence. In some aspects, the IL-10 leader sequence has a nucleotide sequence at least 85%, 86%, 87%, 88%, 89%, 90%, 91%, 92%, 93%, 94%, 95%, 96%, 97%, 98%, 99%, or 100% identical to SEQ ID NO: 30 or 112.

In some aspects, the second leader sequence is an IL-2 leader sequence. In some aspects, the IL-2 leader sequence has a nucleotide sequence at least 85%, 86%, 87%, 88%, 89%, 90%, 91%, 92%, 93%, 94%, 95%, 96%, 97%, 98%, 99%, or 100% identical to any of SEQ ID NO: 29 or 113.

In some aspects, the promoter is a CAG promoter, a CBA promoter, a CMV promoter, an EF1α promoter, a CMV promoter with a CMV enhancer, a CMV promoter with a SV40 intron, an EF1α with a CMV enhancer, or tissue specific promoter. In some aspects, the promoter is a CAG promoter. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 38.

In some aspects, the anti-TNFalpha antibody is a monoclonal antibody. In some aspects, the anti-TNFalpha antibody is adalimumab.

In some aspects, the antibody expression cassette comprises a poly(A) sequence.

In some aspects, the poly(A) sequence is selected from a bGHpA, a hGHpA, a SV40pA, hGHpA, or a synthetic pA. In some aspects, the poly(A) sequence comprises a bGHpA. In some aspects, the poly(A) comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 39, 40, or 114.

In some aspects, the antibody expression cassette comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 42-51 or any combination thereof.

In some aspects, the antibody expression cassette comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 52-61 or any combination thereof.

In some aspects, the antibody expression cassette comprises an open reading frame (ORF) comprising the nucleotide sequence at least 85%, 86%, 87%, 88%, 89%, 90%, 91%, 92%, 93%, 94%, 95%, 96%, 97%, 98%, 99%, or 100% identical to SEQ ID NO: 49 and the nucleotide sequence at least 85%, 86%, 87%, 88%, 89%, 90%, 91%, 92%, 93%, 94%, 95%, 96%, 97%, 98%, 99%, or 100% identical to SEQ ID NO: 56.

In some aspects, the antibody expression cassette comprises a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 62-77, 115-141, or 153-158.

In some aspects, the antibody expression cassette comprises a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 64 or 115.

In some aspects, a vector disclosed herein comprises an inverted terminal repeat (ITR). In some aspects, the AAV ITR comprises a pair of ITRs flanking the antibody expression cassette. In some aspects, the ITRs are AAV2 serotype. In some aspects, the vector is packaged in an AAV capsid. In some aspects, the AAV capsid serotype is selected from the group consisting of AAV1, AAV2, AAV3, AAV4, AAV5, AAV6, AAV7, AAV8, AAVrh8, AAVrh9, AAV9, AAVrh10, AAV10, AAV11, AAV12, or a modified version thereof. In some aspects, the AAV capsid serotype is AAV2 or a modified version thereof. In some aspects, the modified AAV2 capsid is AAV2 Quad Y-F or AAV2 Quad Y-F+T491V. In some aspects, the AAV capsid is AAV2.7m8. In some aspects, the AAV capsid is AAVshH10.

In some aspects, the disclosure is directed to a host cell or composition comprising a rAAV particle or a vector disclosed herein. In some aspects, the composition comprises a carrier (e.g., water or saline).

Certain aspects of the disclosure are directed a method of expressing an anti-TNFalpha antibody or antigen-binding fragment thereof in a cell, comprising administering to the cell a rAAV particle, a vector, or a composition disclosed herein, thereby expressing the anti-TNFalpha antibody or antigen-binding fragment thereof in the cell. In some aspects, the administration is in vitro. In some aspects, the administration is in vivo.

Certain aspects of the disclosure are directed a method of expressing an anti-TNFalpha antibody or antigen-binding fragment thereof in a subject in need thereof, comprising administering to the subject a rAAV particle, a vector, or a composition disclosed herein, thereby expressing the anti-TNFalpha antibody or antigen-binding fragment thereof in the subject.

Certain aspects of the disclosure are directed a method of neutralizing TNFalpha in a subject comprising administering to the subject a rAAV particle, a vector, or a composition disclosed herein, wherein the anti-TNFalpha antibody or antigen-binding fragment thereof expressed in the subject is capable of neutralizing TNFalpha. In some aspects, the TNFalpha neutralization is increased compared to TNFalpha neutralization in a subject administered recombinant adalimumab.

In some aspects, the subject suffers from an immune disease or disorder, an autoimmune disease or disorder, or an ocular disease or disorder. In some aspects, the subject suffers from an ocular disease or disorder.

Certain aspects of the disclosure are directed a method of treating an immune disease or disorder, an autoimmune disease or disorder, or an ocular disease or disorder in a subject in need thereof comprising administering to the subject an effective amount of a rAAV particle, a vector, or a composition disclosed herein, thereby expressing the anti-TNFalpha antibody or antigen-binding fragment thereof in the subject and treating the immune disease or disorder, an autoimmune disease or disorder, or an ocular disease or disorder.

Certain aspects of the disclosure are directed a method of treating an ocular disease or disorder in a subject in need thereof comprising intravitreally administering to the subject an effective amount of a recombinant adeno-associated virus (rAAV) particle comprising a capsid and a vector genome, the vector genome comprising an inverted terminal repeat (ITR) and an antibody expression cassette, wherein the antibody expression cassette comprises (a) a promoter, (b) a nucleic acid sequence encoding a heavy chain variable region (VH) of an anti-tumor necrosis factor alpha (anti-TNFalpha) antibody or an antigen-binding fragment thereof, (c) a linker sequence, and (d) a nucleic acid sequence encoding a light chain variable region (VL) of an anti-TNFalpha antibody or an antigen-binding fragment thereof, optionally, wherein the AAV capsid serotype is AAV2 or a modified version thereof, thereby expressing the anti-TNFalpha antibody or antigen-binding fragment thereof in the subject and treating the ocular disease or disorder.

In some aspects, the ocular disease or disorder is uveitis. In some aspects, the uveitis is non-infectious uveitis. In some aspects, the non-infectious uveitis is selected from the group consisting of intermediate uveitis, posterior uveitis, and panuveitis.

In some aspects, the ocular disease or disorder is a corneal disease. In some aspects, the corneal disease is selected from the group consisting of peripheral ulcerative keratitis, corneal hemangiogenesis, and noninfectious corneal melting.

In some aspects, the administration is suitable for delivery of the rAAV particle or the vector to one or both eyes. In some aspects, the administration is by injection. In some aspects, the administration is intravitreal. In some aspects, the administration is intrastromal or transconjunctival.

In some aspects, the administration is a single dose. In some aspect, the single dose is administered in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye. In some aspect, the single dose is administered in a volume of about 40 μL to 60 μL per eye. In some aspect, the single dose is administered in a volume of about 50 μL per eye.

In some aspects, the administration comprises a single dose within the range of 1E9 vector genomes (vg) to 3E12 vg. In some aspects, the administration comprises a single dose within the range of 1E9 vg to 1E12 vg. In some aspects, the administration comprises a single dose within the range of 1E9 vg to 1E11 vg. In some aspects, the administration comprises a single dose within the range of 1E9 vg to 3E10 vg. In some aspects, the administration comprises a single dose of about 1E9 vg. In some aspects, the administration comprises a single dose of about 1E10 vg. In some aspects, the administration comprises a single dose of about 1E11 vg.

In some aspects, the anti-TNFalpha antibody steady state concentration in the eye (e.g., in ocular fluid, for example, in the aqueous or viterious humor) of the subject after administration is 10 ng/mL to 1000 ng/mL (1 μg/mL). In some aspects, the anti-TNFalpha antibody steady state concentration in the eye (e.g., in ocular fluid, for example, in the aqueous or viterious humor) of the subject after administration is 10 ng/mL to 500 ng/mL. In some aspects, the anti-TNFalpha antibody steady state concentration in the eye (e.g., in ocular fluid, for example, in the aqueous or viterious humor) of the subject after administration is 10 ng/mL to 100 ng/mL. In some aspects, the anti-TNFalpha antibody steady state concentration in the eye (e.g., in ocular fluid, for example, in the aqueous or viterious humor) of the subject after administration is at least 10 ng/mL, at least 20 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 250 ng/mL, at least 300 ng/mL, at least 400 ng/mL, at least 500 ng/mL, at least 600 ng/mL, at least 700 ng/mL, at least 800 ng/mL, at least 900 ng/mL, or at least 1 μg/mL.

In some aspects, the anti-TNFalpha antibody steady state concentration in the serum of the subject after administration is less than 1% of the total anti-TNFalpha antibody concentration after administration (to one or both eyes). In some aspects, the anti-TNFalpha antibody steady state concentration in the serum of the subject after administration is less than 20 ng/mL, less than 15 ng/mL, less than 10 ng/mL, less than 5 ng/mL, less than 1 ng/mL, or less than 0.5 ng/mL. In some aspects, the anti-TNFalpha antibody steady state concentration in the serum of the subject after administration is 0.1 ng/mL to 20 ng/mL (e.g., 0.5 ng/mL to 20 ng/mL, 0.5 ng/mL to 10 ng/mL, or 0.5 ng/mL to 5 ng/mL).

Certain aspects of the disclosure are directed to a gene therapy construct (e.g., a vector) comprising a polynucleotide comprising a promoter sequence, a nucleic acid sequence encoding a heavy chain, and a nucleic acid sequence encoding a light chain.

In some aspects, the gene therapy construct (e.g., the vector) comprises a polynucleotide (e.g., an antibody expression cassette) comprising: (a) a promoter; (b) a nucleic acid sequence encoding a heavy chain variable region (VH); (c) a linker sequence (e.g., comprising a proteolytic cleavage site); (d) a nucleic acid sequence encoding a light chain variable region (VL).

In some aspects, the gene therapy construct (e.g., the vector) comprises a polynucleotide comprising a promoter sequence, a nucleic acid sequence encoding a heavy chain, an IRES, and a nucleic acid sequence encoding a light chain in 5′-3′ orientation.

In some aspects, the polynucleotide comprises a promoter sequence, a nucleic acid sequence encoding a light chain, IRES, and a nucleic acid sequence encoding a heavy chain in 5′-3′ orientation.

In some aspects, the polynucleotide comprises a promoter sequence, a nucleic acid sequence encoding a heavy chain, a furin cleavage site, a 2A cleavage site, and a nucleic acid sequence encoding a light chain in 5′-3′ orientation.

In some aspects, the polynucleotide comprises a promoter sequence, a nucleic acid sequence encoding a light chain, a furin cleavage site, a 2A cleavage site, and a nucleic acid sequence encoding a heavy chain in 5′-3′ orientation.

In some aspects, the polynucleotide further comprises a second promoter sequence.

In some aspects, the polynucleotide comprises a first promoter sequence, a nucleic acid sequence encoding a light chain, a second promoter sequence, and a nucleic acid sequence encoding a heavy chain in 5′-3′ orientation.

In some aspects, the polynucleotide comprises a first promoter sequence, a nucleic acid sequence encoding a heavy chain, a second promoter sequence, and a nucleic acid sequence encoding a light chain in 5′-3′ orientation.

In some aspects, the polynucleotide comprises a nucleic acid sequence encoding a heavy chain, a first promoter sequence, a second promoter sequence, and a nucleic acid sequence encoding a light chain in 5′-3′ orientation.

In some aspects, the polynucleotide comprises a nucleic acid sequence encoding a light chain, a first promoter sequence, a second promoter sequence, and a nucleic acid sequence encoding a heavy chain in 5′-3′ orientation.

In some aspects, the promoter is a constitutively active promoter, a cell-type specific promoter, a synthetic promoter, or an inducible promoter. In some aspects, the promoter is selected from the group consisting of a CAG, CBA, CMV, EF1α, EF1α with a CMV enhancer, CMV, a CMV promoter with a CMV enhancer (CMVe/p), a CMV promoter with a SV40 intron or tissue specific promoter. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38. In some aspects, the nucleic acid sequence comprising the promoter can comprises an intron. In some aspects, the intron is selected from the group consisting of an SV40 intron, MVM intron, or a human betaglobin intron. In some aspects, SV40 intron comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NO: 33.

In some aspects, the first and second promoter are different. In some aspects, the first and second promoter are the same. In some aspects, the first and second promoter initiate transcription in the same direction. In some aspects, the first and second promoter initiate transcription in different directions.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 41.

In some aspects, the heavy chain is a heavy chain of an anti-TNFalpha antibody. In some aspects, the light chain is a light chain of an anti-TNFalpha antibody. In some aspects, the anti-TNFalpha antibody is a monoclonal antibody. In some aspects, the anti-TNFalpha antibody is adalimumab.

In some aspects, the heavy chain comprises a heavy chain variable region (VH) comprising a complementarity determining region (CDR) 1, a VH CDR2, and a VH CDR3. In some aspects, the VH CDRs 1-3 correspond to the CDRs of adalimumab. In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, 87, or 90 the nucleic acid sequence comprising VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, 88, or 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86, 89, 92, 106, or 159.

In some aspects, the light chain comprises a light chain variable region (VL) comprising a complementarity determining region (CDR) 1, a VL CDR2, and a VL CDR3. In some aspects, the VL CDRs 1-3 correspond to the CDRs of adalimumab. In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, 96, 99, or 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, 97, 100, 108, 160, or 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95, 98, 101, or 109.

In some aspects, the nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 7-9. In some aspects, the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 11-13.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 17-19. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 21-23.

In some aspects, the IRES comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 25. In some aspects, the furin cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 26. In some aspects, the 2A cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 28.

In some aspects, the polynucleotide also comprises a leader sequence operably linked to the nucleic acid sequence encoding the heavy chain and/or the nucleic acid sequence encoding the light chain. In some aspects, the leader sequence is an IL-2 or IL-10 leader sequence. In some aspects, the IL-2 leader sequence has a nucleotide sequence at least 85%, 86%, 87%, 88%, 89%, 90%, 91%, 92%, 93%, 94%, 95%, 96%, 97%, 98%, 99%, or 100% identical to SEQ ID NO: 29. In some aspects, the IL-10 leader sequence has a nucleotide sequence at least 85%, 86%, 87%, 88%, 89%, 90%, 91%, 92%, 93%, 94%, 95%, 96%, 97%, 98%, 99%, or 100% identical to SEQ ID NO: 30.

In some aspects, the polynucleotide also comprises a nucleic acid sequence comprising a miRNA binding site. In some aspects, the miRNA binding site is a miR-142 binding site. In some aspects, the miRNA binding site comprises four miR-142 binding sites. In some aspects, the four miR-142 binding sites are separated by spacers (4×miR-142 binding site). In some aspects, the miR-142 binding site has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 31. In some aspects, the 4× miR-142 binding site has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 32.

In some aspects, the nucleic acid sequence encoding the variable heavy chain and the nucleic acid sequence encoding the variable light chain are operably linked. In some aspects, the nucleic acid sequence encoding the variable heavy chain and the nucleic acid sequence encoding the variable light chain are operably linked by a linker sequence. In some aspects, the linker sequence is selected from an IRES sequence, a proteolytic cleavage site (e.g., a furin and/or 2A cleavage site), or a combination thereof.

In some aspects, the polynucleotide comprises a poly(A). In some aspects, the poly(A) sequence has a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39 or 40.

In some aspects, the polynucleotide comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 42-61. In some aspects, the polynucleotide comprises an expression cassette comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 62-77, 115-141, or 153-158.

Certain aspects of the disclosure are directed to an expression cassette comprising a polynucleotide of the disclosure (e.g., a polynucleotide encoding an antibody HC and LC). Certain aspects of the disclosure are directed to an expression cassette comprising a polynucleotide of the disclosure and a heterologous expression control sequence operably linked to the polynucleotide.

Certain aspects of the disclosure are directed to a vector (e.g., viral vector, a non-viral vector, a plasmid, a lipid, or a lysosome) comprising a polynucleotide or an expression cassette (e.g., an antibody expression cassette) of the disclosure. In some aspects, the vector is selected from the group consisting of an adeno-associated viral (AAV) vector, an adenoviral vector, a lentiviral vector, or a retroviral vector. In some aspects, the AAV serotype is selected from the group consisting of AAV1, AAV2, AAV3, AAV4, AAV5, AAV6, AAV7, AAV8, AAVrh8, AAVrh9, AAV9, AAVrh10, AAV10, AAV11, and AAV12.

Certain aspects of the disclosure are directed to a recombinant AAV (rAAV) particle, comprising an AAV capsid and a vector genome comprising the polynucleotide or the expression cassette of the disclosure. In some aspects, the vector genome comprises an ITR (e.g., an AAV 5′ ITR and an AAV 3′ ITR). In some aspects, the AAV serotype is selected from the group consisting of AAV1, AAV2, AAV3, AAV4, AAV5, AAV6, AAV7, AAV8, AAVRH8, AAVrh9, AAV9, AAVrh10, AAV10, AAV11, and AAV12.

In some aspects, the rAAV particle comprises a wild-type or a modified AAV capsid. In some aspects, the AAV serotype is selected from the group consisting of AAV1, AAV2, AAV3, AAV4, AAV5, AAV6, AAV7, AAV8, AAVrh8, AAV9, AAV10, AAVrh10, AAV11, and AAV12. In some aspects, the AAV capsid is AAVshH10. In some aspects, the modified AAV capsid is AAV2 Quad Y-F or AAV2 Quad Y-F+T491V. In some aspects, the AAV capsid is AAV2.7m8.

Certain aspects of the disclosure are directed to a host cell (e.g., a mammalian cell) comprising a polynucleotide, an expression cassette, a vector, or a rAAV particle of the disclosure.

Certain aspects of the disclosure are directed to a method of producing an anti-TNFalpha antibody or antigen-binding fragment thereof in a subject, comprising administering to the subject a polynucleotide, an expression cassette, a vector, or a rAAV particle of the disclosure, thereby producing the anti-TNFalpha antibody or antigen-binding fragment thereof in the subject.

Certain aspects of the disclosure are directed to gene therapy compositions comprising: the polynucleotide of the disclosure and (b) a viral delivery vector. In some aspects, the viral delivery vector is selected from the group consisting of an adeno-associated viral (AAV) vector, an adenoviral vector, a lentiviral vector, or a retroviral vector.

In some aspects, the delivery vector is an adeno-associated viral (AAV) vector. In some aspects, the AAV vector is a recombinant AAV (rAAV) vector comprising an AAV serotype selected from the group consisting of AAV1, AAV2, AAV3, AAV4, AAV5, AAV6, AAV7, AAV8, AAVrh8, AAV9, AAV10, AAVrh10, AAV11, and AAV12. In some aspects, the rAAV comprises a modified AAV capsid. In some aspects, the modified rAAV capsid is AAV2 Quad Y-F or AAV2 Quad Y-F+T491V. In some aspects, the AAV capsid is AAV2.7m8. In some aspects, the AAV capsid is AAVshH10.

In some aspects, the gene therapy composition is suitable for delivery of the polynucleotide to a secretory organ, a secretory-like organ, or any other delivery site disclosed herein. In some aspects, the gene therapy composition is suitable for a single dose administration.

In some aspects, provided herein is an adeno-associated virus (AAV) capsid comprising the polynucleotide disclosed herein, wherein the AAV capsid is suitable for delivering the nucleic acid to a subject in need thereof. In some aspects, the therapeutic protein is an antibody or antigen binding fragment thereof. In some aspects, the antibody or antigen binding fragment thereof is selected from the group consisting of a monoclonal antibody, a bispecific antibody, and a multispecific antibody. In some aspects, the antibody or antigen binding fragment thereof is a monoclonal antibody. In some aspects, the antibody is modified to not bind an Fc receptor.

In some aspects, provided herein is a method of expressing a therapeutic antibody or antigen-binding fragment thereof that binds TNFalpha in a subject in need thereof comprising administering an effective amount of a gene therapy composition disclosed herein or an AAV capsid disclosed herein to the subject. In some aspect, the delivery is administered as a single dose.

In some aspects, provided herein is a method for delivery of a gene therapy to a subject in need thereof, comprising administering to the subject the delivery vector disclosed herein. In some aspect, the delivery is administered as a single dose.

Also provided herein is a method of delivering a nucleic acid to a cell of a subject, comprising administering to a secretory cell of the subject an adeno-associated virus (AAV) capsid as disclosed herein (e.g., comprising a polynucleotide encoding an anti-TNFalpha antibody or an antigen-binding fragment thereof), thereby delivering the nucleic acid to the secretory cell of the subject.

In some aspects, the secretory cell is selected from the group consisting of a lymph node cell, a gall bladder cell, a thymus cell, a hypothalamus cell, a stomach cell, an intestine cell, a liver cell, a pancreas cell, a kidney cell, a skin cell, and a secretory gland cell.

In some aspects, the secretory cell can be selected from a heart muscle cell, a bone cell, a muscle cell (e.g., skeletal muscle), a skin cell, and an adipose cell.

In some aspects, the secretory gland cell is selected from the group consisting of a salivary gland cell, a pineal gland cell, a thyroid gland cell, an adrenal gland cell, and a parathyroid gland cell.

Also provided herein is a method of delivering a therapeutic protein to a subject in need thereof, comprising administering to the subject a delivery vector as disclosed herein. In some aspects, the therapeutic protein is an anti-TNFalpha antibody. In some aspects, the delivery vector is administered a single dose.

Also provided herein is a method of expressing an anti-TNFalpha antibody or an antigen-binding fragment thereof in a subject in need thereof comprising administering an effective amount of the gene therapy composition or the AAV capsid disclosed herein to the subject, wherein the administration is intraductally, intracutaneous, intraocular, intravitreal, intrastromal, transconjunctival, or by direct injection to the secretory organ (e.g., secretory gland). In some aspects, the gene therapy composition or AAV capsid is administered intraductally, by direct injection to the salivary gland. In some aspects, the gene therapy composition or AAV capsid is administered to a secretory cell. In some aspects, the secretory cell is selected from the group consisting of a lymph node cell, a gall bladder cell, a thymus cell, a hypothalamus cell, a stomach cell, an intestine cell, a liver cell, a pancreas cell, a kidney cell, a skin cell, and a secretory gland cell. In some aspects, the secretory cell can be selected from a heart muscle cell, a bone cell, a skeletal muscle cell, a skin cell, and an adipose cell. In some aspects, the secretory gland cell is selected from the group consisting of a salivary gland cell, a pineal gland cell, a thyroid gland cell, an adrenal gland cell, and a parathyroid gland cell.

Also provided herein is a method of delivering a nucleic acid to a cell of a subject, comprising administering to a secretory cell of the subject the gene therapy composition disclosed herein or the AAV capsid disclosed herein, thereby delivering the nucleic acid to the secretory cell of the subject. In some aspects, the secretory cell is selected from the group consisting of a lymph node cell, a gall bladder cell, a thymus cell, a hypothalamus cell, a stomach cell, an intestine cell, a liver cell, a pancreas cell, a kidney cell, a skin cell, and a secretory gland cell. In some aspects, the secretory cell can be selected from a heart muscle cell, a bone cell, a skeletal muscle cell, a skin cell, and an adipose cell. In some aspects, the secretory gland cell is selected from the group consisting of a salivary gland cell, a pineal gland cell, a thyroid gland cell, an adrenal gland cell, and a parathyroid gland cell.

In some aspects, the subject suffers from a disease or disorder selected from the group consisting of an immune disease or disorder, an autoimmune disease or disorder, or an ocular disease or disorder. In some aspects, the disease or disorder is an ocular disease or disorder (e.g., uveitis (e.g., non-infectious uveitis) or a corneal disease (e.g., peripheral ulcerative keratitis, corneal hemangiogenesis, and noninfectious corneal melting)), an inflammatory disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease), and any combination thereof. In some aspects, the disease or disorder is selected from rheumatoid arthritis, juvenile idiopathic arthritis, psoriatic arthritis, ankylosing spondylitis, adult crohn's disease, pediatric crohn's disease, ulcerative colitis, plaque psoriasis, hidradenitis suppurativa, uveitis (e.g., non-infectious uveitis such as intermediate uveitis, posterior uveitis, and panuveitis), peripheral ulcerative keratitis, corneal hemangiogenesis, and noninfectious corneal melting. In some aspects, the disease or disorder is uveitis. In aspects, the disease or disorder is non-infectious uveitis. In some aspects, the non-infectious uveitis is selected from the group consisting of intermediate uveitis, posterior uveitis, and panuveitis. In some aspects, the disease or disorder is a corneal disease. In some aspects, the corneal disease is selected from the group consisting of peripheral ulcerative keratitis, corneal hemangiogenesis, and noninfectious corneal melting.

In some aspects, the subject does not suffer from a disease of a secretory organ. In some aspects, the subject does not suffer from a disease of a secretory gland. In some aspects, the subject does not suffer from a disease of the salivary gland.

In some aspects, the gene therapy composition or AAV capsid disclosed herein is administered intraductally, intracutaneous, or by direct injection to the secretory organ (e.g., secretory gland). In some aspects, the gene therapy composition, vector or AAV capsid disclosed herein is administered to one or both eyes, e.g., intraocularly, intravitreally, intrastromaly, or transconjunctivaly.

### BRIEF DESCRIPTION OF THE FIGURES

FIGS. 1A-1H show exemplary expression cassettes showing alternative designs for polynucleotide sequences including a promoter, a nucleic acid sequence encoding a heavy chain, a linker, and a nucleic acid sequence encoding a light chain (FIGS. 1A-1D) or polynucleotide sequences including a first promoter, a second promoter, a nucleic acid sequence encoding a heavy chain, and a nucleic acid sequence encoding a light chain (FIGS. 1E-1H) for expression of an anti-TNFalpha antibody.

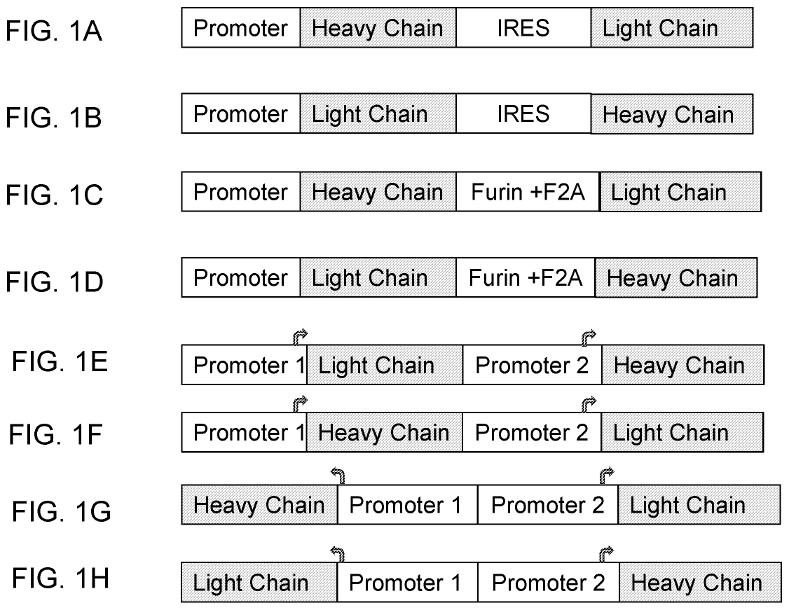

FIG. 2A shows an exemplary plasmid construct designed to include a backbone, flanking ITRs, a promoter, polyA, and open reading frame (ORF).

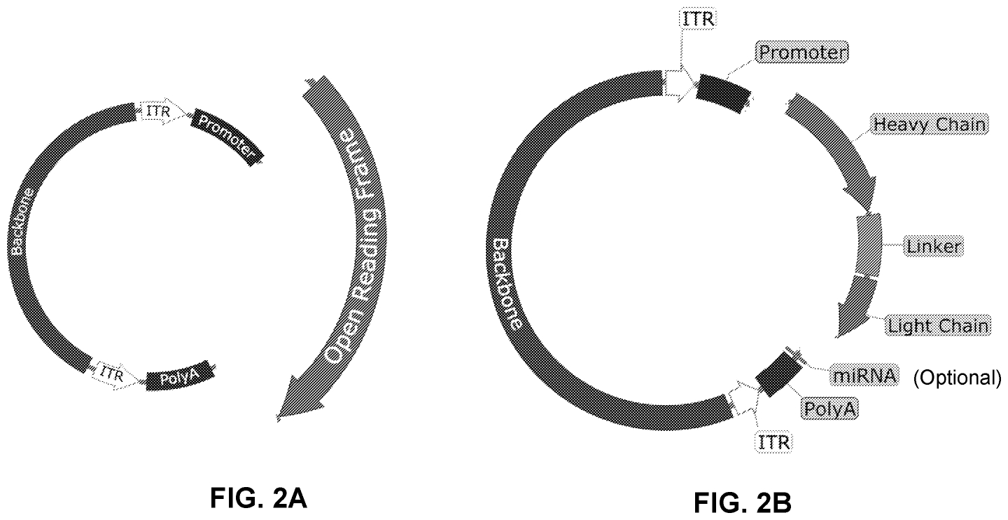

FIG. 2B shows an exemplary plasmid construct designed with an expression cassette comprising promoter, an ORF (comprising a sequence encoding a heavy chain, a linker, a sequence encoding a light chain, which can use any of the alterative designs shown in FIGS. 1A-1D), an optional miRNA, and a hGH poly(A) signal sequence, which is flanked by ITR sequences connected by a plasmid backbone.

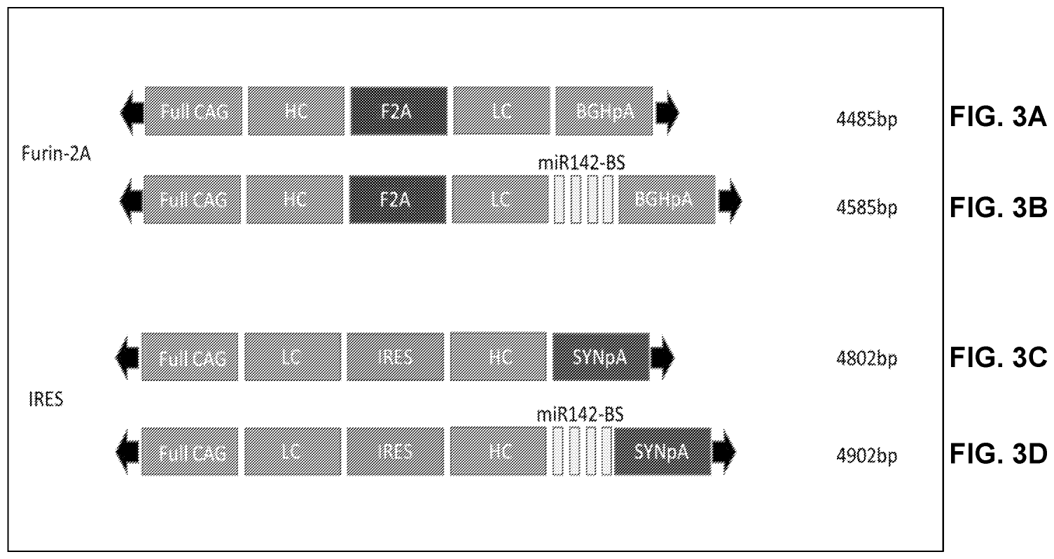

FIGS. 3A-3D show exemplary expression cassettes showing alternative designs for polynucleotide sequences including a promoter, a nucleic acid sequence encoding a heavy or light chain, a linker, a nucleic acid sequence encoding a heavy or light chain, and a poly (A) for expression of an anti-TNFalpha antibody. SYNpA: synthetic poly(A); BGHpA: bovine growth hormone poly(A); HC: heavy chain; LC: light chain.

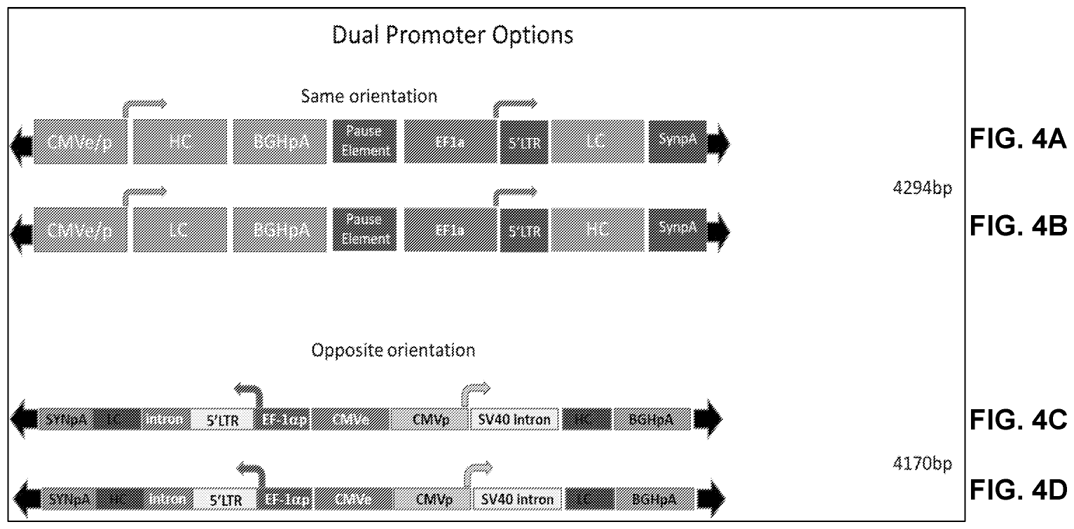

FIGS. 4A-4D show exemplary expression cassettes showing alternative designs for polynucleotide sequences including a first promoter, a second promoter, a nucleic acid sequence encoding a heavy chain, a nucleic acid sequence encoding a light chain and poly (A) sequences for expression of an anti-TNFalpha antibody. HC: heavy chain; LC: light chain; BGHpA: bovine growth hormone poly(A); SynpA: synthetic poly(A); CMVe: CMV enhancer; CMVp: CMV promoter.

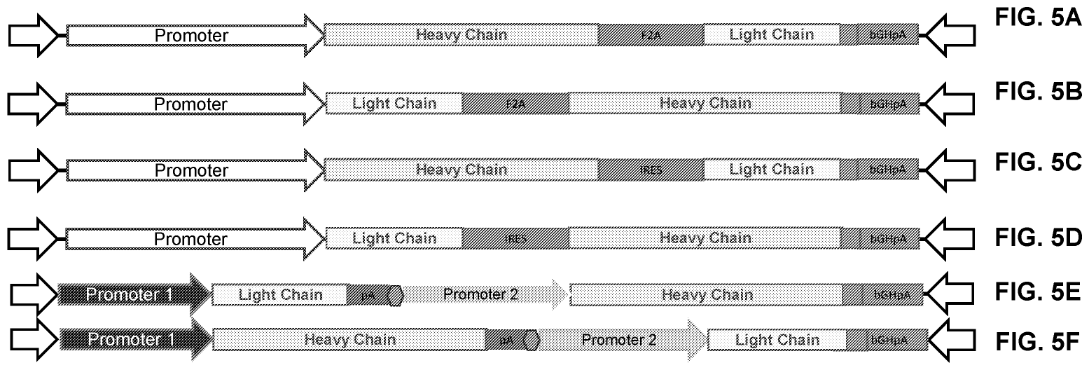

FIGS. 5A-5F show expression cassettes for expression of an anti-TNFalpha antibody. FIGS. 5A-5D show expression cassettes comprising a promoter, an ORF (comprising a sequence encoding a heavy chain, a linker, and a sequence encoding a light chain), and a poly(A). FIGS. 5E-5F show expression cassettes comprising a first promoter, an ORF (comprising a heavy chain or a light chain), a poly(A), a second promoter, an ORF (comprising a heavy chain or a light chain), and a poly(A).

FIG. 6 is a graph showing the amount of adalimumab expressed from HEK-293 transfected with plasmids comprising the expression cassettes of FIGS. 5A-5F. H-F2A-L: heavy chain-F2A-light chain (FIG. 5A); L-F2A-H: light chain-F2A-heavy chain (FIG. 5B); H-IRES-L: heavy chain-IRES-light chain (FIG. 5C); L-IRES-H: light chain-IRES-heavy chain (FIG. 5D); DP-HL: dual promoter heavy chain-light chain (FIG. 5E); DP-LH: dual promoter light chain-heavy chain (FIG. 5F).

FIG. 7 is a graph showing the amount of adalimumab expressed from mice injected with the plasmids comprising the expression cassettes of FIGS. 5A-5E. H-F2A-L: heavy chain-F2A-light chain (FIG. 5A); L-F2A-H: light chain-F2A-heavy chain (FIG. 5B); H-IRES-L: heavy chain-IRES-light chain (FIG. 5C); L-IRES-H: light chain-IRES-heavy chain (FIG. 5D); DP-LH: dual promoter light chain-heavy chain (FIG. 5F).

FIG. 8 is a plasmid map including antibody expression cassette #18.

FIG. 9 is a plasmid map including antibody expression cassette #19.

FIG. 10 is a plasmid map including antibody expression cassette #20.

FIG. 11 is a plasmid map including antibody expression cassette #21.

FIG. 12 is a plasmid map including antibody expression cassette #22.

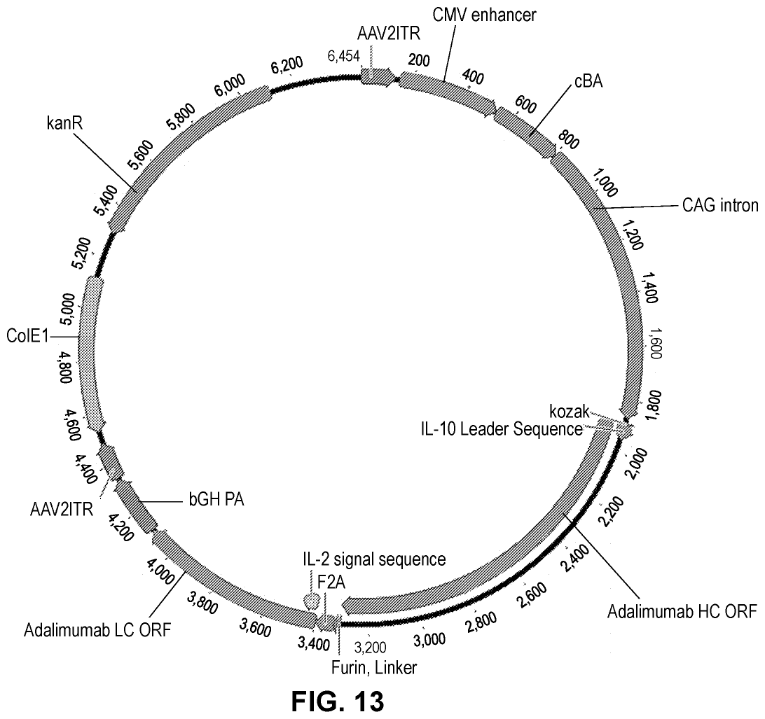

FIG. 13 is a plasmid map including antibody expression cassette #23.

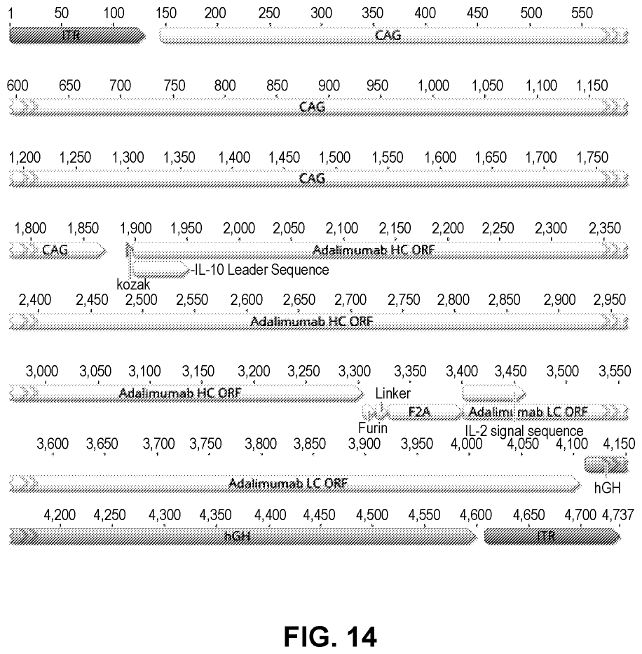

FIG. 14 is a sequence map including antibody expression cassette #24.

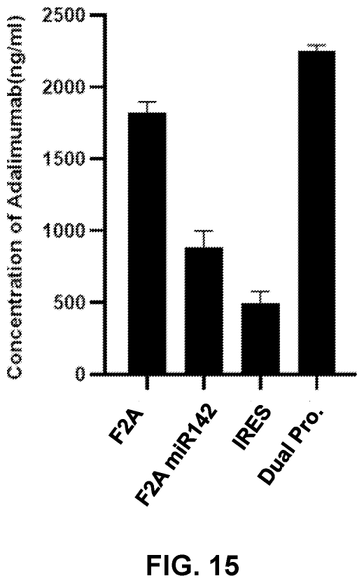

FIG. 15 is a graph showing the amount of adalimumab expressed from HEK-293 cells transduced with AAV particles comprising antibody expression cassettes with different linker sequences (F2A and IRES) or dual promoters (Dual Pro.), and with the microRNA binding sequence miR142 (F2A miR142).

FIG. 16 is a graph showing the TNFa neutralizing capacity in HEK293 cells transfected with plasmids including antibody expression cassettes #17-21 compared to recombinant adalimumab.

FIGS. 17A-17B show expression of adalimumab in SCID mice administered AAV2 particles including antibody expression cassettes #18-20. Expression of adalimumab in the serum (FIG. 17A) and ocular homogenate (FIG. 17B) were analyzed.

FIG. 18 is a graph showing the concentration of adalimumab after intravitreal injection of Humira in mice.

FIG. 19 is a graph showing the concentration of adalimumab after subcutaneous injection of Humira in mice.

FIG. 20 is a graph showing the predicted efficacious human ocular adalimumab concentrations for intravitreal administration of AAV2 particles comprising adalimumab antibody expression cassettes compared to estimated ocular concentrations of Humira in humans after subcutaneous administration.

### DETAILED DESCRIPTION OF THE DISCLOSURE

Certain aspects of the disclosure are directed to a gene therapy construct comprising a polynucleotide comprising a nucleic acid encoding an antibody or antigen-binding fragment thereof which binds tumor necrosis factor (TNF)-α (also referred to as TNFα, TNFalpha, and TNF-alpha herein). In some aspects, the polynucleotide comprises an open reading frame (ORF) comprising a nucleic acid sequence encoding a heavy chain and a nucleic acid sequence encoding a light chain. In some aspects, the ORF is operably linked to a promoter. In some aspects, the ORF is operably linked to a polyadenylation (polyA) element.

In some aspects, the ORF comprises a linker between the nucleic acid sequence encoding the heavy chain and the nucleic acid sequence encoding the light chain. In some aspects, the linker is an internal ribosomal entry sequence (IRES), a proteolytic cleavage site (e.g., a furin and/or 2A cleavage site), or a combination thereof. In some aspects, the OFR further comprises a nucleic acid encoding a signal sequence. In some aspects, the OFR is positioned between two inverted terminal repeats (ITRs).

In some aspects, the ORF comprises a leader sequence operably linked to the nucleic acid sequence encoding the heavy chain and/or the nucleic acid sequence encoding the light chain. In some aspects, the leader sequence is an IL-2 or an IL-10 leader sequence.

In some aspects, the ORF comprises a miRNA binding site. In some aspects, the miRNA binding site is a miR-142 binding site. In some aspects, the miRNA binding site comprises four miR-142 binding sites. In some aspects, the four miR-142 binding sites are separated by spacers.

In some aspects, the polynucleotide comprises a promoter sequence, a nucleic acid sequence encoding a heavy chain region, an IRES, and a nucleic acid sequence encoding a light chain region sequences in 5′-3′ orientation.

In some aspects, the polynucleotide comprises a promoter sequence, a nucleic acid sequence encoding a light chain region, IRES, and a nucleic acid sequence encoding a heavy chain region in 5′-3′ orientation.

In some aspects, the polynucleotide comprises a promoter sequence, a nucleic acid sequence encoding a heavy chain region, a furin cleavage site, a 2A cleavage site, and a nucleic acid sequence encoding a light chain region sequences in 5′-3′ orientation.

In some aspects, the polynucleotide comprises a promoter sequence, a nucleic acid sequence encoding a light chain region, a furin cleavage site, a 2A cleavage site, and a nucleic acid sequence encoding a heavy chain region sequences in 5′-3′ orientation.

In some aspects, the polynucleotide further comprises a second promoter.

In some aspects, the polynucleotide comprises a first promoter sequence, a nucleic acid sequence encoding a light chain, a second promoter sequence, and a nucleic acid sequence encoding a heavy chain in 5′-3′ orientation.

In some aspects, the polynucleotide comprises a first promoter sequence, a nucleic acid sequence encoding a heavy chain, a second promoter sequence, and a nucleic acid sequence encoding a light chain in 5′-3′ orientation.

In some aspects, the polynucleotide comprises a nucleic acid sequence encoding a heavy chain, a first promoter sequence, a second promoter sequence, and a nucleic acid sequence encoding a light chain in 5′-3′ orientation.

In some aspects, the polynucleotide comprises a nucleic acid sequence encoding a light chain, a first promoter sequence, a second promoter sequence, and a nucleic acid sequence encoding a heavy chain in 5′-3′ orientation.

In some aspects, the promoter is a constitutively active promoter, a cell-type specific promoter, a synthetic promoter, or an inducible promoter. In some aspects, the promoter is selected from the group consisting of a CAG, CBA, CMV, EF1α, EF1α with a CMV enhancer, a CMV promoter with a CMV enhancer (CMVe/p), a CMV promoter with a SV40 intron, or a tissue specific promoter. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38. In some aspects, the nucleic acid sequence comprising the promoter can comprises an intron. In some aspects, the intron is selected from the group consisting of an SV40 intron, MVM intron, or a human betaglobin intron. In some aspects, SV40 intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 33.

In some aspects, the first and second promoter are different. In some aspects, the first and second promoter are the same. In some aspects, the first and second promoter initiate transcription in the same direction. In some aspects, the first and second promoter initiate transcription in different directions.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 41.

In some aspects, the heavy chain is a heavy chain of an anti-TNFalpha antibody. In some aspects, the light chain is a light chain of an anti-TNFalpha antibody. In some aspects, the anti-TNFalpha antibody is a monoclonal antibody. In some aspects, the anti-TNFalpha antibody is adalimumab.

In some aspects, the heavy chain comprises a heavy chain variable region (VH) comprising a complementarity determining region (CDR) 1, a VH CDR2, and a VH CDR3. In some aspects, the VH CDRs 1-3 correspond to the CDRs of adalimumab. In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, 87, or 90, the nucleic acid sequence comprising VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, 88, or 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86, 89, 92, 106, or 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 87, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 88, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 89.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 92.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the light chain comprises a light chain variable region (VL) comprising a complementarity determining region (CDR) 1, a VL CDR2, and a VL CDR3. In some aspects, the VL CDRs 1-3 correspond to the CDRs of adalimumab. In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, 96, 99, or 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, 97, 100, 108, 160, or 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95, 98, 101, or 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 96, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 97, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 98.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 100, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 108, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 160, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

In some aspects, the nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 7-9, 102 (or nucleotides 55-417 of SEQ ID NO: 56), 143, or 147. In some aspects, the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 11-13, 103 (or nucleotides 61-381 of SEQ ID NO: 49), 145, or 149.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 7 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 11.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 8 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 12.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 9 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 13.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 102 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 103.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 143 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 145.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 147 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 149.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 17-19, 110 (or nucleotides 55-1407 of SEQ ID NO: 56 or nucleotides 1951-3304 of SEQ ID NO: 64), 142, 146, 150, or 151. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 21-23, 111 (or nucleotides 61-702 of SEQ ID NO: 49 or nucleotides 3460-4105 of SEQ ID NO: 64), 144, or 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 17. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 21.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 18. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 22.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 19. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 23.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 110. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 142. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 144.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 146. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 150. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 151. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the polynucleotide also comprises a leader sequence operably linked to the nucleic acid sequence encoding the heavy chain and/or the nucleic acid sequence encoding the light chain. In some aspects, the leader sequence is an IL-2 or IL-10 leader sequence.

In some aspects, the polynucleotide also comprises a nucleic acid sequence comprising a miRNA binding site. In some aspects, the miRNA binding site is a miR-142 binding site. In some aspects, the miRNA binding site comprises four miR-142 binding sites. In some aspects, the four miR-142 binding sites are separated by spacers.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 17-19, 110 (or nucleotides 55-1407 of SEQ ID NO: 56 or nucleotides 1951-3304 of SEQ ID NO: 64), 142, 146, 150, or 151. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 21-23, 111 (or nucleotides 61-702 of SEQ ID NO: 49 or nucleotides 3460-4105 of SEQ ID NO: 64), 144, or 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 17. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 21.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 18. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 22.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 19. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 23.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 110. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 142. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 144.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 146. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 150. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 151. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the nucleic acid sequence encoding the heavy chain and the nucleic acid sequence encoding the light chain are operably linked. In some aspects, the nucleic acid sequence encoding the heavy chain and the nucleic acid sequence encoding the light chain are operably linked by a linker sequence. In some aspects, the linker sequence is selected from an IRES sequence, a proteolytic cleavage site (e.g., a furin and/or 2A cleavage site), or a combination thereof. In some aspects, the IRES comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 25. In some aspects, the furin cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 26. In some aspects, the 2A cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 28.

In some aspects, the polynucleotide comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 42-61.

In some aspects, the polynucleotide comprises a poly(A). In some aspects, the polynucleotide comprises a poly(A) sequence comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39 or 40.

In some aspects, the polynucleotide comprises a IL-2 leader sequence comprising a nucleotide sequence at least 85%, 86%, 87%, 88%, 89%, 90%, 91%, 92%, 93%, 94%, 95%, 96%, 97%, 98%, 99%, or 100% identical to SEQ ID NO: 29 or 113 (or nucleotides 1-60 of SEQ ID NO: 49 or nucleotides 3401-3459 of SEQ ID NO: 64).

In some aspects, the polynucleotide comprises a IL-10 leader sequence comprising a nucleotide sequence at least 85%, 86%, 87%, 88%, 89%, 90%, 91%, 92%, 93%, 94%, 95%, 96%, 97%, 98%, 99%, or 100% identical to SEQ ID NO: 30 or 112 (or nucleotides 1-53 of SEQ ID NO: 56 or nucleotides 1898-1950 of SEQ ID NO: 64).

In some aspects, the polynucleotide comprises a miR-142 binding site comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 31 and 32.

In some aspects, the polynucleotide comprises an expression cassette comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 62-77, 115-141, or 153-158.

Certain aspects of the disclosure are directed to an expression cassette comprising a polynucleotide of the disclosure. Certain aspects of the disclosure are directed to an expression cassette comprising a polynucleotide of the disclosure and a heterologous expression control sequence operably linked to the polynucleotide.

Some aspects of the present disclosure are directed to a composition comprising: (a) a polynucleotide of the disclosure; and (b) a delivery vector (e.g., a viral vector). In some aspects, the composition is suitable for delivery to a secretory organ selected from the group consisting of lymph node, gall bladder, thymus, hypothalamus, stomach, intestine, liver, pancreas, kidney, skin, and a secretory gland (e.g. a salivary gland, pineal gland, thyroid gland, adrenal gland, and parathyroid gland). In some aspects, the composition is suitable for delivery to the salivary gland. In some aspects, the composition is suitable for delivery to a secretory organ selected from heart, bone, muscle, skin, and/or adipose tissue. In some aspects, the composition is suitable for delivery to a secretory organ or other delivery site selected from the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, the disclosure is directed to an adeno-associated virus (AAV) capsid comprising an expression cassette comprising a promoter operably linked a nucleic acid encoding an anti-TNFalpha antibody (e.g., a monoclonal antibody) or an antigen-binding fragment thereof, wherein the AAV capsid is suitable for delivering to a delivery site disclosed herein. In some aspects, the AAV capsid is delivered to a secretory organ selected from the group consisting of lymph node, gall bladder, thymus, hypothalamus, stomach, intestine, liver, pancreas, kidney, skin, and a secretory gland (e.g., a salivary gland, pineal gland, thyroid gland, adrenal gland, and parathyroid gland). In some aspects, the AAV capsid is delivered to the salivary gland. In some aspects, the AAV capsid is delivered to a secretory organ selected from heart, bone, muscle, skin, and/or adipose tissue. In some aspects, the AAV capsid is delivered to a secretory organ or other delivery site selected from the intestines (e.g., an intestinal wall, a perineal muscle, or an anal wall), adipose tissue, a proximate gland to the intestines, and/or one or both eyes (e.g., intraocular). In some aspects, the administration to one or both eyes is intravitreal (e.g., intravitreal injection). In some aspects, the administration to one or both eyes is intrastromal or transconjunctival.

Some aspects of the disclosure are directed to a method of expressing an anti-TNFalpha antibody in a subject in need thereof comprising administering an effective amount of a composition or an AAV capsid described herein to the secretory organ, secretory-like organ, or other delivery site of the subject. Some aspects of the disclosure are directed to a method of delivering a gene therapy to a subject in need thereof comprising administering to a secretory organ (e.g., the secretory gland) of the subject a delivery vector (e.g., a viral vector) comprising a promoter operably linked to a nucleic acid sequence that encodes an anti-TNFalpha antibody (e.g., a monoclonal antibody) or an antigen-binding fragment thereof. Some aspects of the disclosure are directed to a method of delivering a gene therapy to a subject in need thereof comprising administering to a secretory-like organ or other site of the subject a delivery vector (e.g., a viral vector) comprising a promoter operably linked to a nucleic acid sequence that encodes an anti-TNFalpha antibody (e.g., a monoclonal antibody) or an antigen-binding fragment thereof. Some aspects of the disclosure are directed to a method of delivering a nucleic acid to a secretory cell, secretory-like cell, or other cell, comprising administering to the secretory cell, secretory-like cell, or other cell of a subject an adeno-associated virus (AAV) capsid comprising an expression cassette comprising a promoter operably linked to a nucleic acid encoding an anti-TNFalpha antibody (e.g., a monoclonal antibody) or an antigen-binding fragment thereof, a fusion protein (e.g., an Fc fusion protein), or a therapeutic peptide, thereby delivering the nucleic acid to the secretory cell, secretory-like cell, or other cell of the subject.

In some aspects, the administration is suitable for delivery of a rAAV particle or capsid comprising a vector (e.g., an antibody expression cassette) disclosed herein to one or both eyes. In some aspects, the administration is by injection. In some aspects, the administration is intravitreal. In some aspects, the administration is intrastromal or transconjunctival.

In some aspect, the administration is a single dose. In some aspect, the single dose is administered in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye. In some aspect, the single dose is administered in a volume of about 40 μL to 60 μL per eye. In some aspect, the single dose is administered in a volume of about 50 μL per eye.

In some aspects, the administration comprises a dose within the range of 1E9 vg to 3E12 vg. In some aspects, the administration comprises a dose within the range of 1E9 vg to 1E12 vg. In some aspects, the administration comprises a dose within the range of 1E9 vg to 1E11 vg.

In some aspects, the intravitreal, intrastromal or transconjunctival administration comprises a single dose comprising 1E9 vg to 3E12 vg, 1E9 vg to 1E12 vg, or 1E9 vg to 1E11 vg in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye.

In some aspects, the anti-TNFalpha antibody steady state concentration in the eye (e.g., in ocular fluid, for example, in the aqueous or viterious humor) of the subject after administration is 10 ng/mL to 1000 ng/mL (1 μg/mL). In some aspects, the anti-TNFalpha antibody steady state concentration in the eye (e.g., in ocular fluid, for example, in the aqueous or viterious humor) of the subject after administration is 10 ng/mL to 500 ng/mL. In some aspects, the anti-TNFalpha antibody steady state concentration in the eye (e.g., in ocular fluid, for example, in the aqueous or viterious humor) of the subject after administration is 10 ng/mL to 100 ng/mL. In some aspects, the anti-TNFalpha antibody steady state concentration in the eye (e.g., in ocular fluid, for example, in the aqueous or viterious humor) of the subject after administration is at least 10 ng/mL, at least 20 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 250 ng/mL, at least 300 ng/mL, at least 400 ng/mL, at least 500 ng/mL, at least 600 ng/mL, at least 700 ng/mL, at least 800 ng/mL, at least 900 ng/mL, or at least 1 μg/mL.

In some aspects, the anti-TNFalpha antibody steady state concentration in the serum of the subject after administration is less than 1% of the total anti-TNFalpha antibody concentration after administration (to one or both eyes). In some aspects, the anti-TNFalpha antibody steady state concentration in the serum of the subject after administration is less than 20 ng/mL, less than 15 ng/mL, less than 10 ng/mL, less than 5 ng/mL, less than 1 ng/mL, or less than 0.5 ng/mL. In some aspects, the anti-TNFalpha antibody steady state concentration in the serum of the subject after administration is 0.1 ng/mL to 20 ng/mL (e.g., 0.5 ng/mL to 20 ng/mL, 0.5 ng/mL to 10 ng/mL, or 0.5 ng/mL to 5 ng/mL).

Non-limiting examples of the various aspects are shown in the present disclosure.

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the term “secretory organ” refers to any organ that synthesizes substances needed by the body and releases it through ducts or directly into the bloodstream. In some aspects, the secretory organs include lymph node, gall bladder, thymus, hypothalamus, stomach, intestine, liver, pancreas, kidney, skin, and/or secretory gland. In some aspects, the secretory organ can include heart, bone, muscle, skin, and/or adipose tissue.

As used herein, the term “secretory-like organ” refers to an organ that is not typically considered a secretory organ, but its cells have been modified such that it can secrete and release molecules directly into the bloodstream.

As used herein, the term “secretory gland” refers to an aggregation of cells specialized to secrete or excrete materials not related to the cells' ordinary metabolic needs. In some aspects, secretory glands include salivary gland, pineal gland, thyroid gland, adrenal gland, and parathyroid glands.

As used herein, the term “salivary gland” refers to a gland of the oral cavity which secretes saliva, including the glandulae salivariae majores of the oral cavity (the parotid, sublingual, and submandibular glands) and the glandulae salivariae minores of the tongue, lips, cheeks, and palate (labial, buccal, molar, palatine, lingual, and anterior lingual glands).

As used herein, the term “delivery vector” or “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a protein particle, a bacterial vector, and a lysosome.

Some aspects of the disclosure are directed to biological vectors, which can include viruses, particularly attenuated and/or replication-deficient viruses.

As used herein, the term “promoter” refers to a DNA sequence recognized by the machinery of the cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible by external signals or agents; such elements can be located in the 5′ or 3′ regions of the native gene. In some aspects, the promoter is a constitutively active promoter, a cell-type specific promoter, or an inducible promoter.

In some aspects, microRNA targeting sequences are included to increase specificity of vector-mediated transgene expression. See e.g., Anja Geisler and Henry Fechner, World J Exp Med., 20; 6(2):37-54 (2016).

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

As used herein, the term “regulatable promoter” is any promoter whose activity is affected by a cis or trans acting factor (e.g., an inducible promoter, such as an external signal or agent).

As used herein, the term “constitutive promoter” is any promoter that directs RNA production in many or all tissue/cell types at most times, e.g., the human CMV immediate early enhancer/promoter region that promotes constitutive expression of cloned DNA inserts in mammalian cells.

The terms “transcriptional regulatory protein,” “transcriptional regulatory factor,” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA can be indirect by way of binding to another protein that in turn binds to, or is bound to a DNA response element.

As used herein, the term “termination signal sequence” can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

As used herein, the term “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). “Under translational control of an IRES” as used herein means that translation is associated with the IRES and proceeds in a cap-independent manner.

The term “self-processing cleavage site” or “self-processing cleavage sequence,” as used herein refers to a post-translational or co-translational processing cleavage site or sequence. Such a “self-processing cleavage” site or sequence refers to a DNA or amino acid sequence, exemplified herein by a 2A site, sequence or domain or a 2A-like site, sequence or domain. The term “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

As used herein, the term “additional proteolytic cleavage site,” refers to a sequence that is incorporated into an expression construct of the disclosure adjacent a self-processing cleavage site, such as a 2A or 2A like sequence, and provides a means to remove additional amino acids that remain following cleavage by the self-processing cleavage sequence. Exemplary 2A peptides include, but are not limited to, P2A, E2A, F2A, and T2A. Exemplary “additional proteolytic cleavage sites” are described herein and include, but are not limited to, furin cleavage sites with the consensus sequence RXK(R)R (SEQ ID NO: 27). Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

The term “expression vector” or “expression construct” means any type of genetic construct containing a nucleic acid in which part or all of the nucleic acid encoding sequence is capable of being transcribed. In some aspects, the expression vector or construct can comprise an antibody expression cassette.

As used herein, the term “multicistronic” or “multicistronic vector” refers to a nucleic acid sequence having two or more open reading frames (e.g., genes). An open reading frame in this context is a sequence of codons that is translatable into a polypeptide or protein (e.g. a heavy chain or a light chain). “Bicistronic” or “bicistronic vector” refers to a nucleic acid sequence having two open reading frames (e.g., genes). An open reading frame in this context is a sequence of codons that is translatable into a polypeptide or protein (e.g. a heavy chain or a light chain). In some aspects, the construct of the disclosure is a multicistronic (e.g., bicistronic) construct (e.g., comprising a heavy and a light chain).

A “viral vector” refers to a sequence that comprises one or more polynucleotide regions encoding or comprising a molecule of interest, e.g., a protein, a peptide, and an oligonucleotide or a plurality thereof. Viral vectors can be used to deliver genetic materials into cells. Viral vectors can be modified for specific applications. In some aspects, the delivery vector of the disclosure is a viral vector selected from the group consisting of an adeno-associated viral (AAV) vector, an adenoviral vector, a lentiviral vector, or a retroviral vector.

The term “adeno-associated virus vector” or “AAV vector” as used herein refers to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. In some aspects, the terms “AAV genome” and “AAV vector” can be used interchangeably. In some aspects, the AAV vector is modified relative to the wild-type AAV serotype sequence.

As used herein, a “recombinant AAV particle” or “rAAV particle” is an AAV virus that comprises a capsid protein and an AAV vector or AAV vector genome having at least one payload region (e.g., an expression cassette including a polynucleotide encoding a therapeutic protein (e.g., an antibody or antigen binding fragment thereof) or peptide) and at least one inverted terminal repeat (ITR) region. In some aspects, the terms “AAV vectors of the present disclosure” or “AAV vectors” refer to AAV vectors comprising a polynucleotide encoding an antibody, e.g., encapsulated in an AAV capsid.

A “coding sequence” or a sequence “encoding” a particular molecule (e.g., a therapeutic protein or peptide) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., an AVV vector) that is derived from a second nucleic acid sequence (e.g., another AVV vector) can include a nucleotide sequence that is identical or substantially similar to the nucleotide sequence of the second nucleic acid sequence.

In the case of a polynucleotide disclosed herein, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each. The mutagenesis of a polynucleotide to create a different polynucleotide derived from the first can be a random event (e.g., caused by polymerase infidelity) and the identification of the derived polynucleotide can be made by appropriate screening methods.

As used herein, the term “intravitreal” refers to the space inside the eyeball behind the lens that contains the jelly-like vitreous humor. The term “intravitreal injection” refers to an injection into the eye's vitreous humor between the lens and the retina.

As used herein, the term “mutation” refers to any changing of the structure of a gene, resulting in a variant (also called “mutant”) form that can be transmitted to subsequent generations. Mutations in a gene can be caused by the alternation of single base in DNA, or the deletion, insertion, or rearrangement of larger sections of genes or chromosomes.

As used herein, the term “administration” refers to the administration of a composition of the present disclosure (e.g., an AAV vector, a rAAV particle, or the gene therapy composition disclosed herein) to a subject or system. Administration to an animal subject (e.g., to a human) can be by any appropriate route, such as to a secretory organ.

As used herein, the term “modified” refers to a changed state or structure of a molecule of the disclosure. Molecules can be modified in many ways including chemically, structurally, and functionally.

As used herein, the term “synthetic” means produced, prepared, and/or manufactured by the hand of man. Synthesis of polynucleotides or polypeptides or other molecules of the present disclosure can be chemical or enzymatic.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “mRNA,” as used herein, refers to a single stranded RNA that encodes the amino acid sequence of one or more polypeptide chains.

The term “antisense,” as used herein, refers to a nucleic acid that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides can hybridize to each other even if they are not completely complementary to each other, provided that each has at least one region that is substantially complementary to the other.

The terms “antisense strand” and “guide strand” refer to the strand of a dsRNA, e.g., an shRNA, that includes a region that is substantially complementary to a target sequence, e.g., mRNA. The antisense strand has sequence sufficiently complementary to the desired target mRNA sequence to direct target-specific silencing, e.g., complementarity sufficient to trigger the destruction of the desired target mRNA by the RNAi machinery or process.

The terms “sense strand” and “passenger strand,” as used herein, refer to the strand of a dsRNA, e.g., an shRNA, that includes a region that is substantially complementary to a region of the antisense strand as that term is defined herein. The antisense and sense strands of a dsRNA, e.g., an shRNA, are hybridized to form a duplex structure.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

The terms “antibody” and “antibodies” refer to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab′, F(ab′)2, and Fv fragments, linear antibodies, and single chain antibodies (e.g., nanobodies). An antigen-binding fragment of an antibody can be derived from any animal species, such as rodents (e.g., mouse, rat, or hamster) and humans or can be artificially produced.

The term “nanobody” or “nanobodies” or “single-domain antibody” or “sdAb” refers to a class of antigen-binding fragments that is a single chain immunoglobulin molecule consisting of a momomeric variable antibody domain, which recognizes and specifically binds to an antigen.

The term “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

The term “bispecific” or “bifunctional antibody” or antigen-binding fragment thereof refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “multispecific antibody” refers to an antibody having specificities for more than two different epitopes, typically non-overlapping epitopes or an antibody that contains more than two distinct antigen-binding sites.

The phrase “contacting a cell” (e.g., contacting a cell with an AAV vector, a rAAV particle, or the gene therapy composition of the disclosure) as used herein, includes contacting a cell directly or indirectly. In some aspects, contacting a cell with an AAV vector, a rAAV particle, or the gene therapy composition includes contacting a cell in vitro with the gene therapy composition, the AAV vector, or the rAAV particle or contacting a cell in vivo with the AAV vector, the rAAV particle, or the gene therapy composition. Thus, for example, the AAV vector, the rAAV particle, or the gene therapy composition can be put into physical contact with the cell by the individual performing the method, or alternatively, the AAV vector, the rAAV particle, or the gene therapy composition can be put into a situation that will permit or cause it to subsequently come into contact with the cell.

In some aspects, contacting a cell in vitro can be done, for example, by incubating the cell with the AAV vector. In some aspects, contacting a cell in vivo can be done, for example, by injecting the AAV vector, the rAAV particle, or the gene therapy composition of the disclosure into or near the tissue where the cell is located (e.g., a secretory organ), or by injecting the AAV vector, the rAAV particle, or the gene therapy composition into another area, e.g., the bloodstream or the subcutaneous space, such that the agent will subsequently reach the tissue where the cell to be contacted is located. For example, the AAV vector can be encapsulated and/or coupled to a ligand that directs the AAV vector to a site of interest. Combinations of in vitro and in vivo methods of contacting are also possible. For example, a cell can be contacted in vitro with an AAV vector, a rAAV particle, or the gene therapy composition and subsequently transplanted into a subject.

In some aspects, contacting a cell with an AAV vector, a rAAV particle, or the gene therapy composition of the present disclosure includes “introducing” or “delivering” (directly or indirectly) the AAV vector, the rAAV particle, or the gene therapy composition into the cell by facilitating or effecting uptake or absorption into the cell. Introducing an AAV vector, an rAAV particle, or the gene therapy composition into a cell can be in vitro and/or in vivo. For example, for in vivo introduction, an AAV vector, an rAAV particle, the gene therapy composition can be injected into a specific tissue site (e.g., the locus where a therapeutic effect is desired) or administered systemically (e.g., administering a AAV vector targeted to a locus where a therapeutic effect is desired). In vitro introduction into a cell includes methods known in the art such as electroporation and lipofection.

As used herein, the terms “effective amount,” “therapeutically effective amount,” and a “sufficient amount” of, e.g., an AAV vector, a rAAV particle, or the gene therapy composition disclosed herein refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., an AAV vector, a rAAV particle, the gene therapy composition disclosed herein) is an amount that results in a beneficial or desired result in a subject as compared to a control.

The amount of a given agent (e.g., an AAV vector, a rAAV particle, or the gene therapy composition disclosed herein) will correspond to such an amount will vary depending upon various factors, such as the given agent, the pharmaceutical formulation, the route of administration, the type of disease or disorder, the identity of the subject (e.g., age, sex, and/or weight) or host being treated, and the like.

As used herein, the term “gene therapy” is the insertion of nucleic acid sequences (e.g., a polynucleotide comprising a promoter operably linked to a nucleic acid encoding a therapeutic molecule as disclosed herein) into an individual's cells and/or tissues to treat, reduce the symptoms of, or reduce the likelihood of a disease. Gene therapy also includes insertion of transgene that are inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases are amenable to gene therapy.

The term “prophylactically effective amount,” as used herein, includes the amount of an agent, (e.g., an AAV vector, an rAAV particle, or the gene therapy composition disclosed herein) that, when administered to a subject having or predisposed to have a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis (e.g., non-infectious uveitis such as intermediate uveitis, posterior uveitis, or panuveitis) or a corneal disease (e.g., peripheral ulcerative keratitis, corneal hemangiogenesis, or noninfectious corneal melting)), an inflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease, is sufficient to prevent, reduce the symptoms of, or ameliorate the disease or disorder or one or more symptoms of the disease or disorder. Ameliorating the disease or disorder includes slowing the course of the disease or disorder or reducing the severity of later-developing disease or disorder. The “prophylactically effective amount” can vary depending on the characteristics of the agent, e.g., an AAV vector, a rAAV particle, or the gene therapy composition, how the agent is administered, the degree of risk of disease, and the history, age, weight, family history, genetic makeup, the types of preceding or concomitant treatments, if any, and other individual characteristics of the patient to be treated.

As used herein, “off target” refers to any unintended effect on any one or more target, gene, or cellular transcript.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, e.g., siRNA, shRNA, sense and/or anti-sense sequences, DNA encoding one or more genes and organized into an expression plasmid, e.g., a vector.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

By “level” is meant a level or activity of a protein, or mRNA encoding the protein, optionally as compared to a reference. The reference can be any useful reference, as defined herein. By a “decreased level” or an “increased level” of a protein is meant a decrease or increase in protein level, as compared to a reference.

A level of a protein can be expressed in mass/vol (e.g., g/dL, mg/mL, μg/mL, ng/mL) or percentage relative to total protein or mRNA in a sample.

The term “pharmaceutical composition,” as used herein, represents a composition comprising a compound or molecule described herein, e.g., an AAV vector disclosed herein, formulated with a pharmaceutically acceptable excipient, and can be manufactured or sold with the approval of a governmental regulatory agency as part of a therapeutic regimen for the treatment of disease in a mammal.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration.

As used herein, the term “subject” refers to any organism to which a composition disclosed herein, e.g., an AAV vector of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. In some aspects, treating reduces or lessens the symptoms associated with a disease or disorder. In some aspects, the treating results in a beneficial or desired clinical result.

Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years.

### II. Therapeutic Antibodies

The present disclosure provides gene therapy compositions comprising a polynucleotide (e.g, an antibody expression cassette) comprising a promoter operably linked to a nucleic acid encoding or comprising an antibody or antigen binding fragment thereof that binds tumor necrosis factor (TNF)-alpha (also referred to interchangeably herein as an “anti-TNFalpha antibody”, “anti-TNF-α antibody”, “anti-TNFα antibody”, and “anti-TNF-alpha antibody” herein). In some aspects, the antibody is a monoclonal antibody.

In some aspects, a polynucleotide encoding an anti-TNFalpha antibody can be inserted into a viral vector (e.g., an AAV vector) disclosed herein, wherein the polynucleotide is operably linked with the promoter. In some aspects, the promoter can drive the expression of the anti-TNFalpha antibody in a host cell (e.g., a human secretory cell). In some aspects, the polynucleotide encoding an anti-TNFalpha antibody can be administered to a secretory organ (e.g., a salivary gland). In some aspect, the polynucleotide encoding the anti-TNFalpha antibody is administered to the salivary gland and the anti-TNFalpha antibody is secreted in the saliva. In some aspects, the polynucleotide encoding an anti-TNFalpha antibody can be administered to a secretory-like organ or other organ disclosed herein. In some aspects, the polynucleotide encoding an anti-TNFalpha antibody can be administered intramuscularly, intracutaneously, intravenously, or intraocularly (e.g., intravitreal, intrastromal, or transconjunctival).

In some aspects, the anti-TNFalpha antibody is an antibody or antigen-binding fragment thereof selected from a monoclonal antibody, a bispecific antibody, or a multispecific antibody or antigen-binding fragment thereof. In some aspects, the therapeutic protein is an antibody fragment selected from a Fab, a Fab′, a F(ab′)2, a Fv fragments, a linear antibody, or a single chain antibody (e.g., a nanobody).

In some aspects, a composition comprising a nucleic acid encoding an anti-TNFalpha antibody disclosed herein is suitable for delivery to a secretory organ or secretory-like organ. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the secretory organ is a secretory gland selected from salivary gland, pineal gland, thyroid gland, adrenal gland, and parathyroid gland. In some aspects, the secretory gland is a salivary gland. In some aspects, a composition comprising a nucleic acid encoding a protein or peptide further comprises a nucleic acid sequence encoding a secretory signal and is suitable for delivery to a secretory-like organ. In some aspects, the nucleic acids encoding a protein or peptide disclosed herein is suitable for delivery to other delivery sites disclosed herein.

In some aspects, the composition comprising a nucleic acid encoding an anti-TNFalpha antibody disclosed herein is suitable treating a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis (e.g., non-infectious uveitis such as intermediate uveitis, posterior uveitis, or panuveitis) or a corneal disease (e.g., peripheral ulcerative keratitis, corneal hemangiogenesis, or noninfectious corneal melting)), an inflammatory disease or disorder, a cardiovascular disease or disorder, an oral mucosal disease or disorder, a gastrointestinal disease or disorder, an esophageal disease or disorder, a pulmonary disease or disorder, a rheumatological disease or disorder, or an immune disease or disorder (e.g., inflammatory bowel disease.

In some aspects, a composition comprising a nucleic acid encoding an anti-TNFalpha antibody disclosed herein is suitable for delivery to the salivary gland for treating an oral mucosal disease such as oral lichen planus, mucous membrane pemphigus, bullous pemphigus, pemphigus vulgaris, systemic lupus erythematosus, Behcet's disease, aphthous stomatitis or any combination thereof.

In some aspects, a composition comprising a nucleic acid encoding an anti-TNFalpha antibody disclosed herein is suitable for delivery to the salivary gland for treating an inflammatory and autoimmune disease of the esophagus.

In some aspects, a composition comprising a nucleic acid encoding an anti-TNFalpha antibody disclosed herein is suitable for delivery to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, a composition comprising a nucleic acid encoding an anti-TNFalpha antibody disclosed herein is suitable for delivery to the intestines (e.g., an intestinal wall, a perineal muscle, or an anal wall), adipose tissue, a proximate gland to the intestines, or a combination thereof for treating an inflammatory bowel disease and/or associated complications thereof, e.g., perianal fistulas.

In some aspects, a composition comprising a nucleic acid encoding an anti-TNFalpha antibody disclosed herein is suitable for delivery to one or both eyes for treating an ocular disease or disorder, e.g., uveitis. In some aspects, the uveitis is non-infectious uveitis. In some aspects, the non-infectious uveitis is selected from the group consisting of intermediate uveitis, posterior uveitis, and panuveitis.

In some aspects, a composition comprising a nucleic acid encoding an anti-TNFalpha antibody disclosed herein is suitable for delivery to one or both eyes for treating a corneal disease (e.g., peripheral ulcerative keratitis, corneal hemangiogenesis, and noninfectious corneal melting).

In some aspects, the therapeutic antibody comprises a heavy chain and a light chain. In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 17-19 or 110 (or nucleotides 55-1407 of SEQ ID NO: 56 or nucleotides 1951-3304 of SEQ ID NO: 64). In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 21-23, 111 (or nucleotides 61-702 of SEQ ID NO: 49 or nucleotides 3460-4105 of SEQ ID NO: 64), 144, or 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 17. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 21.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 18. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 22.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 19. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 23.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 110. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 142. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 144.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 146. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 150. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 151. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the therapeutic antibody comprises a modified heavy chain and a modified light chain. In some aspects, the nucleic acid sequence encoding the modified heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 18-19, 110 (or nucleotides 55-1407 of SEQ ID NO: 56 or nucleotides 1951-3304 of SEQ ID NO: 64), 142, 146, 150, or 151. In some aspects, the nucleic acid sequence encoding the modified light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 22-23, 111 (or nucleotides 61-702 of SEQ ID NO: 49 or nucleotides 3460-4105 of SEQ ID NO: 64), 144, or 148.

In some aspects, the nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 7-9, 102 (or nucleotides 55-417 of SEQ ID NO: 56), 143, or 147. In some aspects, the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 11-13, 103 (or nucleotides 61-381 of SEQ ID NO: 49), 145, or 149.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 7 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 11.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 8 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 12.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 9 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 13.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 102 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 103.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 143 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 145.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 147 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 149.

In some aspects, the nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 7-9, 102 (or nucleotides 55-417 of SEQ ID NO: 56), 143, or 147. In some aspects, the nucleic acid sequence encoding the modified light chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 12-13, 103 (or nucleotides 61-381 of SEQ ID NO: 49), 145, or 149.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 7 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 11.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 8 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 12.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 9 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 13.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 102 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 103.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 143 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 145.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 147 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 149.

In some aspects, the heavy chain comprises a modified heavy chain variable region (VH) comprising a complementarity determining region (CDR) 1, a VH CDR2, and a VH CDR3. In some aspects, the VH CDRs 1-3 correspond to the CDRs of adalimumab. In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, 87, and 90, the nucleic acid sequence comprising VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, 88, or 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86, 89, 92, 106, or 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 87, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 88, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 89.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 92.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the light chain comprises a modified light chain variable region (VL) comprising a complementarity determining region (CDR) 1, a VL CDR2, and a VL CDR3. In some aspects, the VL CDRs 1-3 correspond to the CDRs of adalimumab. In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, 96, 99, or 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, 97, 100, 108, 160, or 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95, 98, 101, or 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 96, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 97, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 98.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 100, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 108, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 160, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

### II.A.1 Antibodies

In some aspects, a gene therapy composition comprises a polynucleotide (e.g, an antibody expression cassette) comprising a promoter operably linked to a nucleic acid encoding an immunoglobulin, e.g., an antibody or antigen-binding fragment thereof that binds to TNFα. In some aspects, the antibody is selected from the group consisting of a monoclonal antibody, a bispecific antibody, and a multispecific antibody.

In some aspects, the polynucleotide disclosed herein encodes a heavy chain and a light chain. In some aspects, the polynucleotide disclosed herein encodes a variable heavy chain or a variable light chain. In some aspects, the polynucleotide disclosed herein encodes a nanobody.

The term “immunoglobulin” is used herein to include antibodies, functional fragments thereof, Fabs, scFvs, single domain antibodies (e.g., nanobodies), DARTs, F(ab′)2, BITEs, and immunoadhesins. These antibody fragments or artificial constructs can include a single chain antibody, an Fab fragment, a univalent antibody, a bivalent of multivalent antibody, or an immunoadhesin. The binding or neutralizing antibody construct can be a monoclonal antibody, a “humanized” antibody, a multivalent antibody, or another suitable construct. An “immunoglobulin molecule” is a protein containing the immunologically-active portions of an immunoglobulin heavy chain and immunoglobulin light chain covalently coupled together and capable of specifically combining with an antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” can be used interchangeably herein. An “immunoglobulin heavy chain” is a polypeptide that contains at least a portion of the antigen binding domain of an immunoglobulin and at least a portion of a variable region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain. An “immunoglobulin light chain” is a polypeptide that contains at least a portion of the antigen binding domain of an immunoglobulin and at least a portion of the variable region. Thus, the immunoglobulin-derived light chain has significant regions of amino acid homology with a member of the immunoglobulin gene superfamily. An “immunoadhesin” is a chimeric, antibody-like molecule that combines the functional domain of a binding protein, usually a receptor, ligand, cell-adhesion molecule, or 1-2 immunoglobulin variable domains with immunoglobulin constant domains, usually including the hinge or GS linker and Fc regions. A “fragment antigen-binding” (Fab) fragment” is a region on an antibody that binds to antigens. It is composed of one constant and one variable domain of each of the heavy and the light chain. With respect to immunoglobulins or antibodies as described herein, each fragment of an immunoglobulin coding sequence can be derived from one or more sources, or synthesized. Suitable fragments can include the coding region for one or more of, e.g., a heavy chain, a light chain, and/or fragments thereof such as the constant or variable region of a heavy chain (CH1, CH2 and/or CH3) and/or or the constant or variable region of a light chain. Alternatively, variable regions of a heavy chain or light chain can be utilized. Where appropriate, these sequences can be modified from the “native” sequences from which they are derived, as described herein. As used herein, the term “immunoglobulin construct” refers to any of the above immunoglobulins or fragments thereof which are encoded by and included in the expression cassettes and viral vectors described herein.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B)(CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, the CDRs of the antibodies described herein have been determined according to the Kabat numbering scheme.

As used herein, the term “constant region” or “constant domain” are interchangeable and have the meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (u) or lambda (k) based on the amino acid sequence of the constant domains. Light chain amino acid sequences are well known in the art. In some aspects, the light chain is a human light chain.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system.

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally-occurring variants thereof. Native sequence Fc includes the various allotypes of Fc (see, e.g., Jefferis et al., (2009) mAbs 1:1; Vidarsson G. et al. Front Immunol. 5:520 (published online Oct. 20, 2014)).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Human IgG1 binds to most human Fc receptors and elicits the strongest Fc effector functions. It is considered equivalent to murine IgG2a with respect to theit types of activating Fc receptors that it binds to. Conversely, human IgG4 elicits the least Fc effector functions. Vidarsson G. et al. Front Immunol. 5:520 (published online Oct. 20, 2014).

The constant region can be manipulated, e.g., by recombinant technology, to eliminate one or more effector functions. An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and down regulation of a cell surface receptor (e.g., the B cell receptor; BCR). Such effector functions generally require the Fc region to be combined with a binding domain (e.g., an antibody variable domain). Accordingly, the term “a constant region without the Fc function” include constant regions with reduced or without one or more effector functions mediated by Fc region.

Effector functions of an antibody can be reduced or avoided by different approaches. Effector functions of an antibody can be reduced or avoided by using antibody fragments lacking the Fc region (e.g., such as a Fab, F(ab′)2, single chain Fv (scFv), or a sdAb consisting of a monomeric VH or VL domain). Alternatively, the so-called aglycosylated antibodies can be generated by removing sugars that are linked to particular residues in the Fc region to reduce the effector functions of an antibody while retaining other valuable attributes of the Fc region (e.g., prolonged half-life and heterodimerization). Aglycosylated antibodies can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells). See, e.g., U.S. Pub. No. 20120100140. Another approach is to employ Fc regions from an IgG subclass that have reduced effector function. For example, IgG2 and IgG4 antibodies are characterized by having lower levels of Fc effector functions than IgG1 and IgG3. The residues most proximal to the hinge region in the CH2 domain of the Fc part are responsible for effector functions of antibodies as it contains a largely overlapping binding site for C1q (complement) and IgG-Fc receptors (FcγR) on effector cells of the innate immune system. Vidarsson G. et al. Front Immunol. 5:520 (published online Oct. 20, 2014). Accordingly, antibodies with reduced or without Fc effector functions can be prepared by generating, e.g., a chimeric Fc region which comprises a CH2 domain from an IgG antibody of the IgG4 isotype and a CH3 domain from an IgG antibody of the IgG1 isotype, or a chimeric Fc region which comprises hinge region from IgG2 and CH2 region from IgG4 (see, e.g., Lau C. et al. J. Immunol. 191:4769-4777 (2013)), or an Fc region with mutations that result in altered Fc effector functions, e.g., reduced or no Fc functions. Such Fc regions with mutations are known in the art. See, e.g., U.S. Pub. No. 20120100140 and U.S. and PCT applications cited therein and An et al., mAbs 1:6, 572-579 (2009); the disclosures of which are incorporated by reference to their entirety.

In some aspects, the antibody (e.g., a monoclonal antibody) or antigen-binding fragment thereof is modified so that it does not bind to the Fc region. See e.g., Saunders K., Front. Immunol., 10:1296 (2019).

A “hinge,” “hinge domain,” “hinge region,” or “antibody hinge region” are used interchangeably and refer to the domain of a heavy chain constant region that joins the CH1 domain to the CH2 domain and includes the upper, middle, and lower fragments of the hinge (Roux et al., J. Immunol. 1998 161:4083). The hinge provides varying levels of flexibility between the binding and effector regions of an antibody and also provides sites for intermolecular disulfide bonding between the two heavy chain constant regions.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody which is substantially free of other antibodies having different antigenic. An isolated antibody that specifically binds to an epitope of a protein can, however, have cross-reactivity to other corresponding proteins from different species.

In some aspects, the antibody (e.g., a monoclonal antibody) or antigen-binding fragment thereof is a chimeric antibody. The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

In some aspects, the antibody (e.g., a monoclonal antibody) or antigen-binding fragment thereof is a humanized antibody. The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining regions (CDRs) are replaced by residues from the CDRs of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some aspects, a humanized antibody or antigen-binding fragment thereof can comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

In some aspects, the antibody (e.g., a monoclonal antibody) or antigen-binding fragment thereof is a human antibody. The term “human” antibody (HuMAb) or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

An antibody that is “blocking” or that “blocks” or that is “inhibitory” of that “inhibits” is an antibody that reduces or inhibits (partially or completely) binding of its target protein to one or more ligands when the antibody is bound to the target protein, and/or that reduces or inhibits (partially or completely) one or more activities or functions of the target protein when the antibody is bound to the target protein.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope. The term “epitope mapping” refers to the process of identification of the molecular determinants for antibody-antigen recognition.

In certain aspects, a composition comprising a delivery vector, e.g., a viral vector, comprising nucleic acids encoding an immunoglobulin disclosed herein (e.g., an antibody) is suitable for delivery to a secretory organ. In some aspects, the secretory organ is selected from lymph node, gall bladder, thymus, hypothalamus, stomach, intestine, liver, pancreas, kidney, skin and/or secretory gland. In some aspects, the secretory organ is selected from heart, bone, muscle, skin, and/or adipose tissue. In some aspects, the composition is suitable for delivery to the liver. In some aspects, the secretory gland is a salivary gland, pineal gland, thyroid gland, adrenal gland, and parathyroid gland. In some aspects, the secretory gland is a salivary gland. In some aspects, a composition comprising the delivery vector, e.g., a viral vector, comprising nucleic acids encoding an immunoglobulin disclosed herein (e.g., an antibody) is suitable for delivery to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, a secretory signal sequence (as described in e.g., Sun et al., Mol Ther., 14(6):822-830 (2006)) can be used to enhance secretion of therapeutic proteins (e.g., monoclonal antibodies and fusion proteins) or therapeutic peptides from a secretory organ, secretory-like organ, or other organ disclosed herein. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the polynucleotide disclosed herein comprises a nucleic acid sequence encoding a secretory signal sequence. In some aspects, the gene therapy composition comprising a polynucleotide comprising a nucleic acid encoding a therapeutic protein or peptide further comprises a nucleic acid sequence encoding a secretory signal, wherein the gene therapy composition is suitable for delivery to a secretory-like organ.

In some aspects, provided herein are antibodies (e.g., monoclonal antibodies) and antigen-binding fragments thereof which specifically bind to a tumor necrosis factor (TNF), such as human TNF-α. In some aspects, the encoded anti-TNFalpha antibody comprises the amino acid sequence of adalimumab or a variant thereof.

In some aspects, the gene therapy constructs used in the methods disclosed herein encode an antibody (e.g., monoclonal antibodies or antigen-binding fragments thereof) having the CDR and/or variable region sequences of adalimumab or antibodies having at least 80% identity (e.g., at least about 85%, at least about 90%, at least about 95%, at least about 96%, at least about 97%, at least about 98%, at least about 99%, or about 100% identity) to their variable region or CDR sequences of adalimumab. In some aspects, the gene therapy construct encoding an anti-TNFalpha antibody (e.g., adalimumab) is a multicistronic (e.g., bicistronic) construct (e.g., comprising a heavy chain and a light chain). In some aspects, the multicistronic (e.g., bicistronic) construct further comprises an F2A or IRES element.

In some aspects, the polynucleotide disclosed herein encodes an antibody comprising a heavy chain and a light chain of adalimumab or an antigen-binding fragment thereof. In some aspects, the polynucleotide disclosed herein encodes an antibody comprising modified heavy chain variable region and a modified light chain variable region of adalimumab or an antigen-binding fragment thereof.

In some aspects, the therapeutic antibody comprises a heavy chain and a light chain. In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 17-19, 110 (or nucleotides 55-1407 of SEQ ID NO: 56 or nucleotides 1951-3304 of SEQ ID NO: 64), 142, 146, 150, or 151. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 21-23, 111 (or nucleotides 61-702 of SEQ ID NO: 49 or nucleotides 3460-4105 of SEQ ID NO: 64), 144, or 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 17. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 21.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 18. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 22.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 19. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 23.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 110. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 142. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 144.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 146. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 150. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 111.

In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 151. In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 148.

In some aspects, the therapeutic antibody comprises a modified heavy chain and a modified light chain. In some aspects, the nucleic acid sequence encoding the modified heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 18-19, 110 (or nucleotides 55-1407 of SEQ ID NO: 56 or nucleotides 1951-3304 of SEQ ID NO: 64), 142, 146, 150, or 151. In some aspects, the nucleic acid sequence encoding the modified light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 22-23, 111 (or nucleotides 61-702 of SEQ ID NO: 49 or nucleotides 3460-4105 of SEQ ID NO: 64), 144, or 148.

In some aspects, the nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 7-9 102 (or nucleotides 55-417 of SEQ ID NO: 56), 143, or 147. In some aspects, the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 11-13, 103 (or nucleotides 61-381 of SEQ ID NO: 49), 145, or 149.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 7 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 11.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 8 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 12.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 9 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 13.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 102 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 103.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 143 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 145.

In some aspects, nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 147 and the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 149.

In some aspects, the heavy chain comprises a modified heavy chain variable region (VH) comprising a complementarity determining region (CDR) 1, a VH CDR2, and a VH CDR3. In some aspects, the VH CDRs 1-3 correspond to the CDRs of adalimumab. In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, 87, or 90, the nucleic acid sequence comprising VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, 88, or 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86, 89, 92, 106, or 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 87, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 88, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 89.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 92.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the light chain comprises a modified light chain variable region (VL) comprising a complementarity determining region (CDR) 1, a VL CDR2, and a VL CDR3. In some aspects, the VL CDRs 1-3 correspond to the CDRs of adalimumab. In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, 96, 99, or 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, 97, 100, 108, 160, or 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95, 98, 101, or 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 96, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 97, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 98.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 100, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 108, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 160, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

In some aspects, the polynucleotide (e.g, an antibody expression cassette) disclosed herein encodes a single-domain antibody (e.g., a nanobody) comprising either (i) a heavy chain variable region (VH) comprising a complementarity determining region (CDR) 1, a VH CDR2, and/or a VH CDR3 or (ii) a light chain variable region (VL) comprising a CDR1, a VL CDR2, and/or a VL CDR3. In some aspects, the encoded VH CDRs and/or VL CDRs are selected from the corresponding CDRs of adalimumab.

In some aspects, the polynucleotide (e.g, an antibody expression cassette) disclosed herein encodes an antibody or antigen-binding fragment thereof comprising the six CDRs of an antibody listed in Tables 1 and 2 (i.e., the three VH CDRs of the antibody listed in Table 1 and the three VL CDRs of the same antibody listed in Table 2). In some aspects, the polynucleotide disclosed herein comprises a nucleotide sequence comprising the nucleotide sequences of the six CDRs of an antibody listed in Tables 3 and 4 (i.e., the three VH CDRs of the antibody listed in Table 3 and the three VL CDRs of the same antibody listed in Table 4).

TABLE 1
Variable Heavy Chain CDR (VH CDR)
Amino Acid Sequences

| VH-CDR1 | VH-CDR2 | VH-CDR3 |
|---|---|---|
| DYAMH | AITWNSGHIDYADSVEG | VSYLSTASSLDY |
| (SEQ ID NO: 1) | (SEQ ID NO: 2) | (SEQ ID NO: 3) |

TABLE 2
Variable Light Chain CDR (VL CDR)
Amino Acid Sequences

| VL-CDR1 | VL-CDR2 | VL-CDR3 |
|---|---|---|
| RASQGIRNYLA | AASTLQS | QRYNRAPYT |
| (SEQ ID NO: 4) | (SEQ ID NO: 5) | (SEQ ID NO: 6) |

TABLE 3
Variable Heavy Chain CDR (VH CDR) Nucleic Acid Sequences

| Description | VH-CDR1 | VH-CDR2 | VH-CDR3 |
|---|---|---|---|
| adalimumab | gattacgcaatgcac | gccatcacatggaactcgggcc | gtgagttatctcagcaccgc |
| #1 | (SEQ ID NO: 84) | atattgactatgctgatagcgtgg | acctctctggactac |
| | | aaggt (SEQ ID NO: 85) | (SEQ ID NO: 86) |
| Codon | gattatgcaatgcac | gccatcacatggaacagtggcc | gtgagttatctcagcaccgc |
| modified | (SEQ ID NO: 87) | atattgactatgctgatagtgtgg | atcctctctggactac |
| adalimumab | | aaggt (SEQ ID NO: 88) | (SEQ ID NO: 89) |
| #2 | | | |
| Codon | gattatgcaatgcac | gccatcacatggaacagtggcc | gtgagttatctcagcacagc |
| modified | (SEQ ID NO: 90) | atattgactatgctgatagtgtgg | atcctctctggactac |
| adalimumab #3 | | aaggt (SEQ ID NO: 91) | (SEQ ID NO: 92) |
| Codon | gattatgcaatgcac | gccatcacatggaacagtggcc | gtgtcatatctcagcaccgc |
| modified | (SEQ ID NO: 90) | atattgactatgctgatagtgtgg | atcctctctggactac |
| adalimumab #4 | | aaggt (SEQ ID NO: 91) | (SEQ ID NO: 106) |
| Codon | gattatgcaatgcac | gccatcacatggaacagtggcc | Gtatcatatctcagcacag |
| modified | (SEQ ID NO: 90) | atattgactatgctgatagtgtgg | catcctctctggactac |
| adalimumab #5 | | aaggt (SEQ ID NO: 91) | (SEQ ID NO: 159) |

TABLE 4
Variable Light Chain CDR (VL CDR) Nucleic Acid Sequences

| Description | VL-CDR1 | VL-CDR2 | VL-CDR3 |
|---|---|---|---|
| adalimumab #1 | agagcttcgcagggaa | gccgctagcacgctgcagtcc | cagcgatataatcgtgcac |
| | taaggaactacctogca | (SEQ ID NO: 94) | cttacaca (SEQ ID |
| | (SEQ ID NO: 93) | | NO: 95) |
| Codon | agagcttcccagggaa | gccgctagcactctgcagtca | cagcggtataatcgcgcac |
| modified | taaggaactacctcgcg | (SEQ ID NO: 97) | cttacaca (SEQ ID |
| adalimumab #2 | (SEQ ID NO: 96) | | NO: 98) |
| Codon | agagcttcccagggaa | gctgctagcactctgcagtca | cagaggtataatagagcac |
| modified | taaggaactacttggca | (SEQ ID NO: 100) | cttacaca (SEQ ID |
| adalimumab #3 | (SEQ ID NO: 99) | | NO: 101) |
| Codon | agagcttcccagggaa | gccgccagcactctgcagtca | cagcggtataatcgcgcac |
| modified | taaggaactacctcgcg | (SEQ ID NO: 108) | cttacaca (SEQ ID |
| adalimumab #4 | (SEQ ID NO: | | NO: 109) |
| | 107) | | |
| Codon | agagcttcccagggaa | gctgccagcactctgcagtca | cagaggtataatagagcac |
| modified | taaggaactacttggca | (SEQ ID NO: 160) | cttacaca (SEQ ID |
| adalimumab #5 | (SEQ ID NO: 99) | | NO: 101) |
| Adalimumab | agagcttcgcagggaa | Gccgccagcacgctgcagtcc | cagcgatataatcgtgcac |
| #6 | taaggaactacctcgca | (SEQ ID NO: 161) | cttacaca (SEQ ID |
| | (SEQ ID NO: 93) | | NO: 95) |

In some aspects, the polynucleotide disclosed herein comprises a nucleic acid sequence listed in Table 5. In some aspects, the polynucleotide disclosed herein encodes an antibody variable heavy chain (VH) sequence listed in Table 5 or antigen-binding fragment thereof.

TABLE 5
Variable Heavy Chain (VH) Nucleic Acid Sequences

| | VH Nucleic Acid Sequence (SEQ ID NO) |
|---|---|
| VH #1 | gaggtgcagctggtcgaaagcggcggagggctcgttcaacccggtcggtccttgagactttcttgcgccg |
| adalimumab | cttcaggcttcacctttgacgattacgcaatgcactgggtgaggcaggcgcctggaaaggggctggagtg |
| VH | ggtaagtgccatcacatggaactcgggccatattgactatgctgatagcgtggaaggtcgcttcactatatc |
| | ccgagacaatgccaaaaactctttatacctgcagatgaattcactacgtgcagaggatacggccgtctatta |
| | ctgtgctaaggtgagttatctcagcaccgcatcctctctggactactggggacaagggacattggttactgt |
| | gagctcc (SEQ ID NO: 7) |
| VH #2 | gaggtgcagctggttgaaagcggcggagggcttgttcaacctggtagatccttgagactttcttgcgccgc |
| Codon modi- | ttcaggcttcacctttgatgattatgcaatgcactgggtgaggcaggcgcctggaaaggggctggagtgg |
| fied | gtaagtgccatcacatggaacagtggccatattgactatgctgatagtgtggaaggtagattcactatatccc |
| adalimumab | gcgacaatgccaaaaactctttatacctgcagatgaattcactacgcgcagaggatactgcggtctattact |
| VH | gtgctaaggtgagttatctcagcaccgcatcctctctggactactggggacaagggacattggttactgtga |
| | gctcc (SEQ ID NO: 8) |
| VH #3 | gaggtgcagctggttgaaagtggaggagggcttgttcaacctggtagatccttgagactttcttgtgctgctt |
| Codon modi- | caggcttcacctttgatgattatgcaatgcactgggtgaggcaggcacctggaaaggggctggagtgggt |
| fied | aagtgccatcacatggaacagtggccatattgactatgctgatagtgtggaaggtagattcactatatccag |
| adalimumab | agacaatgccaaaaactctttatacctgcagatgaattcactaagggcagaggatactgctgtctattactgt |
| VH | gctaaggtgagttatctcagcacagcatcctctctggactactggggacaagggacattggttactgtgagc |
| | tct (SEQ ID NO: 9) |
| VH #4 | gaggtgcagctggttgaaagcggcggagggcttgttcaacctggtagatccttgagactttcttgcgccgc |
| Codon modi- | ttctggcttcacctttgatgattatgcaatgcactgggtgaggcaggcgcctggaaaggggctggagtggg |
| fied | tatcagccatcacatggaacagtggccatattgactatgctgatagtgtggaaggtagattcactatatcccg |
| adalimumab | cgacaatgccaaaaactctttatacctgcagatgaattcactacgcgcagaggatactgcggtctattactgt |
| VH | gctaaggtgtcatatctcagcaccgcatcctctctggactactggggacaagggacattggttactgtgagc |
| | tcc (SEQ ID NO: 102) |
| VH #5 | gaggtgcagctggttgaaagtggaggagggcttgttcaacctggtagatccttgagactttcttgtgctgctt |
| Codon modi- | ctggcttcacctttgatgattatgcaatgcactgggtgaggcaggcacctggaaaggggctggagtgggta |
| fied | tcagccatcacatggaacagtggccatattgactatgctgatagtgtggaaggtagattcactatatccaga |
| adalimumab | gacaatgccaaaaactctttatacctgcagatgaattcactaagggcagaggatactgctgtctattactgtg |
| VH | ctaaggtatcatatctcagcacagcatcctctctggactactggggacaagggacattggttactgtgagct |
| | ct (SEQ ID NO: 143) |
| VH #6 | gaggtgcagctggtcgaaagcggcggagggctcgttcaacccggtcggtccttgagactttcttgcgccg |
| adalimumab | cttctggcttcacctttgacgattacgcaatgcactgggtgaggcaggcgcctggaaaggggctggagtg |
| | ggtatcagccatcacatggaactegggccatattgactatgctgatagcgtggaaggtcgcttcactatatc |
| | ccgagacaatgccaaaaactctttatacctgcagatgaattcactacgtgcagaggatacggccgtctatta |
| | ctgtgctaaggtgtcatatctcagcaccgcatcctctctggactactggggacaagggacattggttactgt |
| | gagctc (SEQ ID NO: 147) |

TABLE 6
Variable Heavy Chain (VH) Amino Acid Sequences
VH Amino Acid Sequence (SEQ ID NO)

EVQLVESGGGLVQPGRSLRLSCAASGFTFDDYAMHWVRQAPGKGLE
WVSAITWNSGHIDYADSVEGRFTISRDNAKNSLYLQMNSLRAEDTAV
YYCAKVSYLSTASSLDYWGQGTLVTVSS (SEQ ID NO: 10)

In some aspects, the polynucleotide disclosed herein comprises a nucleic acid sequence listed in Table 7. In some aspects, the polynucleotide disclosed herein encodes an antibody variable light chain (VL) comprising a sequence listed in Table 8 or antigen-binding fragment thereof.

TABLE 7
Variable Light Chain (VL) Nucleic Acid Sequences

| | VL Nucleic Acid Sequence (SEQ ID NO) |
|---|---|
| VL #1 | gacatccagatgacccaaagcccctcctctctgtcagccagtgtgggcgatcgggtcacaattacttgcaga |
| adalimumab | gcttcgcagggaataaggaactacctcgcatggtatcagcaaaagcctgggaaagcgccaaagttgcttat |
| VL | ctacgccgctagcacgctgcagtccggtgttccgtctcgcttctcaggcagtggaagcgggaccgactttac |
| | attaactatttcctctctgcagcccgaggatgtggccacctattactgtcagcgatataatcgtgcaccttacac |
| | attcggccaaggtaccaaagtagaaatcaag (SEQ ID NO: 11) |
| VL #2 | gacatccagatgacccaaagcccctcctctctgtcagccagtgtgggggatcgcgtcacaattacttgcaga |
| Codon modi- | gcttcccagggaataaggaactacctcgcgtggtatcagcaaaagcctgggaaagcgccaaagttgcttat |
| fied | ctatgccgctagcactctgcagtcaggtgttccttctagattctcaggcagtggaagcgggactgactttacat |
| adalimumab | taactatttcctctctgcagcctgaggatgtggccacctattactgtcagcggtataatcgcgcaccttacacat |
| VL | ttggccaaggtaccaaagtagaaatcaag (SEQ ID NO: 12) |
| VL #3 | gacatccagatgacccaaagcccctcctctctgtcagccagtgtgggggatagagtcacaattacttgcaga |
| Codon modi- | gcttcccagggaataaggaactacttggcatggtatcagcaaaagcctgggaaagctccaaagttgcttatc |
| fied | tatgctgctagcactctgcagtcaggtgttccttctagattctcaggcagtggaagtgggactgactttacatta |
| adalimumab | actatttcctctctgcagcctgaggatgtggccacctattactgtcagaggtataatagagcaccttacacattt |
| VL | ggccaaggtaccaaagtagaaatcaag (SEQ ID NO: 13) |
| VL #4 | gacatccagatgacccaaagcccctcctctctgtcagccagtgtgggggatcgcgtcacaattacttgcaga |
| Codon modi- | gcttcccagggaataaggaactacctcgcgtggtatcagcaaaagcctgggaaagcgccaaagttgcttat |
| fied | ctatgccgccagcactctgcagtcaggtgttcctagtagattctctggcagtggaaggggactgactttaca |
| adalimumab | ttaactatttcctctctgcaacctgaggatgtggccacctattactgtcagcggtataatcgcgcaccttacaca |
| VL | tttggccaaggtaccaaagtagaaatcaag |
| | (SEQ ID NO: 103) |
| VL #5 | gacatccagatgacccaaagcccctcctctctgtcagccagtgtgggggatagagtcacaattacttgcaga |
| Codon modi- | gcttcccagggaataaggaactacttggcatggtatcagcaaaagcctgggaaagctccaaagttgcttatc |
| fied | tatgctgccagcactctgcagtcaggtgttcctagtagattctcaggcagtggaagtgggactgactttacatt |
| adalimumab | aactatttcctctctgcaacctgaggatgtggccacctattactgtcagaggtataatagagcaccttacacatt |
| | tggccaaggtaccaaagtagaaatcaag |
| | (SEQ ID NO: 145) |
| VL #6 | gacatccagatgacccaaagcccctcctctctgtcagccagtgtgggcgatcgggtcacaattacttgcaga |
| adalimumab | gcttcgcagggaataaggaactacctcgcatggtatcagcaaaagcctgggaaagcgccaaagttgcttat |
| | ctacgccgccagcacgctgcagtccggtgttccgtctcgcttctctggcagtggaaggggaccgactttac |
| | attaactatttcctctctgcaacccgaggatgtggccacctattactgtcagcgatataatcgtgcaccttacac |
| | attcggccaaggtaccaaagtag |
| | (SEQ ID NO: 149) |

TABLE 8
Variable Light Chain (VL) Amino Acid Sequences

VL Amino Acid Sequence (SEQ ID NO)
DIQMTQSPSSLSASVGDRVTITCRASQGIRNYLAWYQQKPGKAPKL
LIYAASTLQSGVPSRFSGSGSGTDFTLTISSLQPEDVATYYCQRYNR
APYTFGQGTKVEIK (SEQ ID NO: 14)

In some aspects, the polynucleotide disclosed herein encodes an antibody comprising the VH and the VL of an antibody listed in Tables 8 and 10 (i.e., the VH of the antibody listed in Table 6 and the VL of the same antibody listed in Table 8).

In some aspects, the polynucleotide disclosed herein comprises a nucleic acid sequence listed in Table 9.

TABLE 9
Light Chain Constant Region Nucleic Acid Sequences
Light Chain Constant Region Nucleic Acid Sequence (SEQ ID NO)

cgaactgtggctgcaccatctgtcttcatcttcccgccatctgatgagcagttgaaatctggaactgcctctgtt
gtgtgcctgctgaataacttctatcccagagaggccaaagtacagtggaaggtggataacgccctccaatcg
ggtaactcccaggagagtgtcacagagcaggacagcaaggacagcacctacagcctcagcagcaccctg
acgctgagcaaagcagactacgagaaacacaaagtctacgcctgcgaagtcacccatcagggcctgagct
cgcccgtcacaaagagcttcaacaggggagagtgt (SEQ ID NO: 15)

In some aspects, the polynucleotide disclosed herein comprises a nucleic acid sequence listed in Table 10.

TABLE 10
Heavy Chain Constant Region Nucleic Acid Sequences
Heavy Chain Constant Region Nucleic Acid Sequence (SEQ ID NO)

gcctccaccaagggcccatcggtcttccccctggcaccctcctccaagagcacctctgggggcacagcgg
ccctgggctgcctggtcaaggactacttccccgaaccggtgacggtgtcgtggaactcaggcgccctgac
cagcggcgtgcacaccttcccggctgtcctacagtcctcaggactctactccctcagcagcgtggtgaccgt
gccctccagcagcttgggcacccagacctacatctgcaacgtgaatcacaagcccagcaacaccaaggtg
gacaagaaagttgagcccaaatcttgtgacaaaactcacacatgcccaccgtgcccagcacctgaactcct
ggggggaccgtcagtcttcctcttccccccaaaacccaaggacaccctcatgatctcccggacccctgagg
tcacatgcgtggtggtggacgtgagccacgaagaccctgaggtcaagttcaactggtacgtggacggcgt
ggaggtgcataatgccaagacaaagccgcgggaggagcagtacaacagcacgtaccgtgtggtcagcgt
cctcaccgtcctgcaccaggactggctgaatggcaaggagtacaagtgcaaggtctccaacaaagccctc
ccagcccccatcgagaaaaccatctccaaagccaaagggcagccccgagaaccacaggtgtacaccctg
cccccatcccgggatgagctgaccaagaaccaggtcagcctgacctgcctggtcaaaggcttctatcccag
cgacatcgccgtggagtgggagagcaatgggcagccggagaacaactacaagaccacgcctcccgtgct
ggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgg
gtaaa (SEQ ID NO: 16)

In some aspects, the polynucleotide disclosed herein comprises the nucleic acid sequences listed in Table 11. In some aspects, the polynucleotide disclosed herein encodes an antibody comprising the heavy chain (HC) of an antibody listed in Table 12 or antigen-binding fragment thereof.

TABLE 11
Heavy Chain (HC) Nucleic Acid Sequences

| | HC Nucleic Acid Sequence (SEQ ID NO) |
|---|---|
| HC #1 | gaggtgcagctggtcgaaagcggcggagggctcgttcaacccggtcggtccttgagactttcttgcgccgc |
| | ttcaggcttcacctttgacgattacgcaatgcactgggtgaggcaggcgcctggaaaggggctggagtggg |
| | taagtgccatcacatggaactcgggccatattgactatgctgatagcgtggaaggtcgcttcactatatcccg |
| | agacaatgccaaaaactctttatacctgcagatgaattcactacgtgcagaggatacggccgtctattactgtg |
| | ctaaggtgagttatctcagcaccgcatcctctctggactactggggacaagggacattggttactgtgagctc |
| | cgcctccaccaagggcccatcggtcttccccctggcaccctcctccaagagcacctctgggggcacagcg |
| | gccctgggctgcctggtcaaggactacttccccgaaccggtgacggtgtcgtggaactcaggcgccctga |
| | ccagcggcgtgcacaccttcccggctgtcctacagtcctcaggactctactccctcagcagcgtggtgacc |
| | gtgccctccagcagcttgggcacccagacctacatctgcaacgtgaatcacaagcccagcaacaccaagg |
| | tggacaagaaagttgagcccaaatcttgtgacaaaactcacacatgcccaccgtgcccagcacctgaactc |
| | ctggggggaccgtcagtcttcctcttccccccaaaacccaaggacaccctcatgatctcccggacccctga |
| | ggtcacatgcgtggtggtggacgtgagccacgaagaccctgaggtcaagttcaactggtacgtggacggc |
| | gtggaggtgcataatgccaagacaaagccgcgggaggagcagtacaacagcacgtaccgtgtggtcagc |
| | gtcctcaccgtcctgcaccaggactggctgaatggcaaggagtacaagtgcaaggtctccaacaaagccct |
| | cccagcccccategagaaaaccatctccaaagccaaagggcagccccgagaaccacaggtgtacaccct |
| | gcccccatcccgggatgagctgaccaagaaccaggtcagcctgacctgcctggtcaaaggcttctatccca |
| | gcgacatcgccgtggagtgggagagcaatgggcagccggagaacaactacaagaccacgcctcccgtg |
| | ctggactccgacggctccttcttcctctacagcaagctcaccgtggacaagagcaggtggcagcagggga |
| | acgtcttctcatgctccgtgatgcatgaggctctgcacaaccactacacgcagaagagcctctccctgtctcc |
| | gggtaaatga (SEQ ID NO: 17) |
| HC #2 | gaggtgcagctggttgaaagcggcggagggcttgttcaacctggtagatccttgagactttcttgcgccgctt |
| | caggcttcacctttgatgattatgcaatgcactgggtgaggcaggcgcctggaaaggggctggagtgggta |
| | agtgccatcacatggaacagtggccatattgactatgctgatagtgtggaaggtagattcactatatcccgcg |
| | acaatgccaaaaactctttatacctgcagatgaattcactacgcgcagaggatactgcggtctattactgtgct |
| | aaggtgagttatctcagcaccgcatcctctctggactactggggacaagggacattggttactgtgagctcc |
| | gcctccaccaagggcccaagtgtcttccccctggcaccctcctccaagagcacctctgggggcacagcgg |
| | ccctgggctgcctggtcaaggactacttccctgaacctgtgacagtgtcttggaactcaggcgccctgacca |
| | gcggagtgcacaccttcccagctgtcctacagtcctcaggactctactccctcagcagtgtggtgactgtgc |
| | cctccagcagcttgggcacccagacctacatctgcaatgtgaatcacaagcccagcaacaccaaggtgga |
| | caagaaagttgagcccaaatcttgtgacaaaactcacacatgcccaccttgcccagcacctgaactcctggg |
| | gggaccatcagtcttcctcttccccccaaaacccaaggacaccctcatgatctcccgcacccctgaggtcac |
| | atgtgtggtggtggatgtgagccatgaagaccctgaggtcaagttcaactggtatgtggatggtgtggaggt |
| | gcataatgccaagacaaagccgcgggaggagcagtacaacagcacatacagagtggtctctgtcctcact |
| | gtcctgcaccaggactggctgaatggcaaggagtacaagtgcaaggtctccaacaaagccctcccagccc |
| | ccattgagaaaaccatctccaaagccaaagggcagcccagggaaccacaggtgtacaccctgcccccatc |
| | ccgcgatgagctgaccaagaaccaggtcagcctgacctgcctggtcaaaggcttctatcccagtgacattg |
| | ctgtggagtgggagagcaatgggcagcctgagaacaactacaagaccacacctccagtgctggactctga |
| | tggctccttcttcctctacagcaagctcactgtggacaagagcaggtggcagcaggggaatgtcttctcatgc |
| | agtgtgatgcatgaggctctgcacaaccactacacacagaagagcctctccctgtctcctggtaaatga |
| | (SEQ ID NO: 18) |
| HC #3 | gaggtgcagctggttgaaagtggaggagggcttgttcaacctggtagatccttgagactttcttgtgctgcttc |
| | aggcttcacctttgatgattatgcaatgcactgggtgaggcaggcacctggaaaggggctggagtgggtaa |
| | gtgccatcacatggaacagtggccatattgactatgctgatagtgtggaaggtagattcactatatccagaga |
| | caatgccaaaaactctttatacctgcagatgaattcactaagggcagaggatactgctgtctattactgtgcta |
| | aggtgagttatctcagcacagcatcctctctggactactggggacaagggacattggttactgtgagctctgc |
| | ctccaccaagggcccaagtgtcttccccctggcaccctcctccaagagcacctctgggggcacagcagcc |
| | ctgggctgcctggtcaaggactacttccctgaacctgtgactgtgtcttggaactcaggtgccctgaccagtg |
| | gagtgcacaccttcccagctgtcctacagtcctcaggactctactccctcagctctgtggtgacagtgccctc |
| | cagcagcttgggcacccagacctacatctgcaatgtgaatcacaagcccagcaacaccaaggtggacaag |
| | aaagttgagcccaaatcttgtgacaaaactcacacatgcccaccttgcccagcacctgaactcctgggggg |
| | accatcagtcttcctcttccccccaaaacccaaggacaccctcatgatctccagaacccctgaggtcacatgt |
| | gtggtggtggatgtgagccatgaagaccctgaggtcaagttcaactggtatgtggatggtgtggaggtgcat |
| | aatgccaagacaaagcctagggaggagcagtacaacagcacttacagagtggtcagtgtcctcacagtcct |
| | gcaccaggactggctgaatggcaaggagtacaagtgcaaggtctccaacaaagccctcccagcccccatt |
| | gagaaaaccatctccaaagccaaagggcagcccagggaaccacaggtgtacaccctgcccccatccaga |
| | gatgagctgaccaagaaccaggtcagcctgacctgcctggtcaaaggcttctatccctcagacattgctgtg |
| | gagtgggagagcaatgggcagccagagaacaactacaagaccactcctcctgtgctggactctgatggct |
| | ccttcttcctctacagcaagctcacagtggacaagagcaggtggcagcaggggaatgtcttctcatgcagtg |
| | tgatgcatgaggctctgcacaaccactacactcagaagagcctctccctgtctccaggtaaatga (SEQ |
| | ID NO: 19) |
| HC #4 | gaggtgcagctggttgaaagcggcggagggcttgttcaacctggtagatccttgagactttcttgcgccgctt |
| | ctggcttcacctttgatgattatgcaatgcactgggtgaggcaggcgcctggaaaggggctggagtgggtat |
| | cagccatcacatggaacagtggccatattgactatgctgatagtgtggaaggtagattcactatatcccgcga |
| | caatgccaaaaactctttatacctgcagatgaattcactacgcgcagaggatactgcggtctattactgtgcta |
| | aggtgtcatatctcagcaccgcatcctctctggactactggggacaagggacattggttactgtgagctccgc |
| | ctccaccaagggcccaagtgtcttccccctggcaccctcctccaagagcacctctgggggcacagcggcc |
| | ctgggctgcctggtcaaggactacttccctgaacctgtgacagtgtcttggaactcaggcgccctgaccagc |
| | ggagtgcacaccttcccagctgtcctacagtcctcaggactctactccctcagcagtgtggtgactgtgccct |
| | ccagcagcttgggcacccagacctacatctgcaatgtgaatcacaagcccagcaacaccaaggtggacaa |
| | gaaagttgagcccaaatcttgtgacaaaactcacacatgcccaccttgcccagcacctgaactcctggggg |
| | gaccatcagtcttcctcttccccccaaaacccaaggacaccctcatgatctcccgcacccctgaggtcacat |
| | gtgtggtggtggatgtgagccatgaagaccctgaggtcaagttcaactggtatgtggatggtgtggaggtgc |
| | ataatgccaagacaaagccgcgggaggagcagtacaacagcacatacagagtggtctctgtcctcactgtc |
| | ctgcaccaggactggctgaatggcaaggagtacaagtgcaaggtctccaacaaagccctcccagccccta |
| | ttgagaaaacaatctccaaagccaaagggcagcccagggaaccacaggtgtacaccctgcccccatccc |
| | gcgatgagctgaccaagaaccaggtctccctgacctgcctggtcaaaggcttctatcccagtgacattgctg |
| | tggagtgggagagcaatgggcagcctgagaacaactacaagaccacacctccagtgctggactctgatgg |
| | ctccttcttcctctactccaagctcactgtggacaagagcaggtggcagcaggggaatgtcttctcatgcagt |
| | gtgatgcatgaggctctgcacaaccactacacacagaagagcctctccctgtctcctggtaaa |
| | (SEQ ID NO: 110) |
| HC #5 | gaggtgcagctggttgaaagtggaggagggcttgttcaacctggtagatccttgagactttcttgtgctgcttc |
| | tggcttcacctttgatgattatgcaatgcactgggtgaggcaggcacctggaaaggggctggagtgggtatc |
| | agccatcacatggaacagtggccatattgactatgctgatagtgtggaaggtagattcactatatccagagac |
| | aatgccaaaaactctttatacctgcagatgaattcactaagggcagaggatactgctgtctattactgtgctaa |
| | ggtatcatatctcagcacagcatcctctctggactactggggacaagggacattggttactgtgagctctgcc |
| | tccaccaagggcccaagtgtcttccccctggcaccctcctccaagagcacctctgggggcacagcagccc |
| | tgggctgcctggtcaaggactacttccctgaacctgtgactgtgtcttggaactcaggtgccctgaccagtgg |
| | agtgcacaccttcccagctgtcctacagtcctcaggactctactccctgagctctgtggtgacagtgccctcc |
| | agcagcttgggcacccagacctacatctgcaatgtgaatcacaagcccagcaacaccaaggtggacaaga |
| | aagttgagcccaaatcttgtgacaaaactcacacatgcccaccttgcccagcacctgaactcctgggggga |
| | ccatcagtcttcctcttccccccaaaacccaaggacaccctcatgatctccagaacccctgaggtcacatgtg |
| | tggtggtggatgtgagccatgaagaccctgaggtcaagttcaactggtatgtggatggtgtggaggtgcata |
| | atgccaagacaaagcccagggaggagcagtacaacagcacttacagagtggtcagtgtcctcacagtcct |
| | gcaccaggactggctgaatggcaaggagtacaagtgcaaggtctccaacaaagccctcccagcccccatt |
| | gagaaaaccatctccaaagccaaagggcagcccagggaaccacaggtgtacaccctgcccccatccaga |
| | gatgagctgaccaagaaccaggtctccctgacctgcctggtcaaaggcttctatccctctgacattgctgtgg |
| | agtgggagagcaatgggcagccagagaacaactacaagaccactcctcctgtgctggactctgatggctc |
| | cttcttcctctactccaagctcacagtggacaagagcaggtggcagcaggggaatgtcttctcatgcagtgtg |
| | atgcatgaggctctgcacaaccactacactcagaagagcctctccctgtctcctggcaaa |
| | (SEQ ID NO: 142) |
| HC #6 | gaggtgcagctggtcgaaagcggcggagggctcgttcaacccggtcggtccttgagactttcttgcgccgc |
| | ttctggcttcacctttgacgattacgcaatgcactgggtgaggcaggcgcctggaaaggggctggagtggg |
| | tatcagccatcacatggaactcgggccatattgactatgctgatagcgtggaaggtcgcttcactatatcccg |
| | agacaatgccaaaaactctttatacctgcagatgaattcactacgtgcagaggatacggccgtctattactgtg |
| | ctaaggtgtcatatctcagcaccgcatcctctctggactactggggacaagggacattggttactgtgagctc |
| | cgcctccaccaagggcccatcggtcttccccctggcaccctcctccaagagcacctctgggggcacagcg |
| | gccctgggctgcctggtcaaggactacttccccgaaccggtgacggtgtcgtggaactcaggcgccctga |
| | ccagcggcgtgcacaccttcccggctgtcctacagtcctcaggactctactccctcagcagcgtggtgacc |
| | gtgccctccagcagcttgggcacccagacctacatctgcaacgtgaatcacaagcccagcaacaccaagg |
| | tggacaagaaagttgagcccaaatcttgtgacaaaactcacacatgcccaccgtgcccagcacctgaactc |
| | ctggggggaccgtcagtcttcctcttccccccaaaacccaaggacaccctcatgatctcccggacccctga |
| | ggtcacatgcgtggtggtggacgtgagccacgaagaccctgaggtcaagttcaactggtacgtggacggc |
| | gtggaggtgcataatgccaagacaaagccgcgggaggagcagtacaacagcacgtaccgtgtggtcagc |
| | gtcctcaccgtcctgcaccaggactggctgaatggcaaggagtacaagtgcaaggtctccaacaaagccct |
| | cccagcccctatcgagaaaacaatctccaaagccaaagggcagccccgagaaccacaggtgtacaccct |
| | gcccccatcccgggatgagctgaccaagaaccaagtcagcctgacctgcctggtcaaaggcttctatccca |
| | gcgacatcgccgtggagtgggagagcaatgggcagccggagaacaactacaagaccacgcctcccgtg |
| | ctggactccgacggctccttcttcctctactccaagctcaccgtggacaagagcaggtggcagcaggggaa |
| | cgtcttctcatgctccgtgatgcatgaggctctgcacaaccactacacgcagaagagcctctccctgtctccg |
| | ggtaaa (SEQ ID NO: 146) |
| HC #7 | gaggtgcagctggttgaaagcggcggagggcttgttcaacctggtagatccttgagactttcttgcgccgctt |
| | ctggcttcacctttgatgattatgcaatgcactgggtgaggcaggcgcctggaaaggggctggagtgggtat |
| | cagccatcacatggaacagtggccatattgactatgctgatagtgtggaaggtagattcactatatcccgcga |
| | caatgccaaaaactctttatacctgcagatgaattcactacgcgcagaggatactgcggtctattactgtgcta |
| | aggtgtcatatctcagcaccgcatcctctctggactactggggacaagggacattggttactgtgagctccgc |
| | ctccaccaagggcccaagtgtcttccccctggcaccctcctccaagagcacctctgggggcacagcggcc |
| | ctgggctgcctggtcaaggactacttccctgaacctgtgacagtgtcttggaactcaggcgccctgaccagc |
| | ggagtgcacaccttcccagctgtcctacagtcctcaggactctactccctcagcagtgtggtgactgtgccct |
| | ccagcagcttgggcacccagacctacatctgcaatgtgaatcacaagcccagcaacaccaaggtggacaa |
| | gaaagttgagcccaaatcttgtgacaaaactcacacatgcccaccttgcccagcacctgaactcctggggg |
| | gaccatcagtcttcctcttccccccaaaacccaaggacaccctcatgatctcccgcacccctgaggtcacat |
| | gtgtggtggtggatgtgagccatgaagaccctgaggtcaagttcaactggtatgtggatggtgtggaggtgc |
| | ataatgccaagacaaagccgcgggaggagcagtacaacagcacatacagagtggtctctgtcctcactgtc |
| | ctgcaccaggactggctgaatggcaaggagtacaagtgcaaggtctccaacaaagccctcccagccccta |
| | ttgagaaaacaatctccaaagccaaagggcagcccagggaaccacaggtgtacaccctgcccccatccc |
| | gcgatgagctgaccaagaaccaggtctccctgacctgcctggtcaaaggcttctatcccagtgacattgctg |
| | tggagtgggagagcaatgggcagcctgagaacaactacaagaccacacctccagtgctggactctgatgg |
| | ctccttcttcctctactccaagctcactgtggacaagagcaggtggcagcaggggaatgtcttctcatgcagt |
| | gtgatgcatgaggctctgcacaaccactacacacagaagagcctctccctgtctcctggcaaa |
| | (SEQ ID NO: 150) |
| HC #8 | gaggtgcagctggtcgaaagcggcggagggctcgttcaacccggtcggtccttgagactttcttgcgccgc |
| | ttctggcttcacctttgacgattacgcaatgcactgggtgaggcaggcgcctggaaaggggctggagtggg |
| | tatcagccatcacatggaactcgggccatattgactatgctgatagcgtggaaggtcgcttcactatatcccg |
| | agacaatgccaaaaactctttatacctgcagatgaattcactacgtgcagaggatacggccgtctattactgtg |
| | ctaaggtgtcatatctcagcaccgcatcctctctggactactggggacaagggacattggttactgtgagctc |
| | cgcctccaccaagggcccatcggtcttccccctggcaccctcctccaagagcacctctgggggcacagcg |
| | gccctgggctgcctggtcaaggactacttccccgaaccggtgacggtgtcgtggaactcaggcgccctga |
| | ccagcggcgtgcacaccttcccggctgtcctacagtcctcaggactctactccctcagcagcgtggtgacc |
| | gtgccctccagcagcttgggcacccagacctacatctgcaacgtgaatcacaagcccagcaacaccaagg |
| | tggacaagaaagttgagcccaaatcttgtgacaaaactcacacatgcccaccgtgcccagcacctgaactc |
| | ctggggggaccgtcagtcttcctcttccccccaaaacccaaggacaccctcatgatctcccggacccctga |
| | ggtcacatgcgtggtggtggacgtgagccacgaagaccctgaggtcaagttcaactggtacgtggacggc |
| | gtggaggtgcataatgccaagacaaagccgcgggaggagcagtacaacagcacgtaccgtgtggtcagc |
| | gtcctcaccgtcctgcaccaggactggctgaatggcaaggagtacaagtgcaaggtctccaacaaagccct |
| | cccagcccctategagaaaacaatctccaaagccaaagggcagccccgagaaccacaggtgtacaccct |
| | gcccccatcccgggatgagctgaccaagaaccaagtcagcctgacctgcctggtcaaaggcttctatccca |
| | gcgacatcgccgtggagtgggagagcaatgggcagccggagaacaactacaagaccacgcctcccgtg |
| | ctggactccgacggctccttcttcctctactccaagctcaccgtggacaagagcaggtggcagcaggggaa |
| | cgtcttctcatgctccgtgatgcatgaggctctgcacaaccactacacgcagaagagcctctccctgtctccg |
| | ggcaaa |
| | (SEQ ID NO: 151) |

TABLE 12
Heavy Chain (HC) Amino Acid Sequences

HC Amino Acid Sequence (SEQ ID NO)
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPELLGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPAPIEKTISKAKGQPREPQVYTLPPSRD
ELTKNQVSLTCLVKGFYPSDIAVEWESNGQPENNYKTTPPVLDSD
GSFFLYSKLTVDKSRWQQGNVFSCSVMHEALHNHYTQKSLSLSPG
K (SEQ ID NO: 20)

In some aspects, the polynucleotide disclosed herein comprises a nucleic acid sequence listed in Table 13. In some aspects, an antibody the polynucleotide disclosed herein encodes comprising the light chain (LC) of an antibody listed in Table 14 or antigen-binding fragment thereof.

TABLE 13
Light Chain (LC) Nucleic Acid Sequences

| | LC Nucleic Acid Sequence (SEQ ID NO) |
|---|---|
| LC #1 | gacatccagatgacccaaagcccctcctctctgtcagccagtgtgggcgatcgggtcacaattacttgcaga |
| | gcttcgcagggaataaggaactacctcgcatggtatcagcaaaagcctgggaaagcgccaaagttgcttat |
| | ctacgccgctagcacgctgcagtccggtgttccgtctcgcttctcaggcagtggaagcgggaccgactttac |
| | attaactatttcctctctgcagcccgaggatgtggccacctattactgtcagcgatataatcgtgcaccttacac |
| | attcggccaaggtaccaaagtagaaatcaagcgaactgtggctgcaccatctgtcttcatcttcccgccatct |
| | gatgagcagttgaaatctggaactgcctctgttgtgtgcctgctgaataacttctatcccagagaggccaaag |
| | tacagtggaaggtggataacgccctccaatcgggtaactcccaggagagtgtcacagagcaggacagca |
| | aggacagcacctacagcctcagcagcaccctgacgctgagcaaagcagactacgagaaacacaaagtct |
| | acgcctgcgaagtcacccatcagggcctgagctcgcccgtcacaaagagcttcaacaggggagagtgttg |
| | a (SEQ ID NO: 21) |
| LC #2 | gacatccagatgacccaaagcccctcctctctgtcagccagtgtgggggatcgcgtcacaattacttgcaga |
| | gcttcccagggaataaggaactacctcgcgtggtatcagcaaaagcctgggaaagcgccaaagttgcttat |
| | ctatgccgctagcactctgcagtcaggtgttccttctagattctcaggcagtggaagcgggactgactttacat |
| | taactatttcctctctgcagcctgaggatgtggccacctattactgtcagcggtataatcgcgcaccttacacat |
| | ttggccaaggtaccaaagtagaaatcaagcggactgtggctgcaccatctgtcttcatcttcccgccatctga |
| | tgagcagttgaaatctggaactgcctctgttgtgtgcctgctgaataacttctatcccagagaggccaaagtac |
| | agtggaaggtggataatgccctccaaagtggtaactcccaggagagtgtcacagagcaggacagcaagg |
| | acagcacctacagcctcagcagcaccctgaccctgagcaaagcagactatgagaaacacaaagtctacgc |
| | gtgtgaagtcacccatcagggcctgagctccccagtcacaaagagcttcaacaggggagagtgttga |
| | (SEQ ID NO: 22) |
| LC #3 | gacatccagatgacccaaagcccctcctctctgtcagccagtgtgggggatagagtcacaattacttgcaga |
| | gcttcccagggaataaggaactacttggcatggtatcagcaaaagcctgggaaagctccaaagttgcttatc |
| | tatgctgctagcactctgcagtcaggtgttccttctagattctcaggcagtggaagtgggactgactttacatta |
| | actatttcctctctgcagcctgaggatgtggccacctattactgtcagaggtataatagagcaccttacacattt |
| | ggccaaggtaccaaagtagaaatcaagaggactgtggctgcaccatctgtcttcatcttccccccatctgatg |
| | agcagttgaaatctggaactgcctctgttgtgtgcctgctgaataacttctatcccagagaggccaaagtaca |
| | gtggaaggtggataatgccctccaatcaggtaactcccaggagagtgtcacagagcaggacagcaagga |
| | cagcacctacagcctcagcagcaccctgaccctgagcaaagcagactatgagaaacacaaagtctatgcc |
| | tgtgaagtcacccatcagggcctgagctccccagtcacaaagagcttcaacaggggagagtgttga |
| | (SEQ ID NO: 23) |
| LC #4 | gacatccagatgacccaaagcccctcctctctgtcagccagtgtgggggatcgcgtcacaattacttgcaga |
| | gcttcccagggaataaggaactacctcgcgtggtatcagcaaaagcctgggaaagcgccaaagttgcttat |
| | ctatgccgccagcactctgcagtcaggtgttcctagtagattctctggcagtggaagcgggactgactttaca |
| | ttaactatttcctctctgcaacctgaggatgtggccacctattactgtcagcggtataatcgcgcaccttacaca |
| | tttggccaaggtaccaaagtagaaatcaagcggactgtggctgcaccatctgtcttcatcttcccgccatctg |
| | atgagcagttgaaatctggaactgcctctgttgtgtgcctgctgaataacttctatcccagagaggccaaagta |
| | cagtggaaggtggataatgccctccaaagtggcaactcccaggagagtgtcacagagcaggacagcaag |
| | gacagcacctacagcctcagcagcaccctgaccctgagcaaagcagactatgagaaacacaaagtctacg |
| | cgtgtgaagtcacccatcagggcctgagctccccagtcacaaagagcttcaacaggggagagtgttga |
| | (SEQ ID NO: 111) |
| LC #5 | gacatccagatgacccaaagcccctcctctctgtcagccagtgtgggggatagagtcacaattacttgcaga |
| | gcttcccagggaataaggaactacttggcatggtatcagcaaaagcctgggaaagctccaaagttgcttatc |
| | tatgctgccagcactctgcagtcaggtgttcctagtagattctcaggcagtggaagtgggactgactttacatt |
| | aactatttcctctctgcaacctgaggatgtggccacctattactgtcagaggtataatagagcaccttacacatt |
| | tggccaaggtaccaaagtagaaatcaagaggactgtggctgcaccatctgtcttcatcttccccccatctgat |
| | gagcagttgaaatctggaactgcctctgttgtgtgcctgctgaataacttctatcccagagaggccaaagtac |
| | agtggaaggtggataatgccctccaatcaggcaactcccaggagagtgtcacagagcaggacagcaagg |
| | acagcacctacagcctcagcagcaccctgaccctgagcaaagcagactatgagaaacacaaagtctatgc |
| | ctgtgaagtcacccatcagggcctgagctccccagtcacaaagagcttcaacaggggagagtgttga |
| | (SEQ ID NO: 144) |
| LC #6 | gacatccagatgacccaaagcccctcctctctgtcagccagtgtgggcgatcgggtcacaattacttgcaga |
| | gcttcgcagggaataaggaactacctcgcatggtatcagcaaaagcctgggaaagcgccaaagttgcttat |
| | ctacgccgccagcacgctgcagtccggtgttccgtctcgcttctctggcagtggaagcgggaccgactttac |
| | attaactatttcctctctgcaacccgaggatgtggccacctattactgtcagcgatataatcgtgcaccttacac |
| | attcggccaaggtaccaaagtagaaatcaagcgaactgtggctgcaccatctgtcttcatcttcccgccatct |
| | gatgagcagttgaaatctggaactgcctctgttgtgtgcctgctgaataacttctatcccagagaggccaaag |
| | tacagtggaaggtggataacgccctccaatcgggcaactcccaggagagtgtcacagagcaggacagca |
| | aggacagcacctacagcctcagcagcaccctgacgctgagcaaagcagactacgagaaacacaaagtct |
| | acgcctgcgaagtcacccatcagggcctgagctcgcccgtcacaaagagcttcaacaggggagagtgttg |
| | a (SEQ ID NO: 148) |

TABLE 14
Light Chain (LC) Amino Acid Sequences

LC Amino Acid Sequence (SEQ ID NO)
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC (SEQ ID
NO: 24)

In some aspects, the polynucleotide disclosed herein comprises a nucleic acid listed in Tables 11 and 13. In some aspects, the polynucleotide disclosed herein encodes an antibody comprising a HC and a LC of an antibody listed in Tables 12 and 14 (i.e., the HC of the antibody listed in Table 12 and the LC of the same antibody listed in Table 14.

In some aspects, the therapeutic proteins used in the methods disclosed herein are antibodies, (e.g., monoclonal antibodies or antigen-binding fragments thereof) having the VH, VL, HC, and/or LC sequences of adalimumab as well as antibodies having at least 80% identity, at least about 85%, at least about 90%, at least about 95%, at least about 96%, at least about 97%, at least about 98%, at least about 99%, or about 100% identity to the corresponding VH, VL, HC, and/or LC sequences.

In some aspects, the polynucleotide (e.g, an antibody expression cassette) also comprises a leader sequence operably linked to the nucleic acid sequence encoding the heavy chain and/or the nucleic acid sequence encoding the light chain. In some aspects, the leader sequence is an IL-2 or IL-10 leader sequence. In some aspects, the IL-2 leader sequence has a nucleotide sequence at least 85%, 86%, 87%, 88%, 89%, 90%, 91%, 92%, 93%, 94%, 95%, 96%, 97%, 98%, 99%, or 100% identical to SEQ ID NO: 29 or 113 (or nucleotides 1-60 of SEQ ID NO: 49 or nucleotides 3401-3459 of SEQ ID NO: 64). In some aspects, the IL-10 leader sequence has a nucleotide sequence at least 85%, 86%, 87%, 88%, 89%, 90%, 91%, 92%, 93%, 94%, 95%, 96%, 97%, 98%, 99%, or 100% identical to SEQ ID NO: 30 or 112 (or nucleotides 1-53 of SEQ ID NO: 56 or nucleotides 1898-1950 of SEQ ID NO: 64).

### II.A.2. Antigen Binding Fragments

In some aspects, an antigen-binding fragment of an antibody described herein, such as e.g., adalimumab, is encoded by a polynucleotide (e.g, an antibody expression cassette) disclosed herein. Exemplary antigen-binding fragments include but are not limited to Fab, Fab′, F(ab′)2, and scFv, wherein the Fab, Fab′, F(ab′)2, or scFv comprises a heavy chain variable region sequence and a light chain variable region sequence of adalimumab as described herein. A Fab, Fab′, F(ab′)2, or scFv can be produced by any technique known to those of skill in the art. In some aspects, an antigen-binding fragment, such as a Fab, Fab′, F(ab′)2, or scFv, further comprises a moiety that extends the half-life of the antibody in vivo. The moiety is also termed a “half-life extending moiety.” Any moiety known to those of skill in the art for extending the half-life of a an antigen-binding fragment, such as a Fab, Fab′, F(ab′)2, or scFv, in vivo can be used. For example, the half-life extending moiety can include an Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, an antigen-binding fragment, such as an Fab, Fab′, F(ab′)2, or scFv, can be modified by the addition of one or more C-terminal amino acids for attachment of the half-life extending moiety. In some aspects the half-life extending moiety is polyethylene glycol or human serum albumin. In some aspects, an antigen-binding fragment, such as a Fab, Fab′, F(ab′)2, or scFv, is fused to a Fc region.

In some aspects, the antibody or antigen-binding fragments thereof specifically binds to a tumor necrosis factor (TNF), such as human TNF-α. In some aspects, the encoded anti-TNF antibody comprises the amino acid sequence of adalimumab or an antigen-binding fragment thereof.

In some aspects, the antibody (e.g., a monoclonal antibody) or antigen binding fragment thereof disclosed herein is modified so that it has enhanced half life and/or reduced toxicity.

In some aspects, the encoded antibody or antigen-binding fragment thereof is a human antibody, a humanized antibody or a chimeric antibody. In some aspects, the antibody or antigen-binding fragment thereof can be selected from any class of immunoglobulins, including IgM, IgG, IgD, IgA and IgE, and any isotype, including IgG1, IgG2, IgG3 and IgG4. In some aspects, the antibody or antigen-binding fragment thereof is bispecific or multispecific.

### II.A.3. Polynucleotides

In certain aspects, provided herein are polynucleotides (e.g, an antibody expression cassette) comprising a nucleotide sequence encoding an antibody or antigen-binding fragment thereof described herein or a domain thereof (e.g., a light chain, a heavy chain, a variable light chain region and/or variable heavy chain region) that specifically binds to a tumor necrosis factor (TNF) antigen-binding fragments thereof, or any combination thereof, and vectors, e.g., vectors comprising such polynucleotides for expression in a cell, e.g., a secretory cell.

In some aspects, provided herein are polynucleotides (e.g, an antibody expression cassette) comprising nucleotide sequences encoding antibodies or antigen-binding fragments thereof, which specifically bind to a tumor necrosis factor (TNF) antigen-binding fragments thereof, or any combination thereof and comprise an amino acid sequence as described herein, as well as antibodies or antigen-binding fragments that compete with such antibodies or antigen-binding fragments for binding a tumor necrosis factor (TNF) antigen-binding fragments thereof, or any combination thereof (e.g., in a dose-dependent manner), or which bind to the same epitope as that of such antibodies or antigen-binding fragments. In some aspects, the polynucleotides encodes an antibody that competes for binding to the same epitope as adalimumab.

Also provided herein is a polynucleotide (e.g, an antibody expression cassette) comprising a nucleotide sequence encoding a polypeptide comprising a sequence of any one of SEQ ID NOs: 1-6, 10, 14, 20, or 24. In some aspects, an antibody or antigen-binding fragment thereof comprising the polypeptide specifically binds to a tumor necrosis factor (TNF) antigen-binding fragments thereof, or any combination thereof.

Also provided herein are kits, vectors, or host cells comprising (i) a first polynucleotide comprising a expression cassette comprising a nucleotide sequence encoding any of SEQ ID NOs: 62-77, 115-141, or 153-158 and (ii) a delivery vector.

In some aspects, provided herein are polynucleotides (e.g, an antibody expression cassette) comprising a nucleotide sequence comprising three VH domain CDRs, e.g., a nucleotide sequence containing VH CDR1, VH CDR2, and VH CDR3 of any one of the antibodies described herein (e.g., see Table 3), e.g., wherein the three VH domain CDRs are in the context of a VH. In some aspects, provided herein are polynucleotides (e.g, an antibody expression cassette) comprising a nucleotide sequence comprising three VL domain CDRs, e.g., a nucleotide sequence containing VL CDR1, VL CDR2, and VL CDR3 of any one of the antibodies described herein (e.g., see Table 4), e.g., wherein the three VL domain CDRs are in the context of a VL. In some aspects, provided herein are polynucleotides (e.g, an antibody expression cassette)(or combinations of polynucleotides) comprising a nucleotide sequence comprising an antibody or antigen-binding fragment thereof comprising (i) three VH domain CDRs, e.g., a nucleotide sequence containing VH CDR1, VH CDR2, and VH CDR3 of any one of antibodies described herein (e.g., see Table 3) e.g., wherein the three VH domain CDRs are in the context of a VH and (ii) three VL domain CDRs, e.g., a nucleotide sequence containing VL CDR1, VL CDR2, and VL CDR3 of any one of antibodies described herein (e.g., see Table 4), e.g., wherein the three VL domain CDRs are in the context of a VL.

In some aspects, provided herein are polynucleotides (e.g, an antibody expression cassette) comprising a nucleotide sequence encoding three VH domain CDRs, e.g., a polypeptide containing VH CDR1, VH CDR2, and VH CDR3 of any one of the antibodies described herein (e.g., see Table 1), e.g., wherein the three VH domain CDRs are in the context of a VH. In some aspects, provided herein are polynucleotides (e.g, an antibody expression cassette) comprising a nucleotide sequence encoding three VL domain CDRs, e.g., a polypeptide containing VL CDR1, VL CDR2, and VL CDR3 of any one of the antibodies described herein (e.g., see Table 2), e.g., wherein the three VL domain CDRs are in the context of a VL. In some aspects, provided herein are polynucleotides (e.g, an antibody expression cassette) (or combinations of polynucleotides) comprising a nucleotide sequence encoding an antibody or antigen-binding fragment thereof comprising (i) three VH domain CDRs, e.g., a polypeptide containing VH CDR1, VH CDR2, and VH CDR3 of any one of antibodies described herein (e.g., see Table 1) e.g., wherein the three VH domain CDRs are in the context of a VH and (ii) three VL domain CDRs, e.g., a polypeptide containing VL CDR1, VL CDR2, and VL CDR3 of any one of antibodies described herein (e.g., see Table 2), e.g., wherein the three VL domain CDRs are in the context of a VL.

In some aspects, the heavy chain comprises a modified heavy chain variable region (VH) comprising a complementarity determining region (CDR) 1, a VH CDR2, and a VH CDR3. In some aspects, the modified VH CDRs 1-3 correspond to the CDRs of adalimumab. In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, 87, or 90, the nucleic acid sequence comprising VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, 88, or 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86, 89, 92, 106, or 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 86.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 87, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 88, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 89.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 92.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 90, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 91, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 159.

In some aspects, the nucleic acid sequence comprising VH CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 84, the VH CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 85, and the nucleic acid sequence comprising VH CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 106.

In some aspects, the light chain comprises a modified light chain variable region (VL) comprising a complementarity determining region (CDR) 1, a VL CDR2, and a VL CDR3. In some aspects, the VL CDRs 1-3 correspond to the CDRs of adalimumab. In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, 96, 99, or 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, 97, 100, 108, 160, or 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95, 98, 101, or 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 94, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 96, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 97, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 98.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 100, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 107, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 108, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 109.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 99, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 160, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 101.

In some aspects, the nucleic acid sequence comprising VL CDR1 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 93, the nucleic acid sequence comprising VL CDR2 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 161, and the nucleic acid sequence comprising VL CDR3 has a nucleotide sequence with at least 85%, 86%, 87%, 88%, 89%, 90%, 91%, 92%, 93%, 94%, 95%, 96%, 97%, 98%, 99%, or 100% identity to SEQ ID NO: 95.

Also provided herein are polynucleotides (e.g, an antibody expression cassette) encoding an antibody or antigen-binding fragment thereof described herein or a domain thereof that are optimized, e.g., by codon/RNA optimization, replacement with heterologous signal sequences, and elimination of mRNA instability elements. Methods to generate optimized nucleic acids encoding an antibody or antigen-binding fragment thereof described herein or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, each of which is incorporated herein by reference in its entirety.

A polynucleotide (e.g, an antibody expression cassette) encoding an anti-TNFalpha antibody or antigen-binding fragment thereof described herein or a domain thereof can be generated from nucleic acid from a suitable source using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Polynucleotides (e.g, an antibody expression cassette) provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

In some aspects, a viral vector disclosed herein comprises a polynucleotide (e.g, an antibody expression cassette) comprising coding regions for two or more polypeptides, e.g., a heavy chain and a light chain.

When it is desired the polynucleotide (e.g, an antibody expression cassette) includes coding regions for two or more individual polypeptide chains, each additional coding region beyond the first is preferably linked to an element that facilitates co-expression of the proteins in host cells, such as an internal ribosomal entry sequence (IRES) element (See e.g., U.S. Pat. No. 4,937,190), furin cleavage site, a 2A element, or promoter(s). In some aspects, IRES furin cleavage sites, or 2A elements can be used when a single vector comprises sequences encoding each subunit of a multi-subunit protein. In the case when the protein of interest is immunoglobulin with a desired specificity, for example, the first coding region (encoding either the heavy or light chain of immunoglobulin) is located downstream from the promoter. The second coding region (encoding the remaining chain of immunoglobulin) can be located downstream from the first coding region, and an IRES, furin cleavage site, or 2A element can be disposed between the two coding regions, e.g., immediately preceding the second coding region. In some aspects, the incorporation of an IRES, furin cleavage site, or 2A element between the sequences of a first and second gene (encoding the heavy and light chains, respectively) can allow both chains to be expressed from the same promoter at about the same level in the cell.

In some aspects, the protein of interest comprises two or more subunits, for example an immunoglobulin (Ig). In some aspects, a delivery vector of the disclosure can include a coding region for each of the subunits. For example, the viral vector can include both the coding region for the Ig heavy chain (or the variable region of the Ig heavy chain) and the coding region for the Ig light chain (or the variable region of the Ig light chain). In some aspects, the vectors include a first coding region for the heavy chain variable region of an antibody, and a second coding region for the light chain variable region of the antibody. In some aspects, the two coding regions can be separated, for example, by a 2A self-processing sequence to allow multi-cistronic transcription of the two coding regions.

The viral vector can include coding regions for two or more proteins of interest. For example, the viral vector can include the coding region for a first protein of interest and the coding region for a second protein of interest. The first protein of interest and the second protein of interest can be the same or different.

The Kozak consensus sequence, Kozak consensus or Kozak sequence, is known as a sequence which occurs on eukaryotic mRNA and has the consensus (gcc)gccRccAUGG, where R is a purine (adenine or guanine) three bases upstream of the start codon (AUG), which is followed by another “G.” In some aspects, the vector comprises a nucleotide sequence having at least about 85%, at least about 90%, at least about 95% sequence identity, or more to the Kozak consensus sequence. In some aspects, the vector comprises a Kozak consensus sequence. In some aspects, the vector includes a Kozak consensus sequence after the polynucleotide encoding one or more proteins of interest is inserted into the vector, e.g., at the restrict site downstream of the promoter. For example, the vector can include a nucleotide sequence of GCCGCCATG (SEQ ID NO: 78), where the ATG is the start codon of the protein of interest. In some aspects, the vector comprises a nucleotide sequence of GCGGCCGCCATG (SEQ ID NO: 79), where the ATG is the start codon of the protein of interest.

In certain aspects, a composition comprising a delivery vector, e.g., a viral vector, comprising nucleic acids encoding an anti-TNFalpha antibody or an antigen binding fragment.

In some aspects, the delivery vector, e.g., a viral vector, comprises a nucleic acid encoding an anti-TNFalpha antibody (e.g., a monoclonal antibody) or an antigen-binding fragment thereof disclosed herein is secreted from the salivary gland and swallowed. In some aspects, the therapeutic effect of the secreted anti-TNFalpha antibody or antigen-binding fragment thereof is local, systemic, or both.

In some aspects, the delivery vector (e.g., a delivery vector comprising an antibody or antigen-binding fragments thereof specifically binds to tumor necrosis factor (TNF), such as human TNF-α) is suitable for delivery to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, the polynucleotide (e.g, an antibody expression cassette) comprises a nucleic acid sequence encoding an anti-TNFalpha antibody or antigen-binding fragment thereof comprising (i) coding sequences for the VH and VL CDRs of adalimumab (e.g., SEQ ID NOs: 87-92, 96-101 and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the polynucleotide (e.g., antibody expression cassette) comprises any of SEQ ID NOs: 42-51, 52-61, 62-73, 74-77, 115-141, or 153-158; any of the sequences disclosed in Table 16 or Table 17; or any combination thereof.

### III. Constructs

Some aspects of the disclosure are directed to a nucleic acid construct (e.g., vector) or an expression construct (e.g., comprising an antibody expression cassette) having a eukaryotic promoter operably linked to a DNA of interest that encodes an anti-TNFalpha antibody (e.g., a monoclonal antibody) or an antigen binding fragment thereof disclosed herein. In some aspects, the constructs containing the DNA sequence (or the corresponding RNA sequence) which can be used in accordance with the disclosure can be any eukaryotic expression construct containing the DNA or the RNA sequence of interest. For example, a plasmid or viral construct (e.g. an AAV vector) can be cleaved to provide linear DNA having ligatable termini. These termini are bound to exogenous DNA having complementary, like ligatable termini to provide a biologically functional recombinant DNA molecule having an intact replicon and a desired phenotypic property. In some aspects, the construct is capable of replication in both eukaryotic and prokaryotic hosts, which constructs are known in the art and are commercially available.

In some aspects, the construct of the disclosure encoding an anti-TNFalpha antibody (e.g., adalimumab) is a multicistronic (e.g., bicistronic) construct (e.g., comprising a heavy chain and a light chain). In some aspects, the multicistronic (e.g., bicistronic) construct further comprises an F2A or IRES element.

The exogenous (i.e., donor) DNA used in the disclosure is obtained from suitable cells, and the constructs prepared using techniques well known in the art. Likewise, techniques for obtaining expression of exogenous DNA or RNA sequences in a genetically altered host cell are known in the art (see e.g., Kormal et al., Proc. Natl. Acad. Sci. USA, 84:2150-2154 (1987); Sambrook et al. Molecular Cloning: a Laboratory Manual, 2nd Ed., 1989, Cold Spring Harbor Laboratory Press, Cold Spring Harbor, N.Y.; each of which are hereby incorporated by reference with respect to methods and compositions for eukaryotic expression of a DNA of interest).

In some aspects, the construct contains a promoter to facilitate expression of the DNA of interest within a secretory cell. In some aspects, the promoter is a strong, eukaryotic promoter such as a promoter from cytomegalovirus (CMV), mouse mammary tumor virus (MMTV), Rous sarcoma virus (RSV), or adenovirus. Exemplary promoters include, but are not limited to the promoter from the immediate early gene of human CMV (Boshart et al., Cell 41:521-530 (1985) and the promoter from the long terminal repeat (LTR) of RSV (Gorman et al., Proc. Natl. Acad. Sci. USA 79:6777-6781 (1982)). In some aspects, the promoter is a CMV early enhancer/chicken β actin (CBA) promoter, CAG promoter, CMV, EF1α, EF1α with a CMV enhancer, a CMV promoter with a CMV enhancer (CMVe/p), a CMV promoter with a SV40 intron or tissue specific promoters.

In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38. In some aspects, the nucleic acid sequence comprising the promoter can comprises an intron. In some aspects, the intron is selected from the group consisting of an SV40 intron, MVM intron, or a human betaglobin intron. In some aspects a CMVp promoter is fused to a SV40 intron. In some aspects, SV40 intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 33.

Alternatively, the promoter used can be a tissue-specific promoter. For example, where the secretory gland is a salivary gland, the tissue-specific promoter can be a salivary α-amylase promoter or mumps viral gene promoter and where the secretory gland is the pancreas, the promoter used in the vector can be a pancreas specific promoter, e.g., an insulin promoter or a pancreas α-amylase promoter. Both salivary and pancreatic α-amylase genes have been identified and characterized in both mice and humans (see e.g., Jones et al., Nucleic Acids Res., 17:6613-6623 (1989); Pittet et al., J. Mol. Biol., 182:359-365 (1985); Hagenbuchle et al., J. Mol. Biol., 185:285-293 (1985); Schibler et al., Oxf. Surv. Eukaryot. Genes, 3:210-234 (1986); and Sierra et al., Mol. Cell. Biol., 6:4067-4076 (1986) for murine pancreatic and salivary α-amylase genes and promoters; Samuelson et al., Nucleic Acids Res., 16:8261-8276 (1988); Groot et al., Genomics, 5:29-42 (1989); and Tomita et al., Gene, 76:11-18 (1989) for human pancreatic and salivary α-amylase genes and their promoters; Ting et al., Genes Dev. 6:1457-65 (1992) for human salivary α-amylase AMY1C promoter sequences).

In some aspects, the construct contains a first promoter and a second promoter. In some aspects, the first and second promoter are different. In some aspects, the first and second promoter are the same. In some aspects, the first and second promoter initiate transcription in the same direction. In some aspects, the first and second promoter initiate transcription in different directions.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 41.

In some aspects, the constructs of the disclosure can also include sequences in addition to promoters that enhance secretory gland specific expression. For example, where pancreas specific expression of the DNA of interest is desired, the construct can include a PTF-1 recognition sequence (Cockell et al., Mol. Cell. Biol., 9:2464-2476 (1989)). Sequences which enhance salivary gland specific expression are also well known in the art (see e.g., Robins et al., Genetica 86:191-201 (1992)).

Given the genome size limitations of AAV, expression of multicistronic vectors (multiple genes or multiple open reading frames) of large proteins under the control of a single promoter can be a challenge.

In some aspects, the multicistronic vectors disclose herein comprise an internal ribosome entry site (IRES) sequences or 2A peptides. In some aspects, the constructs of the disclosure can also include proteolytic cleavage sites. In some aspects, the proteolytic cleavage sites are furin cleavage sites and/or 2A cleavage sites.

In some aspects, the constructs of the disclosures can also include an miRNA binding site. In some aspects, the miRNA binding site is an miR-142 binding site. In some aspects, the miRNA binding site comprises four miR-142 binding sites. In some aspects, the four miR-142 binding sites are separated by spacers. In some aspects, the miR-142 binding site has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 31. In some aspects, the 4× miR-142 binding site has a nucleic acid sequence at least 85%, 86%, 87%, 88%, 89%, 90%, 91%, 92%, 93%, 94%, 95%, 96%, 97%, 98%, 99%, or 100% identical to SEQ ID NO: 32.

In some aspects, the constructs of the disclosure can also include other components such as a marker (e.g., an antibiotic resistance gene (such as an ampicillin resistance gene) or β-galactosidase) to aid in selection of cells containing and/or expressing the construct, an origin of replication for stable replication of the construct in a bacterial cell (preferably, a high copy number origin of replication), a nuclear localization signal, or other elements which facilitate production of the DNA construct, the protein encoded thereby, or both.

For eukaryotic expression, the construct can comprise at a minimum a eukaryotic promoter operably linked to a DNA of interest, which is in turn operably linked to a polyadenylation sequence. The polyadenylation signal sequence can be selected from any of a variety of polyadenylation signal sequences known in the art. In some aspects, the polyadenylation signal sequence is the SV40 early polyadenylation signal sequence. The construct can also include one or more introns, which can increase levels of expression of the DNA of interest, particularly where the DNA of interest is a cDNA (e.g., contains no introns of the naturally-occurring sequence). Any of a variety of introns known in the art can be used (e.g., the human β-globin intron, which is inserted in the construct at a position 5′ to the DNA of interest). In some aspects, the intron is an SV40 intron. In some aspects, the intron is from an immunoglobulin heavy chain. In some aspects, the intron is a chimera between the human β-globin and immunoglobin heavy chain gene. In some aspects, the intron comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 33 and 82.

In some aspects, the polynucleotide (e.g, an antibody expression cassette) comprises a poly(A). In some aspects, the poly(A) is a synthetic poly(A) or a bovine growth hormone (BGH) poly(A). In some aspects, the polynucleotide comprises a poly(A) sequence comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39-40, 114, and 152.

When it is desired to include coding regions for two or more individual polypeptide chains, or two or more subunits of a protein of interest in one viral vector, each additional coding region beyond the first is preferably linked to an element that facilitates co-expression of the proteins in host cells, such as an internal ribosomal entry sequence (IRES) element (See e.g., U.S. Pat. No. 4,937,190), or a 2A element. In some aspects, IRES, furin cleavage site, or 2A elements can be used when a single vector comprises sequences encoding each subunit of a multi-subunit protein. In the case when the protein of interest is immunoglobulin with a desired specificity, for example, the first coding region (encoding either the heavy or light chain of immunoglobulin) is located downstream from the promoter. The second coding region (encoding the remaining chain of immunoglobulin) can be located downstream from the first coding region, and an IRES, furin cleavage site, or 2A element can be disposed between the two coding regions, e.g., immediately preceding the second coding region. In some aspects, the incorporation of an IRES, furin cleavage site, or 2A element between the sequences of a first and second gene (encoding the heavy and light chains, respectively) can allow both chains to be expressed from the same promoter at about the same level in the cell.

In some aspects, the nucleic acid sequence of the construct comprises a promoter, heavy chain, IRES, and light chain sequences in 5′-3′ orientation. In some aspects, the nucleic acid sequence of the construct comprises a promoter, light chain, IRES, and heavy chain sequences in 5′-3′ orientation.

In some aspects, the nucleic acid sequence construct comprises proteolytic cleavage sites. For example, the nucleic acid sequence may comprise a sequence that is incorporated into an expression construct of the disclosure adjacent a self-processing cleavage site, such as a 2A or 2A like sequence, and provides a means to remove additional amino acids that remain following cleavage by the self-processing cleavage sequence. Exemplary proteolytic cleavage sites are described herein and include, but are not limited to, furin cleavage sites with the consensus sequence RXK(R)R (SEQ ID NO: 27). Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

In some aspects, the nucleic acid sequence construct comprises a promoter, heavy chain, furin cleavage site, 2A cleavage site, and light chain sequences in 5′-3′ orientation. In some aspects, the nucleic acid sequence construct comprises a promoter, light chain, furin cleavage site, 2A cleavage site, and heavy chain sequences in 5′-3′ orientation.

In some aspects, the polynucleotide (e.g, an antibody expression cassette) comprises a first promoter, a nucleic acid sequence encoding a light chain, a second promoter, and a nucleic acid sequence encoding a heavy chain in 5′-3′ orientation.

In some aspects, the polynucleotide (e.g, an antibody expression cassette) comprises a first promoter, a nucleic acid sequence encoding a heavy chain, a second promoter, and a nucleic acid sequence encoding a light chain in 5′-3′ orientation.

In some aspects, the polynucleotide (e.g, an antibody expression cassette) comprises a nucleic acid sequence encoding a heavy chain, a first promoter sequence, a second promoter sequence, and a nucleic acid sequence encoding a light chain in 5′-3′ orientation.

In some aspects, the polynucleotide (e.g, an antibody expression cassette) comprises a nucleic acid sequence encoding a light chain, a first promoter sequence, a second promoter sequence, and a nucleic acid sequence encoding a heavy chain in 5′-3′ orientation.

In some aspects, the promoter is selected from the group consisting of a CAG, CBA, CMV, EF1α, EF1α with a CMV enhancer, a CMV promoter with a CMV enhancer (CMVe/p), a CMV promoter with a SV40 intron or tissue specific promoter.

In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38. In some aspects, the nucleic acid sequence comprising the promoter can comprises an intron. In some aspects, the intron is selected from the group consisting of an SV40 intron, MVM intron, or a human betaglobin intron. In some aspects, a CMVp is fused to a SV40 intron. In some aspects, SV40 intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 33.

In some aspects, the first and second promoter are different. In some aspects, the first and second promoter are the same. In some aspects, the first and second promoter initiate transcription in the same direction. In some aspects, the first and second promoter initiate transcription in different directions.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 41.

The vectors for delivery of the DNA of interest can be either viral or non-viral, or can be composed of naked DNA admixed with an adjuvant such as viral particles (e.g., AAV particle) or cationic lipids or liposomes. An “adjuvant” is a substance that does not by itself produce the desired effect, but acts to enhance or otherwise improve the action of the active compound. The precise vector and vector formulation used will depend upon several factors such as the secretory gland targeted for gene transfer.

In some aspects, a composition comprising a delivery vector, e.g., a viral vector, comprising a nucleic acid construct or an expression construct comprising a nucleic acid encoding an anti-TNFalpha antibody (e.g., a monoclonal antibody) or an antigen binding fragment thereof disclosed herein. In some aspects, the delivery vector is suitable for delivery to a secretory organ. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the composition is suitable for delivery to the liver. In some aspects, the secretory gland is a salivary gland, pineal gland, thyroid gland, adrenal gland, and parathyroid gland. In some aspects, the secretory gland is a salivary gland.

In some aspects, the construct comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the construct (e.g., antibody expression cassette) comprises any of SEQ ID NOs: 42-51, 52-61, 62-73, 74-77, 115-141, or 153-158; any of the sequences disclosed in Table 16 or Table 17; or any combination thereof.

In some aspects, the nucleic acid construct or expression construct comprises a polynucleotide comprising a first promoter, nucleic acid sequence encoding a heavy chain, a poly(A), a pause element, a second promoter, a 5′ LTR, nucleic acid sequence encoding a light chain, and a poly(A) in the 5′-3′ orientation. In some aspects, the nucleic acid construct or expression construct comprises a polynucleotide comprising a CMV enhancer, a CMV promoter, a nucleic acid encoding a heavy chain, a BGHpA, a pause element, a EF1α promoter, a 5′ LTR, a nucleic acid encoding a light chain, and a SynpA in the 5′-3′ orientation.

In some aspects, the nucleic acid construct or expression construct comprises a polynucleotide comprising a first promoter, nucleic acid sequence encoding a light chain, a poly(A), a pause element, a second promoter, a 5′ LTR, nucleic acid sequence encoding a heavy chain, and a poly(A) in the 5′-3′ orientation. In some aspects, the nucleic acid construct or expression construct comprises a polynucleotide comprising a CMV enhancer, a CMV promoter, a nucleic acid encoding a light chain, a BGHpA, a pause element, a EF1α promoter, a 5′ LTR, a nucleic acid encoding a heavy chain, and a SynpA in the 5′-3′ orientation.

In some aspects, the nucleic acid construct or expression construct comprises a polynucleotide comprising a poly(A), a nucleic acid sequence encoding a light chain, an intron, a 5′ LTR, a first promoter, a second promoter, an intron, a nucleic acid sequence encoding a heavy chain, and a poly(A) in the 5′-3′ orientation. In some aspects, the nucleic acid construct or expression construct comprises a polynucleotide comprising a SYNpA, a nucleic acid encoding a light chain, a chimera of a betaglobin intron and a immunoglobulin heavy chain intron, a 5′LTR, a EF1α promoter fused to a CMV enhancer, a CMV promoter fused to a SV40 intron, a nucleic acid sequence encoding a heavy chain, and a BGHpA in the 5′-3′ orientation.

In some aspects, the nucleic acid construct or expression construct comprises a polynucleotide comprising a poly(A), a nucleic acid sequence encoding a heavy chain, an intron, a 5′ LTR, a first promoter, a second promoter, an intron, a nucleic acid sequence encoding a light chain, and a poly(A) in the 5′-3′ orientation. In some aspects, the nucleic acid construct or expression construct comprises a polynucleotide comprising a SYNpA, a nucleic acid encoding a heavy chain, a chimera of a betaglobin intron and a immunoglobulin heavy chain intron, a 5′LTR, a EF1α promoter fused to a CMV enhancer, a CMV promoter fused to a SV40 intron, a nucleic acid sequence encoding a light chain, and a BGHpA in the 5′-3′ orientation.

In some aspects, the constructs (e.g., vectors or antibody expression constructs) disclosed herein comprise one or more of the elements listed in Table 15.

TABLE 15

| SEQ ID | | |
|---|---|---|
| NOs | Name | Nucleic Acid Sequence |
| (SEQ ID | IRES | gcccctctccctcccccccccctaacgttactggccgaagccgcttggaataaggc |
| NO: 25) | | cggtgtgcgtttgtctatatgttattttccaccatattgccgtcttttggcaatgtgaggg |
| | | cccggaaacctggccctgtcttcttgacgagcattcctaggggtctttcccctctcgc |
| | | caaaggaatgcaaggtctgttgaatgtcgtgaaggaagcagttcctctggaagctt |
| | | cttgaagacaaacaacgtctgtagcgaccctttgcaggcagcggaaccccccacc |
| | | tggcgacaggtgcctctgcggccaaaagccacgtgtataagatacacctgcaaag |
| | | gcggcacaaccccagtgccacgttgtgagttggatagttgtggaaagagtcaaat |
| | | ggctctcctcaagcgtattcaacaaggggctgaaggatgcccagaaggtacccca |
| | | ttgtatgggatctgatctggggcctcggtacacatgctttacatgtgtttagtcgaggt |
| | | taaaaaaacgtctaggccccccgaaccacggggacgtggttttcctttgaaaaaca |
| | | cgatgataatatggccaca |
| (SEQ ID | Furin | agaaagagaaggagtggctcagga |
| NO: 26) | cleavage site | |
| (SEQ ID | 2A site | gcccctgtgaaacagaccctgaactttgacctcttgaagcttgctggggatgtgga |
| NO: 28) | | gtctaatcctggtcca |
| (SEQ ID | IL-2 leader | atgtacaggatgcaactcctgtcttgcattgcactaagtcttgcacttgtcacaaactc |
| NO: 29) | | g |
| (SEQ ID | IL-2 leader | atgtacaggatgcaactcctgtcttgcattgcactaagtcttgcacttgtcacaaactc |
| NO: 113) | | a |
| (SEQ ID | IL-10 leader | atgcacagctcagcactgctctgttgcctggtcctcctgactggggtgagggcc |
| NO: 30) | | |
| (SEQ ID | IL-10 leader | atgcacagctcagcactgctctgttgcctggtcctcctgactggggtgagggct |
| NO: 112) | | |
| (SEQ ID | miR-142 | tccataaagtaggaaacactaca |
| NO: 31) | binding site | |
| (SEQ ID | 4x miR-142 | tccataaagtaggaaacactacactattccataaagtaggaaacactacatcactcc |
| NO: 32) | binding site | ataaagtaggaaacactacaagtctccataaagtaggaaacactaca |
| SEQ ID | SV40 intron | gtaagtttagtctttttgtcttttatttcaggtcccggatccggtggtggtgcaaatcaa |
| NO: 33 | | agaactgctcctcagtggatgttgcctttacttctag |
| SEQ ID | EF-1α | gctccggtgcccgtcagtgggcagagcgcacatcgcccacagtccccgagaagt |
| NO: 34 | | tggggggaggggtcggcaattgaaccggtgcctagagaaggtggcgcggggta |
| | | aactgggaaagtgatgtcgtgtactggctccgcctttttcccgaggggggggaga |
| | | accgtatataagtgcagtagtcgccgtgaacgttctttttcgcaacgggtttgccgcc |
| | | agaacacag |
| SEQ ID | CMV | gacattgattattgactagttattaatagtaatcaattacggggtcattagttcatagcc |
| NO: 35 | enhancer | catatatggagttccgcgttacataacttacggtaaatggcccgcctggctgaccgc |
| | | ccaacgacccccgcccattgacgtcaataatgacgtatgttcccatagtaacgcca |
| | | atagggactttccattgacgtcaatgggtggactatttacggtaaactgcccacttgg |
| | | cagtacatcaagtgtatcatatgccaagtacgccccctattgacgtcaatgacggta |
| | | aatggcccgcctggcattatgcccagtacatgaccttatgggactttcctacttggca |
| | | gtacatctacgtattagtcatcgctattaccatg |
| SEQ ID | CMVp | gtgatgcggttttggcagtacatcaatgggcgtggatagcggtttgactcacgggg |
| NO: 36 | | atttccaagtctccaccccattgacgtcaatgggagtttgttttggcaccaaaatcaa |
| | | cgggactttccaaaatgtcgtaacaactccgccccattgacgcaaatgggcggtag |
| | | gcgtgtacggtgggaggtctatataagcagagctcgtttagtgaaccg |
| SEQ ID | CMV | gacattgattattgactagttattaatagtaatcaattacggggtcattagttcatagcc |
| NO: 37 | enhancer and | catatatggagttccgcgttacataacttacggtaaatggcccgcctggctgaccgc |
| | promoter | ccaacgacccccgcccattgacgtcaataatgacgtatgttcccatagtaacgcca |
| | (CMVe/p) | atagggactttccattgacgtcaatgggtggactatttacggtaaactgcccacttgg |
| | | cagtacatcaagtgtatcatatgccaagtacgccccctattgacgtcaatgacggta |
| | | aatggcccgcctggcattatgcccagtacatgaccttatgggactttcctacttggca |
| | | gtacatctacgtattagtcatcgctattaccatggtgatgcggttttggcagtacatca |
| | | atgggcgtggatagcggtttgactcacggggatttccaagtctccaccccattgac |
| | | gtcaatgggagtttgttttggcaccaaaatcaacgggactttccaaaatgtcgtaaca |
| | | actccgccccattgacgcaaatgggcggtaggcgtgtacggtgggaggtctatat |
| | | aagcagagctcgtttagtgaaccg |
| SEQ ID | CAG | gtcgacattgattattgactagttattaatagtaatcaattacggggtcattagttcata |
| NO: 38 | | gcccatatatggagttccgcgttacataacttacggtaaatggcccgcctggctgac |
| | | cgcccaacgacccccgcccattgacgtcaataatgacgtatgttcccatagtaacg |
| | | ccaatagggactttccattgacgtcaatgggtggactatttacggtaaactgcccact |
| | | tggcagtacatcaagtgtatcatatgccaagtacgccccctattgacgtcaatgacg |
| | | gtaaatggcccgcctggcattatgcccagtacatgaccttatgggactttcctacttg |
| | | gcagtacatctacgtattagtcatcgctattaccatgggtcgaggtgagccccacgt |
| | | tctgcttcactctccccatctcccccccctccccacccccaattttgtatttatttatttttt |
| | | aattattttgtgcagcgatgggggcggggggggggggggcgcgcgccaggcg |
| | | gggcggggcggggcgaggggcggggggggcgaggcggagaggtgcggc |
| | | ggcagccaatcagagcggcgcgctccgaaagtttccttttatggcgaggcggcg |
| | | gcggcggcggccctataaaaagcgaagcgcgcggggggggagtcgctgcg |
| | | cgctgccttcgccccgtgccccgctccgccgccgcctcgcgccgcccgccccgg |
| | | ctctgactgaccgcgttactcccacaggtgagcggggggacggcccttctcctc |
| | | cgggctgtaattagcgcttggtttaatgacggctcgtttcttttctgtggctgcgtgaa |
| | | agccttgaggggctccgggagggccctttgtgcggggggagcggctcgggggg |
| | | tgcgtgcgtgtgtgtgtgcgtggggagcgccgcgtgcggctccgcgctgcccgg |
| | | cggctgtgagcgctgcgggcgcggcgcggggctttgtgcgctccgcagtgtgcg |
| | | cgagaggagcgcggccgggggcggtgccacgcggtgcggggggggctgcga |
| | | ggggaacaaaggctgcgtgcggggtgtgtgcgtggggggggtgagcaggggg |
| | | tgtgggcgcggcggtcgggctgtaacccccccctgcacccccctccccgagttg |
| | | ctgagcacggcccggcttcgggtgcggggctccgtgcggggcgtggcgcggg |
| | | gctcgccgtgccgggcggggggggcggcgggtgggggtgccgggcggggc |
| | | ggggccgcctcgggccggggagggctcgggggaggggcgcggcggccccc |
| | | ggagcgccggcggctgtcgaggcgcggcgagccgcagccattgccttttatggt |
| | | aatcgtgcgagagggcgcagggacttcctttgtcccaaatctgtgcggagccgaa |
| | | atctgggaggcgccgccgcaccccctctagcgggcgcggggcgaagcggtgc |
| | | ggcgccggcaggaaggaaatgggcggggagggccttcgtgcgtcgccgcgcc |
| | | gccgtccccttctccctctccagcctcggggctgtccgcggggggacggctgcct |
| | | tcgggggggacggggcagggcggggttcggcttctggcgtgtgaccggcggtc |
| | | tagactctgctaaccatgttcatgccttcttctctttcctacagctcctgggcaacgtg |
| | | ctggttgttgtgctgtctcatcattttggcaaa |
| SEQ ID | Synthetic | aataaaagatctttattttcattagatctgtgtgttggttttttgtgtg |
| NO: 39 | poly(A) | |
| SEQ ID | BGH poly(A) | ctgtgccttctagttgccagccatctgttgtttgcccctcccccgtgccttccttgacc |
| NO: 40 | | ctggaaggtgccactcccactgtcctttcctaataaaatgaggaaattgcatcgcatt |
| | | gtctgagtaggtgtcattctattctggggggtggggggggcaggacagcaaggg |
| | | ggaggattgggaagacaatagcaggcatgctggggatgcggtgggctctatg |
| SEQ ID | bGH PolyA | ctgtgccttctagttgccagccatctgttgtttgcccctcccccgtgccttccttgacc |
| NO: 152 | | ctggaaggtgccactcccactgtcctttcctaataaaatgaggaaattgcatcgcatt |
| | | gtctgagtaggtgtcattctattctggggggtggggtggggcaggacagcaaggg |
| | | ggaggatt |
| SEQ ID | HGH poly(A) | aggaacccctagtgatggagttggccactccctctctgcgcgctcgctcgctcact |
| NO: 114 | | gaggccgggcgaccaaaggtcgcccgacgcccgggctttgcccgggggcct |
| | | cagtgagcgagcgagcgcgcag |
| SEQ ID | Pause | aacatacgctctccatcaaaacaaaacgaaacaaaacaaactagcaaaataggct |
| NO: 41 | element | gtccccagtgcaagtgcaggtgccagaacatttctct |
| SEQ ID | 5′ LTR | ggggctcgcatctctccttcacgcgcccgccgccctacctgaggccgccatccac |
| NO: 81 | | gccggttgagtcgcgttctgccgcctcccgcctgtggtgcctcctgaactgcgtcc |
| | | gccgtctaggtaagtttaaagctcaggtcgagaccgggcctttgtccggcgctccc |
| | | ttggagcctacctagactcagccggctctccacgctttgcctgaccctgcttgctca |
| | | actctacgtctttgtttcgttttctgttctgcgccgttacagatc |
| SEQ ID | Intron | gtaagtatcaaggttacaagacaggtttaaggagaccaatagaaactgggcttgtc |
| NO: 82 | (chimera of | gagacagagaagactcttgcgtttctgataggcacctattggtcttactgacatccac |
| | human | tttgcctttctctccacag |
| | betaglobin | |
| | intron and | |
| | immunoglobu | |
| | lin heavy | |
| | chain intron) | |

In some aspects, the vector or construct of the disclosure comprises a backbone, e.g., including the replication origin (oriR) and/or antibiotic resistance gene. In some aspects, the backbone comprises a colicin E1 gene (ColE1) origin of replication and/or a kanamycin resistance gene (KanR). In some aspects, the backbone is a suitable for use in an AAV payload vector (e.g., comprising an antibody expression cassette flanked by 5′ and 3′ ITRs). In some aspects, the backbone can be a puc57 backbone (Addgene) or a modified version thereof. In some aspects, the backbone can comprise a filler sequence.

### III.A. Delivery Vectors

In some aspects, the delivery vector is a viral vector, a non-viral vectors, a plasmid, a lipid, or a lysosome.

In certain aspects, a composition comprising a delivery vector (e.g., a viral vector, a non-viral vectors, a plasmid, a lipid, a protein particle, a bacterial vector, or a lysosome) comprising a nucleic acid encoding or comprising an antibody (e.g., a monoclonal antibody) or an antigen binding fragment thereof disclosed herein. In some aspects, the delivery vector is suitable for delivery to a secretory organ. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the secretory gland is a salivary gland, pineal gland, thyroid gland, adrenal gland, and parathyroid gland. In some aspects, the secretory gland is a salivary gland.

In some aspects, the composition comprising a delivery vector (e.g., a viral vector, a non-viral vectors, a plasmid, a lipid, a protein particle, a bacterial vector, or a lysosome) comprises a nucleic acid encoding an antibody (e.g., a monoclonal antibody) or an antigen-binding fragment thereof disclosed herein is secreted from the salivary gland and swallowed. In some aspects, the therapeutic effect of the secreted antibody or antigen-binding fragment thereof is local, systemic, or both.

In some aspects, a composition comprising the delivery vector (e.g., a viral vector, a non-viral vectors, a plasmid, a lipid, a protein particle, a bacterial vector, or a lysosome) comprising a nucleic acid encoding or comprising an antibody (e.g., a monoclonal antibody) or an antigen binding fragment thereof disclosed herein is suitable for delivery to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, the delivery vector comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the delivery vector comprises any of SEQ ID NOs: 42-51, 52-61, 62-73, 74-77, 115-141, or 153-158; any of the sequences disclosed in Table 16 or Table 17; or any combination thereof.

### III.A.1 Non-Viral Vectors

The DNA of interest can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, the non-viral vector is a bacterial vector. See e.g., Baban et al., Bioeng Bugs., 1(6):385-394 (2010).

In some aspects, the DNA of interest can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3-β-ol 3-urethanyl)-N′,N′-dimethylethylene diamine).

Lipids which can be used in accordance with the disclosure include, but are not limited to, DOPE (Dioleoyl phosphatidylethanolamine), cholesterol, and CUDMEDA (N-(5-cholestrum-3-ol 3 urethanyl)-N′,N′-dimethylethylenediamine). As an example, DNA can be administered in a solution containing one of the following cationic liposome formulations: Lipofectin™ (LTI/BRL), Transfast™ (Promega Corp), Tfx50™ (Promega Corp), Tfx10™ (Promega Corp), or Tfx20™ (Promega Corp). The concentration of the liposome solutions range from about 2.5% to 15% volume:volume, preferably about 6% to 12% volume:volume. Further exemplary methods and compositions for formulation of nucleic acid (e.g., DNA, including DNA or RNA not contained within a viral particle) for delivery according to the method of the disclosure are described in U.S. Pat. Nos. 5,892,071; 5,744,625; 5,925,623; 5,527,928; 5,824,812; 5,869,715.

Polymer particles can be used in accordance with the disclosure for polymer-based gene delivery. See e.g., Putnam et al., PNAS 98 (3): 1200-1205 (2001).

The DNA of interest can also be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof. Exemplary protein carrier molecules include antibodies specific to the cells of a targeted secretory gland or receptor ligands, i.e., molecules capable of interacting with receptors associated with a cell of a targeted secretory gland (e.g., salivary gland).

In certain aspects, a composition comprising a non-viral delivery vector comprising a nucleic acid encoding or comprising an antibody (e.g., a monoclonal antibody) or an antigen binding fragment thereof disclosed herein suitable for delivery to a secretory organ. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the secretory gland is a salivary gland, pineal gland, thyroid gland, adrenal gland, and parathyroid gland. In some aspects, the secretory gland is a salivary gland.

In some aspects, the composition comprising a non-viral delivery vector comprises a nucleic acid encoding an antibody (e.g., a monoclonal antibody) or an antigen-binding fragment thereof disclosed herein, or a therapeutic peptide that is secreted from the salivary gland and swallowed. In some aspects, the therapeutic effect of the secreted antibody or antigen-binding fragment thereof is local, systemic, or both.

In some aspects, a composition comprising a non-viral delivery vector comprising a nucleic acid encoding or comprising an antibody (e.g., a monoclonal antibody) or an antigen binding fragment thereof disclosed herein is suitable for delivery to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, the non-viral vector comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the non-viral vector comprises any of SEQ ID NOs: 42-51, 52-61, 62-73, 74-77, 115-141, or 153-158; any of the sequences disclosed in Table 16 or Table 17; or any combination thereof.

### III.A.2 Viral Vectors

In general, viral vectors used in accordance with the disclosure are composed of a viral particle derived from a naturally-occurring virus which has been genetically altered to render the virus replication-defective and to express a recombinant gene of interest in accordance with the disclosure. Once the virus delivers its genetic material to a cell, it does not generate additional infectious virus but does introduce exogenous recombinant genes into the cell, preferably into the genome of the cell.

Numerous viral vectors are well known in the art, including, for example, retrovirus, adenovirus, adeno-associated virus (AAV), herpes simplex virus (HSV), cytomegalovirus (CMV), vaccinia and poliovirus vectors. Retroviral vectors are less preferred since retroviruses require replicating cells and secretory glands are composed of mostly slowly replicating and/or terminally differentiated cells. Adenovirus and AAV are preferred viral vectors since this virus efficiently infects slowly replicating and/or terminally differentiated cells. In some aspects, the delivery vector (e.g., viral vector) is selected from the group consisting of an adeno-associated viral (AAV) vector, an adenoviral vector, a lentiviral vector, or a retroviral vector.

Where a replication-deficient virus is used as the viral vector, the production of infective virus particles containing either DNA or RNA corresponding to the DNA of interest can be produced by introducing the viral construct into a recombinant cell line which provides the missing components essential for viral replication. In some aspects, transformation of the recombinant cell line with the recombinant viral vector will not result in production of replication-competent viruses, e.g., by homologous recombination of the viral sequences of the recombinant cell line into the introduced viral vector. Methods for production of replication-deficient viral particles containing a nucleic acid of interest are well known in the art and are described in, e.g., Rosenfeld et al., Science 252:431-434 (1991) and Rosenfeld et al., Cell 68:143-155 (1992) (adenovirus); U.S. Pat. No. 5,139,941 (adeno-associated virus); U.S. Pat. No. 4,861,719 (retrovirus); and U.S. Pat. No. 5,356,806 (vaccinia virus).

In certain aspects, the viral delivery vector comprising a nucleic acid encoding or comprising an antibody (e.g., a monoclonal antibody) or an antigen binding fragment thereof disclosed herein suitable for delivery to a secretory organ. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the secretory gland is a salivary gland, pancreas, a mammary gland, thyroid gland, parathyroid, an adrenal gland, a pineal body gland, thymus gland, pituitary gland, or hypothalamus. In some aspects, the secretory gland is a salivary gland.

In some aspects, the viral delivery vector comprises a nucleic acid encoding a therapeutic protein, e.g., an antibody (e.g., a monoclonal antibody) or an antigen-binding fragment thereof disclosed herein is secreted from the salivary gland and swallowed. In some aspects, the therapeutic effect of the secreted therapeutic antibody or antigen-binding fragment thereof is local, systemic, or both.

In some aspects, the viral delivery vector comprising a nucleic acid encoding or comprising an antibody (e.g., a monoclonal antibody) or an antigen binding fragment thereof disclosed herein is suitable for delivery to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, the viral vector comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the viral vector comprises any of SEQ ID NOs: 42-51, 52-61, 62-73, 74-77, 115-141, or 153-158; any of the sequences disclosed in Table 16 or Table 17; or any combination thereof.

### IV. Adeno-Associated Virus (AAV)-Mediated Gene Therapy

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce recombinant AAV (rAAV) virions containing the DNA, a suitable host cell line is transfected with an AAV vector containing the DNA, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the heterologous gene. The heterologous gene is then replicated and packaged as though it were a wt AAV genome, forming a recombinant AAV virion. When a patient's cells are transduced with the resulting rAAV virion, the DNA enters and is expressed in the patient's cells. Because the patient's cells lack the rep and cap genes, as well as the accessory function genes, the rAAV virion cannot further replicate and package its genomes. Moreover, without a source of rep and cap genes, wt AAV virions cannot be formed in the patient's cells. See e.g., U.S. Appl. Publ. No. 2003/0147853.

In some aspects, AAV vectors of the present disclosure can comprise or be derived from any natural or recombinant AAV serotype. According to the present disclosure, the AAV serotype can be, but is not limited to, AAV1, AAV2, AAV3, AAV4, AAV5, AAV6, AAV7, AAV8, AAVrh8, AAV9, AAV10, AAVrh10, AAV11, and AAV12. In some aspect, the AAV vector is modified relative to the wild-type AAV serotype sequence. In some aspects, the modified AAV capsid is AAV2 Quad Y-F or AAV2 Quad Y-F+T491V. In some aspects, the AAV capsid is AAV2.7m8. In some aspects, the AAV capsid is AAVshH10.

In some aspects, the AAV serotype is AAV2 or a modified version derived therefrom. In some aspects, the AAV serotype is wild-type AAV2. In some aspects, the AAV2 capsid is modified relative to wild-type AAV2. In some aspects, the modified AAV2 comprises a mutated AAV2 VP3 capsid protein comprising phenylalanines (F) at each of the positions corresponding to Y272, Y444, Y500, and Y730 in a wild type AAV2 VP3 capsid protein (also referred to as the “AAV2 Quad Y-F”). In some aspects, the modified AAVs comprises a mutated AAV2 VP3 capsid protein comprising phenylalanines (F) at each of the positions corresponding to Y272, Y444, Y500, and Y730 in a wild type AAV2 VP3 capsid protein and a mutation of threonine (T) to valine (V) at position T491 in a wild type AAV2 VP3 capsid protein (also referred to as the “AAV2 Quad Y-F+T491V”). See, e.g., U.S. Pat. Nos. 10,426,844 and 9,725,485, each of which is incorporated herein by reference in its entirety.

In certain aspects, a composition comprising an AAV delivery vector comprising a nucleic acid encoding or comprising an antibody (e.g., a monoclonal antibody) or an antigen binding fragment thereof disclosed herein. In some aspects, the AAV delivery vector is suitable for delivery to a secretory organ. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the secretory gland is a salivary gland, pineal gland, thyroid gland, adrenal gland, and parathyroid gland. In some aspects, the secretory gland is a salivary gland.

In some aspects, the AAV delivery vector comprises a nucleic acid encoding an antibody (e.g., a monoclonal antibody) or an antigen-binding fragment thereof disclosed herein is secreted from the salivary gland and swallowed. In some aspects, the therapeutic effect of the secreted antibody or antigen-binding fragment thereof is local, systemic, or both.

In some aspects, a composition comprising an AAV delivery vector comprising a nucleic acid encoding or comprising a therapeutic protein, e.g., an antibody (e.g., a monoclonal antibody) or an antigen binding fragment thereof or a fusion protein (e.g., an Fc fusion protein) disclosed herein, or a therapeutic peptide is suitable for delivery to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival).

In some aspects, the AAV vector comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the AAV vector comprises any of SEQ ID NOs: 42-51, 52-61, 62-73, 74-77, 115-141, or 153-158; any of the sequences disclosed in Table 16 or Table 17; or any combination thereof.

### IV.A. AAV Vector Components

### IV.A.1 Inverted Terminal Repeats (ITRs)

The AAV vectors of the present disclosure comprise a viral genome with at least one ITR region and a payload region, e.g., a polynucleotide encoding a therapeutic protein, e.g., an antibody (e.g., a monoclonal antibody) or an antigen binding fragment thereof or a fusion protein (e.g., an Fc fusion protein) disclosed herein, or a therapeutic peptide. In some aspects, the AAV vector comprises an antibody expression cassette disclosed herein. In some aspects the AAV vector has two ITRs. These two ITRs flank the payload region (e.g., antibody expression cassette) at the 5′ and 3′ ends. The ITRs function as origins of replication comprising recognition sites for replication. ITRs comprise sequence regions, which can be complementary and symmetrically arranged. ITRs incorporated into AAV vectors of the disclosure can be comprised of naturally occurring polynucleotide sequences or recombinantly derived polynucleotide sequences.

The ITRs can be derived from the same serotype as the capsid, selected from any of the serotypes listed herein, or a derivative thereof. The ITR can be of a different serotype from the capsid. In some aspects, the AAV vector has more than one ITR. In a non-limiting example, the AAV vector has a viral genome comprising two ITRs. In some aspects, the ITRs are of the same serotype as one another. In some aspects, the ITRs are of different serotypes. Non-limiting examples include zero, one or both of the ITRs having the same serotype as the capsid. In some aspects both ITRs of the AAV vector are AAV2 ITRs.

Independently, each ITR can be about 75 to about 175 nucleotides in length. An ITR can be about 100-105 nucleotides in length, about 106-110 nucleotides in length, about 111-115 nucleotides in length, about 116-120 nucleotides in length, about 121-125 nucleotides in length, about 126-130 nucleotides in length, about 131-135 nucleotides in length, about 136-140 nucleotides in length, about 141-145 nucleotides in length or about 146-150 nucleotides in length. In some aspects, the ITRs are about 140-142 nucleotides in length. Non-limiting examples of ITR length are about 102, about 140, about 141, about 142, about 145 nucleotides in length, and those having at least 95% identity thereto.

In some aspects, the AAV vector comprises at least one inverted terminal repeat having a length such as, but not limited to, about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, or about 170-175 nucleotides.

In some aspects, the length of a first and/or a second ITR regions for the AAV vector can be about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, and about 170-175 nucleotides.

In some aspects, the AAV vector comprises a nucleic acid sequence encoding an antibody (e.g., a monoclonal antibody) or an antigen binding fragment thereof disclosed herein which can be located near the 5′ end of the flip ITR in the vector. In some aspects, the AAV vector comprises a nucleic acid sequence encoding an antibody (e.g., a monoclonal antibody) or an antigen binding fragment thereof disclosed herein, which can be located near the 3′ end of the flip ITR in the vector. In some aspects, the AAV vector comprises a nucleic acid sequence encoding an antibody (e.g., a monoclonal antibody) or an antigen binding fragment thereof disclosed herein, which can be located near the 5′ end of the flop ITR in the vector. In some aspects, the AAV vector comprises a nucleic acid sequence encoding an antibody (e.g., a monoclonal antibody) or an antigen binding fragment thereof disclosed herein, which can be located near the 3′ end of the flop ITR in the vector. In some aspects, the AAV vector comprises a nucleic acid sequence encoding an antibody (e.g., a monoclonal antibody) or an antigen binding fragment thereof disclosed herein, which can be located between the 5′ end of the flip ITR and the 3′ end of the flop ITR in the vector. In some aspects, the AAV vector comprises a nucleic acid sequence encoding an antibody (e.g., a monoclonal antibody) or an antigen binding fragment thereof disclosed herein, which can be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in the vector.

In some aspects, the AAV vector comprises a nucleic acid sequence encoding an antibody (e.g., a monoclonal antibody) or an antigen binding fragment thereof disclosed herein, which can be located within about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or more than about 30 nucleotides downstream or upstream from the 5′ or 3′ end of an ITR (e.g., Flip or Flop ITR) in the vector.

As another non-limiting example, the AAV vector comprises a nucleic acid sequence encoding an antibody (e.g., a monoclonal antibody) or an antigen binding fragment thereof disclosed herein, which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream or upstream from the 5′ or 3′ end of an ITR (e.g., Flip or Flop ITR) in the vector.

In some aspects, the AAV vector comprises a nucleic acid sequence encoding an antibody (e.g., a monoclonal antibody) or an antigen binding fragment thereof disclosed herein, which can be located within the first about 1%, about 2%, about 3%, about 4%, about 5%, about 6%, about 7%, about 8%, about 9%, about 10%, about 15%, about 20%, about 25% or more than about 25% of the nucleotides upstream from the 5′ or 3′ end of an ITR (e.g., Flip or Flop ITR) in the vector.

As another non-limiting example, the AAV vector comprises a nucleic acid sequence encoding an antibody (e.g., a monoclonal antibody) or an antigen binding fragment thereof disclosed herein, which can be located with the first about 1-5%, about 1-10%, about 1-15%, about 1-20%, about 1-25%, about 5-10%, about 5-15%, about 5-20%, about 5-25%, about 10-15%, about 10-20%, about 10-25%, about 15-20%, about 15-25%, or about 20-25% downstream from the 5′ or 3′ end of an ITR (e.g., Flip or Flop ITR) in the vector.

### IV.A.2 Promoters

In some aspects, the payload region of the AAV vector comprises at least one element to enhance the nucleic acid specificity and/or expression. Non-limiting examples of elements to enhance the nucleic acid specificity and expression include, e.g., promoters, endogenous miRNAs, post-transcriptional regulatory elements (PREs), polyadenylation (Poly A) signal sequences and upstream enhancers (USEs), CMV enhancers, and introns. In some aspects, the enhancer is a CMV enhancer. In some aspects, the CMV enhancer comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 35.

Expression of nucleic acid of the present disclosure after delivery to or integration in the genomic DNA of a target cell can require a specific promoter, including but not limited to, a promoter that is species specific, inducible, tissue-specific, or cell cycle-specific (Parr et al., Nat. Med. 3: 1145-9 (1997); the contents of which are herein incorporated by reference in their entirety).

In some aspects, the promoter is deemed to be efficient when it drives expression of an antibody (e.g., a monoclonal antibody) or an antigen binding fragment thereof disclosed herein carried in the payload region of the AAV vector. In some aspects, the promoter is a promoter deemed to be efficient when it drives expression of the therapeutic molecule of the present disclosure in the cell being targeted (e.g., secretory cell).

Promoters can be naturally occurring or non-naturally occurring. Non-limiting examples of promoters include viral promoters and mammalian promoters. In some aspects, the promoters can be human promoters. In some aspects, the promoter can be truncated. Promoters which drive or promote expression in most tissues include, but are not limited to, human elongation factor 1a-subunit (EF1a), cytomegalovirus (CMV) immediate-early enhancer and/or promoter, chicken β-actin (CBA) and its derivative CAG, β glucuronidase (GUSB), or ubiquitin C (UBC). In some aspects, the promoter is a CMV early enhancer/chicken β actin (CAG) promoter, CAG, CBA, CMV, EF1α, EF1α with a CMV enhancer, a CMV promoter with a CMV enhancer (CMVe/p), a CMV promoter with a SV40 intron, or tissue specific promoter.

In some aspects, tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the contents of which are herein incorporated by reference in their entirety). Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene ηβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some aspects, the promoter can be less than 1 kb. In some aspects, the promoter can have a length between about 15-20, about 10-50, about 20-30, about 30-40, about 40-50, about 50-60, about 50-100, about 60-70, about 70-80, about 80-90, about 90-100, about 100-110, about 100-150, about 110-120, about 120-130, about 130-140, about 140-150, about 150-160, about 150-200, about 160-170, about 170-180, about 180-190, about 190-200, about 200-210, about 200-250, about 210-220, about 220-230, about 230-240, about 240-250, about 250-260, about 250-300, about 260-270, about 270-280, about 280-290, about 290-300, about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides.

In some aspects, the promoter can be a combination of two or more components of the same or different starting or parental promoters such as, but not limited to, CMV, CAG, EF1a, and CBA. In some aspects, the promoter is a CMV early enhancer/chicken β actin (CAG) promoter, CAG, CBA, CMV, EF1α, EF1α with a CMV enhancer, a CMV promoter with a CMV enhancer (CMVe/p), a CMV promoter with a SV40 intron. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38.

In some aspects, each component in the promoter can have a length between about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides. In some aspects, the promoter is a combination of a 382 nucleotide CMV-enhancer sequence and a 260 nucleotide CBA-promoter sequence.

In some aspects, the AAV vector comprises a ubiquitous promoter. Non-limiting examples of ubiquitous promoters include, e.g., CMV, CBA (including derivatives CAG, CBh, etc.), EF-1a, PGK, UBC, GUSB (hGBp), and UCOE (promoter of HNRPA2B1-CBX3).

In some aspects, the promoter is not cell specific. In some aspects, the promoter is a ubiquitin c (UBC) promoter. The UBC promoter can have a size of 300-350 nucleotides. In some aspects, the UBC promoter is 332 nucleotides. In some aspects, the promoter is a β-glucuronidase (GUSB) promoter. The GUSB promoter can have a size of 350-400 nucleotides. In some aspects, the GUSB promoter is 378 nucleotides. In some aspects, the promoter is a neurofilament light (NFL) promoter. The NFL promoter can have a size of 600-700 nucleotides. In some aspects, the NFL promoter is 650 nucleotides. In some aspects, the construct can be AAV-promoter-CMV/globin intron-modulatory polynucleotide-RBG, where the AAV can be self-complementary and the AAV can be the DJ serotype.

In some aspects, the AAV vector comprises a Pol III promoter. In some aspects, the AAV vector comprises a PI promoter. In some aspects, the AAV vector comprises a FXN promoter. In some aspects, the promoter is a phosphogly cerate kinase 1 (PGK) promoter. In some aspects, the promoter is a chicken β-actin (CBA) promoter. In some aspects, the promoter is a CAG promoter which is a construct comprising the cytomegalovirus (CMV) enhancer fused to the chicken beta-actin (CBA) promoter with a chimeric intron. In some aspects, the promoter is a cytomegalovirus (CMV) promoter. In some aspects, the promoter is a CBA promoter. In some aspects, the promoter is an EF1α promoter. In some aspects, the promoter is an EF1α promoter fused to a CMV enhancer. In some aspects, the promoter is a CMV promoter fused to a CMV enhancer. In some aspects, the promoter is a CMV promoter fused to a SV40 intron. In some aspects, the AAV vector comprises a HI promoter. In some aspects, the AAV vector comprises a U6 promoter. In some aspects, the AAV vector comprises a SP6 promoter. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38.

In some aspects, the promoter is a liver or a skeletal muscle promoter. Non-limiting examples of liver promoters include human a-1-antitrypsin (hAAT) and thyroxine binding globulin (TBG). Non-limiting examples of skeletal muscle promoters include Desmin, MCK or synthetic C5-12. In some aspects, the promoter is an RNA pol III promoter. In some aspects, the RNA pol III promoter is U6. In some aspects, the RNA pol III promoter is HI. In some aspects, the AAV vector comprises two promoters. In some aspects, the promoters are an EF1a promoter and a CMV promoter.

In some aspects, the AAV vector comprises an enhancer element, a promoter and/or a 5′UTR intron. The enhancer element, also referred to herein as an “enhancer,” can be, but is not limited to, a CMV enhancer, the promoter can be, but is not limited to, a EF1α, CMV, CBA, UBC, GUSB, NSE, Synapsin, MeCP2, and GFAP promoter and the 5′UTR/intron can be, but is not limited to, SV40, CBA-MVM (Minute virus of mice), human β-globin, immunoglobulin heavy chain, a chimera between the human β-globin and immunoglobin heavy chain gene. In some aspects, the intron comprises a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 33 and 82. In some aspects, the enhancer is a CMV enhancer. In some aspects, the CMV enhancer comprises a comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 35. In some aspects, the enhancer, promoter and/or intron used in combination can be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter, (9) GFAP promoter, (10) HI promoter; (11) U6 promoter; (12) CMV promoter, CMV enhancer; (13) EF1α promoter, CMV enhancer; or (14) CMV promoter, SV40 intron; (15) human β-globin intron and immunoglobin heavy chain intron chimera, EF1α promoter, CMV enhancer, CMV promoter, SV40 intron. In some aspects, the promoter is a cytomegalovirus (CMV) promoter. In some aspects the intron is a SV40 intron, MVM intron or a human betaglobin intron in the vector. In some aspects, the promoter is a CBA promoter. In some aspects, the promoter is an EF1α promoter. In some aspects, the promoter is a CMV promoter fused to a CMV enhancer. In some aspects, the promoter is a CMV enhancer fused to a EF1α promoter. In some aspects, the promoter is a CMV promoter fused to a SV40 intron. In some aspects, the AA vector comprises an engineered promoter. In some aspects, the AAV vector comprises a CMV early enhancer/chicken β actin (CAG) promoter. In some aspects the AAV vector comprises a promoter from a naturally expressed protein.

### IV.A.3 Untranslated Regions (UTRs)

By definition, wild-type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs can be engineered into UTRs to enhance transcribed product stability and production. In some aspects, a 5′ UTR from mRNA normally expressed in the liver (e.g., albumin, serum amyloid A, Apolipoprotein A/B/E, transferrin, alpha fetoprotein, erythropoietin, or Factor VIII) can be used in AAV vector of the disclosure to enhance expression, e.g., in brain tissue, and specifically in neuronal cells.

Wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG (SEQ ID NO: 80), where R is a purine (adenine or guanine) three bases upstream of the start codon (ATG), which is followed by another ‘G. In some aspects, the 5′UTR in a AAV vector of the present disclosure includes a Kozak sequence. In some aspects, the 5′UTR in a AAV vector of the present disclosure does not include a Kozak sequence.

Wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety). Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/AXU/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some aspects, the 3′ UTR of an AAV vector of the present disclosure can include an oligo(dT) sequence for addition of a poly-A tail. In some aspects, an AAV vector of the present disclosure can include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In some aspects, an AAV vector of the present disclosure can be engineered to include, alter or remove at least one miRNA binding site, sequence or seed region.

Any UTR from any gene known in the art can be incorporated into an AAV vector of the present disclosure. These UTRs, or portions thereof, can be placed in the same orientation as in the gene from which they were selected or they can be altered in orientation or location. In some aspects, the UTR used in an AAV vector of the present disclosure can be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR can be altered relative to a wild-type or native UTR by the change in orientation or location as taught above or can be altered by the inclusion of additional nucleotides, deletion of nucleotides, swapping or transposition of nucleotides. In some aspects, an AAV vector of the present disclosure comprises at least one artificial UTRs, which is not a variant of a wild-type UTR. In some aspects, an AAV vector of the present disclosure comprises UTRs, which have been selected from a family of transcripts whose proteins share a common function, structure, feature or property.

### IV.A.4 Polyadenylation Sequence

In some aspects, the AAV vectors of the present disclosure comprise at least one polyadenylation sequence. The AAV vectors of the present disclosure can comprise a polyadenylation sequence between the 3′ end of the payload coding sequence and the 5′ end of the 3′ ITR.

In some aspects, the polyadenylation sequence or “polyA sequence” can range from absent to about 500 nucleotides in length.

In some aspects, the polyadenylation sequence is about 10-100, about 10-90, about 10-80, about 10-70, about 10-60, about 10-55, about 10-50, about 20-100, about 20-90, about 20-80, about 20-70, about 20-60, about 20-55, about 20-50, about 30-100, about 30-90, about 30-80, about 30-70, about 30-60, about 30-55, about 30-50, about 40-100, about 40-90, about 40-80, about 40-70, about 40-60, about 40-55, about 40-50, about 45-100, about 45-90, about 45-80, or about 45-70 about 45-60, about 45-55, about 45-50 nucleotides in length. In some aspects, the polyadenylation sequence is about 49 nucleotides in length.

In some aspects, the AAV vector comprises a nucleic acid sequence encoding an antibody (e.g., a monoclonal antibody) or an antigen binding fragment thereof disclosed herein, which can be located upstream of the polyadenylation sequence in the vector. In some aspects, the AAV vector comprises a nucleic acid sequence encoding an antibody (e.g., a monoclonal antibody) or an antigen binding fragment thereof disclosed herein, which can be located downstream of a promoter such as, but not limited to, EF1α, CMV, U6, CAG, CBA EF1α with a CMV enhancer, CMV promoter with a SV40 intron, CMV promoter with a CMV enhancer, or a CBA promoter with a SV40 intron, MVM intron a human betaglobin intron, immunoglobulin heavy chain intron, or a chimera of a human betaglobin intron and a immunoglobulin heavy chain intron in the vector.

In some aspects, the AAV vector of the present disclosure comprises a nucleic acid sequence encoding an antibody (e.g., a monoclonal antibody) or an antigen binding fragment thereof disclosed herein, which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

In some aspects, the AAV vector comprises a rabbit globin polyadenylation (poly A) signal sequence. In some aspects, the AAV vector comprises a human growth hormone polyadenylation (poly A) signal sequence. In some aspects, the AAV vector comprises a bovine growth hormone polyadenylation (poly A) signal sequence. In some aspects, the poly A signal sequence has a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39, 40, 114, or 152.

### IV.A.5 Introns

In some aspects, the payload region of an AAV vector of the present disclosure comprises at least one element to enhance the expression such as one or more introns or portions thereof. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some aspects, the intron or intron portion can be between about 100 and about 500 nucleotides in length. In some aspects, the intron can have a length between about 80-100, about 80-120, about 80-140, about 80-160, about 80-180, about 80-200, about 80-250, about 80-300, about 80-350, about 80-400, about 80-450, about 80-500, about 200-300, about 200-400, about 200-500, about 300-400, about 300-500, or about 400-500 nucleotides.

In some aspects, the AAV vector can comprise a promoter such as, but not limited to, CMV or U6. In some aspects, the promoter for an AAV vector of the present disclosure is a CMV promoter. In some aspects, the promoter for an AAV vector of the present disclosure is a CMV early enhancer/chicken β actin (CAG) promoter. As another non-limiting example, the promoter for an AAV vector of the present disclosure is a U6 promoter. In some aspects, the AAV vector can comprise a CMV and a U6 promoter. In some aspects, the AAV vector can comprise a HI promoter. In some aspects, the AAV vector can comprise a CBA promoter. In some aspects, the AAV vector can comprise a chimeric intron. In some aspects, the AAV vector can comprise a SV40 intron. In some aspects, the AAV vector can comprise a immunoglobulin heavy chain intron. In some aspects, the AAV vector can comprise a human betaglobin intron. In some aspects, the AAV vector can comprise a chimera of a human betaglobin intron and a immunoglobulin heavy chain intron.

In some aspects, the promoter is a CMV early enhancer/chicken β actin (CAG) promoter, EF1α, CMV, CMV, EF1α promoter fused to CMV enhancer, CMV promoter fused to a SV40 intron, CMV promoter fused to a CMV enhancer, or a tissue specific promoters. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38.

In some aspects, the encoded antibody (e.g., a monoclonal antibody) or antigen binding fragment thereof disclosed herein can be located downstream of a promoter in an expression vector such as, but not limited to, CMV, U6, HI, CBA, CAG, or a CBA promoter with an intron such as SV40, MVM intron, a human betaglobin intron, human immunoglobulin heavy chain intron, a chimera of a human betaglobin intron and a human immunoglobulin heavy chain intron, or others known in the art. In some aspects, the intron is selected from the group consisting of an SV40 intron, MVM intron, a human betaglobin intron, a human immunoglobulin heavy chain intron, or a chimera of a human immunoglobulin heavy chain intron and a human betaglobin intron. In some aspects, the intron comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 33 and 82.

Further, the encoded antibody or antigen-binding fragment thereof can also be located upstream of the polyadenylation sequence in an expression vector. In some aspects, the encoded a therapeutic protein, e.g., antibody (e.g., a monoclonal antibody) or antigen binding fragment thereof or the fusion protein (e.g., the Fc fusion protein) disclosed herein, or therapeutic peptide can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

In some aspects, the vector or construct of the disclosure comprises a backbone. In some aspects, the backbone is a suitable for use in an AAV payload vector (e.g., comprising an antibody expression cassette flanked by 5′ and 3′ ITRs). In some aspects, the backbone can be a puc57 backbone (Addgene) or a modified version thereof. In some aspects, the backbone can comprise a filler sequence.

### IV.A.6 Filler Sequences

In some aspects, the AAV vector comprises one or more filler sequences (also referred to as “stuffer sequences”). In some aspects, the AAV vector comprises one or more filler sequences in order to have the length of the AAV vector be the optimal size for packaging. In some aspects, the AAV vector comprises at least one filler sequence in order to have the length of the AAV vector be about 2.0-2.5 kb, e.g., about 2.3 kb. In some aspects, the AAV vector comprises at least one filler sequence in order to have the length of the AAV vector be about 4.6 kb. In some aspects, the vector backbone comprises a filler sequence.

In some aspects, the AAV vector comprises one or more filler sequences in order to reduce the likelihood that a hairpin structure of the vector genome (e.g., a modulatory polynucleotide described herein) can be read as an inverted terminal repeat (ITR) during expression and/or packaging. In some aspects, the AAV vector comprises at least one filler sequence in order to have the length of the AAV vector be about 2.0-2.5 kb, e.g., about 2.3 kb. In some aspects, the AAV vector comprises at least one filler sequence in order to have the length of the AAV vector be about 4.6 kb.

In some aspects, the AAV vector is a single stranded (ss) AAV vector and comprises one or more filler sequences which have a length about between 0.1 kb and about 3.8 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, about 3.3 kb, about 3.4 kb, about 3.5 kb, about 3.6 kb, about 3.7 kb, or about 3.8 kb.

In some aspects, the AAV vector is a self-complementary (sc) AAV vector and comprises one or more filler sequences which have a length about between about 0.1 kb and about 1.5 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, or about 1.5 kb.

In some aspects, the AAV vector comprises any portion of a filler sequence. The vector can comprise, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of a filler sequence.

In some aspects, the AAV vector is a single stranded (ss) AAV vector and comprises one or more filler sequences in order to have the length of the AAV vector be about 4.6 kb. In some aspects, the AAV vector comprises at least one filler sequence and the filler sequence is located 3′ to the 5′ ITR sequence. In some aspects, the AAV vector comprises at least one filler sequence and the filler sequence is located 5′ to a promoter sequence. In some aspects, the AAV vector comprises at least one filler sequence and the filler sequence is located 3′ to the polyadenylation signal sequence. In some aspects, the AAV vector comprises at least one filler sequence and the filler sequence is located 5′ to the 3′ ITR sequence. In some aspects, the AAV vector comprises at least one filler sequence, and the filler sequence is located between two intron sequences. In some aspects, the AAV vector comprises at least one filler sequence, and the filler sequence is located within an intron sequence. In some aspects, the AAV vector comprises two filler sequences, and the first filler sequence is located 3′ to the 5′ ITR sequence and the second filler sequence is located 3′ to the polyadenylation signal sequence. In some aspects, the AAV vector comprises two filler sequences, and the first filler sequence is located 5′ to a promoter sequence and the second filler sequence is located 3′ to the polyadenylation signal sequence. In some aspects, the AAV vector comprises two filler sequences, and the first filler sequence is located 3′ to the 5′ ITR sequence and the second filler sequence is located 5′ to the 5′ ITR sequence.

In some aspects, the AAV vector is a self-complementary (sc) AAV vector and comprises one or more filler sequences in order to have the length of the AAV vector be about 2.3 kb. In some aspects, the AAV vector comprises at least one filler sequence and the filler sequence is located 3′ to the 5′ ITR sequence. In some aspects, the AAV vector comprises at least one filler sequence and the filler sequence is located 5′ to a promoter sequence. In some aspects, the AAV vector comprises at least one filler sequence and the filler sequence is located 3′ to the polyadenylation signal sequence. In some aspects, the AAV vector comprises at least one filler sequence and the filler sequence is located 5′ to the 3′ ITR sequence.

In some aspects, the AAV vector comprises at least one filler sequence, and the filler sequence is located between two intron sequences. In some aspects, the AAV vector comprises at least one filler sequence, and the filler sequence is located within an intron sequence. In some aspects, the AAV vector comprises two filler sequences, and the first filler sequence is located 3′ to the 5′ ITR sequence and the second filler sequence is located 3′ to the polyadenylation signal sequence. In some aspects, the AAV vector comprises two filler sequences, and the first filler sequence is located 5′ to a promoter sequence and the second filler sequence is located 3′ to the polyadenylation signal sequence. In some aspects, the AAV vector comprises two filler sequences, and the first filler sequence is located 3′ to the 5′ ITR sequence and the second filler sequence is located 5′ to the 5′ ITR sequence.

In some aspects, the AAV vector can comprise one or more filler sequences between one of more regions of the AAV vector. In some aspects, the filler region can be located before a region such as, but not limited to, a payload region, an ITR, a promoter region, an intron region, an enhancer region, and/or a polyadenylation signal sequence region. In some aspects, the filler region can be located after a region such as, but not limited to, a payload region, an ITR, a promoter region, an intron region, an enhancer region, and/or a polyadenylation signal sequence region. In some aspects, the filler region can be located before and after a region such as, but not limited to, a payload region, an ITR, a promoter region, an intron region, an enhancer region, and/or a polyadenylation signal sequence region.

In some aspects, the AAV vector can comprise one or more filler sequences which bifurcates at least one region of the AAV vector. The bifurcated region of the AVV vector can comprise about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the of the region to the 5′ of the filler sequence region.

In some aspects, the filler sequence can bifurcate at least one region so that about 10% of the region is located 5′ to the filler sequence and about 90% of the region is located 3′ to the filler sequence. In some aspects, the filler sequence can bifurcate at least one region so that about 20% of the region is located 5′ to the filler sequence and about 80% of the region is located 3′ to the filler sequence. In some aspects, the filler sequence can bifurcate at least one region so that about 30% of the region is located 5′ to the filler sequence and about 70% of the region is located 3′ to the filler sequence. In some aspects, the filler sequence can bifurcate at least one region so that about 40% of the region is located 5′ to the filler sequence and about 60% of the region is located 3′ to the filler sequence. In some aspects, the filler sequence can bifurcate at least one region so that about 50% of the region is located 5′ to the filler sequence and about 50% of the region is located 3′ to the filler sequence. In some aspects, the filler sequence can bifurcate at least one region so that about 60% of the region is located 5′ to the filler sequence and about 40% of the region is located 3′ to the filler sequence. In some aspects, the filler sequence can bifurcate at least one region so that about 70% of the region is located 5′ to the filler sequence and about 30% of the region is located 3′ to the filler sequence. In some aspects, the filler sequence can bifurcate at least one region so that about 80% of the region is located 5′ to the filler sequence and about 20% of the region is located 3′ to the filler sequence. In some aspects, the filler sequence can bifurcate at least one region so that about 90% of the region is located 5′ to the filler sequence and about 10% of the region is located 3′ to the filler sequence.

In some aspects, the AAV vector comprises a filler sequence after the 5′ ITR. In some aspects, the AAV vector comprises a filler sequence after the promoter region. In some aspects, the AAV vector comprises a filler sequence after the payload region. In some aspects, the AAV vector comprises a filler sequence after the intron region. In some aspects, the AAV vector comprises a filler sequence after the enhancer region. In some aspects, the AAV vector comprises a filler sequence after the polyadenylation signal sequence region. In some aspects, the AAV vector comprises a filler sequence before the promoter region. In some aspects, the AAV vector comprises a filler sequence before the payload region. In some aspects, the AAV vector comprises a filler sequence before the intron region.

In some aspects, the AAV vector comprises a filler sequence before the enhancer region. In some aspects, the AAV vector comprises a filler sequence before the polyadenylation signal sequence region. In some aspects, the AAV vector comprises a filler sequence before the 3′ ITR. In some aspects, a filler sequence can be located between two regions, such as, but not limited to, the 5′ ITR and the promoter region. In some aspects, a filler sequence can be located between two regions, such as, but not limited to, the 5′ ITR and the payload region.

In some aspects, a filler sequence can be located between two regions, such as, but not limited to, the 5′ ITR and the intron region. In some aspects, a filler sequence can be located between two regions, such as, but not limited to, the 5′ ITR and the enhancer region. In some aspects, a filler sequence can be located between two regions, such as, but not limited to, the 5′ ITR and the polyadenylation signal sequence region. In some aspects, a filler sequence can be located between two regions, such as, but not limited to, the promoter region and the payload region.

In some aspects, a filler sequence can be located between two regions, such as, but not limited to, the promoter region and the intron region. In some aspects, a filler sequence can be located between two regions, such as, but not limited to, the promoter region and the enhancer region. In some aspects, a filler sequence can be located between two regions, such as, but not limited to, the promoter region and the polyadenylation signal sequence region. In some aspects, a filler sequence can be located between two regions, such as, but not limited to, the promoter region and the 3′ ITR.

In some aspects, a filler sequence can be located between two regions, such as, but not limited to, the payload region and the intron region. In some aspects, a filler sequence can be located between two regions, such as, but not limited to, the payload region and the enhancer region. In some aspects, a filler sequence can be located between two regions, such as, but not limited to, the payload region and the polyadenylation signal sequence region. In some aspects, a filler sequence can be located between two regions, such as, but not limited to, the payload region and the 3′ ITR.

In some aspects, a filler sequence can be located between two regions, such as, but not limited to, the intron region and the enhancer region. In some aspects, a filler sequence can be located between two regions, such as, but not limited to, the intron region and the polyadenylation signal sequence region. In some aspects, a filler sequence can be located between two regions, such as, but not limited to, the intron region and the 3′ ITR. In some aspects, a filler sequence can be located between two regions, such as, but not limited to, the enhancer region and the polyadenylation signal sequence region. In some aspects, a filler sequence can be located between two regions, such as, but not limited to, the enhancer region and the 3′ ITR. In some aspects, a filler sequence can be located between two regions, such as, but not limited to, the polyadenylation signal sequence region and the 3′ ITR.

In some aspects, an AAV vector can comprise two filler sequences. The two filler sequences can be located between two regions as described herein.

### IV.A.7 Method for Producing Recombinant AAVs

The present disclosure provides also methods for the generation of AAV particles, by viral genome replication in a viral replication cell comprising contacting the viral replication cell with an AAV polynucleotide or AAV genome (e.g., an AAV vector of the present disclosure). In the context of the present disclosure, the AAV vectors disclosed herein, e.g., AAV vectors comprising at least one polynucleotide (e.g, an antibody expression cassette) encoding an antibody (e.g., a monoclonal antibody) or an antigen binding fragment thereof disclosed herein are considered AAV payload construct vectors.

In some aspects, an AAV particle is produced by a method comprising the steps of: (1) co-transfecting competent bacterial cells with a bacmid vector and either a viral construct vector and/or AAV payload construct vector, (2) isolating the resultant viral construct expression vector and AAV payload construct expression vector and separately transfecting viral replication cells, (3) isolating and purifying resultant payload and viral construct particles comprising viral construct expression vector or AAV payload construct expression vector, (4) co-infecting a viral replication cell with both the AAV payload and viral construct particles comprising viral construct expression vector or AAV payload construct expression vector, and (5) harvesting and purifying the viral particle comprising a parvoviral genome.

In one aspect, the present disclosure provides a method for producing an AAV particle comprising the steps of (1) simultaneously co-transfecting mammalian cells, such as, but not limited to HEK293 cells, with a payload region (e.g., polynucleotide encoding a therapeutic protein or therapeutic peptide of the disclosure), a construct expressing rep and cap genes and a helper construct, and (2) harvesting and purifying the AAV particle comprising a viral genome.

In some aspects, the AAV particles can be produced in a viral replication cell that comprises an insect cell. Growing conditions for insect cells in culture, and production of heterologous products in insect cells in culture are well-known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T1/2, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload, e.g. a recombinant viral construct, which comprises a polynucleotide (e.g, an antibody expression cassette) sequence encoding a payload such as an antibody (e.g., a monoclonal antibody) or an antigen binding fragment thereof disclosed herein.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct, parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV payload construct vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see, e.g., Urabe, M. et al., J Virol. 2006 February; 80 (4): 1874-85, the contents of which are herein incorporated by reference in their entirety.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif. 2009 June; 65(2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production. In some cases, bioreactors comprise stirred tank reactors.

### IV.A.8 Cell Lysis

Cells of the disclosure, including, but not limited to viral production cells, can be subjected to cell lysis according to any methods known in the art. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107.

### IV.A.9 AAV Purification

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

### V. Methods of Treatment and Use

Some aspects of the present disclosure are directed to a method of delivering a gene therapy encoding an anti-TNFalpha antibody or antigen-binding fragment thereof to a subject in need thereof. In some aspects, the administration is suitable for delivery of a gene therapy (e.g., a vector or rAAV particle disclosed herein) to one or both eyes. In some aspects, the administration is intraocular. In some aspects, the administration is by injection. In some aspects, the administration is intravitreal. In some aspects, the administration is intrastromal or transconjunctival. In some aspects, the method comprises administering to a secretory organ (e.g., a lymph node, gall bladder, thymus, hypothalamus, stomach, intestine, liver, pancreas, kidney, skin, muscle, and a secretory gland, e.g., a salivary gland) of the subject a delivery vector (e.g., a viral vector, a non-viral vectors, a plasmid, a lipid, protein particle, a bacterial vector, or a lysosome). In some aspects, the secretory gland is a salivary gland, pineal gland, thyroid gland, adrenal gland, or parathyroid gland. In some aspects, the secretory gland is a salivary gland. In some aspects, the anti-TNFalpha antibody or antigen-binding fragment thereof is expressed in the secretory organ or the secretory-like organ. In some aspects, the anti-TNFalpha antibody or antigen-binding fragment thereof is secreted from secretory organ or the secretory-like organ. In some aspects, the anti-TNFalpha antibody or antigen-binding fragment thereof is expressed in the salivary gland and secreted therefrom.

Certain aspects of the disclosure are directed a method of expressing an anti-TNFalpha antibody or antigen-binding fragment thereof in a subject in need thereof, comprising administering to the subject a rAAV particle, a vector, or a composition disclosed herein, thereby expressing the anti-TNFalpha antibody or antigen-binding fragment thereof in the subject.

Certain aspects of the disclosure are directed a method of neutralizing TNFalpha in a subject comprising administering to the subject a rAAV particle, a vector, or a composition disclosed herein, wherein the anti-TNFalpha antibody or antigen-binding fragment thereof expressed in the subject is capable of neutralizing TNFalpha. In some aspects, the TNFalpha neutralization is increased compared to TNFalpha neutralization in a subject administered recombinant adalimumab.

In some aspects, the subject suffers from an immune disease or disorder, an autoimmune disease or disorder, or an ocular disease or disorder. In some aspects, the subject suffers from an ocular disease or disorder.

Certain aspects of the disclosure are directed a method of treating an immune disease or disorder, an autoimmune disease or disorder, or an ocular disease or disorder in a subject in need thereof comprising administering to the subject an effective amount of a rAAV particle, a vector, or a composition disclosed herein, thereby expressing the anti-TNFalpha antibody or antigen-binding fragment thereof in the subject and treating the immune disease or disorder, an autoimmune disease or disorder, or an ocular disease or disorder.

Certain aspects of the disclosure are directed a method of treating an ocular disease or disorder in a subject in need thereof comprising intravitreally administering to the subject an effective amount of a recombinant adeno-associated virus (rAAV) particle comprising a capsid and a vector genome, the vector genome comprising an inverted terminal repeat (ITR) and an antibody expression cassette, wherein the antibody expression cassette comprises (a) a promoter, (b) a nucleic acid sequence encoding a heavy chain variable region (VH) of an anti-tumor necrosis factor alpha (anti-TNFalpha) antibody or an antigen-binding fragment thereof, (c) a linker sequence, and (d) a nucleic acid sequence encoding a light chain variable region (VL) of an anti-TNFalpha antibody or an antigen-binding fragment thereof, optionally, wherein the AAV capsid serotype is AAV2 or a modified version thereof, thereby expressing the anti-TNFalpha antibody or antigen-binding fragment thereof in the subject and treating the ocular disease or disorder.

In some aspects, the ocular disease or disorder is uveitis. In some aspects, the uveitis is non-infectious uveitis. In some aspects, the non-infectious uveitis is selected from the group consisting of intermediate uveitis, posterior uveitis, and panuveitis. In some aspects, the ocular disease or disorder is a corneal disease. In some aspects, the corneal disease is selected from the group consisting of peripheral ulcerative keratitis, corneal hemangiogenesis, and noninfectious corneal melting.

Certain aspects of the disclosure are directed to obtaining an effective steady state concentration of an anti-TNFalpha antibody (e.g., adalimumab) in the eye (e.g., in ocular fluid, for example, in the aqueous or viterious humor) of a subject in need thereof comprising intravitrial, intrastromal or transconjunctival administration of an single dose of an rAAV particle or vector disclosed herein to the subject, wherein the subject suffers from an ocular disease or disorder. In some aspects, the single dose comprises 1E9 vector genomes (vg) to 3E12 vg, 1E9 vg to 1E12 vg, 1E9 vg to 1E11 vg, or 1E9 vg to 3E10 vg. In some aspects, the administration comprises a single dose of about 1E9 vg. In some aspects, the administration comprises a single dose of about 1E10 vg. In some aspects, the administration comprises a single dose of about 1E11 vg. In some aspects, the single dose is administered in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye.

In some aspects, the administration is suitable for delivery of the rAAV particle or the vector to one or both eyes. In some aspects, the administration is by injection. In some aspects, the administration is intravitreal (e.g., for treating uveitis). In some aspects, the administration is intrastromal or transconjunctival (e.g., for treating a corneal disease).

In some aspect, the single dose is administered in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye. In some aspect, the single dose is administered in a volume of about 40 μL to 60 μL per eye. In some aspect, the single dose is administered in a volume of about 50 μL per eye.

In some aspects, the administration comprises a single dose within the range of 1E9 vector genomes (vg) to 3E12 vg. In some aspects, the administration comprises a single dose within the range of 1E9 vg to 1E12 vg. In some aspects, the administration comprises a single dose within the range of 1E9 vg to 1E11 vg. In some aspects, the administration comprises a single dose within the range of 1E9 vg to 3E10 vg. In some aspects, the administration comprises a single dose of about 1E9 vg. In some aspects, the administration comprises a single dose of about 1E10 vg. In some aspects, the administration comprises a single dose of about 1E11 vg.

In some aspects, the anti-TNFalpha antibody steady state concentration in the eye (e.g., in ocular fluid, for example, in the aqueous or viterious humor) of the subject after administration is 10 ng/mL to 1000 ng/mL (1 μg/mL). In some aspects, the anti-TNFalpha antibody steady state concentration in the eye (e.g., in ocular fluid, for example, in the aqueous or viterious humor) of the subject after administration is 10 ng/mL to 500 ng/mL. In some aspects, the anti-TNFalpha antibody steady state concentration in the eye (e.g., in ocular fluid, for example, in the aqueous or viterious humor) of the subject after administration is 10 ng/mL to 250 ng/mL. In some aspects, the anti-TNFalpha antibody steady state concentration in the eye (e.g., in ocular fluid, for example, in the aqueous or viterious humor) of the subject after administration is 10 ng/mL to 100 ng/mL. In some aspects, the anti-TNFalpha antibody steady state concentration in the eye (e.g., in ocular fluid, for example, in the aqueous or viterious humor) of the subject after administration is 20 ng/mL to 80 ng/mL. In some aspects, the anti-TNFalpha antibody steady state concentration in the eye (e.g., in ocular fluid, for example, in the aqueous or viterious humor) of the subject after administration is 10 ng/mL to 1000 ng/mL; 10 ng/mL to 800 ng/mL; 10 ng/mL to 500 ng/mL; 10 ng/mL to 100 ng/mL; 20 ng/mL to 1000 ng/mL; 20 ng/mL to 800 ng/mL; 20 ng/mL to 500 ng/mL; 20 ng/mL to 100 ng/mL; 50 ng/mL to 1000 ng/mL; 50 ng/mL to 800 ng/mL; 50 ng/mL to 500 ng/mL; or 50 ng/mL to 100 ng/mL.

In some aspects, the anti-TNFalpha antibody steady state concentration in the eye (e.g., in ocular fluid, for example, in the aqueous or viterious humor) of the subject after administration is at least 10 ng/mL, at least 20 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 250 ng/mL, at least 300 ng/mL, at least 400 ng/mL, at least 500 ng/mL, at least 600 ng/mL, at least 700 ng/mL, at least 800 ng/mL, at least 900 ng/mL, or at least 1 μg/mL. In some aspects, the anti-TNFalpha antibody steady state concentration in the eye (e.g., in ocular fluid, for example, in the aqueous or viterious humor) of the subject after administration is at least 5 ng/mL. In some aspects, the anti-TNFalpha antibody steady state concentration in the eye (e.g., in ocular fluid, for example, in the aqueous or viterious humor) of the subject after administration is at least 10 ng/mL. In some aspects, the anti-TNFalpha antibody steady state concentration in the eye (e.g., in ocular fluid, for example, in the aqueous or viterious humor) of the subject after administration is at least 15 ng/mL. In some aspects, the anti-TNFalpha antibody steady state concentration in the eye (e.g., in ocular fluid, for example, in the aqueous or viterious humor) of the subject after administration is at least 25 ng/mL. In some aspects, the anti-TNFalpha antibody steady state concentration in the eye (e.g., in ocular fluid, for example, in the aqueous or viterious humor) of the subject after administration is at least 50 ng/mL.

In some aspects, the anti-TNFalpha antibody steady state concentration in the serum of the subject after administration is less than 1% of the total anti-TNFalpha antibody concentration after administration (to one or both eyes). In some aspects, the anti-TNFalpha antibody steady state concentration in the serum of the subject after administration is less than 20 ng/mL, less than 15 ng/mL, less than 10 ng/mL, less than 5 ng/mL, less than 1 ng/mL, or less than 0.5 ng/mL. In some aspects, the anti-TNFalpha antibody steady state concentration in the serum of the subject after administration is 0.1 ng/mL to 20 ng/mL (e.g., 0.5 ng/mL to 20 ng/mL, 0.5 ng/mL to 10 ng/mL, or 0.5 ng/mL to 5 ng/mL).

In some aspects, the steady state concentration of antibody in the eye (e.g., in ocular fluid, for example, in the aqueous humor or vitrious humor) or serum can be determined according to any methods known in the art (see, e.g., Sugita et al., IOVS, July 2007, Vol. 48, No. 7).

In some aspects, methods comprising administering a gene therapy encoding an anti-TNFalpha antibody or antigen-binding fragment thereof comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the construct comprises any of SEQ ID NOs: 42-51, 52-61, 62-73, 74-77, 115-141, or 153-158; any of the sequences disclosed in Table 16 or Table 17; or any combination thereof.

In some aspects, methods comprise administering a gene therapy construct encoding an anti-TNFalpha antibody (e.g., adalimumab) is a multicistronic (e.g., bicistronic) construct (e.g., comprising a heavy chain and a light chain). In some aspects, the multicistronic (e.g., bicistronic) construct further comprises an F2A or IRES element.

In some aspects, the disclosure is directed to a method of delivering a gene therapy to a mucosal tissue (e.g., mouth, esophagus, lungs, stomach, and/or intestines). In some aspects, the gene therapy is administered to the salivary gland (e.g., by injection) and thereafter an antibody or an antigen binding fragment thereof is secreted from the salivary gland and swallowed. In some aspects, the therapeutic effect of the secreted anti-TNFalpha antibody or antigen-binding fragment thereof is local, systemic, or both.

In some aspects, the disclosure is directed to a method of delivering a gene therapy to a subject in need thereof, comprising administering to a secretory-like organ or other delivery site disclosed herein.

In some aspects, the disclosure is directed to a method of delivering a gene therapy to a subject in need thereof, comprising administering to the intestines (e.g., an intestinal wall, a perineal muscle, or an anal wall), adipose tissue, a proximate gland to the intestines, and/or one or both eyes (e.g., intraocular). In some aspects, the administration is suitable for delivery of a gene therapy (e.g., the rAAV particle or vector disclosed herein) to one or both eyes. In some aspects, the administration is intraocular. In some aspects, the administration is by injection. In some aspects, the administration is intravitreal. In some aspects, the administration is intrastromal or transconjunctival.

Some aspects of the present disclosure are directed to a method of delivering a nucleic acid to a cell of a subject, comprising administering to a secretory cell of the subject an adeno-associated virus (AAV) capsid comprising a nucleic acid comprising a promoter operably linked a polynucleotide encoding an anti-TNFalpha antibody or antigen-binding fragment thereof (e.g., a monoclonal antibody or an antigen binding fragment thereof), thereby delivering the nucleic acid to the secretory cell of the subject. In some aspects, the anti-TNFalpha antibody or antigen-binding fragment thereof is expressed in the secretory cell. In some aspects, the anti-TNFalpha antibody or antigen-binding fragment thereof is secreted from secretory cell. In some aspects, the anti-TNFalpha antibody or antigen-binding fragment thereof is expressed in a salivary gland cell and secreted therefrom.

In some aspects, the anti-TNFalpha antibody or antigen-binding fragment thereof is expressed in an intestinal cell, an adipose cell, a proximate gland cell, and/or an eye cell. In some aspects, the anti-TNFalpha antibody or antigen-binding fragment thereof is secreted from an intestinal cell, a perineal muscle cell, an anal wall cell, adipose cell, a proximate gland cell, and/or an eye cell. In some aspects, the anti-TNFalpha antibody or antigen-binding fragment thereof is secreted from the intestines (e.g., an intestinal wall, a perineal muscle, or an anal wall), adipose tissue, a proximate gland to the intestines, and/or one or both eyes (e.g., intraocular). In some aspects, the administration is suitable for delivery of a gene therapy (e.g., the rAAV particle or vector disclosed herein) to one or both eyes. In some aspects, the administration is intraocular. In some aspects, the administration is by injection. In some aspects, the administration is intravitreal. In some aspects, the administration is intrastromal or transconjunctival.

Some aspects of the present disclosure are directed to a method of delivering a gene therapy to a subject in need thereof, comprising administering to a secretory organ of the subject a delivery vector (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein. In some aspects, the secretory organ is selected from lymph node, gall bladder, thymus, hypothalamus, stomach, intestine, liver, pancreas, kidney, skin and/or secretory gland. In some aspects, the secretory organ is selected from heart, bone, muscle, skin, and/or adipose tissue. In some aspects, the secretory gland is a salivary gland, pineal gland, thyroid gland, adrenal gland, and parathyroid gland. In some aspects, the secretory gland is a salivary gland.

In some aspects, the disclosure is directed to a method of delivering a gene therapy to a subject in need thereof, comprising administering to the intestines (e.g., an intestinal wall, a perineal muscle, or an anal wall), adipose tissue, a proximate gland to the intestines, and/or one or both eyes (e.g., intraocular, intravitreal injection, intrastromal, or transconjunctival) of the subject a delivery vector (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein.

Some aspects of the present disclosure are directed to a method of delivering a nucleic acid to a cell of a subject, comprising administering to a secretory cell of the subject an adeno-associated virus (AAV) capsid comprising a nucleic acid comprising a promoter operably linked a polynucleotide encoding an antibody (e.g., a monoclonal antibody) or an antigen binding fragment thereof disclosed herein, thereby delivering the nucleic acid to the secretory cell of the subject.

Some aspects of the present disclosure are directed to a method of delivering a nucleic acid to a cell of a subject, comprising administering to the intestines (e.g., an intestinal wall, a perineal muscle, or an anal wall), adipose tissue, a proximate gland to the intestines, and/or one or both eyes (e.g., intraocular, intravitreal, intrastromal, or transconjunctival) of the subject an adeno-associated virus (AAV) capsid comprising a nucleic acid comprising a promoter operably linked a polynucleotide encoding an antibody (e.g., a monoclonal antibody) or an antigen binding fragment thereof disclosed herein, thereby delivering the nucleic acid to the subject.

In some aspects, the methods disclosed herein can be practiced through the administration of the gene therapy composition comprising the AAV vector, the AAV vector, the rAAV particle, a cell comprising an AAV vector of the present disclosure, a cell comprising the rAAV particle of the present disclosure, a cell comprising a polynucleotide encoding an anti-TNFalpha antibody or antigen-binding fragment thereof of the present disclosure integrated into its genomic DNA, or a pharmaceutical compositions comprising any of the above. Thus, methods disclosed herein reciting the administration of an AAV vector of the present disclosure can be also practiced by administering any of these compositions.

In some aspects, methods disclosed herein can be practiced through the administration of a gene therapy composition comprising a nucleic acid encoding an antibody or antigen binding fragment thereof that binds to a tumor necrosis factor (TNF or antigen-binding fragments thereof.

In some aspects, methods disclosed herein can be practiced through the administration of a gene therapy composition comprising a nucleic acid encoding an antibody or antigen binding fragment thereof comprising (i) a heavy chain variable region (VH) comprising a complementarity determining region (CDR) 1, a VH CDR2, and a VH CDR3 and (ii) a light chain variable region (VL) comprising a CDR1, a VL CDR2, and a VL CDR3. In some aspects, the VH CDRs 1-3 and VL CDRs 1-3 is from the corresponding CDRs of adalimumab.

In some aspects, methods disclosed herein can be practiced through the administration of a gene therapy composition comprising a nucleic acid encoding the anti-TNFalpha antibody or antigen-binding fragment thereof having the same amino acid sequence as adalimumab or a variant thereof.

Based on the methods disclosed herein, the gene therapy composition comprising an AAV vector, an AAV vector, or an rAAV particle of the present disclosure for use in therapy, or for use as a medicament, or for use in treating a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis (e.g., non-infectious uveitis such as intermediate uveitis, posterior uveitis, or panuveitis) or a corneal disease (e.g., peripheral ulcerative keratitis, corneal hemangiogenesis, or noninfectious corneal melting)), an inflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease in a subject in need thereof is contemplated.

In some aspect, the gene therapy composition is administered to a subject for treating an ocular disease or disorder. In some aspects, the ocular disease or disorder is uveitis. In some aspects, the uveitis is non-infectious uveitis. In some aspects, the non-infectious uveitis is selected from the group consisting of intermediate uveitis, posterior uveitis, and panuveitis. In some aspects, the ocular disease or disorder is a corneal disease. In some aspects, the corneal disease is selected from the group consisting of peripheral ulcerative keratitis, corneal hemangiogenesis, and noninfectious corneal melting.

In some aspects, an immune disease or disorder is selected from the group consisting of arthritis, ankylosing spondylitis, Crohn's disease, psoriasis, ulcerative colitis, inflammatory bowel disease, inflammation of the esophagus, Behçet's disease, relapsing polychondritis, checkpoint inhibitor induced colitis, diabetes, multiple sclerosis, hidradenitis suppurativa, uveitis, neuromyelitis optica, atypical hemolytic uremic syndrome, autoimmune hemolytic anemia, pure red cell aplasia, thrombocytopenic purpura, Evans syndrome, vasculitis, bullous skin disorder, Sjögren syndrome, anti-NMDA receptor encephalitis, Devic's disease, Graves' ophthalmopathy, autoimmune pancreatitis, opsoclonus myoclonus syndrome, myasthenia gravis, an IgG4-related disease, or any combination thereof.

In some aspects, the arthritis is rheumatoid arthritis, psoriatic arthritis, or juvenile idiopathic arthritis.

In some aspects, the psoriasis is plaque psoriasis.

In some aspects, the diabetes is type 1 diabetes mellitus. In some aspects, the diabetes is type 2 diabetes mellitus.

In some aspects, the vasculitis is granulomatosis with polyangiitis.

In some aspects, the thrombocytopenic purpura is thrombotic thrombocytopenic purpura. In some aspects, the thrombocytopenic purpura is idiopathic thrombocytopenic purpura.

In some aspects, bullous skin disorder is pemphigus vulgaris or bullous pemphigoid.

In some aspects, the inflammatory disease or disorder is atopic dermatitis, sinusitis, giant cell arteritis, cytokine release syndrome, or any combination thereof.

In some aspects, the bone disorder is selected from the group consisting of osteoporosis, treatment-induced bone loss, metastases to bone, giant cell tumor of bone, bone fracture, and bone fracture nonunions.

In some aspects, the disease or disorder associated with the sensitivity to allergens is asthma, chronic idiopathic urticarial, or a combination thereof.

In some aspects, the disease or disorder is an ocular disease or disorder. In some aspects, the ocular disease or disorder is uveitis. In some aspects, the uveitis is non-infectious uveitis. In some aspects, the non-infectious uveitis is selected from the group consisting of intermediate uveitis, posterior uveitis, and panuveitis. In some aspects, the ocular disease or disorder is a corneal disease. In some aspects, the corneal disease is selected from the group consisting of peripheral ulcerative keratitis, corneal hemangiogenesis, and noninfectious corneal melting.

In some aspects, the ocular disease or disorder is age-related macular degeneration (AMD), diabetic retinopathy, choroidal neovascularization, neovascular glaucoma, diabetic macular edema, retinopathy of prematurity, macular edema secondary to retinal vein occlusions, Graves' ophthalmopathy, macular degeneration, diabetic retinopathy, uveitis (e.g., a non-infectious uveitis selected from the group consisting of intermediate uveitis, posterior uveitis, and panuveitis), a corneal disease (e.g., peripheral ulcerative keratitis, corneal hemangiogenesis, or noninfectious corneal melting), or any combination thereof.

In some aspects, the oral mucosal disease or disorder is oral lichen planus, mucous membrane pemphigus, bullous pemphigus, pemphigus vulgaris, systemic lupus erythematosus, Behcet's disease, recurrent aphthous stomatitis, oral mucosal dermatitis, aphthous stomatitis, other oral mucosal diseases or disorders, or any combination thereof.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered intramuscularly, intracutaneously, intraocularly, intravitreally, intrastromaly, or transconjunctivaly. In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered to secretory organ (e.g., lymph node, gall bladder, thymus, hypothalamus, stomach, intestine, liver, pancreas, kidney, skin, and a secretory gland) by intraductal injection. In some aspects, the subject suffers from a disease or disorder selected from the group consisting of an immune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis (e.g., non-infectious uveitis such as intermediate uveitis, posterior uveitis, or panuveitis) or a corneal disease (e.g., peripheral ulcerative keratitis, corneal hemangiogenesis, and noninfectious corneal melting)), an inflammatory disease or disorder, an autoinflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease and any combination thereof.

In some aspects, the delivery vector for any of the uses disclosed herein comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77, 115-141, or 153-158, wherein the construct comprises one or more of IRES, furin cleavage site, 2a site, or a dual promoter (e.g., promoter-VH-IRES-VL, promoter-VH-furin-2A-VL, etc.). In some aspects, the delivery vector comprises any of SEQ ID NOs: 42-51, 52-61, 62-73, 74-77, 115-141, or 153-158; any of the sequences disclosed in Table 16 or 17; or any combination thereof.

In some aspects, a composition or delivery vector disclosed herein comprising a nucleic acid encoding an anti-TNFα antibody or antigen-binding fragment thereof is suitable for delivery to the salivary gland for treating an oral mucosal disease. In some aspects, a composition or delivery vector disclosed herein comprising a nucleic acid encoding an anti-TNFα antibody or antigen-binding fragment thereof is suitable for delivery to a subject for treating an inflammatory and autoimmune disease of the esophagus. In some aspects, a composition or delivery vector disclosed herein comprising a nucleic acid encoding an anti-TNFα antibody or antigen-binding fragment is suitable for delivery to the intestines (e.g., an intestinal wall, a perineal muscle, or an anal wall), adipose tissue, a proximate gland, or a combination thereof to the intestines for treating an inflammatory bowel disease and/or associated complications thereof, e.g., perianal fistulas. In some aspects, a composition or delivery vector disclosed herein comprising a nucleic acid encoding an anti-TNFα antibody or antigen-binding fragment thereof is suitable for delivery to one or both eyes for treating an ocular disease or disorder, e.g., uveitis. In some aspects, the uveitis is non-infectious uveitis. In some aspects, the non-infectious uveitis is selected from the group consisting of intermediate uveitis, posterior uveitis, and panuveitis. In some aspects, a composition or delivery vector disclosed herein comprising a nucleic acid encoding an anti-TNFα antibody or antigen-binding fragment thereof is suitable for delivery to one or both eyes for treating a corneal disease (e.g., peripheral ulcerative keratitis, corneal hemangiogenesis, and noninfectious corneal melting).

In some aspects, the gene therapy composition or rAAV particle disclosed herein is administered intraductally, by direct injection to the secretory organ (e.g., secretory gland), or both. In some aspects, the gene therapy composition or rAAV particle disclosed herein is administered intraductally, by direct injection to the salivary gland, or both.

In some aspects, the encoded antibody (e.g., a monoclonal antibody) or an antigen-binding fragment thereof disclosed herein is secreted from the salivary gland and swallowed. In some aspects, the therapeutic effect of the secreted antibody or antigen-binding fragment thereof or peptide is local, systemic, or both.

In some aspects, the gene therapy composition or rAAV particle disclosed herein is administered by direct injection to the salivary gland for treatment of an inflammatory or autoimmune disease of the esophagus.

In some aspects, the gene therapy composition or rAAV particle disclosed herein is administered by direct injection to the intestines (e.g., an intestinal wall, a perineal muscle, or an anal wall), adipose tissue, a proximate gland to the intestines, and/or one or both eyes (e.g., intraocular). In some aspects, the gene therapy composition or rAAV particle disclosed herein is administered intraocularly, by direct injection to the eye (e.g., intravitreal injection). In some aspects the administration is to the cornea, e.g., intrastromal or transconjunctival injection.

In some aspects, the subject does not suffer from a disease of a secretory organ. In some aspects, the subject does not suffer from a disease of a secretory gland. In some aspects, the subject does not suffer from a disease of the salivary gland.

In some aspects, the gene therapy composition (e.g., comprising the rAAV particle or vector disclosed herein) is suitable for delivery to one or both eyes. In some aspects, the administration is intraocular. In some aspects, the administration is by injection. In some aspects, the administration is intravitreal, intrastromal, or transconjunctival.

### VI. Pharmaceutical Compositions

In some aspects, a pharmaceutical composition disclosed herein comprises a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein and a pharmaceutically-acceptable excipient or carrier. Pharmaceutically acceptable excipients or carriers are determined in part by the particular composition being administered, as well as by the particular method used to administer the composition.

Accordingly, there is a wide variety of suitable formulations of pharmaceutical compositions comprising a delivery vector of the present disclosure (e.g., an AAV vector) or a plurality thereof (see, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises more than one AAV vector of the present disclosure, wherein each vector comprises at least one polynucleotide (e.g, an antibody expression cassette) encoding at least one therapeutic molecule disclosed herein.

In some aspects, a pharmaceutical composition comprises (i) one or more delivery vectors disclosed herein (e.g., AAV vectors or rAAV particle), and (ii) one or more therapeutic agents for the treatment of a disorder. In some aspects, the one or more delivery vectors disclosed herein (e.g., AAV vectors or rAAV particle) and the one or more therapeutic agents for a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis (e.g., non-infectious uveitis such as intermediate uveitis, posterior uveitis, or panuveitis) or a corneal disease (e.g., peripheral ulcerative keratitis, corneal hemangiogenesis, and noninfectious corneal melting)), an inflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease are co-administered in a single pharmaceutical composition.

In some aspects, the pharmaceutical composition disclosed herein is suitable for treating an ocular disease or disorder. In some aspects, the ocular disease or disorder is uveitis. In some aspects, the uveitis is non-infectious uveitis. In some aspects, the non-infectious uveitis is selected from the group consisting of intermediate uveitis, posterior uveitis, and panuveitis. In some aspects, the ocular disease or disorder is a corneal disease. In some aspects, the corneal disease is selected from the group consisting of peripheral ulcerative keratitis, corneal hemangiogenesis, and noninfectious corneal melting.

In some aspects, the pharmaceutical composition comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77 or 115, wherein the construct comprises one or more of IRES, furin cleavage site, 2a site, or a dual promoter (e.g., promoter-VH-IRES-VL, promoter-VH-furin-2A-VL, etc.). In some aspects, the pharmaceutical composition construct comprises any of SEQ ID NOs: 42-51, 52-61, 62-73, 74-77, 115; any of the sequences disclosed in Table 16 or 17; or any combination thereof.

In some aspects, the one or more delivery vectors disclosed herein (e.g., AAV vectors or AAV capsids) and the one or more therapeutic agents for the treatment of a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis (e.g., non-infectious uveitis such as intermediate uveitis, posterior uveitis, and panuveitis) or a corneal disease (e.g., peripheral ulcerative keratitis, corneal hemangiogenesis, and noninfectious corneal melting)), an inflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease are co-administered as separate pharmaceutical compositions. In some aspects, the pharmaceutical composition (e.g., comprising the rAAV particle or vector disclosed herein) is suitable for delivery to one or both eyes. In some aspects, the administration is intraocular. In some aspects, the administration is by injection. In some aspects, the administration is intravitreal. In some aspects, the administration is intrastromal or transconjunctival.

In some aspects, a pharmaceutical composition comprising one or more delivery vectors disclosed herein (e.g., AAV vectors or AAV capsids) is administered prior to the administration of a pharmaceutical composition comprising one or more therapeutic agents for the treatment of a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis), a corneal disease (e.g., peripheral ulcerative keratitis, corneal hemangiogenesis, and noninfectious corneal melting), an inflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease. In some aspects, the uveitis is non-infectious uveitis. In some aspects, the non-infectious uveitis is selected from the group consisting of intermediate uveitis, posterior uveitis, and panuveitis.

In some aspects, a pharmaceutical composition comprising one or more delivery vectors disclosed herein (e.g., AAV vectors or AAV capsids) is administered after the administration of a pharmaceutical composition comprising one or more therapeutic agents for the treatment of a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis), a corneal disease (e.g., peripheral ulcerative keratitis, corneal hemangiogenesis, and noninfectious corneal melting), an inflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease. In some aspects, the uveitis is non-infectious uveitis. In some aspects, the non-infectious uveitis is selected from the group consisting of intermediate uveitis, posterior uveitis, and panuveitis.

In some aspects, a pharmaceutical composition comprising one or more delivery vectors disclosed herein (e.g., AAV vectors or AAV capsids) is administered concurrently with a pharmaceutical composition comprising one or more therapeutic agents for the treatment of a disease or disorder (e.g., an immune disease or disorder, an autoimmune disease or disorder, a bone disease or disorder, a sensitivity to an allergen, cancer, a metabolic disease or disorder, a blood disease or disorder (also referred to as a hematological disease or disorder), a neurological disease or disorder, a neuromuscular disease or disorder, an ocular disease or disorder (e.g., uveitis), a corneal disease (e.g., peripheral ulcerative keratitis, corneal hemangiogenesis, and noninfectious corneal melting), an inflammatory disease or disorder, a cardiovascular disease or disorder, an oral mucosal disease or disorder, an esophageal disease or disorder, a gastrointestinal disease or disorder, a pulmonary disease or disorder, a rheumatological disease or disorder, an immune disease or disorder (e.g., inflammatory bowel disease. In some aspects, the uveitis is non-infectious uveitis. In some aspects, the non-infectious uveitis is selected from the group consisting of intermediate uveitis, posterior uveitis, and panuveitis.

In some aspects, an immune disease or disorder is selected from the group consisting of arthritis, ankylosing spondylitis, Crohn's disease, psoriasis, ulcerative colitis, Behçet's disease, relapsing polychondritis, checkpoint inhibitor induced colitis, diabetes, multiple sclerosis, hidradenitis suppurativa, uveitis, neuromyelitis optica, atypical hemolytic uremic syndrome, autoimmune hemolytic anemia, pure red cell aplasia, thrombocytopenic purpura, Evans syndrome, vasculitis, bullous skin disorder, Sjögren syndrome, anti-NMDA receptor encephalitis, Devic's disease, Graves' ophthalmopathy, autoimmune pancreatitis, opsoclonus myoclonus syndrome, myasthenia gravis, an IgG4-related disease, lichen planus, mucous membrane pemphigus, bullous pemphigus, pemphigus vulgaris, systemic lupus erythematosus, or any combination thereof. In some aspects, disease or disorder is an ocular disease or disorder. In some aspects, the ocular disease or disorder is uveitis. In some aspects, the uveitis is non-infectious uveitis. In some aspects, the non-infectious uveitis is selected from the group consisting of intermediate uveitis, posterior uveitis, and panuveitis. In some aspects, the ocular disease or disorder is a corneal disease. In some aspects, the corneal disease is selected from the group consisting of peripheral ulcerative keratitis, corneal hemangiogenesis, and noninfectious corneal melting

In some aspects, the pharmaceutical composition of the disclosure is formulated for intraductal administration. In some aspects, the pharmaceutical composition of the disclosure is formulated for direct injection. In some aspects, the pharmaceutical composition is formulated for direct injection to the salivary gland, one or both eyes (e.g., intravitreal injection), the intestines (e.g., an intestinal wall, a perineal muscle, or an anal wall), adipose tissue, a proximate gland to the intestines, intramuscularly, or any combination thereof. In some aspects, the pharmaceutical composition is formulated for intraocular administration. In some aspects, the pharmaceutical composition is formulated for administration is by injection. In some aspects, the pharmaceutical composition is formulated for intravitreal injection. In some aspects, the pharmaceutical composition is formulated for intrastromal or transconjunctival injection.

Also provided herein are pharmaceutical compositions comprising delivery vectors disclosed herein (e.g., AAV vectors or rAAV particle) having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of vectors, e.g., AAV vectors described herein. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

Acceptable carriers, excipients, or stabilizers are nontoxic to recipients (e.g., animals or humans) at the dosages and concentrations employed.

Examples of carriers or diluents include, but are not limited to, water, saline, Ringer's solutions, dextrose solution, and 5% human serum albumin. Except insofar as any conventional media or compound is incompatible with the delivery vectors disclosed herein (e.g., AAV vectors or rAAV particle), use thereof in the compositions is contemplated. In some aspects, a pharmaceutical composition is formulated to be compatible with its intended route of administration. The delivery vectors disclosed herein (e.g., AAV vectors or rAAV particle) can be administered by parenteral, topical, intravenous, oral, subcutaneous, intra-arterial, intradermal, intraductal (e.g., salivary gland), transdermal, rectal, intracranial, intravitreal, intrastromal, transconjunctival, intraperitoneal, intranasal, intratumoral, intramuscular route, or as inhalants. In some aspects, the pharmaceutical composition comprising the delivery vectors disclosed herein (e.g., AAV vectors or rAAV particle) is administered intravenously, e.g. by injection. In some aspects, the pharmaceutical composition comprising the delivery vectors disclosed herein (e.g., AAV vectors or rAAV particle) is administered intramuscularly. In some aspects, the pharmaceutical composition comprising the delivery vectors disclosed herein (e.g., AAV vectors or rAAV particle) is administered intravitreally (intraocularly). In some aspects, the pharmaceutical composition comprising the delivery vectors disclosed herein (e.g., AAV vectors or rAAV particle) is administered intrastromaly or transconjunctivaly. In some aspects, the pharmaceutical composition comprising the delivery vectors disclosed herein (e.g., AAV vectors or rAAV particle) is administered intraductally, by direct injection to the salivary gland. In some aspects, the pharmaceutical composition comprising the delivery vectors disclosed herein (e.g., AAV vectors or rAAV particle) is administered intracutaneously. The delivery vectors disclosed herein (e.g., AAV vectors or rAAV particle) can optionally be administered in combination with other therapeutic agents that are at least partly effective in treating the disease, disorder or condition for which the delivery vectors disclosed herein (e.g., AAV vectors or rAAV particle) are intended.

In some aspects, the pharmaceutical composition of the disclosure is formulated to achieve an effective steady state concentration of an anti-TNFalpha antibody (e.g., adalimumab) in the eye (e.g., in ocular fluid, for example, in the aqueous or viterious humor) of a subject in need thereof. In some aspects, the pharmaceutical composition is formulated for intravitrial, intrastromal or transconjunctival administration of an single dose of an rAAV particle or vector disclosed herein. In some aspects, the single dose of the pharmaceutical composition comprises 1E9 vector genomes (vg) to 3E12 vg, 1E9 vg to 1E12 vg, 1E9 vg to 1E11 vg, or 1E9 vg to 3E10 vg. In some aspects, the administration comprises a single dose of about 1E9 vg. In some aspects, the administration comprises a single dose of about 1E10 vg. In some aspects, the administration comprises a single dose of about 1E11 vg. In some aspects, the single dose of the composition is administered in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye.

In some aspects, the administration is suitable for delivery of a single dose of the pharmaceutical composition comprising the rAAV particle or the vector to one or both eyes. In some aspects, the administration is by injection. In some aspects, the administration of the pharmaceutical composition is intravitreal (e.g., for treating uveitis). In some aspects, the administration is intrastromal or transconjunctival (e.g., for treating a corneal disease).

In some aspect, the single dose of the pharmaceutical composition is administered in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye. In some aspect, the single dose of the pharmaceutical composition is administered in a volume of about 40 μL to 60 μL per eye. In some aspect, the single dose of the pharmaceutical composition is administered in a volume of about 50 μL per eye.

In some aspects, the administration comprises a single dose of the pharmaceutical composition comprises 1E9 vector genomes (vg) to 3E12 vg. In some aspects, a single dose of the pharmaceutical composition comprises 1E9 vg to 1E12 vg. In some aspects, a single dose of the pharmaceutical composition comprises 1E9 vg to 1E11 vg. In some aspects, a single dose of the pharmaceutical composition comprises 1E9 vg to 3E10 vg. In some aspects, a single dose of the pharmaceutical composition comprises about 1E9 vg. In some aspects, a single dose of the pharmaceutical composition comprises about 1E10 vg. In some aspects, a single dose of the pharmaceutical composition comprises about 1E11 vg.

The delivery vectors disclosed herein (e.g., AAV vectors or rAAV particle) can be formulated using one or more excipients to (1) increase stability; (2) increase cell transfection or transduction; (3) permit the sustained or delayed release; or (4) alter the biodistribution (e.g., target the AAV vector to specific tissues or cell types such as secretory cells).

### VII. Administration

The gene therapy compositions and delivery vectors disclosed herein (e.g., AAV vectors or rAAV particle) can be administered by any route which results in a therapeutically effective outcome, e.g., for therapeutic expression of an anti-TNFalpha antibody or antigen-binding fragment thereof disclosed herein. In some aspects, the delivery can be intramuscular (IM), intravenous (IV), intraductal (e.g., direct injection to the salivary gland), intravitreal, intrastromal, transconjunctival, or direct injection to a secretory organ or a secretory-like organ.

In some aspects, compositions of delivery vectors disclosed herein (e.g., AAV vectors) or AAV capsids can be administered in a way which facilitates the vectors to enter a secretory organ of the subject. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the muscle is skeletal muscle.

In certain aspects, the administration is intraductal. In some aspects, the delivery vectors disclosed herein (e.g., the non-viral vectors (including naked DNA)) comprising a nucleic acid is introduced into the secretory organ (e.g., a secretory gland) in vivo via the duct system (e.g., by retrograde intraductal administration, which can be accomplished by perfusion (e.g., continuous injection), or by a single, discontinuous injection). Intraductal administration can also be accomplished by cannulation, which can be accomplished for the pancreas and the liver by, for example, insertion of the cannula through a lumen of the gastrointestinal tract, by insertion of the cannula through an external orifice, insertion of the cannula through the common bile duct. Retrograde ductal administration can be accomplished in the pancreas and liver by endoscopic retrograde chalangio-pancreatography (ECRP). The methods of the disclosure can involve delivery to the pancreas, the liver, the salivary gland, or to any combination thereof. In some aspects, ductal administration provides advantages, e.g., because the vector is presented to the cells from “outside” the body (from the lumen), the immunological and inflammatory reactions that are commonly observed as a result of the administration of transforming formulations and their adjuvants into blood and interstitial fluid can be avoided.

Moreover, the cells of secretory glands form a monolayer that encloses the duct system. In some aspects, virtually all of the cells of the glands can be accessed by a single administration into the duct. In this way, it can be possible to transfect large masses of cells with a single procedure. The nucleic acid of interest can thus also be administered without substantial dilution (it is only diluted by the fluid in the duct system) and without the-need to develop organ specific targeting signals. In contrast, intravenous administration necessarily greatly dilutes the material and requires that it be targeted to the organ of interest in some fashion. In some aspects, the secretory gland cells are derived from a salivary gland, pineal gland, thyroid gland, adrenal gland, and parathyroid gland. In some aspects, the secretory gland cells are salivary gland cells.

In some aspects, the therapeutic molecule is administered, expressed and secreted from the salivary gland and swallowed. In some aspects, the therapeutic effect of the secreted therapeutic protein or peptide is local, systemic, or both.

The amount of nucleic acid to transform a sufficient number of secretory gland cells and provide for expression of therapeutic levels of the protein can be assessed using an animal model (e.g., a rodent (mouse or rat) or other mammalian animal model) to assess factors such as the efficiency of transformation, the levels of protein expression achieved, the susceptibility of the targeted secretory gland cells to transformation, and the amounts of vector and/or nucleic acid required to transform secretory gland cells.

In some aspects, the administration is intravitreal injection.

In some aspects, the administration is intrastromal or transconjunctival injection.

In some aspects, the administration is injection to the intestines (e.g., an intestinal wall, a perineal muscle, or an anal wall), adipose tissue, a proximate gland to the intestines, or a combination thereof.

The precise amount of vector and/or nucleic acid administered will vary greatly according to a number of factors including the susceptibility of the target cells to transformation, the size and weight of the subject, the levels of protein expression desired, and the condition to be treated.

In some aspects, a delivery vector of the present disclosure (e.g., a viral vector, a non-viral vectors, a plasmid, a lipid, a protein particle, a bacterial vector, or a lysosome) comprising a promoter operably linked to a nucleic acid sequence that encodes an antibody (e.g., a monoclonal antibody) or an antigen binding fragment thereof disclosed herein can be administered to secretory organ (e.g., secretory gland) by intraductal injection. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue. In some aspects, the delivery is by intraductal injection to the salivary gland.

In some aspects, a delivery vector of the present disclosure (e.g., a viral vector, a non-viral vectors, a plasmid, a lipid, a protein particle, a bacterial vector, or a lysosome) comprising a promoter operably linked to a nucleic acid sequence that encodes an antibody (e.g., a monoclonal antibody) or an antigen binding fragment thereof disclosed herein is administered by direct injection, e.g., to the secretory organ. In some aspects, the secretory organ is selected from lymph node, gall bladder, thymus, hypothalamus, stomach, intestine, liver, pancreas, kidney, skin, and/or secretory gland. In some aspects, the secretory organ is selected from heart, bone, muscle, skin, and/or adipose tissue.

In some aspects, the gene therapy composition, delivery vector, or rAAV particle disclosed herein is administered intraductally, by direct injection to the secretory organ, or both. In some aspects, the gene therapy composition, delivery vector, or rAAV particle disclosed herein is administered intraductally, by direct injection to the secretory gland, or both. In some aspects, the gene therapy composition, delivery vector, or rAAV particle disclosed herein is administered intraductally, by direct injection to the salivary gland, or both.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered intraductally, e.g., by direct injection to the salivary gland.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered by intramuscular injection.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered intravenously.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered by intradermal injection.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered intravitreally.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered intrastromaly or transconjunctivaly.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered by direct injection to the intestines (e.g., an intestinal wall, a perineal muscle, or an anal wall), adipose tissue, a proximate gland to the intestines, or any combination thereof.

The delivery vectors disclosed herein (e.g., AAV vectors or rAAV particle) can be administered in any suitable form, either as a liquid solution or suspension, as a solid form suitable for liquid solution or suspension in a liquid solution.

In some aspects, the delivery vector comprises a nucleic acid sequence encoding an anti-TNFalpha antibody or antigen-binding fragment thereof comprising: (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (e.g., SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77 or 115, wherein the construct comprises one or more of IRES, furin cleavage site, 2a site, or a dual promoter (e.g., promoter-VH-IRES-VL, promoter-VH-furin-2A-VL, etc.). In some aspects, the delivery vector the construct comprises any of SEQ ID NOs: 42-51, 52-61, 62-73, 74-77, 115; any of the sequences disclosed in Table 16 or 17; or any combination thereof.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered by intravitreal injection, intrastromal injection or transconjunctival injection. In some aspects, the delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered as a single dose by intravitreal injection, intrastromal injection or transconjunctival injection.

Clinical dosing of recombinant AAV (rAAV) therapeutics are usually based on vector genome (vg) titer per dose. In some aspects, a single dose of the rAAV disclosed herein comprises between 1E9 to 3E12 vector genomes/dose (alternatively, recited as between 1×10⁹ vg to 3×10¹² vg). As used herein, “between” includes the starting and ending dose in the range as well as all doses in between.

In some aspects, a single dose of the rAAV disclosed herein comprises between 1E9 vg to 3E12 vg, 1E9 vg to 2E12 vg, 1E9 vg to 1E12 vg, 1E9 vg to 5E11 vg, or 1E9 vg to 1E11 vg.

In some aspects, the administration comprises a dose within the range of 1E9 vg to 3E12 vg. In some aspects, the administration comprises a dose within the range of 1E9 vg to 1E12 vg. In some aspects, the administration comprises a dose within the range of 1E9 vg to 5E11 vg. In some aspects, the administration comprises a dose within the range of 1E9 vg to 1E11 vg. In some aspects, the administration comprises a dose within the range of 1E9 vg to 5E10 vg. In some aspects, the administration comprises a dose within the range of 1E9 vg to 3E10 vg. In some aspects, the administration comprises a dose within the range of 1E9 vg to 1E10 vg. In some aspects, the administration comprises a dose within the range of 1E9 vg to 5E9 vg.

In some aspects, the administration comprises a dose within the range of 1E10 vg to 3E12 vg. In some aspects, the administration comprises a dose within the range of 5E10 vg to 3E12 vg. In some aspects, the administration comprises a dose within the range of 1E11 vg to 3E12 vg. In some aspects, the administration comprises a dose within the range of 5E11 vg to 3E12 vg. In some aspects, the administration comprises a dose within the range of 1E11 vg to 1E12 vg. In some aspects, the administration comprises a dose within the range of 5E11 vg to 1E12 vg.

In some aspects, the administration comprises a single dose per eye. In some aspects, the administration comprises a single dose of about 1E9 vg. In some aspects, the administration comprises a single dose of about 5E9 vg. In some aspects, the administration comprises a single dose of about 1E10 vg. In some aspects, the administration comprises a single dose of about 5E10 vg. In some aspects, the administration comprises a single dose of about 1E11 vg. In some aspects, the administration comprises a single dose of about 5E11 vg. In some aspects, the administration comprises a single dose of about 1E12 vg.

In some aspect, the single dose is administered in a volume of 25 μL to 100 μL (e.g., 25 μL to 75 μL; 25 μL to 70 μL; 25 μL to 65 μL; 25 μL to 60 μL; 25 μL to 55 μL; or 25 μL to 50 μL) per eye. In some aspect, the single dose is administered in a volume of about 40 μL to 60 μL per eye. In some aspect, the single dose is administered in a volume of about 50 μL per eye.

In some aspects, the administration comprises a single dose (e.g., 25 μL to 100 μL) within the range of 1E9 vg to 3E12 vg to the eye (e.g., by intravitreal injection, intrastromal injection or transconjunctival injection). In some aspects, the administration comprises a single dose (e.g., 25 μL to 100 μL) within the range of 1E9 vg to 1E12 vg to the eye (e.g., by intravitreal injection, intrastromal injection or transconjunctival injection). In some aspects, the administration comprises a single dose (e.g., 25 μL to 100 μL) within the range of 1E9 vg to 1E11 vg to the eye (e.g., by intravitreal injection, intrastromal injection or transconjunctival injection).

### VIII. Kits

The present disclosure also provides kits, or products of manufacture, comprising (i) the delivery vector of the present disclosure, or a pharmaceutical composition of the present disclosure, and (ii) optionally instructions for use (e.g., a package insert with instructions to perform any of the methods described herein).

In some aspects, the kit or product of manufacture comprises (i) comprising the delivery vectors of the present disclosure (e.g., an AAV vector comprising a polynucleotide or an antibody expression cassette encoding a anti-TNFalpha antibody or antigen-binding fragment thereof disclosed herein), or a pharmaceutical composition of the present disclosure, (ii) optionally, an additional therapeutic agent, and (iii) optionally, instructions for use (e.g., a package insert with instructions to perform any of the methods described herein are also contemplated).

In some aspects, the components of a kit or product of manufacture disclosed herein are in one or more containers. In some aspects, the kit or product of manufacture comprises (i) an AAV vector comprising a polynucleotide (e.g, an antibody expression cassette) encoding an anti-TNFalpha antibody or antigen-binding fragment thereof disclosed herein, and (ii) a brochure with instructions to insert the polynucleotide in the AAV vector.

In some aspects, a kit or product of manufacture of the present disclosure comprises at least one delivery vector (e.g., AAV vector or rAAV particle). In some aspects, a kit or product of manufacture of the present disclosure comprises at least one polynucleotide (e.g, an antibody expression cassette) encoding at least one anti-TNFalpha antibody or antigen-binding fragment thereof disclosed herein. In some aspects, the kit comprises a nucleic acid sequence encoding an anti-TNFalpha antibody or antigen-binding fragment thereof comprising (i) coding sequences for the VH and VL CDRs of adalimumab (e.g., SEQ ID NOs: 87-92, 96-101, and 106-109; any of the CDR encoding sequences disclosed in Table 3 or Table 4; or any combination thereof); (ii) coding sequences for the VH and VL of adalimumab (e.g., SEQ ID NOs: 7-9, 11-13, 102 (or nucleotides 55-417 of SEQ ID NO: 56), 103 (or nucleotides 61-381 of SEQ ID NO: 49), 143, 147, 145, or 149; any of the VH or VL encoding sequences disclosed in Table 5 or Table 7; or any combination thereof); (iii) coding sequences for the HC and LC of adalimumab (SEQ ID NOs: 17-19, 21-23, 110 (or nucleotides 55-1407 of SEQ ID NO: 56 or nucleotides 1951-3304 of SEQ ID NO: 64), 111 (or nucleotides 61-702 of SEQ ID NO: 49 or nucleotides 3460-4105 of SEQ ID NO: 64), 142, 146, 150, 151, 144, or 148; any of the HC or LC encoding sequences disclosed in Table 11 or Table 13; or any combination thereof); or (iv) a construct comprising any of SEQ ID NOs: 62-77 or 115, wherein the construct comprises one or more of IRES, furin cleavage site, 2a site, or a dual promoter (e.g., promoter-VH-IRES-VL, promoter-VH-furin-2A-VL, etc.). In some aspects, the construct comprises any of SEQ ID NOs: 42-51, 52-61, 62-73, 74-77, 115; any of the sequences disclosed in Table 16 or 17; or any combination thereof.

One skilled in the art will readily recognize that vectors, polynucleotides, and pharmaceutical compositions of the present disclosure, or combinations thereof, can be readily incorporated into one of the established kit formats which are well known in the art.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

The following examples are offered by way of illustration and not by way of limitation.

### EXAMPLES

### Example 1: Modified Anti-TNFalpha Antibody Nucleic Acids

The following modified anti-TNFalpha antibody ORF nucleic acid sequences (shown in Table 16) corresponding to SEQ ID NOs: 42-61 were designed in silico. The OFRs include nucleic acid molecules encoding an anti-TNFalpha heavy chain and a light chain.

TABLE 16
Modified ORF Nucleic Acid Sequences Encoding anti-TNFalpha Antibody

| SEQ ID NO | Name | Sequence |
|---|---|---|
| Full: 705 | LC | atgtacaggatgcaactcctgtcttgcattgcactaagtcttgcacttgtcacaaactcggacatcca |
| IL-2 Leader: | ORF | gatgacccaaagcccctcctctctgtcagccagtgtgggcgatcgggtcacaattacttgcagag |
| 1-60 | #1 | cttcgcagggaataaggaactacctcgcatggtatcagcaaaagcctgggaaagcgccaaagtt |
| LC Var 61-381 | | gcttatctacgccgctagcacgctgcagtccggtgttccgtctcgcttctcaggcagtggaagcgg |
| LC Constant: | | gaccgactttacattaactatttcctctctgcagcccgaggatgtggccacctattactgtcagcgat |
| 382-702 | | ataatcgtgcaccttacacattcggccaaggtaccaaagtagaaatcaagcgaactgtggctgca |
| (SEQ ID NO: | | ccatctgtcttcatcttcccgccatctgatgagcagttgaaatctggaactgcctctgttgtgtgcctg |
| 42) | | ctgaataacttctatcccagagaggccaaagtacagtggaaggtggataacgccctccaatcggg |
| | | taactcccaggagagtgtcacagagcaggacagcaaggacagcacctacagcctcagcagcac |
| | | cctgacgctgagcaaagcagactacgagaaacacaaagtctacgcctgcgaagtcacccatcag |
| | | ggcctgagctcgcccgtcacaaagagcttcaacaggggagagtgttga |
| Full: 705 | LC | atgtacaggatgcaactcctgtcttgcattgcactaagtcttgcacttgtcacaaactcagacatcca |
| IL-2 Leader: | ORF | gatgacccaaagcccctcctctctgtcagccagtgtgggggatcgcgtcacaattacttgcagag |
| 1-60 | #2 | cttcccagggaataaggaactacctcgcgtggtatcagcaaaagcctgggaaagcgccaaagtt |
| LC Var 61-381 | | gcttatctatgccgctagcactctgcagtcaggtgttccttctagattctcaggcagtggaagcggg |
| LC Constant: | | actgactttacattaactatttcctctctgcagcctgaggatgtggccacctattactgtcagcggtat |
| 382-702 | | aatcgcgcaccttacacatttggccaaggtaccaaagtagaaatcaagcggactgtggctgcacc |
| (SEQ ID NO: | | atctgtcttcatcttcccgccatctgatgagcagttgaaatctggaactgcctctgttgtgtgcctgct |
| 43) | | gaataacttctatcccagagaggccaaagtacagtggaaggtggataatgccctccaaagtggta |
| | | actcccaggagagtgtcacagagcaggacagcaaggacagcacctacagcctcagcagcacc |
| | | ctgaccctgagcaaagcagactatgagaaacacaaagtctacgcgtgtgaagtcacccatcagg |
| | | gcctgagctccccagtcacaaagagcttcaacaggggagagtgttga |
| Full: 705 | LC | atgtacaggatgcaactcctgtcttgcattgcactaagtcttgcacttgtcacaaactcagacatcca |
| IL-2 Leader: | ORF | gatgacccaaagcccctcctctctgtcagccagtgtgggggatagagtcacaattacttgcagag |
| 1-60 | #3 | cttcccagggaataaggaactacttggcatggtatcagcaaaagcctgggaaagctccaaagttg |
| LC Var 61-381 | | cttatctatgctgctagcactctgcagtcaggtgttccttctagattctcaggcagtggaagtgggac |
| LC Constant: | | tgactttacattaactatttcctctctgcagcctgaggatgtggccacctattactgtcagaggtataat |
| 382-702 | | agagcaccttacacatttggccaaggtaccaaagtagaaatcaagaggactgtggctgcaccatc |
| (SEQ ID NO: | | tgtcttcatcttccccccatctgatgagcagttgaaatctggaactgcctctgttgtgtgcctgctgaa |
| 44) | | taacttctatcccagagaggccaaagtacagtggaaggtggataatgccctccaatcaggtaactc |
| | | ccaggagagtgtcacagagcaggacagcaaggacagcacctacagcctcagcagcaccctga |
| | | ccctgagcaaagcagactatgagaaacacaaagtctatgcctgtgaagtcacccatcagggcctg |
| | | agctccccagtcacaaagagcttcaacaggggagagtgttga |
| Full: 705 | LC | atgtacaggatgcaactcctgtcttgcattgcactaagtcttgcacttgtcacaaactcggacatcca |
| IL-2 Leader: | ORF | gatgacccaaagcccctcctctctgtcagccagtgtgggcgatcgggtcacaattacttgcagag |
| 1-60 | #4 | cttcgcagggaataaggaactacctcgcatggtatcagcaaaagcctgggaaagcgccaaagtt |
| LC Var 61-381 | | gcttatctacgccgccagcacgctgcagtccggtgttccgtctcgcttctctggcagtggaagcgg |
| LC Constant: | | gaccgactttacattaactatttcctctctgcaacccgaggatgtggccacctattactgtcagcgat |
| 382-702 | | ataatcgtgcaccttacacattcggccaaggtaccaaagtagaaatcaagcgaactgtggctgca |
| (SEQ ID NO: | | ccatctgtcttcatcttcccgccatctgatgagcagttgaaatctggaactgcctctgttgtgtgcctg |
| 45_ | | ctgaataacttctatcccagagaggccaaagtacagtggaaggtggataacgccctccaatcggg |
| | | caactcccaggagagtgtcacagagcaggacagcaaggacagcacctacagcctcagcagca |
| | | ccctgacgctgagcaaagcagactacgagaaacacaaagtctacgcctgcgaagtcacccatca |
| | | gggcctgagctcgcccgtcacaaagagcttcaacaggggagagtgttga |
| Full: 705 | LC | atgtacaggatgcaactcctgtcttgcattgcactaagtcttgcacttgtcacaaactcagacatcca |
| IL-2 Leader: | ORF | gatgacccaaagcccctcctctctgtcagccagtgtgggggatcgcgtcacaattacttgcagag |
| 1-60 | #5 | cttcccagggaataaggaactacctcgcgtggtatcagcaaaagcctgggaaagcgccaaagtt |
| LC Var 61- | | gcttatctatgccgccagcactctgcagtcaggtgttcctagtagattctctggcagtggaagcggg |
| 381 | | actgactttacattaactatttcctctctgcaacctgaggatgtggccacctattactgtcagcggtat |
| LC Constant: | | aatcgcgcaccttacacatttggccaaggtaccaaagtagaaatcaagcggactgtggctgcacc |
| 382-702 | | atctgtcttcatcttcccgccatctgatgagcagttgaaatctggaactgcctctgttgtgtgcctgct |
| (SEQ ID NO: | | gaataacttctatcccagagaggccaaagtacagtggaaggtggataatgccctccaaagtggca |
| 46) | | actcccaggagagtgtcacagagcaggacagcaaggacagcacctacagcctcagcagcacc |
| | | ctgaccctgagcaaagcagactatgagaaacacaaagtctacgcgtgtgaagtcacccatcagg |
| | | gcctgagctccccagtcacaaagagcttcaacaggggagagtgttga |
| Full: 705 | LC | atgtacaggatgcaactcctgtcttgcattgcactaagtcttgcacttgtcacaaactcagacatcca |
| IL-2 Leader: | ORF | gatgacccaaagcccctcctctctgtcagccagtgtgggggatagagtcacaattacttgcagag |
| 1-60 | #6 | cttcccagggaataaggaactacttggcatggtatcagcaaaagcctgggaaagctccaaagttg |
| LC Var 61- | | cttatctatgctgccagcactctgcagtcaggtgttcctagtagattctcaggcagtggaagtggga |
| 381 | | ctgactttacattaactatttcctctctgcaacctgaggatgtggccacctattactgtcagaggtata |
| LC Constant: | | atagagcaccttacacatttggccaaggtaccaaagtagaaatcaagaggactgtggctgcacca |
| 382-702 | | tctgtcttcatcttccccccatctgatgagcagttgaaatctggaactgcctctgttgtgtgcctgctg |
| (SEQ ID NO: | | aataacttctatcccagagaggccaaagtacagtggaaggtggataatgccctccaatcaggcaa |
| 47) | | ctcccaggagagtgtcacagagcaggacagcaaggacagcacctacagcctcagcagcaccct |
| | | gaccctgagcaaagcagactatgagaaacacaaagtctatgcctgtgaagtcacccatcagggc |
| | | ctgagctccccagtcacaaagagcttcaacaggggagagtgttga |
| Full: 705 | LC | atgtacaggatgcaactcctgtcttgcattgcactaagtcttgcacttgtcacaaactcggacatcca |
| IL-2 Leader: | ORF | gatgacccaaagcccctcctctctgtcagccagtgtgggcgatcgggtcacaattacttgcagag |
| 1-60 | #7 | cttcgcagggaataaggaactacctcgcatggtatcagcaaaagcctgggaaagcgccaaagtt |
| LC Var 61- | | gcttatctacgccgccagcacgctgcagtccggtgttccgtctcgcttctctggcagtggaagcgg |
| 381 | | gaccgactttacattaactatttcctctctgcaacccgaggatgtggccacctattactgtcagcgat |
| LC Constant: | | ataatcgtgcaccttacacattcggccaaggtaccaaagtagaaatcaagcgaactgtggctgca |
| 382-702 | | ccatctgtcttcatcttcccgccatctgatgagcagttgaaatctggaactgcctctgttgtgtgcctg |
| (SEQ ID NO: | | ctgaataacttctatcccagagaggccaaagtacagtggaaggtggataacgccctccaatcggg |
| 48) | | caactcccaggagagtgtcacagagcaggacagcaaggacagcacctacagcctcagcagca |
| | | ccctgacgctgagcaaagcagactacgagaaacacaaagtctacgcctgcgaagtcacccatca |
| | | gggcctgagctcgcccgtcacaaagagcttcaacaggggagagtgttga |
| Full: 705 | LC | atgtacaggatgcaactcctgtcttgcattgcactaagtcttgcacttgtcacaaactcagacatcca |
| IL-2 Leader: | ORF | gatgacccaaagcccctcctctctgtcagccagtgtgggggatcgcgtcacaattacttgcagag |
| 1-60 | #8 | cttcccagggaataaggaactacctcgcgtggtatcagcaaaagcctgggaaagcgccaaagtt |
| LC Var 61- | | gcttatctatgccgccagcactctgcagtcaggtgttcctagtagattctctggcagtggaagcggg |
| 381 | | actgactttacattaactatttcctctctgcaacctgaggatgtggccacctattactgtcagcggtat |
| LC Constant: | | aatcgcgcaccttacacatttggccaaggtaccaaagtagaaatcaagcggactgtggctgcacc |
| 382-702 | | atctgtcttcatcttcccgccatctgatgagcagttgaaatctggaactgcctctgttgtgtgcctgct |
| (SEQ ID NO: | | gaataacttctatcccagagaggccaaagtacagtggaaggtggataatgccctccaaagtggca |
| 49) | | actcccaggagagtgtcacagagcaggacagcaaggacagcacctacagcctcagcagcacc |
| | | ctgaccctgagcaaagcagactatgagaaacacaaagtctacgcgtgtgaagtcacccatcagg |
| | | gcctgagctccccagtcacaaagagcttcaacaggggagagtgttga |
| Full: 705 | LC | atgtacaggatgcaactcctgtcttgcattgcactaagtcttgcacttgtcacaaactcagacatcca |
| IL-2 Leader: | ORF | gatgacccaaagcccctcctctctgtcagccagtgtgggggatagagtcacaattacttgcagag |
| 1-60 | #9 | cttcccagggaataaggaactacttggcatggtatcagcaaaagcctgggaaagctccaaagttg |
| LC Var 61- | | cttatctatgctgccagcactctgcagtcaggtgttcctagtagattctcaggcagtggaagtggga |
| 381 | | ctgactttacattaactatttcctctctgcaacctgaggatgtggccacctattactgtcagaggtata |
| LC Constant: | | atagagcaccttacacatttggccaaggtaccaaagtagaaatcaagaggactgtggctgcacca |
| 382-702 | | tctgtcttcatcttccccccatctgatgagcagttgaaatctggaactgcctctgttgtgtgcctgctg |
| (SEQ ID NO: | | aataacttctatcccagagaggccaaagtacagtggaaggtggataatgccctccaatcaggcaa |
| 50) | | ctcccaggagagtgtcacagagcaggacagcaaggacagcacctacagcctcagcagcaccct |
| | | gaccctgagcaaagcagactatgagaaacacaaagtctatgcctgtgaagtcacccatcagggc |
| | | ctgagctccccagtcacaaagagcttcaacaggggagagtgttga |
| Full: 705 | LC | atgtacaggatgcaactcctgtcttgcattgcactaagtcttgcacttgtcacaaactcggacatcca |
| IL-2 Leader: | ORF | gatgacccaaagcccctcctctctgtcagccagtgtgggcgatcgggtcacaattacttgcagag |
| 1-60 | #10 | cttcgcagggaataaggaactacctcgcatggtatcagcaaaagcctgggaaagcgccaaagtt |
| LC Var 61- | | gcttatctacgccgccagcacgctgcagtccggtgttccgtctcgcttctctggcagtggaagcgg |
| 381 | | gaccgactttacattaactatttcctctctgcaacccgaggatgtggccacctattactgtcagcgat |
| LC Constant: | | ataatcgtgcaccttacacattcggccaaggtaccaaagtagaaatcaagcgaactgtggctgca |
| 382-702 | | ccatctgtcttcatcttcccgccatctgatgagcagttgaaatctggaactgcctctgttgtgtgcctg |
| (SEQ ID NO: | | ctgaataacttctatcccagagaggccaaagtacagtggaaggtggataacgccctccaatcggg |
| 51) | | caactcccaggagagtgtcacagagcaggacagcaaggacagcacctacagcctcagcagca |
| | | ccctgacgctgagcaaagcagactacgagaaacacaaagtctacgcctgcgaagtcacccatca |
| | | gggcctgagctcgcccgtcacaaagagcttcaacaggggagagtgttga |
| Full: 1410 | HC | atgcacagctcagcactgctctgttgcctggtcctcctgactggggtgagggccgaggtgcagct |
| IL-10 leader: | ORF | ggtcgaaagcggcggagggctcgttcaacccggtcggtccttgagactttcttgcgccgcttcag |
| 1-54 | #1 | gcttcacctttgacgattacgcaatgcactgggtgaggcaggcgcctggaaaggggctggagtg |
| HC Var: 55- | | ggtaagtgccatcacatggaactcgggccatattgactatgctgatagcgtggaaggtcgcttcac |
| 417 | | tatatcccgagacaatgccaaaaactctttatacctgcagatgaattcactacgtgcagaggatacg |
| HC Constant: | | gccgtctattactgtgctaaggtgagttatctcagcaccgcatcctctctggactactggggacaag |
| 418-1407 | | ggacattggttactgtgagctccgcctccaccaagggcccatcggtcttccccctggcaccctcct |
| (SEQ ID NO: | | ccaagagcacctctgggggcacagcggccctgggctgcctggtcaaggactacttccccgaac |
| 52) | | cggtgacggtgtcgtggaactcaggcgccctgaccagcggcgtgcacaccttcccggctgtcct |
| | | acagtcctcaggactctactccctcagcagcgtggtgaccgtgccctccagcagcttgggcaccc |
| | | agacctacatctgcaacgtgaatcacaagcccagcaacaccaaggtggacaagaaagttgagcc |
| | | caaatcttgtgacaaaactcacacatgcccaccgtgcccagcacctgaactcctggggggaccgt |
| | | cagtcttcctcttccccccaaaacccaaggacaccctcatgatctcccggacccctgaggtcacat |
| | | gcgtggtggtggacgtgagccacgaagaccctgaggtcaagttcaactggtacgtggacggcgt |
| | | ggaggtgcataatgccaagacaaagccgcgggaggagcagtacaacagcacgtaccgtgtggt |
| | | cagcgtcctcaccgtcctgcaccaggactggctgaatggcaaggagtacaagtgcaaggtctcc |
| | | aacaaagccctcccagcccccatcgagaaaaccatctccaaagccaaagggcagccccgaga |
| | | accacaggtgtacaccctgcccccatcccgggatgagctgaccaagaaccaggtcagcctgac |
| | | ctgcctggtcaaaggcttctatcccagcgacatcgccgtggagtgggagagcaatgggcagccg |
| | | gagaacaactacaagaccacgcctcccgtgctggactccgacggctccttcttcctctacagcaa |
| | | gctcaccgtggacaagagcaggtggcagcaggggaacgtcttctcatgctccgtgatgcatgag |
| | | gctctgcacaaccactacacgcagaagagcctctccctgtctccgggtaaatga |
| Full: | HC | atgcacagctcagcactgctctgttgcctggtcctcctgactggggtgagggctgaggtgcagct |
| 1410 | ORF | ggttgaaagcggcggagggcttgttcaacctggtagatccttgagactttcttgcgccgcttcagg |
| IL-10 | #2 | cttcacctttgatgattatgcaatgcactgggtgaggcaggcgcctggaaaggggctggagtggg |
| leader: 1-54 | | taagtgccatcacatggaacagtggccatattgactatgctgatagtgtggaaggtagattcactat |
| HC | | atcccgcgacaatgccaaaaactctttatacctgcagatgaattcactacgcgcagaggatactgc |
| Var: 55-417 | | ggtctattactgtgctaaggtgagttatctcagcaccgcatcctctctggactactggggacaaggg |
| HC | | acattggttactgtgagctccgcctccaccaagggcccaagtgtcttccccctggcaccctcctcc |
| Constant: | | aagagcacctctgggggcacagcggccctgggctgcctggtcaaggactacttccctgaacctg |
| 418-1407 | | tgacagtgtcttggaactcaggcgccctgaccagcggagtgcacaccttcccagctgtcctacag |
| (SEQ ID NO: | | tcctcaggactctactccctcagcagtgtggtgactgtgccctccagcagcttgggcacccagacc |
| 53) | | tacatctgcaatgtgaatcacaagcccagcaacaccaaggtggacaagaaagttgagcccaaat |
| | | cttgtgacaaaactcacacatgcccaccttgcccagcacctgaactcctggggggaccatcagtct |
| | | tcctcttccccccaaaacccaaggacaccctcatgatctcccgcacccctgaggtcacatgtgtgg |
| | | tggtggatgtgagccatgaagaccctgaggtcaagttcaactggtatgtggatggtgtggaggtg |
| | | cataatgccaagacaaagccgcgggaggagcagtacaacagcacatacagagtggtctctgtcc |
| | | tcactgtcctgcaccaggactggctgaatggcaaggagtacaagtgcaaggtctccaacaaagc |
| | | cctcccagcccccattgagaaaaccatctccaaagccaaagggcagcccagggaaccacaggt |
| | | gtacaccctgcccccatcccgcgatgagctgaccaagaaccaggtcagcctgacctgcctggtc |
| | | aaaggcttctatcccagtgacattgctgtggagtgggagagcaatgggcagcctgagaacaacta |
| | | caagaccacacctccagtgctggactctgatggctccttcttcctctacagcaagctcactgtggac |
| | | aagagcaggtggcagcaggggaatgtcttctcatgcagtgtgatgcatgaggctctgcacaacca |
| | | ctacacacagaagagcctctccctgtctcctggtaaatga |
| Full: 1410 | HC | atgcacagctcagcactgctctgttgcctggtcctcctgactggggtgagggctgaggtgcagct |
| IL-10 leader: | ORF | ggttgaaagtggaggagggcttgttcaacctggtagatccttgagactttcttgtgctgcttcaggct |
| 1-54 | #3 | tcacctttgatgattatgcaatgcactgggtgaggcaggcacctggaaaggggctggagtgggta |
| HC Var: 55- | | agtgccatcacatggaacagtggccatattgactatgctgatagtgtggaaggtagattcactatat |
| 417 | | ccagagacaatgccaaaaactctttatacctgcagatgaattcactaagggcagaggatactgctg |
| HC Constant: | | tctattactgtgctaaggtgagttatctcagcacagcatcctctctggactactggggacaagggac |
| 418-1407 | | attggttactgtgagctctgcctccaccaagggcccaagtgtcttccccctggcaccctcctccaag |
| (SEQ ID NO: | | agcacctctgggggcacagcagccctgggctgcctggtcaaggactacttccctgaacctgtga |
| 54) | | ctgtgtcttggaactcaggtgccctgaccagtggagtgcacaccttcccagctgtcctacagtcctc |
| | | aggactctactccctcagctctgtggtgacagtgccctccagcagcttgggcacccagacctacat |
| | | ctgcaatgtgaatcacaagcccagcaacaccaaggtggacaagaaagttgagcccaaatcttgtg |
| | | acaaaactcacacatgcccaccttgcccagcacctgaactcctggggggaccatcagtcttcctct |
| | | tccccccaaaacccaaggacaccctcatgatctccagaacccctgaggtcacatgtgtggtggtg |
| | | gatgtgagccatgaagaccctgaggtcaagttcaactggtatgtggatggtgtggaggtgcataat |
| | | gccaagacaaagcctagggaggagcagtacaacagcacttacagagtggtcagtgtcctcaca |
| | | gtcctgcaccaggactggctgaatggcaaggagtacaagtgcaaggtctccaacaaagccctcc |
| | | cagcccccattgagaaaaccatctccaaagccaaagggcagcccagggaaccacaggtgtaca |
| | | ccctgcccccatccagagatgagctgaccaagaaccaggtcagcctgacctgcctggtcaaagg |
| | | cttctatccctcagacattgctgtggagtgggagagcaatgggcagccagagaacaactacaaga |
| | | ccactcctcctgtgctggactctgatggctccttcttcctctacagcaagctcacagtggacaagag |
| | | caggtggcagcaggggaatgtcttctcatgcagtgtgatgcatgaggctctgcacaaccactaca |
| | | ctcagaagagcctctccctgtctccaggtaaatga |
| Full: 1407 | HC | atgcacagctcagcactgctctgttgcctggtcctcctgactggggtgagggccgaggtgcagct |
| IL-10 leader: | ORF | ggtcgaaagcggcggagggctcgttcaacccggtcggtccttgagactttcttgcgccgcttctg |
| 1-54 | #4 | gcttcacctttgacgattacgcaatgcactgggtgaggcaggcgcctggaaaggggctggagtg |
| HC Var: 55- | | ggtatcagccatcacatggaactcgggccatattgactatgctgatagcgtggaaggtcgcttcac |
| 417 | | tatatcccgagacaatgccaaaaactctttatacctgcagatgaattcactacgtgcagaggatacg |
| HC constant: | | gccgtctattactgtgctaaggtgtcatatctcagcaccgcatcctctctggactactggggacaag |
| 418-1407 | | ggacattggttactgtgagctccgcctccaccaagggcccatcggtcttccccctggcaccctcct |
| (SEQ ID NO: | | ccaagagcacctctgggggcacagcggccctgggctgcctggtcaaggactacttccccgaac |
| 55) | | cggtgacggtgtcgtggaactcaggcgccctgaccagcggcgtgcacaccttcccggctgtcct |
| | | acagtcctcaggactctactccctcagcagcgtggtgaccgtgccctccagcagcttgggcaccc |
| | | agacctacatctgcaacgtgaatcacaagcccagcaacaccaaggtggacaagaaagttgagcc |
| | | caaatcttgtgacaaaactcacacatgcccaccgtgcccagcacctgaactcctggggggaccgt |
| | | cagtcttcctcttccccccaaaacccaaggacaccctcatgatctcccggacccctgaggtcacat |
| | | gcgtggtggtggacgtgagccacgaagaccctgaggtcaagttcaactggtacgtggacggcgt |
| | | ggaggtgcataatgccaagacaaagccgcgggaggagcagtacaacagcacgtaccgtgtggt |
| | | cagcgtcctcaccgtcctgcaccaggactggctgaatggcaaggagtacaagtgcaaggtctcc |
| | | aacaaagccctcccagcccctatcgagaaaacaatctccaaagccaaagggcagccccgagaa |
| | | ccacaggtgtacaccctgcccccatcccgggatgagctgaccaagaaccaagtcagcctgacct |
| | | gcctggtcaaaggcttctatcccagcgacatcgccgtggagtgggagagcaatgggcagccgg |
| | | agaacaactacaagaccacgcctcccgtgctggactccgacggctccttcttcctctactccaagc |
| | | tcaccgtggacaagagcaggtggcagcaggggaacgtcttctcatgctccgtgatgcatgaggc |
| | | tctgcacaaccactacacgcagaagagcctctccctgtctccgggtaaa |
| Full: 1407 | HC | atgcacagctcagcactgctctgttgcctggtcctcctgactggggtgagggctgaggtgcagct |
| IL-10 leader: | ORF | ggttgaaagcggcggagggcttgttcaacctggtagatccttgagactttcttgcgccgcttctggc |
| 1-53 | #5 | ttcacctttgatgattatgcaatgcactgggtgaggcaggcgcctggaaaggggctggagtgggt |
| HC Var: 55- | | atcagccatcacatggaacagtggccatattgactatgctgatagtgtggaaggtagattcactata |
| 417 | | tcccgcgacaatgccaaaaactctttatacctgcagatgaattcactacgcgcagaggatactgcg |
| HC constant: | | gtctattactgtgctaaggtgtcatatctcagcaccgcatcctctctggactactggggacaaggga |
| 418-1407 | | cattggttactgtgagctccgcctccaccaagggcccaagtgtcttccccctggcaccctcctcca |
| (SEQ ID NO: | | agagcacctctgggggcacagcggccctgggctgcctggtcaaggactacttccctgaacctgt |
| 56) | | gacagtgtcttggaactcaggcgccctgaccagcggagtgcacaccttcccagctgtcctacagt |
| | | cctcaggactctactccctcagcagtgtggtgactgtgccctccagcagcttgggcacccagacct |
| | | acatctgcaatgtgaatcacaagcccagcaacaccaaggtggacaagaaagttgagcccaaatc |
| | | ttgtgacaaaactcacacatgcccaccttgcccagcacctgaactcctggggggaccatcagtctt |
| | | cctcttccccccaaaacccaaggacaccctcatgatctcccgcacccctgaggtcacatgtgtggt |
| | | ggtggatgtgagccatgaagaccctgaggtcaagttcaactggtatgtggatggtgtggaggtgc |
| | | ataatgccaagacaaagccgcgggaggagcagtacaacagcacatacagagtggtctctgtcct |
| | | cactgtcctgcaccaggactggctgaatggcaaggagtacaagtgcaaggtctccaacaaagcc |
| | | ctcccagcccctattgagaaaacaatctccaaagccaaagggcagcccagggaaccacaggtgt |
| | | acaccctgcccccatcccgcgatgagctgaccaagaaccaggtctccctgacctgcctggtcaa |
| | | aggcttctatcccagtgacattgctgtggagtgggagagcaatgggcagcctgagaacaactaca |
| | | agaccacacctccagtgctggactctgatggctccttcttcctctactccaagctcactgtggacaa |
| | | gagcaggtggcagcaggggaatgtcttctcatgcagtgtgatgcatgaggctctgcacaaccact |
| | | acacacagaagagcctctccctgtctcctggtaaa |
| Full: 1407 | HC | atgcacagctcagcactgctctgttgcctggtcctcctgactggggtgagggctgaggtgcagct |
| IL-10 leader: | ORF | ggttgaaagtggaggagggcttgttcaacctggtagatccttgagactttcttgtgctgcttctggctt |
| 1-53 | #6 | cacctttgatgattatgcaatgcactgggtgaggcaggcacctggaaaggggctggagtgggtat |
| HC Var: 55- | | cagccatcacatggaacagtggccatattgactatgctgatagtgtggaaggtagattcactatatc |
| 416 | | cagagacaatgccaaaaactctttatacctgcagatgaattcactaagggcagaggatactgctgt |
| HC constant: | | ctattactgtgctaaggtatcatatctcagcacagcatcctctctggactactggggacaagggaca |
| 418-1407 | | ttggttactgtgagctctgcctccaccaagggcccaagtgtcttccccctggcaccctcctccaaga |
| (SEQ ID NO: | | gcacctctgggggcacagcagccctgggctgcctggtcaaggactacttccctgaacctgtgact |
| 57) | | gtgtcttggaactcaggtgccctgaccagtggagtgcacaccttcccagctgtcctacagtcctca |
| | | ggactctactccctgagctctgtggtgacagtgccctccagcagcttgggcacccagacctacatc |
| | | tgcaatgtgaatcacaagcccagcaacaccaaggtggacaagaaagttgagcccaaatcttgtga |
| | | caaaactcacacatgcccaccttgcccagcacctgaactcctggggggaccatcagtcttcctctt |
| | | ccccccaaaacccaaggacaccctcatgatctccagaacccctgaggtcacatgtgtggtggtgg |
| | | atgtgagccatgaagaccctgaggtcaagttcaactggtatgtggatggtgtggaggtgcataatg |
| | | ccaagacaaagcccagggaggagcagtacaacagcacttacagagtggtcagtgtcctcacagt |
| | | cctgcaccaggactggctgaatggcaaggagtacaagtgcaaggtctccaacaaagccctccca |
| | | gcccccattgagaaaaccatctccaaagccaaagggcagcccagggaaccacaggtgtacacc |
| | | ctgcccccatccagagatgagctgaccaagaaccaggtctccctgacctgcctggtcaaaggctt |
| | | ctatccctctgacattgctgtggagtgggagagcaatgggcagccagagaacaactacaagacc |
| | | actcctcctgtgctggactctgatggctccttcttcctctactccaagctcacagtggacaagagca |
| | | ggtggcagcaggggaatgtcttctcatgcagtgtgatgcatgaggctctgcacaaccactacactc |
| | | agaagagcctctccctgtctcctggcaaa |
| Full: 1407 | HC | atgcacagctcagcactgctctgttgcctggtcctcctgactggggtgagggccgaggtgcagct |
| IL-10 leader: | ORF | ggtcgaaagcggcggagggctcgttcaacccggtcggtccttgagactttcttgcgccgcttctg |
| 1-54 | #7 | gcttcacctttgacgattacgcaatgcactgggtgaggcaggcgcctggaaaggggctggagtg |
| HC Var: 55- | | ggtatcagccatcacatggaactcgggccatattgactatgctgatagcgtggaaggtcgcttcac |
| 417 | | tatatcccgagacaatgccaaaaactctttatacctgcagatgaattcactacgtgcagaggatacg |
| HC constant: | | gccgtctattactgtgctaaggtgtcatatctcagcaccgcatcctctctggactactggggacaag |
| 418-1407 | | ggacattggttactgtgagctccgcctccaccaagggcccatcggtcttccccctggcaccctcct |
| (SEQ ID NO: | | ccaagagcacctctgggggcacagcggccctgggctgcctggtcaaggactacttccccgaac |
| 58) | | cggtgacggtgtcgtggaactcaggcgccctgaccagcggcgtgcacaccttcccggctgtcct |
| | | acagtcctcaggactctactccctcagcagcgtggtgaccgtgccctccagcagcttgggcaccc |
| | | agacctacatctgcaacgtgaatcacaagcccagcaacaccaaggtggacaagaaagttgagcc |
| | | caaatcttgtgacaaaactcacacatgcccaccgtgcccagcacctgaactcctggggggaccgt |
| | | cagtcttcctcttccccccaaaacccaaggacaccctcatgatctcccggacccctgaggtcacat |
| | | gcgtggtggtggacgtgagccacgaagaccctgaggtcaagttcaactggtacgtggacggcgt |
| | | ggaggtgcataatgccaagacaaagccgcgggaggagcagtacaacagcacgtaccgtgtggt |
| | | cagcgtcctcaccgtcctgcaccaggactggctgaatggcaaggagtacaagtgcaaggtctcc |
| | | aacaaagccctcccagcccctatcgagaaaacaatctccaaagccaaagggcagccccgagaa |
| | | ccacaggtgtacaccctgcccccatcccgggatgagctgaccaagaaccaagtcagcctgacct |
| | | gcctggtcaaaggcttctatcccagcgacatcgccgtggagtgggagagcaatgggcagccgg |
| | | agaacaactacaagaccacgcctcccgtgctggactccgacggctccttcttcctctactccaagc |
| | | tcaccgtggacaagagcaggtggcagcaggggaacgtcttctcatgctccgtgatgcatgaggc |
| | | tctgcacaaccactacacgcagaagagcctctccctgtctccgggcaaa |
| Full: 1407 | HC | atgcacagctcagcactgctctgttgcctggtcctcctgactggggtgagggctgaggtgcagct |
| IL-10 leader: | ORF | ggttgaaagcggcggagggcttgttcaacctggtagatccttgagactttcttgcgccgcttctggc |
| 1-54 | #8 | ttcacctttgatgattatgcaatgcactgggtgaggcaggcgcctggaaaggggctggagtgggt |
| HC Var: 55- | | atcagccatcacatggaacagtggccatattgactatgctgatagtgtggaaggtagattcactata |
| 417 | | tcccgcgacaatgccaaaaactctttatacctgcagatgaattcactacgcgcagaggatactgcg |
| HC constant: | | gtctattactgtgctaaggtgtcatatctcagcaccgcatcctctctggactactggggacaaggga |
| 418-1407 bp | | cattggttactgtgagctccgcctccaccaagggcccaagtgtcttccccctggcaccctcctcca |
| (SEQ ID NO: | | agagcacctctgggggcacagcggccctgggctgcctggtcaaggactacttccctgaacctgt |
| 59) | | gacagtgtcttggaactcaggcgccctgaccagcggagtgcacaccttcccagctgtcctacagt |
| | | cctcaggactctactccctcagcagtgtggtgactgtgccctccagcagcttgggcacccagacct |
| | | acatctgcaatgtgaatcacaagcccagcaacaccaaggtggacaagaaagttgagcccaaatc |
| | | ttgtgacaaaactcacacatgcccaccttgcccagcacctgaactcctggggggaccatcagtctt |
| | | cctcttccccccaaaacccaaggacaccctcatgatctcccgcacccctgaggtcacatgtgtggt |
| | | ggtggatgtgagccatgaagaccctgaggtcaagttcaactggtatgtggatggtgtggaggtgc |
| | | ataatgccaagacaaagccgcgggaggagcagtacaacagcacatacagagtggtctctgtcct |
| | | cactgtcctgcaccaggactggctgaatggcaaggagtacaagtgcaaggtctccaacaaagcc |
| | | ctcccagcccctattgagaaaacaatctccaaagccaaagggcagcccagggaaccacaggtgt |
| | | acaccctgcccccatcccgcgatgagctgaccaagaaccaggtctccctgacctgcctggtcaa |
| | | aggcttctatcccagtgacattgctgtggagtgggagagcaatgggcagcctgagaacaactaca |
| | | agaccacacctccagtgctggactctgatggctccttcttcctctactccaagctcactgtggacaa |
| | | gagcaggtggcagcaggggaatgtcttctcatgcagtgtgatgcatgaggctctgcacaaccact |
| | | acacacagaagagcctctccctgtctcctggcaaa |
| Full: 1407 | HC | atgcacagctcagcactgctctgttgcctggtcctcctgactggggtgagggctgaggtgcagct |
| IL-10 leader: | ORF | ggttgaaagtggaggagggcttgttcaacctggtagatccttgagactttcttgtgctgcttctggctt |
| 1-53 | #9 | cacctttgatgattatgcaatgcactgggtgaggcaggcacctggaaaggggctggagtgggtat |
| HC Var: 55- | | cagccatcacatggaacagtggccatattgactatgctgatagtgtggaaggtagattcactatatc |
| 416 | | cagagacaatgccaaaaactctttatacctgcagatgaattcactaagggcagaggatactgctgt |
| HC constant: | | ctattactgtgctaaggtatcatatctcagcacagcatcctctctggactactggggacaagggaca |
| 418-1407 | | ttggttactgtgagctctgcctccaccaagggcccaagtgtcttccccctggcaccctcctccaaga |
| (SEQ ID NO: | | gcacctctgggggcacagcagccctgggctgcctggtcaaggactacttccctgaacctgtgact |
| 60) | | gtgtcttggaactcaggtgccctgaccagtggagtgcacaccttcccagctgtcctacagtcctca |
| | | ggactctactccctgagctctgtggtgacagtgccctccagcagcttgggcacccagacctacatc |
| | | tgcaatgtgaatcacaagcccagcaacaccaaggtggacaagaaagttgagcccaaatcttgtga |
| | | caaaactcacacatgcccaccttgcccagcacctgaactcctggggggaccatcagtcttcctctt |
| | | ccccccaaaacccaaggacaccctcatgatctccagaacccctgaggtcacatgtgtggtggtgg |
| | | atgtgagccatgaagaccctgaggtcaagttcaactggtatgtggatggtgtggaggtgcataatg |
| | | ccaagacaaagcccagggaggagcagtacaacagcacttacagagtggtcagtgtcctcacagt |
| | | cctgcaccaggactggctgaatggcaaggagtacaagtgcaaggtctccaacaaagccctccca |
| | | gcccccattgagaaaaccatctccaaagccaaagggcagcccagggaaccacaggtgtacacc |
| | | ctgcccccatccagagatgagctgaccaagaaccaggtctccctgacctgcctggtcaaaggctt |
| | | ctatccctctgacattgctgtggagtgggagagcaatgggcagccagagaacaactacaagacc |
| | | actcctcctgtgctggactctgatggctccttcttcctctactccaagctcacagtggacaagagca |
| | | ggtggcagcaggggaatgtcttctcatgcagtgtgatgcatgaggctctgcacaaccactacactc |
| | | agaagagcctctccctgtctcctggcaaa |
| Full: 1407 | HC | atgcacagctcagcactgctctgttgcctggtcctcctgactggggtgagggccgaggtgcagct |
| IL-10 leader: | ORF | ggtcgaaagcggcggagggctcgttcaacccggtcggtccttgagactttcttgcgccgcttctg |
| 1-54 | #10 | gcttcacctttgacgattacgcaatgcactgggtgaggcaggcgcctggaaaggggctggagtg |
| HC Var: 55- | | ggtatcagccatcacatggaactcgggccatattgactatgctgatagcgtggaaggtcgcttcac |
| 417 | | tatatcccgagacaatgccaaaaactctttatacctgcagatgaattcactacgtgcagaggatacg |
| HC constant: | | gccgtctattactgtgctaaggtgtcatatctcagcaccgcatcctctctggactactggggacaag |
| 418-1407 | | ggacattggttactgtgagctccgcctccaccaagggcccatcggtcttccccctggcaccctcct |
| (SEQ ID NO: | | ccaagagcacctctgggggcacagcggccctgggctgcctggtcaaggactacttccccgaac |
| 61) | | cggtgacggtgtcgtggaactcaggcgccctgaccagcggcgtgcacaccttcccggctgtcct |
| | | acagtcctcaggactctactccctcagcagcgtggtgaccgtgccctccagcagcttgggcaccc |
| | | agacctacatctgcaacgtgaatcacaagcccagcaacaccaaggggacaagaaagttgagcc |
| | | caaatcttgtgacaaaactcacacatgcccaccgtgcccagcacctgaactcctggggggaccgt |
| | | cagtcttcctcttccccccaaaacccaaggacaccctcatgatctcccggacccctgaggtcacat |
| | | gcgtggtggtggacgtgagccacgaagaccctgaggtcaagttcaactggtacgtggacggcgt |
| | | ggaggtgcataatgccaagacaaagccgcgggaggagcagtacaacagcacgtaccgtgtggt |
| | | cagcgtcctcaccgtcctgcaccaggactggctgaatggcaaggagtacaagtgcaaggtctcc |
| | | aacaaagccctcccagcccctatcgagaaaacaatctccaaagccaaagggcagccccgagaa |
| | | ccacaggtgtacaccctgcccccatcccgggatgagctgaccaagaaccaagtcagcctgacct |
| | | gcctggtcaaaggcttctatcccagcgacatcgccgtggagtgggagagcaatgggcagccgg |
| | | agaacaactacaagaccacgcctcccgtgctggactccgacggctccttcttcctctactccaagc |
| | | tcaccgtggacaagagcaggtggcagcaggggaacgtcttctcatgctccgtgatgcatgaggc |
| | | tctgcacaaccactacacgcagaagagcctctccctgtctccgggcaaa |

The following modified anti-TNFalpha antibody expression cassette nucleic acid sequences (shown in Table 17) corresponding to SEQ ID NOs: 62-77 were designed in silico. The expression constructs includes nucleic acid molecules encoding a promoter, a heavy chain and a light chain. Some sequences also include a linker, miR-142 binding sites, and a second promoter.

TABLE 17
Expression Cassette encoding a TNF-Alpha Antibody

| SEQ ID NO | Name | Sequence |
|---|---|---|
| Full: 4485 | EC #1 | ctgcgcgctcgctcgctcactgaggccgcccgggcaaagcccgggcgtcgggcgacctttgg |
| CAG | H- | tcgcccggcctcagtgagcgagcgagcgcgcagagagggagtggccaactccatcactagg |
| Promoter: | F2A-L | ggttccttagtattaaacgcgtgtcgacattgattattgactagttattaatagtaatcaattacgggg |
| 146-1870 | | tcattagttcatagcccatatatggagttccgcgttacataacttacggtaaatggcccgcctggct |
| IL-10 leader: | | gaccgcccaacgacccccgcccattgacgtcaataatgacgtatgttcccatagtaacgccaata |
| 1898-1951 | | gggactttccattgacgtcaatgggtggactatttacggtaaactgcccacttggcagtacatcaa |
| HC: 1952- | | gtgtatcatatgccaagtacgccccctattgacgtcaatgacggtaaatggcccgcctggcattat |
| 3304 | | gcccagtacatgaccttatgggactttcctacttggcagtacatctacgtattagtcatcgctattac |
| Furin | | catgggtcgaggtgagccccacgttctgcttcactctccccatctcccccccctccccaccccca |
| cleavage site: | | attttgtatttatttattttttaattattttgtgcagcgatgggggcggggggggggggggcgcgcg |
| 3304-3328 | | ccaggcggggcggggcggggcgaggggcggggcggggcgaggcggagaggtgcggcg |
| 2a: 3329- | | gcagccaatcagagcggcgcgctccgaaagtttccttttatggcgaggcggcggcggcggcg |
| 3400 | | gccctataaaaagcgaagcgcgcggggggggagtcgctgcgcgctgccttcgccccgtgc |
| IL-2 leader: | | cccgctccgccgccgcctcgcgccgcccgccccggctctgactgaccgcgttactcccacagg |
| 3401-3460 | | tgagcgggcgggacggcccttctcctccgggctgtaattagcgcttggtttaatgacggctcgttt |
| LC var: | | cttttctgtggctgcgtgaaagccttgaggggctccgggagggccctttgtgcggggggagcg |
| 3461-4102 | | gctcggggggtgcgtgcgtgtgtgtgtgcgtggggagcgccgcgtgcggctccgcgctgccc |
| BGHpA: | | ggcggctgtgagcgctgcgggcgcggcgcggggctttgtgcgctccgcagtgtgcgcgaga |
| 4112-4335 | | ggagcgcggccgggggcggtgccacgcggtgcggggggggctgcgaggggaacaaagg |
| (SEQ ID | | ctgcgtgcggggtgtgtgcgtggggggggtgagcagggggtgtgggcgcggcggtcgggct |
| NO: 62 | | gtaacccccccctgcacccccctccccgagttgctgagcacggcccggcttcgggtgcggggc |
| (including | | tccgtgcggggcgtggcgcggggctcgccgtgccgggcggggggtggcggcgggtgggg |
| ITRs)) | | gtgccgggcggggggggccgcctcgggccggggagggctcgggggaggggcgcggcg |
| Antibody | | gcccccggagcgccggcggctgtcgaggcgcggcgagccgcagccattgccttttatggtaat |
| expression | | cgtgcgagagggcgcagggacttcctttgtcccaaatctgtgcggagccgaaatctgggaggc |
| cassette (w/o | | gccgccgcaccccctctagcgggcgcggggcgaagcggtgcggcgccggcaggaaggaa |
| ITRs | | atgggcggggagggccttcgtgcgtcgccgcgccgccgtccccttctccctctccagcctcgg |
| (nucleotides | | ggctgtccgcggggggacggctgccttcgggggggacggggcagggcggggttcggcttct |
| 146-4335; | | ggcgtgtgaccggcggtctagactctgctaaccatgttcatgccttcttctctttcctacagctcctg |
| SEQ ID NO: | | ggcaacgtgctggttgttgtgctgtctcatcattttggcaaagaattcaagcttccagctagcgcca |
| 115) | | ccatgcacagctcagcactgctctgttgcctggtcctcctgactggggtgagggccgaggtgca |
| | | gctggtcgaaagcggcggagggctcgttcaacccggtcggtccttgagactttcttgcgccgctt |
| | | ctggcttcacctttgacgattacgcaatgcactgggtgaggcaggcgcctggaaaggggctgga |
| | | gtgggtatcagccatcacatggaactcgggccatattgactatgctgatagcgtggaaggtcgct |
| | | tcactatatcccgagacaatgccaaaaactctttatacctgcagatgaattcactacgtgcagagg |
| | | atacggccgtctattactgtgctaaggtgtcatatctcagcaccgcatcctctctggactactgggg |
| | | acaagggacattggttactgtgagctccgcctccaccaagggcccatcggtcttccccctggca |
| | | ccctcctccaagagcacctctgggggcacagcggccctgggctgcctggtcaaggactacttc |
| | | cccgaaccggtgacggtgtcgtggaactcaggcgccctgaccagcggcgtgcacaccttccc |
| | | ggctgtcctacagtcctcaggactctactccctcagcagcgtggtgaccgtgccctccagcagct |
| | | tgggcacccagacctacatctgcaacgtgaatcacaagcccagcaacaccaaggtggacaag |
| | | aaagttgagcccaaatcttgtgacaaaactcacacatgcccaccgtgcccagcacctgaactcct |
| | | ggggggaccgtcagtcttcctcttccccccaaaacccaaggacaccctcatgatctcccggacc |
| | | cctgaggtcacatgcgtggtggtggacgtgagccacgaagaccctgaggtcaagttcaactggt |
| | | acgtggacggcgtggaggtgcataatgccaagacaaagccgcgggaggagcagtacaacag |
| | | cacgtaccgtgtggtcagcgtcctcaccgtcctgcaccaggactggctgaatggcaaggagtac |
| | | aagtgcaaggtctccaacaaagccctcccagcccctatcgagaaaacaatctccaaagccaaa |
| | | gggcagccccgagaaccacaggtgtacaccctgcccccatcccgggatgagctgaccaagaa |
| | | ccaagtcagcctgacctgcctggtcaaaggcttctatcccagcgacatcgccgtggagtgggag |
| | | agcaatgggcagccggagaacaactacaagaccacgcctcccgtgctggactccgacggctc |
| | | cttcttcctctactccaagctcaccgtggacaagagcaggtggcagcaggggaacgtcttctcat |
| | | gctccgtgatgcatgaggctctgcacaaccactacacgcagaagagcctctccctgtctccggg |
| | | taaaagaaagagaaggagtggctcaggagcccctgtgaaacagaccctgaactttgacctcttg |
| | | aagcttgctggggatgtggagtctaatcctggtccaatgtacaggatgcaactcctgtcttgcattg |
| | | cactaagtcttgcacttgtcacaaactcggacatccagatgacccaaagcccctcctctctgtcag |
| | | ccagtgtgggcgatcgggtcacaattacttgcagagcttcgcagggaataaggaactacctcgc |
| | | atggtatcagcaaaagcctgggaaagcgccaaagttgcttatctacgccgccagcacgctgcag |
| | | tccggtgttccgtctcgcttctctggcagtggaagcgggaccgactttacattaactatttcctctct |
| | | gcaacccgaggatgtggccacctattactgtcagcgatataatcgtgcaccttacacattcggcc |
| | | aaggtaccaaagtagaaatcaagcgaactgtggctgcaccatctgtcttcatcttcccgccatctg |
| | | atgagcagttgaaatctggaactgcctctgttgtgtgcctgctgaataacttctatcccagagaggc |
| | | caaagtacagtggaaggtggataacgccctccaatcgggcaactcccaggagagtgtcacaga |
| | | gcaggacagcaaggacagcacctacagcctcagcagcaccctgacgctgagcaaagcagac |
| | | tacgagaaacacaaagtctacgcctgcgaagtcacccatcagggcctgagctcgcccgtcaca |
| | | aagagcttcaacaggggagagtgttgacaattgctgtgccttctagttgccagccatctgttgtttg |
| | | cccctcccccgtgccttccttgaccctggaaggtgccactcccactgtcctttcctaataaaatgag |
| | | gaaattgcatcgcattgtctgagtaggtgtcattctattctggggggtggggggggcaggacag |
| | | caagggggaggattgggaagacaatagcaggcatgctggggatgcggtgggctctatggagc |
| | | tcgagaggaacccctagtgatggagttggccactccctctctgcgcgctcgctcgctcactgag |
| | | gccgggcgaccaaaggtcgcccgacgcccgggctttgcccgggcggcctcagtgagcgagc |
| | | gagcgcgcagctgcagatctg |
| Full: 4595 | EC #2 | ctgcgcgctcgctcgctcactgaggccgcccgggcaaagcccgggcgtcgggcgacctttgg |
| CAG | H- | tcgcccggcctcagtgagcgagcgagcgcgcagagagggagtggccaactccatcactagg |
| Promoter: | F2A- | ggttccttagtattaaacgcgtgtcgacattgattattgactagttattaatagtaatcaattacgggg |
| 146-1870 | L- | tcattagttcatagcccatatatggagttccgcgttacataacttacggtaaatggcccgcctggct |
| IL-10 leader: | 4xmiR142 | gaccgcccaacgacccccgcccattgacgtcaataatgacgtatgttcccatagtaacgccaata |
| 1898-1951 | | gggactttccattgacgtcaatgggtggactatttacggtaaactgcccacttggcagtacatcaa |
| HC: 1952- | | gtgtatcatatgccaagtacgccccctattgacgtcaatgacggtaaatggcccgcctggcattat |
| 3304 | | gcccagtacatgaccttatgggactttcctacttggcagtacatctacgtattagtcatcgctattac |
| Furin | | catgggtcgaggtgagccccacgttctgcttcactctccccatctcccccccctccccaccccca |
| cleavage site: | | attttgtatttatttattttttaattattttgtgcagcgatgggggcggggggggggggggcgcgcg |
| 3304-3328 | | ccaggcggggcggggggggcgaggggggggcggggcgaggcggagaggtgcggcg |
| 2a: 3329- | | gcagccaatcagagcggcgcgctccgaaagtttccttttatggcgaggcggcggcggcggcg |
| 3400 | | gccctataaaaagcgaagcgcgcggggggggagtcgctgcgcgctgccttcgccccgtgc |
| IL-2 leader: | | cccgctccgccgccgcctcgcgccgcccgccccggctctgactgaccgcgttactcccacagg |
| 3401-3460 | | tgagcggggggacggcccttctcctccgggctgtaattagcgcttggtttaatgacggctcgttt |
| LC: 3461- | | cttttctgtggctgcgtgaaagccttgaggggctccgggagggccctttgtgcggggggagcg |
| 4105 | | gctcggggggtgcgtgcgtgtgtgtgtgcgtggggagcgccgcgtgcggctccgcgctgccc |
| 4xmiR-142: | | ggcggctgtgagcgctgcgggcgcggcgcggggctttgtgcgctccgcagtgtgcgcgaga |
| 4112-4215 | | ggagcgcggccgggggcggtgccacgcggtgcggggggggctgcgaggggaacaaagg |
| BGHpoly(A): | | ctgcgtgcggggtgtgtgcgtggggggggtgagcagggggtgtgggcgcggcggtcgggct |
| 4222-4445 | | gtaacccccccctgcacccccctccccgagttgctgagcacggcccggcttcgggtgcggggc |
| (SEQ ID | | tccgtgcggggcgtggcgcggggctcgccgtgccgggcggggggtggcggcgggtgggg |
| NO: 63 | | gtgccgggcggggcggggccgcctcgggccggggagggctcgggggaggggcgcggcg |
| (including | | gcccccggagcgccggcggctgtcgaggcgcggcgagccgcagccattgccttttatggtaat |
| ITRs)) | | cgtgcgagagggcgcagggacttcctttgtcccaaatctgtgcggagccgaaatctgggaggc |
| Antibody | | gccgccgcaccccctctagcgggcgcggggcgaagcggtgcggcgccggcaggaaggaa |
| expression | | atgggcggggagggccttcgtgcgtcgccgcgccgccgtccccttctccctctccagcctcgg |
| cassette (w/o | | ggctgtccgcgggggggggggacggctgccttcgggggggacggggcagggcggggttcggcttct |
| ITRs | | ggcgtgtgaccggcggtctagactctgctaaccatgttcatgccttcttctctttcctacagctcctg |
| (nucleotides | | ggcaacgtgctggttgttgtgctgtctcatcattttggcaaagaattcaagcttccagctagcgcca |
| 146-4445; | | ccatgcacagctcagcactgctctgttgcctggtcctcctgactggggtgagggccgaggtgca |
| SEQ ID NO: | | gctggtcgaaagcggcggagggctcgttcaacccggtcggtccttgagactttcttgcgccgctt |
| 116) | | ctggcttcacctttgacgattacgcaatgcactgggtgaggcaggcgcctggaaaggggctgga |
| | | gtgggtatcagccatcacatggaactcgggccatattgactatgctgatagcgtggaaggtcgct |
| | | tcactatatcccgagacaatgccaaaaactctttatacctgcagatgaattcactacgtgcagagg |
| | | atacggccgtctattactgtgctaaggtgtcatatctcagcaccgcatcctctctggactactgggg |
| | | acaagggacattggttactgtgagctccgcctccaccaagggcccatcggtcttccccctggca |
| | | ccctcctccaagagcacctctgggggcacagcggccctgggctgcctggtcaaggactacttc |
| | | cccgaaccggtgacggtgtcgtggaactcaggcgccctgaccagcggcgtgcacaccttccc |
| | | ggctgtcctacagtcctcaggactctactccctcagcagcgtggtgaccgtgccctccagcagct |
| | | tgggcacccagacctacatctgcaacgtgaatcacaagcccagcaacaccaaggtggacaag |
| | | aaagttgagcccaaatcttgtgacaaaactcacacatgcccaccgtgcccagcacctgaactcct |
| | | ggggggaccgtcagtcttcctcttccccccaaaacccaaggacaccctcatgatctcccggacc |
| | | cctgaggtcacatgcgtggtggtggacgtgagccacgaagaccctgaggtcaagttcaactggt |
| | | acgtggacggcgtggaggtgcataatgccaagacaaagccgcgggaggagcagtacaacag |
| | | cacgtaccgtgtggtcagcgtcctcaccgtcctgcaccaggactggctgaatggcaaggagtac |
| | | aagtgcaaggtctccaacaaagccctcccagcccctatcgagaaaacaatctccaaagccaaa |
| | | gggcagccccgagaaccacaggtgtacaccctgcccccatcccgggatgagctgaccaagaa |
| | | ccaagtcagcctgacctgcctggtcaaaggcttctatcccagcgacatcgccgtggagtgggag |
| | | agcaatgggcagccggagaacaactacaagaccacgcctcccgtgctggactccgacggctc |
| | | cttcttcctctactccaagctcaccgtggacaagagcaggtggcagcaggggaacgtcttctcat |
| | | gctccgtgatgcatgaggctctgcacaaccactacacgcagaagagcctctccctgtctccggg |
| | | taaaagaaagagaaggagtggctcaggagcccctgtgaaacagaccctgaactttgacctcttg |
| | | aagcttgctggggatgtggagtctaatcctggtccaatgtacaggatgcaactcctgtcttgcattg |
| | | cactaagtcttgcacttgtcacaaactcggacatccagatgacccaaagcccctcctctctgtcag |
| | | ccagtgtgggcgatcgggtcacaattacttgcagagcttcgcagggaataaggaactacctcgc |
| | | atggtatcagcaaaagcctgggaaagcgccaaagttgcttatctacgccgccagcacgctgcag |
| | | tccggtgttccgtctcgcttctctggcagtggaagcgggaccgactttacattaactatttcctctct |
| | | gcaacccgaggatgtggccacctattactgtcagcgatataatcgtgcaccttacacattcggcc |
| | | aaggtaccaaagtagaaatcaagcgaactgtggctgcaccatctgtcttcatcttcccgccatctg |
| | | atgagcagttgaaatctggaactgcctctgttgtgtgcctgctgaataacttctatcccagagaggc |
| | | caaagtacagtggaaggtggataacgccctccaatcgggcaactcccaggagagtgtcacaga |
| | | gcaggacagcaaggacagcacctacagcctcagcagcaccctgacgctgagcaaagcagac |
| | | tacgagaaacacaaagtctacgcctgcgaagtcacccatcagggcctgagctcgcccgtcaca |
| | | aagagcttcaacaggggagagtgttgacctaggtccataaagtaggaaacactacactattccat |
| | | aaagtaggaaacactacatcactccataaagtaggaaacactacaagtctccataaagtaggaa |
| | | acactacacaattgctgtgccttctagttgccagccatctgttgtttgcccctcccccgtgccttcctt |
| | | gaccctggaaggtgccactcccactgtcctttcctaataaaatgaggaaattgcatcgcattgtctg |
| | | agtaggtgtcattctattctggggggtggggtggggcaggacagcaagggggaggattggga |
| | | agacaatagcaggcatgctggggatgcggtgggctctatggagctcgagaggaacccctagtg |
| | | atggagttggccactccctctctgcgcgctcgctcgctcactgaggccgggcgaccaaaggtcg |
| | | cccgacgcccgggctttgcccgggcggcctcagtgagcgagcgagcgcgcagctgcagatct |
| | | g |
| Full: 4485 | EC #3 | ctgcgcgctcgctcgctcactgaggccgcccgggcaaagcccgggcgtcgggcgacctttgg |
| CAG | H- | tcgcccggcctcagtgagcgagcgagcgcgcagagagggagtggccaactccatcactagg |
| Promoter: | F2A-L | ggttccttagtattaaacgcgtgtcgacattgattattgactagttattaatagtaatcaattacgggg |
| 146-1870 | | tcattagttcatagcccatatatggagttccgcgttacataacttacggtaaatggcccgcctggct |
| IL-10 leader: | | gaccgcccaacgacccccgcccattgacgtcaataatgacgtatgttcccatagtaacgccaata |
| 1898-1950 | | gggactttccattgacgtcaatgggtggactatttacggtaaactgcccacttggcagtacatcaa |
| HC: 1951- | | gtgtatcatatgccaagtacgccccctattgacgtcaatgacggtaaatggcccgcctggcattat |
| 3304 | | gcccagtacatgaccttatgggactttcctacttggcagtacatctacgtattagtcatcgctattac |
| Furin | | catgggtcgaggtgagccccacgttctgcttcactctccccatctcccccccctccccaccccca |
| cleavage site: | | attttgtatttatttattttttaattattttgtgcagcgatgggggcggggggggggggggcgcgcg |
| 3304-3328 | | ccaggcggggggggcggggcgaggggggggcggggcgaggcggagaggtgcggcg |
| 2a: 3329- | | gcagccaatcagagcggcgcgctccgaaagtttccttttatggcgaggcggcggcggcggcg |
| 3400 | | gccctataaaaagcgaagcgcgcggggggggagtcgctgcgcgctgccttcgccccgtgc |
| IL-2 leader: | | cccgctccgccgccgcctcgcgccgcccgccccggctctgactgaccgcgttactcccacagg |
| 3401-3459 | | tgagcgggcgggacggcccttctcctccgggctgtaattagcgcttggtttaatgacggctcgttt |
| LC: 3460- | | cttttctgtggctgcgtgaaagccttgaggggctccgggagggccctttgtgcggggggagcg |
| 4105 | | gctcggggggtgcgtgcgtgtgtgtgtgcgtggggagcgccgcgtgcggctccgcgctgccc |
| BGH | | ggcggctgtgagcgctgcgggcgcggcgcggggctttgtgcgctccgcagtgtgcgcgaga |
| poly(A): | | ggagcgcggccgggggcggtgccacgcggtgcggggggggctgcgaggggaacaaagg |
| 4112-4335 | | ctgcgtgcggggtgtgtgcgtggggggggtgagcagggggtgtgggcgcggcggtcgggct |
| (SEQ ID NO: | | gtaacccccccctgcacccccctccccgagttgctgagcacggcccggcttcgggtgcggggc |
| 64 | | tccgtgcggggcgtggcgcggggctcgccgtgccgggcggggggtggcggcgggtgggg |
| (including | | gtgccgggcggggcggggccgcctcgggccggggagggctcgggggaggggcgcggcg |
| ITRs)) | | gcccccggagcgccggcggctgtcgaggcgcggcgagccgcagccattgccttttatggtaat |
| Antibody | | cgtgcgagagggcgcagggacttcctttgtcccaaatctgtgcggagccgaaatctgggaggc |
| expression | | gccgccgcaccccctctagcgggcgcggggcgaagcggtgcggcgccggcaggaaggaa |
| cassette (w/o | | atgggggggagggccttcgtgcgtcgccgcgccgccgtccccttctccctctccagcctcgg |
| ITRs | | ggctgtccgcggggggacggctgccttcgggggggacggggcagggcggggttcggcttct |
| (nucleotides | | ggcgtgtgaccggcggtctagactctgctaaccatgttcatgccttcttctctttcctacagctcctg |
| 146-4335; | | ggcaacgtgctggttgttgtgctgtctcatcattttggcaaagaattcaagcttccagctagcgcca |
| SEQ ID NO: | | ccatgcacagctcagcactgctctgttgcctggtcctcctgactggggtgagggctgaggtgca |
| 117) | | gctggttgaaagcggcggagggcttgttcaacctggtagatccttgagactttcttgcgccgcttc |
| | | tggcttcacctttgatgattatgcaatgcactgggtgaggcaggcgcctggaaaggggctggagt |
| | | gggtatcagccatcacatggaacagtggccatattgactatgctgatagtgtggaaggtagattca |
| | | ctatatcccgcgacaatgccaaaaactctttatacctgcagatgaattcactacgcgcagaggata |
| | | ctgcggtctattactgtgctaaggtgtcatatctcagcaccgcatcctctctggactactggggaca |
| | | agggacattggttactgtgagctccgcctccaccaagggcccaagtgtcttccccctggcaccct |
| | | cctccaagagcacctctgggggcacagcggccctgggctgcctggtcaaggactacttccctg |
| | | aacctgtgacagtgtcttggaactcaggcgccctgaccagcggagtgcacaccttcccagctgt |
| | | cctacagtcctcaggactctactccctcagcagtgtggtgactgtgccctccagcagcttgggca |
| | | cccagacctacatctgcaatgtgaatcacaagcccagcaacaccaaggtggacaagaaagttg |
| | | agcccaaatcttgtgacaaaactcacacatgcccaccttgcccagcacctgaactcctgggggg |
| | | accatcagtcttcctcttccccccaaaacccaaggacaccctcatgatctcccgcacccctgagg |
| | | tcacatgtgtggtggtggatgtgagccatgaagaccctgaggtcaagttcaactggtatgtggat |
| | | ggtgtggaggtgcataatgccaagacaaagccgcgggaggagcagtacaacagcacatacag |
| | | agtggtctctgtcctcactgtcctgcaccaggactggctgaatggcaaggagtacaagtgcaag |
| | | gtctccaacaaagccctcccagcccctattgagaaaacaatctccaaagccaaagggcagccc |
| | | agggaaccacaggtgtacaccctgcccccatcccgcgatgagctgaccaagaaccaggtctcc |
| | | ctgacctgcctggtcaaaggcttctatcccagtgacattgctgtggagtgggagagcaatgggca |
| | | gcctgagaacaactacaagaccacacctccagtgctggactctgatggctccttcttcctctactc |
| | | caagctcactgtggacaagagcaggtggcagcaggggaatgtcttctcatgcagtgtgatgcat |
| | | gaggctctgcacaaccactacacacagaagagcctctccctgtctcctggtaaaagaaagagaa |
| | | ggagtggctcaggagcccctgtgaaacagaccctgaactttgacctcttgaagcttgctggggat |
| | | gtggagtctaatcctggtccaatgtacaggatgcaactcctgtcttgcattgcactaagtcttgcact |
| | | tgtcacaaactcagacatccagatgacccaaagcccctcctctctgtcagccagtgtgggggatc |
| | | gcgtcacaattacttgcagagcttcccagggaataaggaactacctcgcgtggtatcagcaaaa |
| | | gcctgggaaagcgccaaagttgcttatctatgccgccagcactctgcagtcaggtgttcctagta |
| | | gattctctggcagtggaagcgggactgactttacattaactatttcctctctgcaacctgaggatgt |
| | | ggccacctattactgtcagcggtataatcgcgcaccttacacatttggccaaggtaccaaagtag |
| | | aaatcaagcggactgtggctgcaccatctgtcttcatcttcccgccatctgatgagcagttgaaatc |
| | | tggaactgcctctgttgtgtgcctgctgaataacttctatcccagagaggccaaagtacagtggaa |
| | | ggtggataatgccctccaaagtggcaactcccaggagagtgtcacagagcaggacagcaagg |
| | | acagcacctacagcctcagcagcaccctgaccctgagcaaagcagactatgagaaacacaaa |
| | | gtctacgcgtgtgaagtcacccatcagggcctgagctccccagtcacaaagagcttcaacaggg |
| | | gagagtgttgacaattgctgtgccttctagttgccagccatctgttgtttgcccctcccccgtgcctt |
| | | ccttgaccctggaaggtgccactcccactgtcctttcctaataaaatgaggaaattgcatcgcattg |
| | | tctgagtaggtgtcattctattctggggggtggggggggcaggacagcaagggggaggattg |
| | | ggaagacaatagcaggcatgctggggatgcggtgggctctatggagctcgagaggaacccct |
| | | agtgatggagttggccactccctctctgcgcgctcgctcgctcactgaggccgggcgaccaaag |
| | | gtcgcccgacgcccgggctttgcccgggcggcctcagtgagcgagcgagcgcgcagctgca |
| | | gatctg |
| Full: 4595 | EC #4 | ctgcgcgctcgctcgctcactgaggccgcccgggcaaagcccgggcgtcgggcgacctttgg |
| CAG | H- | tcgcccggcctcagtgagcgagcgagcgcgcagagagggagtggccaactccatcactagg |
| Promoter: | F2A- | ggttccttagtattaaacgcgtgtcgacattgattattgactagttattaatagtaatcaattacgggg |
| 146-1725 | L- | tcattagttcatagcccatatatggagttccgcgttacataacttacggtaaatggcccgcctggct |
| IL-10 leader: | 4xmiR142 | gaccgcccaacgacccccgcccattgacgtcaataatgacgtatgttcccatagtaacgccaata |
| 1898-1950 | | gggactttccattgacgtcaatgggtggactatttacggtaaactgcccacttggcagtacatcaa |
| HC: 1951- | | gtgtatcatatgccaagtacgccccctattgacgtcaatgacggtaaatggcccgcctggcattat |
| 3304 | | gcccagtacatgaccttatgggactttcctacttggcagtacatctacgtattagtcatcgctattac |
| Furin | | catgggtcgaggtgagccccacgttctgcttcactctccccatctcccccccctccccaccccca |
| cleavage site: | | attttgtatttatttattttttaattattttgtgcagcgatgggggcggggggggggggggcgcgcg |
| 3304-3328 | | ccaggcggggcggggcggggcgaggggcggggggggcgaggcggagaggtgcggcg |
| 2a: 3329- | | gcagccaatcagagcggcgcgctccgaaagtttccttttatggcgaggcggcggcggcggcg |
| 3400 | | gccctataaaaagcgaagcgcgcggggggggagtcgctgcgcgctgccttcgccccgtgc |
| IL-2 leader: | | cccgctccgccgccgcctcgcgccgcccgccccggctctgactgaccgcgttactcccacagg |
| 3401-3459 | | tgagcgggcgggacggcccttctcctccgggctgtaattagcgcttggtttaatgacggctcgttt |
| LC: 3460- | | cttttctgtggctgcgtgaaagccttgaggggctccgggagggccctttgtgcggggggagcg |
| 4105 | | gctcggggggtgcgtgcgtgtgtgtgtgcgtggggagcgccgcgtgcggctccgcgctgccc |
| 4xmiR-142: | | ggcggctgtgagcgctgcgggcgcggcgcggggctttgtgcgctccgcagtgtgcgcgaga |
| 4112-4215 | | ggagcgcggccgggggcggtgccacgcggtgcggggggggctgcgaggggaacaaagg |
| BGH | | ctgcgtgcggggtgtgtgcgtggggggggtgagcagggggtgtgggcgcggcggtcgggct |
| poly(A): | | gtaacccccccctgcacccccctccccgagttgctgagcacggcccggcttcgggtgcggggc |
| 4222-4445 | | tccgtgcggggcgtggcgcggggctcgccgtgccgggcggggggtggcggcgggtgggg |
| (SEQ ID | | gtgccgggcggggcggggccgcctcgggccggggagggctcgggggaggggcgcggcg |
| NO: 65 | | gcccccggagcgccggcggctgtcgaggcgcggcgagccgcagccattgccttttatggtaat |
| (including | | cgtgcgagagggcgcagggacttcctttgtcccaaatctgtgcggagccgaaatctgggaggc |
| ITRs)) | | gccgccgcaccccctctagcgggcgcggggcgaagcggtgcggcgccggcaggaaggaa |
| Antibody | | atgggcggggagggccttcgtgcgtcgccgcgccgccgtccccttctccctctccagcctcgg |
| expression | | ggctgtccgcggggggacggctgccttcgggggggacggggcagggcggggttcggcttct |
| cassette (w/o | | ggcgtgtgaccggcggtctagactctgctaaccatgttcatgccttcttctctttcctacagctcctg |
| ITRs | | ggcaacgtgctggttgttgtgctgtctcatcattttggcaaagaattcaagcttccagctagcgcca |
| (nucleotides | | ccatgcacagctcagcactgctctgttgcctggtcctcctgactggggtgagggctgaggtgca |
| 146-4445; | | gctggttgaaagcggcggagggcttgttcaacctggtagatccttgagactttcttgcgccgcttc |
| SEQ ID NO: | | tggcttcacctttgatgattatgcaatgcactgggtgaggcaggcgcctggaaaggggctggagt |
| 118) | | gggtatcagccatcacatggaacagtggccatattgactatgctgatagtgtggaaggtagattca |
| | | ctatatcccgcgacaatgccaaaaactctttatacctgcagatgaattcactacgcgcagaggata |
| | | ctgcggtctattactgtgctaaggtgtcatatctcagcaccgcatcctctctggactactggggaca |
| | | agggacattggttactgtgagctccgcctccaccaagggcccaagtgtcttccccctggcaccct |
| | | cctccaagagcacctctgggggcacagcggccctgggctgcctggtcaaggactacttccctg |
| | | aacctgtgacagtgtcttggaactcaggcgccctgaccagcggagtgcacaccttcccagctgt |
| | | cctacagtcctcaggactctactccctcagcagtgtggtgactgtgccctccagcagcttgggca |
| | | cccagacctacatctgcaatgtgaatcacaagcccagcaacaccaaggtggacaagaaagttg |
| | | agcccaaatcttgtgacaaaactcacacatgcccaccttgcccagcacctgaactcctgggggg |
| | | accatcagtcttcctcttccccccaaaacccaaggacaccctcatgatctcccgcacccctgagg |
| | | tcacatgtgtggtggtggatgtgagccatgaagaccctgaggtcaagttcaactggtatgtggat |
| | | ggtgtggaggtgcataatgccaagacaaagccgcgggaggagcagtacaacagcacatacag |
| | | agtggtctctgtcctcactgtcctgcaccaggactggctgaatggcaaggagtacaagtgcaag |
| | | gtctccaacaaagccctcccagcccctattgagaaaacaatctccaaagccaaagggcagccc |
| | | agggaaccacaggtgtacaccctgcccccatcccgcgatgagctgaccaagaaccaggtctcc |
| | | ctgacctgcctggtcaaaggcttctatcccagtgacattgctgtggagtgggagagcaatgggca |
| | | gcctgagaacaactacaagaccacacctccagtgctggactctgatggctccttcttcctctactc |
| | | caagctcactgtggacaagagcaggtggcagcaggggaatgtcttctcatgcagtgtgatgcat |
| | | gaggctctgcacaaccactacacacagaagagcctctccctgtctcctggtaaaagaaagagaa |
| | | ggagtggctcaggagcccctgtgaaacagaccctgaactttgacctcttgaagcttgctggggat |
| | | gtggagtctaatcctggtccaatgtacaggatgcaactcctgtcttgcattgcactaagtcttgcact |
| | | tgtcacaaactcagacatccagatgacccaaagcccctcctctctgtcagccagtgtgggggatc |
| | | gcgtcacaattacttgcagagcttcccagggaataaggaactacctcgcgtggtatcagcaaaa |
| | | gcctgggaaagcgccaaagttgcttatctatgccgccagcactctgcagtcaggtgttcctagta |
| | | gattctctggcagtggaagcgggactgactttacattaactatttcctctctgcaacctgaggatgt |
| | | ggccacctattactgtcagcggtataatcgcgcaccttacacatttggccaaggtaccaaagtag |
| | | aaatcaagcggactgtggctgcaccatctgtcttcatcttcccgccatctgatgagcagttgaaatc |
| | | tggaactgcctctgttgtgtgcctgctgaataacttctatcccagagaggccaaagtacagtggaa |
| | | ggtggataatgccctccaaagtggcaactcccaggagagtgtcacagagcaggacagcaagg |
| | | acagcacctacagcctcagcagcaccctgaccctgagcaaagcagactatgagaaacacaaa |
| | | gtctacgcgtgtgaagtcacccatcagggcctgagctccccagtcacaaagagcttcaacaggg |
| | | gagagtgttgacctaggtccataaagtaggaaacactacactattccataaagtaggaaacacta |
| | | catcactccataaagtaggaaacactacaagtctccataaagtaggaaacactacacaattgctgt |
| | | gccttctagttgccagccatctgttgtttgcccctcccccgtgccttccttgaccctggaaggtgcc |
| | | actcccactgtcctttcctaataaaatgaggaaattgcatcgcattgtctgagtaggtgtcattctatt |
| | | ctggggggtggggtggggcaggacagcaagggggaggattgggaagacaatagcaggcat |
| | | gctggggatgcggtgggctctatggagctcgagaggaacccctagtgatggagttggccactc |
| | | cctctctgcgcgctcgctcgctcactgaggccgggcgaccaaaggtcgcccgacgcccgggc |
| | | tttgcccgggcggcctcagtgagcgagcgagcgcgcagctgcagatctg |
| Full: 4485 | EC #5 | ctgcgcgctcgctcgctcactgaggccgcccgggcaaagcccgggcgtcgggcgacctttgg |
| CAG | H- | tcgcccggcctcagtgagcgagcgagcgcgcagagagggagtggccaactccatcactagg |
| Promoter: | F2A-L | ggttccttagtattaaacgcgtgtcgacattgattattgactagttattaatagtaatcaattacgggg |
| 146-1870 | | tcattagttcatagcccatatatggagttccgcgttacataacttacggtaaatggcccgcctggct |
| IL-10 leader: | | gaccgcccaacgacccccgcccattgacgtcaataatgacgtatgttcccatagtaacgccaata |
| 1898-1950 | | gggactttccattgacgtcaatgggggactatttacggtaaactgcccacttggcagtacatcaa |
| HC: 1951- | | gtgtatcatatgccaagtacgccccctattgacgtcaatgacggtaaatggcccgcctggcattat |
| 3304 | | gcccagtacatgaccttatgggactttcctacttggcagtacatctacgtattagtcatcgctattac |
| Furin | | catgggtcgaggtgagccccacgttctgcttcactctccccatctcccccccctccccaccccca |
| cleavage site: | | attttgtatttatttattttttaattattttgtgcagcgatgggggcggggggggggggggcgcgcg |
| 3304-3328 | | ccaggcggggcggggcggggcgaggggggggcggggcgaggcggagaggtgcggcg |
| 2a: 3329- | | gcagccaatcagagcggcgcgctccgaaagtttccttttatggcgaggcggcggcggcggcg |
| 3400 | | gccctataaaaagcgaagcgcgcggggggggagtcgctgcgcgctgccttcgccccgtgc |
| IL-2 leader: | | cccgctccgccgccgcctcgcgccgcccgccccggctctgactgaccgcgttactcccacagg |
| 3401-3459 | | tgagcgggcgggacggcccttctcctccgggctgtaattagcgcttggtttaatgacggctcgttt |
| LC: 3460- | | cttttctgtggctgcgtgaaagccttgaggggctccgggagggccctttgtgcggggggagcg |
| 4105 | | gctcggggggtgcgtgcgtgtgtgtgtgcgtggggagcgccgcgtgcggctccgcgctgccc |
| BGH | | ggcggctgtgagcgctgcgggcgcggcgcggggctttgtgcgctccgcagtgtgcgcgaga |
| poly(A): | | ggagcgcggccgggggcggtgccacgcggtgcggggggggctgcgaggggaacaaagg |
| 4112-4335 | | ctgcgtgcggggtgtgtgcgtggggggggtgagcagggggtgtgggcgcggcggtcgggct |
| (SEQ ID | | gtaacccccccctgcacccccctccccgagttgctgagcacggcccggcttcgggtgcggggc |
| NO: 66 | | tccgtgcggggcgtggcgcggggctcgccgtgccgggcggggggtggcggcgggtgggg |
| (including | | gtgccgggcggggcggggccgcctcgggccggggagggctcgggggaggggcgcggcg |
| ITRs)) | | gcccccggagcgccggcggctgtcgaggcgcggcgagccgcagccattgccttttatggtaat |
| Antibody | | cgtgcgagagggcgcagggacttcctttgtcccaaatctgtgcggagccgaaatctgggaggc |
| expression | | gccgccgcaccccctctagcgggcgcggggcgaagcggtgcggcgccggcaggaaggaa |
| cassette (w/o | | atgggcggggagggccttcgtgcgtcgccgcgccgccgtccccttctccctctccagcctcgg |
| ITRs | | ggctgtccgcggggggacggctgccttcgggggggacggggcagggcggggttcggcttct |
| (nucleotides | | ggcgtgtgaccggcggtctagactctgctaaccatgttcatgccttcttctctttcctacagctcctg |
| 146-4335; | | ggcaacgtgctggttgttgtgctgtctcatcattttggcaaagaattcaagcttccagctagcgcca |
| SEQ ID NO: | | ccatgcacagctcagcactgctctgttgcctggtcctcctgactggggtgagggctgaggtgca |
| 119) | | gctggttgaaagtggaggagggcttgttcaacctggtagatccttgagactttcttgtgctgcttct |
| | | ggcttcacctttgatgattatgcaatgcactgggtgaggcaggcacctggaaaggggctggagt |
| | | gggtatcagccatcacatggaacagtggccatattgactatgctgatagtgtggaaggtagattca |
| | | ctatatccagagacaatgccaaaaactctttatacctgcagatgaattcactaagggcagaggata |
| | | ctgctgtctattactgtgctaaggtatcatatctcagcacagcatcctctctggactactggggaca |
| | | agggacattggttactgtgagctctgcctccaccaagggcccaagtgtcttccccctggcaccct |
| | | cctccaagagcacctctgggggcacagcagccctgggctgcctggtcaaggactacttccctg |
| | | aacctgtgactgtgtcttggaactcaggtgccctgaccagtggagtgcacaccttcccagctgtc |
| | | ctacagtcctcaggactctactccctgagctctgtggtgacagtgccctccagcagcttgggcac |
| | | ccagacctacatctgcaatgtgaatcacaagcccagcaacaccaaggtggacaagaaagttga |
| | | gcccaaatcttgtgacaaaactcacacatgcccaccttgcccagcacctgaactcctgggggga |
| | | ccatcagtcttcctcttccccccaaaacccaaggacaccctcatgatctccagaacccctgaggt |
| | | cacatgtgtggtggtggatgtgagccatgaagaccctgaggtcaagttcaactggtatgtggatg |
| | | gtgtggaggtgcataatgccaagacaaagcccagggaggagcagtacaacagcacttacaga |
| | | gtggtcagtgtcctcacagtcctgcaccaggactggctgaatggcaaggagtacaagtgcaag |
| | | gtctccaacaaagccctcccagcccccattgagaaaaccatctccaaagccaaagggcagccc |
| | | agggaaccacaggtgtacaccctgcccccatccagagatgagctgaccaagaaccaggtctcc |
| | | ctgacctgcctggtcaaaggcttctatccctctgacattgctgtggagtgggagagcaatgggca |
| | | gccagagaacaactacaagaccactcctcctgtgctggactctgatggctccttcttcctctactcc |
| | | aagctcacagtggacaagagcaggtggcagcaggggaatgtcttctcatgcagtgtgatgcatg |
| | | aggctctgcacaaccactacactcagaagagcctctccctgtctcctggcaaaagaaagagaag |
| | | gagtggctcaggagcccctgtgaaacagaccctgaactttgacctcttgaagcttgctggggatg |
| | | tggagtctaatcctggtccaatgtacaggatgcaactcctgtcttgcattgcactaagtcttgcactt |
| | | gtcacaaactcagacatccagatgacccaaagcccctcctctctgtcagccagtgtgggggata |
| | | gagtcacaattacttgcagagcttcccagggaataaggaactacttggcatggtatcagcaaaag |
| | | cctgggaaagctccaaagttgcttatctatgctgccagcactctgcagtcaggtgttcctagtagat |
| | | tctcaggcagtggaagtgggactgactttacattaactatttcctctctgcaacctgaggatgtggc |
| | | cacctattactgtcagaggtataatagagcaccttacacatttggccaaggtaccaaagtagaaat |
| | | caagaggactgtggctgcaccatctgtcttcatcttccccccatctgatgagcagttgaaatctgg |
| | | aactgcctctgttgtgtgcctgctgaataacttctatcccagagaggccaaagtacagtggaaggt |
| | | ggataatgccctccaatcaggcaactcccaggagagtgtcacagagcaggacagcaaggaca |
| | | gcacctacagcctcagcagcaccctgaccctgagcaaagcagactatgagaaacacaaagtct |
| | | atgcctgtgaagtcacccatcagggcctgagctccccagtcacaaagagcttcaacaggggag |
| | | agtgttgacaattgctgtgccttctagttgccagccatctgttgtttgcccctcccccgtgccttcctt |
| | | gaccctggaaggtgccactcccactgtcctttcctaataaaatgaggaaattgcatcgcattgtctg |
| | | agtaggtgtcattctattctggggggtggggtggggcaggacagcaagggggaggattggga |
| | | agacaatagcaggcatgctggggatgcggtgggctctatggagctcgagaggaacccctagtg |
| | | atggagttggccactccctctctgcgcgctcgctcgctcactgaggccgggcgaccaaaggtcg |
| | | cccgacgcccgggctttgcccgggcggcctcagtgagcgagcgagcgcgcagctgcagatct |
| | | g |
| Full: 4595 | EC #6 | ctgcgcgctcgctcgctcactgaggccgcccgggcaaagcccgggcgtcgggcgacctttgg |
| CAG | H- | tcgcccggcctcagtgagcgagcgagcgcgcagagagggagtggccaactccatcactagg |
| Promoter: | F2A- | ggttccttagtattaaacgcgtgtcgacattgattattgactagttattaatagtaatcaattacgggg |
| 146-1870 | L- | tcattagttcatagcccatatatggagttccgcgttacataacttacggtaaatggcccgcctggct |
| IL-10 leader: | 4xmiR142 | gaccgcccaacgacccccgcccattgacgtcaataatgacgtatgttcccatagtaacgccaata |
| 1898-1950 | | gggactttccattgacgtcaatgggtggactatttacggtaaactgcccacttggcagtacatcaa |
| HC: 1951- | | gtgtatcatatgccaagtacgccccctattgacgtcaatgacggtaaatggcccgcctggcattat |
| 3340 | | gcccagtacatgaccttatgggactttcctacttggcagtacatctacgtattagtcatcgctattac |
| Furin | | catgggtcgaggtgagccccacgttctgcttcactctccccatctcccccccctccccaccccca |
| cleavage site: | | attttgtatttatttattttttaattattttgtgcagcgatgggggcggggggggggggggcgcgcg |
| 3304-3328 | | ccaggcggggcggggcggggcgaggggcggggcggggcgaggcggagaggtgcggcg |
| 2a: 3329- | | gcagccaatcagagcggcgcgctccgaaagtttccttttatggcgaggcggcggcggcggcg |
| 3440 | | gccctataaaaagcgaagcgcgcggggggggagtcgctgcgcgctgccttcgccccgtgc |
| IL-2 leader: | | cccgctccgccgccgcctcgcgccgcccgccccggctctgactgaccgcgttactcccacagg |
| 3401-3459 | | tgagcgggcgggacggcccttctcctccgggctgtaattagcgcttggtttaatgacggctcgttt |
| LC: 3460- | | cttttctgtggctgcgtgaaagccttgaggggctccgggagggccctttgtgcggggggagcg |
| 4105 | | gctcggggggggggtgcgtgcgtgtgtgtgtgcgtggggagcgccgcgtgcggctccgcgctgccc |
| 4xmiR-142: | | ggcggctgtgagcgctgcgggcgcggcgcggggctttgtgcgctccgcagtgtgcgcgaga |
| 4112-4215 | | ggagcgcggccgggggcggtgccacgcggtgcggggggggctgcgaggggaacaaagg |
| BGH | | ctgcgtgcggggtgtgtgcgtggggggggtgagcagggggtgtgggcgcggcggtcgggct |
| poly(A): | | gtaacccccccctgcacccccctccccgagttgctgagcacggcccggcttcgggtgcggggc |
| 4222-4445 | | tccgtgcggggcgtggcgcggggctcgccgtgccgggcggggggggcggcgggtgggg |
| (SEQ ID | | gtgccgggcggggggggccgcctcgggccggggagggctcgggggaggggcgcggcg |
| NO: 67 | | gcccccggagcgccggcggctgtcgaggcgcggcgagccgcagccattgccttttatggtaat |
| (including | | cgtgcgagagggcgcagggacttcctttgtcccaaatctgtgcggagccgaaatctgggaggc |
| ITRs)) | | gccgccgcaccccctctagcgggcgcggggcgaagcggtgcggcgccggcaggaaggaa |
| Antibody | | atgggcggggagggccttcgtgcgtcgccgcgccgccgtccccttctccctctccagcctcgg |
| expression | | ggctgtccgcggggggacggctgccttcgggggggacggggcagggggggttcggcttct |
| cassette (w/o | | ggcgtgtgaccggcggtctagactctgctaaccatgttcatgccttcttctctttcctacagctcctg |
| ITRs | | ggcaacgtgctggttgttgtgctgtctcatcattttggcaaagaattcaagcttccagctagcgcca |
| (nucleotides | | ccatgcacagctcagcactgctctgttgcctggtcctcctgactggggtgagggctgaggtgca |
| 146-4445; | | gctggttgaaagtggaggagggcttgttcaacctggtagatccttgagactttcttgtgctgcttct |
| SEQ ID NO: | | ggcttcacctttgatgattatgcaatgcactgggtgaggcaggcacctggaaaggggctggagt |
| 121) | | gggtatcagccatcacatggaacagtggccatattgactatgctgatagtgtggaaggtagattca |
| | | ctatatccagagacaatgccaaaaactctttatacctgcagatgaattcactaagggcagaggata |
| | | ctgctgtctattactgtgctaaggtatcatatctcagcacagcatcctctctggactactggggaca |
| | | agggacattggttactgtgagctctgcctccaccaagggcccaagtgtcttccccctggcaccct |
| | | cctccaagagcacctctgggggcacagcagccctgggctgcctggtcaaggactacttccctg |
| | | aacctgtgactgtgtcttggaactcaggtgccctgaccagtggagtgcacaccttcccagctgtc |
| | | ctacagtcctcaggactctactccctgagctctgtggtgacagtgccctccagcagcttgggcac |
| | | ccagacctacatctgcaatgtgaatcacaagcccagcaacaccaaggtggacaagaaagttga |
| | | gcccaaatcttgtgacaaaactcacacatgcccaccttgcccagcacctgaactcctgggggga |
| | | ccatcagtcttcctcttccccccaaaacccaaggacaccctcatgatctccagaacccctgaggt |
| | | cacatgtgtggtggtggatgtgagccatgaagaccctgaggtcaagttcaactggtatgtggatg |
| | | gtgtggaggtgcataatgccaagacaaagcccagggaggagcagtacaacagcacttacaga |
| | | gtggtcagtgtcctcacagtcctgcaccaggactggctgaatggcaaggagtacaagtgcaag |
| | | gtctccaacaaagccctcccagcccccattgagaaaaccatctccaaagccaaagggcagccc |
| | | agggaaccacaggtgtacaccctgcccccatccagagatgagctgaccaagaaccaggtctcc |
| | | ctgacctgcctggtcaaaggcttctatccctctgacattgctgtggagtgggagagcaatgggca |
| | | gccagagaacaactacaagaccactcctcctgtgctggactctgatggctccttcttcctctactcc |
| | | aagctcacagtggacaagagcaggtggcagcaggggaatgtcttctcatgcagtgtgatgcatg |
| | | aggctctgcacaaccactacactcagaagagcctctccctgtctcctggcaaaagaaagagaag |
| | | gagtggctcaggagcccctgtgaaacagaccctgaactttgacctcttgaagcttgctggggatg |
| | | tggagtctaatcctggtccaatgtacaggatgcaactcctgtcttgcattgcactaagtcttgcactt |
| | | gtcacaaactcagacatccagatgacccaaagcccctcctctctgtcagccagtgtgggggata |
| | | gagtcacaattacttgcagagcttcccagggaataaggaactacttggcatggtatcagcaaaag |
| | | cctgggaaagctccaaagttgcttatctatgctgccagcactctgcagtcaggtgttcctagtagat |
| | | tctcaggcagtggaagtgggactgactttacattaactatttcctctctgcaacctgaggatgtggc |
| | | cacctattactgtcagaggtataatagagcaccttacacatttggccaaggtaccaaagtagaaat |
| | | caagaggactgtggctgcaccatctgtcttcatcttccccccatctgatgagcagttgaaatctgg |
| | | aactgcctctgttgtgtgcctgctgaataacttctatcccagagaggccaaagtacagtggaaggt |
| | | ggataatgccctccaatcaggcaactcccaggagagtgtcacagagcaggacagcaaggaca |
| | | gcacctacagcctcagcagcaccctgaccctgagcaaagcagactatgagaaacacaaagtct |
| | | atgcctgtgaagtcacccatcagggcctgagctccccagtcacaaagagcttcaacaggggag |
| | | agtgttgacctaggtccataaagtaggaaacactacactattccataaagtaggaaacactacatc |
| | | actccataaagtaggaaacactacaagtctccataaagtaggaaacactacacaattgctgtgcct |
| | | tctagttgccagccatctgttgtttgcccctcccccgtgccttccttgaccctggaaggtgccactc |
| | | ccactgtcctttcctaataaaatgaggaaattgcatcgcattgtctgagtaggtgtcattctattctgg |
| | | ggggtggggtggggcaggacagcaagggggaggattgggaagacaatagcaggcatgctg |
| | | gggatgcggtgggctctatggagctcgagaggaacccctagtgatggagttggccactccctct |
| | | ctgcgcgctcgctcgctcactgaggccgggcgaccaaaggtcgcccgacgcccgggctttgc |
| | | ccgggcggcctcagtgagcgagcgagcgcgcagctgcagatctg |
| Full: 4802 | EC #7 | ctgcgcgctcgctcgctcactgaggccgcccgggcaaagcccgggcgtcgggcgacctttgg |
| CAG | L- | tcgcccggcctcagtgagcgagcgagcgcgcagagagggagtggccaactccatcactagg |
| Promoter: | IRES- | ggttccttagtattaaacgcgtgtcgacattgattattgactagttattaatagtaatcaattacgggg |
| 146-1870 | H | tcattagttcatagcccatatatggagttccgcgttacataacttacggtaaatggcccgcctggct |
| IL-2 leader: | | gaccgcccaacgacccccgcccattgacgtcaataatgacgtatgttcccatagtaacgccaata |
| 1898-1957 | | gggactttccattgacgtcaatgggtggactatttacggtaaactgcccacttggcagtacatcaa |
| LC: 1958- | | gtgtatcatatgccaagtacgccccctattgacgtcaatgacggtaaatggcccgcctggcattat |
| 2602 | | gcccagtacatgaccttatgggactttcctacttggcagtacatctacgtattagtcatcgctattac |
| IRES: 2603- | | catgggtcgaggtgagccccacgttctgcttcactctccccatctcccccccctccccaccccca |
| 3187 | | attttgtatttatttattttttaattattttgtgcagcgatgggggcggggggggggggggcgcgcg |
| IL-10 leader: | | ccaggcggggcggggcggggcgaggggcggggcggggcgaggcggagaggtgcggcg |
| 3188-3241 | | gcagccaatcagagcggcgcgctccgaaagtttccttttatggcgaggcggcggcggcggcg |
| HC: 3242- | | gccctataaaaagcgaagcgcgcggggggggagtcgctgcgcgctgccttcgccccgtgc |
| 4597 | | cccgctccgccgccgcctcgcgccgcccgccccggctctgactgaccgcgttactcccacagg |
| Synthetic | | tgagcgggcgggacggcccttctcctccgggctgtaattagcgcttggtttaatgacggctcgttt |
| poly(A): | | cttttctgtggctgcgtgaaagccttgaggggctccgggagggccctttgtgcggggggagcg |
| 4604-4652 | | gctcggggggtgcgtgcgtgtgtgtgtgcgtggggagcgccgcgtgcggctccgcgctgccc |
| (SEQ ID | | ggcggctgtgagcgctgcgggcgcggcgcggggctttgtgcgctccgcagtgtgcgcgaga |
| NO: 68 | | ggagcgcggccgggggcggtgccacgcggtgcggggggggctgcgaggggaacaaagg |
| (including | | ctgcgtgcggggtgtgtgcgtggggggggtgagcagggggtgtgggcgcggcggtcgggct |
| ITRs)) | | gtaacccccccctgcacccccctccccgagttgctgagcacggcccggcttcgggtgcggggc |
| Antibody | | tccgtgcggggcgtggcgcggggctcgccgtgccgggcggggggtggcggcgggtgggg |
| expression | | gtgccgggcggggcggggccgcctcgggccggggagggctcgggggaggggcgcggcg |
| cassette (w/o | | gcccccggagcgccggcggctgtcgaggcgcggcgagccgcagccattgccttttatggtaat |
| ITRs | | cgtgcgagagggcgcagggacttcctttgtcccaaatctgtgcggagccgaaatctgggaggc |
| (nucleotides | | gccgccgcaccccctctagcgggcgcggggcgaagcggtgcggcgccggcaggaaggaa |
| 146-4652; | | atgggcggggagggccttcgtgcgtcgccgcgccgccgtccccttctccctctccagcctcgg |
| SEQ ID NO: | | ggctgtccgcggggggacggctgccttcgggggggacggggcagggcggggttcggcttct |
| 122) | | ggcgtgtgaccggcggtctagactctgctaaccatgttcatgccttcttctctttcctacagctcctg |
| | | ggcaacgtgctggttgttgtgctgtctcatcattttggcaaagaattcaagcttccagctagcgcca |
| | | ccatgtacaggatgcaactcctgtcttgcattgcactaagtcttgcacttgtcacaaactcggacat |
| | | ccagatgacccaaagcccctcctctctgtcagccagtgtgggcgatcgggtcacaattacttgca |
| | | gagcttcgcagggaataaggaactacctcgcatggtatcagcaaaagcctgggaaagcgccaa |
| | | agttgcttatctacgccgccagcacgctgcagtccggtgttccgtctcgcttctctggcagtggaa |
| | | gcgggaccgactttacattaactatttcctctctgcaacccgaggatgtggccacctattactgtca |
| | | gcgatataatcgtgcaccttacacattcggccaaggtaccaaagtagaaatcaagcgaactgtgg |
| | | ctgcaccatctgtcttcatcttcccgccatctgatgagcagttgaaatctggaactgcctctgttgtg |
| | | tgcctgctgaataacttctatcccagagaggccaaagtacagtggaaggtggataacgccctcc |
| | | aatcgggcaactcccaggagagtgtcacagagcaggacagcaaggacagcacctacagcctc |
| | | agcagcaccctgacgctgagcaaagcagactacgagaaacacaaagtctacgcctgcgaagt |
| | | cacccatcagggcctgagctcgcccgtcacaaagagcttcaacaggggagagtgttgagcccc |
| | | tctccctcccccccccctaacgttactggccgaagccgcttggaataaggccggtgtgcgtttgtc |
| | | tatatgttattttccaccatattgccgtcttttggcaatgtgagggcccggaaacctggccctgtcttc |
| | | ttgacgagcattcctaggggtctttcccctctcgccaaaggaatgcaaggtctgttgaatgtcgtg |
| | | aaggaagcagttcctctggaagcttcttgaagacaaacaacgtctgtagcgaccctttgcaggca |
| | | gcggaaccccccacctggcgacaggtgcctctgcggccaaaagccacgtgtataagatacacc |
| | | tgcaaaggcggcacaaccccagtgccacgttgtgagttggatagttgtggaaagagtcaaatgg |
| | | ctctcctcaagcgtattcaacaaggggctgaaggatgcccagaaggtaccccattgtatgggatc |
| | | tgatctggggcctcggtacacatgctttacatgtgtttagtcgaggttaaaaaaacgtctaggcccc |
| | | ccgaaccacggggacgtggttttcctttgaaaaacacgatgataatatggccacaatgcacagct |
| | | cagcactgctctgttgcctggtcctcctgactggggtgagggccgaggtgcagctggtcgaaag |
| | | cggcggagggctcgttcaacccggtcggtccttgagactttcttgcgccgcttctggcttcaccttt |
| | | gacgattacgcaatgcactgggtgaggcaggcgcctggaaaggggctggagtgggtatcagc |
| | | catcacatggaactcgggccatattgactatgctgatagcgtggaaggtcgcttcactatatcccg |
| | | agacaatgccaaaaactctttatacctgcagatgaattcactacgtgcagaggatacggccgtcta |
| | | ttactgtgctaaggtgtcatatctcagcaccgcatcctctctggactactggggacaagggacatt |
| | | ggttactgtgagctccgcctccaccaagggcccatcggtcttccccctggcaccctcctccaaga |
| | | gcacctctgggggcacagcggccctgggctgcctggtcaaggactacttccccgaaccggtga |
| | | cggtgtcgtggaactcaggcgccctgaccagcggcgtgcacaccttcccggctgtcctacagtc |
| | | ctcaggactctactccctcagcagcgtggtgaccgtgccctccagcagcttgggcacccagacc |
| | | tacatctgcaacgtgaatcacaagcccagcaacaccaaggtggacaagaaagttgagcccaaa |
| | | tcttgtgacaaaactcacacatgcccaccgtgcccagcacctgaactcctggggggaccgtcag |
| | | tcttcctcttccccccaaaacccaaggacaccctcatgatctcccggacccctgaggtcacatgc |
| | | gtggtggtggacgtgagccacgaagaccctgaggtcaagttcaactggtacgtggacggcgtg |
| | | gaggtgcataatgccaagacaaagccgcgggaggagcagtacaacagcacgtaccgtgtggt |
| | | cagcgtcctcaccgtcctgcaccaggactggctgaatggcaaggagtacaagtgcaaggtctc |
| | | caacaaagccctcccagcccctatcgagaaaacaatctccaaagccaaagggcagccccgag |
| | | aaccacaggtgtacaccctgcccccatcccgggatgagctgaccaagaaccaagtcagcctga |
| | | cctgcctggtcaaaggcttctatcccagcgacatcgccgtggagtgggagagcaatgggcagc |
| | | cggagaacaactacaagaccacgcctcccgtgctggactccgacggctccttcttcctctactcc |
| | | aagctcaccgtggacaagagcaggtggcagcaggggaacgtcttctcatgctccgtgatgcat |
| | | gaggctctgcacaaccactacacgcagaagagcctctccctgtctccgggcaaatgacaattga |
| | | ataaaagatctttattttcattagatctgtgtgttggttttttgtgtggagctcgagaggaacccctagt |
| | | gatggagttggccactccctctctgcgcgctcgctcgctcactgaggccgggcgaccaaaggtc |
| | | gcccgacgcccgggctttgcccgggcggcctcagtgagcgagcgagcgcgcagctgcagat |
| | | ctg |
| Full: 4914 | EC #8 | ctgcgcgctcgctcgctcactgaggccgcccgggcaaagcccgggcgtcgggcgacctttgg |
| CAG | L- | tcgcccggcctcagtgagcgagcgagcgcgcagagagggagtggccaactccatcactagg |
| Promoter: | IRES- | ggttccttagtattaaacgcgtgtcgacattgattattgactagttattaatagtaatcaattacgggg |
| 146-1870 | H- | tcattagttcatagcccatatatggagttccgcgttacataacttacggtaaatggcccgcctggct |
| IL-2 leader: | miR142 | gaccgcccaacgacccccgcccattgacgtcaataatgacgtatgttcccatagtaacgccaata |
| 1898-1957 | | gggactttccattgacgtcaatgggggactatttacggtaaactgcccacttggcagtacatcaa |
| LC: 1958- | | gtgtatcatatgccaagtacgccccctattgacgtcaatgacggtaaatggcccgcctggcattat |
| 2602 | | gcccagtacatgaccttatgggactttcctacttggcagtacatctacgtattagtcatcgctattac |
| IRES: 2603- | | catgggtcgaggtgagccccacgttctgcttcactctccccatctcccccccctccccaccccca |
| 3187 | | attttgtatttatttattttttaattattttgtgcagcgatgggggcggggggggggggggcgcgcg |
| IL-10 leader: | | ccaggcggggggggcggggcgaggggcggggcggggcgaggcggagaggtgcggcg |
| 3188-3241 | | gcagccaatcagagcggcgcgctccgaaagtttccttttatggcgaggcggcggcggcggcg |
| HC: 3242- | | gccctataaaaagcgaagcgcgcggggggggagtcgctgcgcgctgccttcgccccgtgc |
| 4597 | | cccgctccgccgccgcctcgcgccgcccgccccggctctgactgaccgcgttactcccacagg |
| 4xmiR-142: | | tgagcgggcgggacggcccttctcctccgggctgtaattagcgcttggtttaatgacggctcgttt |
| 4606-4709 | | cttttctgtggctgcgtgaaagccttgaggggctccgggagggccctttgtgcggggggagcg |
| Synthetic | | gctcggggggtgcgtgcgtgtgtgtgtgcgtggggagcgccgcgtgcggctccgcgctgccc |
| poly(A): | | ggcggctgtgagcgctgcgggcgcggcgcggggctttgtgcgctccgcagtgtgcgcgaga |
| 4716-4764 | | ggagcgcggccgggggcggtgccacgcggtgcggggggggctgcgaggggaacaaagg |
| (SEQ ID | | ctgcgtgcggggtgtgtgcgtggggggggtgagcagggggtgtgggcgcggcggtcgggct |
| NO: 69 | | gtaacccccccctgcacccccctccccgagttgctgagcacggcccggcttcgggtgcggggc |
| (including | | tccgtgcggggcgtggcgcggggctcgccgtgccgggcggggggggcggcgggtgggg |
| ITRs)) | | gtgccgggcggggcggggccgcctcgggccggggagggctcgggggaggggcgcggcg |
| Antibody | | gcccccggagcgccggcggctgtcgaggcgcggcgagccgcagccattgccttttatggtaat |
| expression | | cgtgcgagagggcgcagggacttcctttgtcccaaatctgtgcggagccgaaatctgggaggc |
| cassette (w/o | | gccgccgcaccccctctagcgggcgcggggcgaagcggtgcggcgccggcaggaaggaa |
| ITRs | | atgggcggggagggccttcgtgcgtcgccgcgccgccgtccccttctccctctccagcctcgg |
| (nucleotides | | ggctgtccgcggggggacggctgccttcgggggggacggggcagggcggggttcggcttct |
| 146-4764; | | ggcgtgtgaccggcggtctagactctgctaaccatgttcatgccttcttctctttcctacagctcctg |
| SEQ ID NO: | | ggcaacgtgctggttgttgtgctgtctcatcattttggcaaagaattcaagcttccagctagcgcca |
| 123) | | ccatgtacaggatgcaactcctgtcttgcattgcactaagtcttgcacttgtcacaaactcggacat |
| | | ccagatgacccaaagcccctcctctctgtcagccagtgtgggcgatcgggtcacaattacttgca |
| | | gagcttcgcagggaataaggaactacctcgcatggtatcagcaaaagcctgggaaagcgccaa |
| | | agttgcttatctacgccgccagcacgctgcagtccggtgttccgtctcgcttctctggcagtggaa |
| | | gcgggaccgactttacattaactatttcctctctgcaacccgaggatgtggccacctattactgtca |
| | | gcgatataatcgtgcaccttacacattcggccaaggtaccaaagtagaaatcaagcgaactgtgg |
| | | ctgcaccatctgtcttcatcttcccgccatctgatgagcagttgaaatctggaactgcctctgttgtg |
| | | tgcctgctgaataacttctatcccagagaggccaaagtacagtggaaggtggataacgccctcc |
| | | aatcgggcaactcccaggagagtgtcacagagcaggacagcaaggacagcacctacagcctc |
| | | agcagcaccctgacgctgagcaaagcagactacgagaaacacaaagtctacgcctgcgaagt |
| | | cacccatcagggcctgagctcgcccgtcacaaagagcttcaacaggggagagtgttgagcccc |
| | | tctccctcccccccccctaacgttactggccgaagccgcttggaataaggccggtgtgcgtttgtc |
| | | tatatgttattttccaccatattgccgtcttttggcaatgtgagggcccggaaacctggccctgtcttc |
| | | ttgacgagcattcctaggggtctttcccctctcgccaaaggaatgcaaggtctgttgaatgtcgtg |
| | | aaggaagcagttcctctggaagcttcttgaagacaaacaacgtctgtagcgaccctttgcaggca |
| | | gcggaaccccccacctggcgacaggtgcctctgcggccaaaagccacgtgtataagatacacc |
| | | tgcaaaggcggcacaaccccagtgccacgttgtgagttggatagttgtggaaagagtcaaatgg |
| | | ctctcctcaagcgtattcaacaaggggctgaaggatgcccagaaggtaccccattgtatgggatc |
| | | tgatctggggcctcggtacacatgctttacatgtgtttagtcgaggttaaaaaaacgtctaggcccc |
| | | ccgaaccacggggacgtggttttcctttgaaaaacacgatgataatatggccacaatgcacagct |
| | | cagcactgctctgttgcctggtcctcctgactggggtgagggccgaggtgcagctggtcgaaag |
| | | cggcggagggctcgttcaacccggtcggtccttgagactttcttgcgccgcttctggcttcaccttt |
| | | gacgattacgcaatgcactgggtgaggcaggcgcctggaaaggggctggagtgggtatcagc |
| | | catcacatggaactcgggccatattgactatgctgatagcgtggaaggtcgcttcactatatcccg |
| | | agacaatgccaaaaactctttatacctgcagatgaattcactacgtgcagaggatacggccgtcta |
| | | ttactgtgctaaggtgtcatatctcagcaccgcatcctctctggactactggggacaagggacatt |
| | | ggttactgtgagctccgcctccaccaagggcccatcggtcttccccctggcaccctcctccaaga |
| | | gcacctctgggggcacagcggccctgggctgcctggtcaaggactacttccccgaaccggtga |
| | | cggtgtcgtggaactcaggcgccctgaccagcggcgtgcacaccttcccggctgtcctacagtc |
| | | ctcaggactctactccctcagcagcgtggtgaccgtgccctccagcagcttgggcacccagacc |
| | | tacatctgcaacgtgaatcacaagcccagcaacaccaaggtggacaagaaagttgagcccaaa |
| | | tcttgtgacaaaactcacacatgcccaccgtgcccagcacctgaactcctggggggaccgtcag |
| | | tcttcctcttccccccaaaacccaaggacaccctcatgatctcccggacccctgaggtcacatgc |
| | | gtggtggtggacgtgagccacgaagaccctgaggtcaagttcaactggtacgtggacggcgtg |
| | | gaggtgcataatgccaagacaaagccgcgggaggagcagtacaacagcacgtaccgtgtggt |
| | | cagcgtcctcaccgtcctgcaccaggactggctgaatggcaaggagtacaagtgcaaggtctc |
| | | caacaaagccctcccagcccctatcgagaaaacaatctccaaagccaaagggcagccccgag |
| | | aaccacaggtgtacaccctgcccccatcccgggatgagctgaccaagaaccaagtcagcctga |
| | | cctgcctggtcaaaggcttctatcccagcgacatcgccgtggagtgggagagcaatgggcagc |
| | | cggagaacaactacaagaccacgcctcccgtgctggactccgacggctccttcttcctctactcc |
| | | aagctcaccgtggacaagagcaggtggcagcaggggaacgtcttctcatgctccgtgatgcat |
| | | gaggctctgcacaaccactacacgcagaagagcctctccctgtctccgggcaaatgagtttaaa |
| | | ctccataaagtaggaaacactacactattccataaagtaggaaacactacatcactccataaagta |
| | | ggaaacactacaagtctccataaagtaggaaacactacacaattgaataaaagatctttattttcatt |
| | | agatctgtgtgttggttttttgtgtggagctcgagaggaacccctagtgatggagttggccactccc |
| | | tctctgcgcgctcgctcgctcactgaggccgggcgaccaaaggtcgcccgacgcccgggcttt |
| | | gcccgggcggcctcagtgagcgagcgagcgcgcagctgcagatctg |
| Full: 4802 | EC #9 | ctgcgcgctcgctcgctcactgaggccgcccgggcaaagcccgggcgtcgggcgacctttgg |
| CAG | L- | tcgcccggcctcagtgagcgagcgagcgcgcagagagggagtggccaactccatcactagg |
| Promoter: | IRES- | ggttccttagtattaaacgcgtgtcgacattgattattgactagttattaatagtaatcaattacgggg |
| 146-1870 | H | tcattagttcatagcccatatatggagttccgcgttacataacttacggtaaatggcccgcctggct |
| IL-2 leader: | | gaccgcccaacgacccccgcccattgacgtcaataatgacgtatgttcccatagtaacgccaata |
| 1898-1956 | | gggactttccattgacgtcaatgggtggactatttacggtaaactgcccacttggcagtacatcaa |
| LC: 1957- | | gtgtatcatatgccaagtacgccccctattgacgtcaatgacggtaaatggcccgcctggcattat |
| 2602 | | gcccagtacatgaccttatgggactttcctacttggcagtacatctacgtattagtcatcgctattac |
| IRES: 2603- | | catgggtcgaggtgagccccacgttctgcttcactctccccatctcccccccctccccaccccca |
| 3187 | | attttgtatttatttattttttaattattttgtgcagcgatgggggcggggggggggggggcgcgcg |
| IL-10 leader: | | ccaggcggggcggggcggggcgaggggcggggcggggcgaggcggagaggtgcggcg |
| 3188-3240 | | gcagccaatcagagcggcgcgctccgaaagtttccttttatggcgaggcggcggcggcggcg |
| HC: 3241- | | gccctataaaaagcgaagcgcgcggggggggagtcgctgcgcgctgccttcgccccgtgc |
| 4597 | | cccgctccgccgccgcctcgcgccgcccgccccggctctgactgaccgcgttactcccacagg |
| Synthetic | | tgagcgggcgggacggcccttctcctccgggctgtaattagcgcttggtttaatgacggctcgttt |
| poly(A): | | cttttctgtggctgcgtgaaagccttgaggggctccgggagggccctttgtgcggggggagcg |
| 4604-4652 | | gctcggggggtgcgtgcgtgtgtgtgtgcgtggggagcgccgcgtgcggctccgcgctgccc |
| (SEQ ID | | ggcggctgtgagcgctgcgggcgcggcgcggggctttgtgcgctccgcagtgtgcgcgaga |
| NO: 70 | | ggagcgcggccgggggcggtgccacgcggtgcggggggggctgcgaggggaacaaagg |
| (including | | ctgcgtgcggggtgtgtgcgtggggggggtgagcagggggtgtgggcgcggcggtcgggct |
| ITRs)) | | gtaacccccccctgcacccccctccccgagttgctgagcacggcccggcttcgggtgcggggc |
| Antibody | | tccgtgcggggcgtggcgcggggctcgccgtgccgggggggggtggcggcgggtgggg |
| expression | | gtgccgggcggggggggccgcctcgggccggggagggctcgggggaggggcgcggcg |
| cassette (w/o | | gcccccggagcgccggcggctgtcgaggcgcggcgagccgcagccattgccttttatggtaat |
| ITRs | | cgtgcgagagggcgcagggacttcctttgtcccaaatctgtgcggagccgaaatctgggaggc |
| (nucleotides | | gccgccgcaccccctctagcgggcgcggggcgaagcggtgcggcgccggcaggaaggaa |
| 146-4652; | | atgggcggggagggccttcgtgcgtcgccgcgccgccgtccccttctccctctccagcctcgg |
| SEQ ID NO: | | ggctgtccgcggggggacggctgccttcgggggggacggggcagggcggggttcggcttct |
| 124) | | ggcgtgtgaccggcggtctagactctgctaaccatgttcatgccttcttctctttcctacagctcctg |
| | | ggcaacgtgctggttgttgtgctgtctcatcattttggcaaagaattcaagcttccagctagcgcca |
| | | ccatgtacaggatgcaactcctgtcttgcattgcactaagtcttgcacttgtcacaaactcagacat |
| | | ccagatgacccaaagcccctcctctctgtcagccagtgtgggggatcgcgtcacaattacttgca |
| | | gagcttcccagggaataaggaactacctcgcgtggtatcagcaaaagcctgggaaagcgccaa |
| | | agttgcttatctatgccgccagcactctgcagtcaggtgttcctagtagattctctggcagtggaag |
| | | cgggactgactttacattaactatttcctctctgcaacctgaggatgtggccacctattactgtcagc |
| | | ggtataatcgcgcaccttacacatttggccaaggtaccaaagtagaaatcaagcggactgtggct |
| | | gcaccatctgtcttcatcttcccgccatctgatgagcagttgaaatctggaactgcctctgttgtgtg |
| | | cctgctgaataacttctatcccagagaggccaaagtacagtggaaggtggataatgccctccaaa |
| | | gtggcaactcccaggagagtgtcacagagcaggacagcaaggacagcacctacagcctcagc |
| | | agcaccctgaccctgagcaaagcagactatgagaaacacaaagtctacgcgtgtgaagtcacc |
| | | catcagggcctgagctccccagtcacaaagagcttcaacaggggagagtgttgagcccctctcc |
| | | ctcccccccccctaacgttactggccgaagccgcttggaataaggccggtgtgcgtttgtctatat |
| | | gttattttccaccatattgccgtcttttggcaatgtgagggcccggaaacctggccctgtcttcttga |
| | | cgagcattcctaggggtctttcccctctcgccaaaggaatgcaaggtctgttgaatgtcgtgaagg |
| | | aagcagttcctctggaagcttcttgaagacaaacaacgtctgtagcgaccctttgcaggcagcgg |
| | | aaccccccacctggcgacaggtgcctctgcggccaaaagccacgtgtataagatacacctgca |
| | | aaggcggcacaaccccagtgccacgttgtgagttggatagttgtggaaagagtcaaatggctct |
| | | cctcaagcgtattcaacaaggggctgaaggatgcccagaaggtaccccattgtatgggatctgat |
| | | ctggggcctcggtacacatgctttacatgtgtttagtcgaggttaaaaaaacgtctaggccccccg |
| | | aaccacggggacgtggttttcctttgaaaaacacgatgataatatggccacaatgcacagctcag |
| | | cactgctctgttgcctggtcctcctgactggggtgagggctgaggtgcagctggttgaaagcgg |
| | | cggagggcttgttcaacctggtagatccttgagactttcttgcgccgcttctggcttcacctttgatg |
| | | attatgcaatgcactgggtgaggcaggcgcctggaaaggggctggagtgggtatcagccatca |
| | | catggaacagtggccatattgactatgctgatagtgtggaaggtagattcactatatcccgcgaca |
| | | atgccaaaaactctttatacctgcagatgaattcactacgcgcagaggatactgcggtctattactg |
| | | tgctaaggtgtcatatctcagcaccgcatcctctctggactactggggacaagggacattggttac |
| | | tgtgagctccgcctccaccaagggcccaagtgtcttccccctggcaccctcctccaagagcacc |
| | | tctgggggcacagcggccctgggctgcctggtcaaggactacttccctgaacctgtgacagtgt |
| | | cttggaactcaggcgccctgaccagcggagtgcacaccttcccagctgtcctacagtcctcagg |
| | | actctactccctcagcagtgtggtgactgtgccctccagcagcttgggcacccagacctacatct |
| | | gcaatgtgaatcacaagcccagcaacaccaaggtggacaagaaagttgagcccaaatcttgtg |
| | | acaaaactcacacatgcccaccttgcccagcacctgaactcctggggggaccatcagtcttcctc |
| | | ttccccccaaaacccaaggacaccctcatgatctcccgcacccctgaggtcacatgtgtggtggt |
| | | ggatgtgagccatgaagaccctgaggtcaagttcaactggtatgtggatggtgtggaggtgcata |
| | | atgccaagacaaagccgcgggaggagcagtacaacagcacatacagagtggtctctgtcctca |
| | | ctgtcctgcaccaggactggctgaatggcaaggagtacaagtgcaaggtctccaacaaagccct |
| | | cccagcccctattgagaaaacaatctccaaagccaaagggcagcccagggaaccacaggtgt |
| | | acaccctgcccccatcccgcgatgagctgaccaagaaccaggtctccctgacctgcctggtcaa |
| | | aggcttctatcccagtgacattgctgtggagtgggagagcaatgggcagcctgagaacaactac |
| | | aagaccacacctccagtgctggactctgatggctccttcttcctctactccaagctcactgtggac |
| | | aagagcaggtggcagcaggggaatgtcttctcatgcagtgtgatgcatgaggctctgcacaacc |
| | | actacacacagaagagcctctccctgtctcctggcaaatgacaattgaataaaagatctttattttca |
| | | ttagatctgtgtgttggttttttgtgtggagctcgagaggaacccctagtgatggagttggccactc |
| | | cctctctgcgcgctcgctcgctcactgaggccgggcgaccaaaggtcgcccgacgcccgggc |
| | | tttgcccgggcggcctcagtgagcgagcgagcgcgcagctgcagatctg |
| Full: 4914 | EC | ctgcgcgctcgctcgctcactgaggccgcccgggcaaagcccgggcgtcgggcgacctttgg |
| CAG | #10 | tcgcccggcctcagtgagcgagcgagcgcgcagagagggagtggccaactccatcactagg |
| Promoter: | L- | ggttccttagtattaaacgcgtgtcgacattgattattgactagttattaatagtaatcaattacgggg |
| 146-1870 | IRES- | tcattagttcatagcccatatatggagttccgcgttacataacttacggtaaatggcccgcctggct |
| IL-2 leader: | H- | gaccgcccaacgacccccgcccattgacgtcaataatgacgtatgttcccatagtaacgccaata |
| 1898-1956 | 4xmiR142 | gggactttccattgacgtcaatgggtggactatttacggtaaactgcccacttggcagtacatcaa |
| LC: 1957- | | gtgtatcatatgccaagtacgccccctattgacgtcaatgacggtaaatggcccgcctggcattat |
| 2602 | | gcccagtacatgaccttatgggactttcctacttggcagtacatctacgtattagtcatcgctattac |
| IRES: 2603- | | catgggtcgaggtgagccccacgttctgcttcactctccccatctcccccccctccccaccccca |
| 3187 | | attttgtatttatttattttttaattattttgtgcagcgatgggggcggggggggggggggcgcgcg |
| IL-10 leader: | | ccaggcggggcggggcggggcgaggggcggggcggggcgaggcggagaggtgcggcg |
| 3188-3240 | | gcagccaatcagagcggcgcgctccgaaagtttccttttatggcgaggcggcggcggcggcg |
| HC: 3241- | | gccctataaaaagcgaagcgcgcggggggggagtcgctgcgcgctgccttcgccccgtgc |
| 4597 | | cccgctccgccgccgcctcgcgccgcccgccccggctctgactgaccgcgttactcccacagg |
| 4xmiR-142: | | tgagcgggcgggacggcccttctcctccgggctgtaattagcgcttggtttaatgacggctcgttt |
| 4606-4709 | | cttttctgtggctgcgtgaaagccttgaggggctccgggagggccctttgtgcggggggagcg |
| Synthetic | | gctcggggggtgcgtgcgtgtgtgtgtgcgtggggagcgccgcgtgcggctccgcgctgccc |
| poly(A): | | ggcggctgtgagcgctgcgggcgcggcgcggggctttgtgcgctccgcagtgtgcgcgaga |
| 4716-4764 | | ggagcgcggccgggggcggtgccacgcggtgcggggggggctgcgaggggaacaaagg |
| (SEQ ID | | ctgcgtgcggggtgtgtgcgtggggggggtgagcagggggtgtgggcgcggcggtcgggct |
| NO: 71 | | gtaacccccccctgcacccccctccccgagttgctgagcacggcccggcttcgggtgcggggc |
| (including | | tccgtgcggggcgtggcgcggggctcgccgtgccgggcggggggtggcggcgggtgggg |
| ITRs)) | | gtgccgggcggggcggggccgcctcgggccggggagggctcgggggaggggcgcggcg |
| Antibody | | gcccccggagcgccggcggctgtcgaggcgcggcgagccgcagccattgccttttatggtaat |
| expression | | cgtgcgagagggcgcagggacttcctttgtcccaaatctgtgcggagccgaaatctgggaggc |
| cassette (w/o | | gccgccgcaccccctctagcgggcgcggggcgaagcggtgcggcgccggcaggaaggaa |
| ITRs | | atgggcggggagggccttcgtgcgtcgccgcgccgccgtccccttctccctctccagcctcgg |
| (nucleotides | | ggctgtccgcggggggacggctgccttcgggggggacggggcagggcggggttcggcttct |
| 146-4764; | | ggcgtgtgaccggcggtctagactctgctaaccatgttcatgccttcttctctttcctacagctcctg |
| SEQ ID NO: | | ggcaacgtgctggttgttgtgctgtctcatcattttggcaaagaattcaagcttccagctagcgcca |
| 125) | | ccatgtacaggatgcaactcctgtcttgcattgcactaagtcttgcacttgtcacaaactcagacat |
| | | ccagatgacccaaagcccctcctctctgtcagccagtgtgggggatcgcgtcacaattacttgca |
| | | gagcttcccagggaataaggaactacctcgcgtggtatcagcaaaagcctgggaaagcgccaa |
| | | agttgcttatctatgccgccagcactctgcagtcaggtgttcctagtagattctctggcagtggaag |
| | | cgggactgactttacattaactatttcctctctgcaacctgaggatgtggccacctattactgtcagc |
| | | ggtataatcgcgcaccttacacatttggccaaggtaccaaagtagaaatcaagcggactgtggct |
| | | gcaccatctgtcttcatcttcccgccatctgatgagcagttgaaatctggaactgcctctgttgtgtg |
| | | cctgctgaataacttctatcccagagaggccaaagtacagtggaaggtggataatgccctccaaa |
| | | gtggcaactcccaggagagtgtcacagagcaggacagcaaggacagcacctacagcctcagc |
| | | agcaccctgaccctgagcaaagcagactatgagaaacacaaagtctacgcgtgtgaagtcacc |
| | | catcagggcctgagctccccagtcacaaagagcttcaacaggggagagtgttgagcccctctcc |
| | | ctcccccccccctaacgttactggccgaagccgcttggaataaggccggtgtgcgtttgtctatat |
| | | gttattttccaccatattgccgtcttttggcaatgtgagggcccggaaacctggccctgtcttcttga |
| | | cgagcattcctaggggtctttcccctctcgccaaaggaatgcaaggtctgttgaatgtcgtgaagg |
| | | aagcagttcctctggaagcttcttgaagacaaacaacgtctgtagcgaccctttgcaggcagcgg |
| | | aaccccccacctggcgacaggtgcctctgcggccaaaagccacgtgtataagatacacctgca |
| | | aaggcggcacaaccccagtgccacgttgtgagttggatagttgtggaaagagtcaaatggctct |
| | | cctcaagcgtattcaacaaggggctgaaggatgcccagaaggtaccccattgtatgggatctgat |
| | | ctggggcctcggtacacatgctttacatgtgtttagtcgaggttaaaaaaacgtctaggccccccg |
| | | aaccacggggacgtggttttcctttgaaaaacacgatgataatatggccacaatgcacagctcag |
| | | cactgctctgttgcctggtcctcctgactggggtgagggctgaggtgcagctggttgaaagcgg |
| | | cggagggcttgttcaacctggtagatccttgagactttcttgcgccgcttctggcttcacctttgatg |
| | | attatgcaatgcactgggtgaggcaggcgcctggaaaggggctggagtgggtatcagccatca |
| | | catggaacagtggccatattgactatgctgatagtgtggaaggtagattcactatatcccgcgaca |
| | | atgccaaaaactctttatacctgcagatgaattcactacgcgcagaggatactgcggtctattactg |
| | | tgctaaggtgtcatatctcagcaccgcatcctctctggactactggggacaagggacattggttac |
| | | tgtgagctccgcctccaccaagggcccaagtgtcttccccctggcaccctcctccaagagcacc |
| | | tctgggggcacagcggccctgggctgcctggtcaaggactacttccctgaacctgtgacagtgt |
| | | cttggaactcaggcgccctgaccagcggagtgcacaccttcccagctgtcctacagtcctcagg |
| | | actctactccctcagcagtgtggtgactgtgccctccagcagcttgggcacccagacctacatct |
| | | gcaatgtgaatcacaagcccagcaacaccaaggggacaagaaagttgagcccaaatcttgtg |
| | | acaaaactcacacatgcccaccttgcccagcacctgaactcctggggggaccatcagtcttcctc |
| | | ttccccccaaaacccaaggacaccctcatgatctcccgcacccctgaggtcacatgtgtggtggt |
| | | ggatgtgagccatgaagaccctgaggtcaagttcaactggtatgtggatggtgtggaggtgcata |
| | | atgccaagacaaagccgcgggaggagcagtacaacagcacatacagagtggtctctgtcctca |
| | | ctgtcctgcaccaggactggctgaatggcaaggagtacaagtgcaaggtctccaacaaagccct |
| | | cccagcccctattgagaaaacaatctccaaagccaaagggcagcccagggaaccacaggtgt |
| | | acaccctgcccccatcccgcgatgagctgaccaagaaccaggtctccctgacctgcctggtcaa |
| | | aggcttctatcccagtgacattgctgtggagtgggagagcaatgggcagcctgagaacaactac |
| | | aagaccacacctccagtgctggactctgatggctccttcttcctctactccaagctcactgtggac |
| | | aagagcaggtggcagcaggggaatgtcttctcatgcagtgtgatgcatgaggctctgcacaacc |
| | | actacacacagaagagcctctccctgtctcctggcaaatgagtttaaactccataaagtaggaaa |
| | | cactacactattccataaagtaggaaacactacatcactccataaagtaggaaacactacaagtct |
| | | ccataaagtaggaaacactacacaattgaataaaagatctttattttcattagatctgtgtgttggtttt |
| | | ttgtgtggagctcgagaggaacccctagtgatggagttggccactccctctctgcgcgctcgctc |
| | | gctcactgaggccgggcgaccaaaggtcgcccgacgcccgggctttgcccgggcggcctca |
| | | gtgagcgagcgagcgcgcagctgcagatctg |
| Full: 4802 | EC | ctgcgcgctcgctcgctcactgaggccgcccgggcaaagcccgggcgtcgggcgacctttgg |
| CAG | #11 | tcgcccggcctcagtgagcgagcgagcgcgcagagagggagtggccaactccatcactagg |
| Promoter: | L- | ggttccttagtattaaacgcgtgtcgacattgattattgactagttattaatagtaatcaattacgggg |
| 146-1870 | IRES- | tcattagttcatagcccatatatggagttccgcgttacataacttacggtaaatggcccgcctggct |
| IL-2 leader: | H | gaccgcccaacgacccccgcccattgacgtcaataatgacgtatgttcccatagtaacgccaata |
| 1898-1956 | | gggactttccattgacgtcaatgggtggactatttacggtaaactgcccacttggcagtacatcaa |
| LC: 1957- | | gtgtatcatatgccaagtacgccccctattgacgtcaatgacggtaaatggcccgcctggcattat |
| 2602 | | gcccagtacatgaccttatgggactttcctacttggcagtacatctacgtattagtcatcgctattac |
| IRES: 2603- | | catgggtcgaggtgagccccacgttctgcttcactctccccatctcccccccctccccaccccca |
| 3187 | | attttgtatttatttattttttaattattttgtgcagcgatgggggcggggggggggggggcgcgcg |
| IL-10 leader: | | ccaggcggggcggggcggggcgaggggcggggcggggcgaggcggagaggtgcggcg |
| 3188-3240 | | gcagccaatcagagcggcgcgctccgaaagtttccttttatggcgaggcggcggcggcggcg |
| HC: 3189- | | gccctataaaaagcgaagcgcgcggggggggagtcgctgcgcgctgccttcgccccgtgc |
| 4597 | | cccgctccgccgccgcctcgcgccgcccgccccggctctgactgaccgcgttactcccacagg |
| Synthetic | | tgagcgggcgggacggcccttctcctccgggctgtaattagcgcttggtttaatgacggctcgttt |
| poly(A): | | cttttctgtggctgcgtgaaagccttgaggggctccgggagggccctttgtgcggggggagcg |
| 4604-4652 | | gctcggggggtgcgtgcgtgtgtgtgtgcgtggggagcgccgcgtgcggctccgcgctgccc |
| (SEQ ID | | ggcggctgtgagcgctgcgggcgcggcgcggggctttgtgcgctccgcagtgtgcgcgaga |
| NO: 72 | | ggagcgcggccgggggcggtgccacgcggtgcggggggggctgcgaggggaacaaagg |
| (including | | ctgcgtgcggggtgtgtgcgtggggggggtgagcagggggtgtgggcgcggcggtcgggct |
| ITRs)) | | gtaacccccccctgcacccccctccccgagttgctgagcacggcccggcttcgggtgcggggc |
| ORF | | tccgtgcggggcgtggcgcggggctcgccgtgccgggcggggggtggcggcgggtgggg |
| (nucleotides | | gtgccgggcggggggggccgcctcgggccggggagggctcgggggaggggcgcggcg |
| 146-4652; | | gcccccggagcgccggcggctgtcgaggcgcggcgagccgcagccattgccttttatggtaat |
| SEQ ID NO: | | cgtgcgagagggcgcagggacttcctttgtcccaaatctgtgcggagccgaaatctgggaggc |
| 126) | | gccgccgcaccccctctagcgggcgcggggcgaagcggtgcggcgccggcaggaaggaa |
| | | atgggcggggagggccttcgtgcgtcgccgcgccgccgtccccttctccctctccagcctcgg |
| | | ggctgtccgcggggggacggctgccttcgggggggacggggcagggcggggttcggcttct |
| | | ggcgtgtgaccggcggtctagactctgctaaccatgttcatgccttcttctctttcctacagctcctg |
| | | ggcaacgtgctggttgttgtgctgtctcatcattttggcaaagaattcaagcttccagctagcgcca |
| | | ccatgtacaggatgcaactcctgtcttgcattgcactaagtcttgcacttgtcacaaactcagacat |
| | | ccagatgacccaaagcccctcctctctgtcagccagtgtgggggatagagtcacaattacttgca |
| | | gagcttcccagggaataaggaactacttggcatggtatcagcaaaagcctgggaaagctccaaa |
| | | gttgcttatctatgctgccagcactctgcagtcaggtgttcctagtagattctcaggcagtggaagt |
| | | gggactgactttacattaactatttcctctctgcaacctgaggatgtggccacctattactgtcagag |
| | | gtataatagagcaccttacacatttggccaaggtaccaaagtagaaatcaagaggactgtggctg |
| | | caccatctgtcttcatcttccccccatctgatgagcagttgaaatctggaactgcctctgttgtgtgc |
| | | ctgctgaataacttctatcccagagaggccaaagtacagtggaaggtggataatgccctccaatc |
| | | aggcaactcccaggagagtgtcacagagcaggacagcaaggacagcacctacagcctcagc |
| | | agcaccctgaccctgagcaaagcagactatgagaaacacaaagtctatgcctgtgaagtcaccc |
| | | atcagggcctgagctccccagtcacaaagagcttcaacaggggagagtgttgagcccctctccc |
| | | tcccccccccctaacgttactggccgaagccgcttggaataaggccggtgtgcgtttgtctatatg |
| | | ttattttccaccatattgccgtcttttggcaatgtgagggcccggaaacctggccctgtcttcttgac |
| | | gagcattcctaggggtctttcccctctcgccaaaggaatgcaaggtctgttgaatgtcgtgaagga |
| | | agcagttcctctggaagcttcttgaagacaaacaacgtctgtagcgaccctttgcaggcagcgga |
| | | accccccacctggcgacaggtgcctctgcggccaaaagccacgtgtataagatacacctgcaa |
| | | aggcggcacaaccccagtgccacgttgtgagttggatagttgtggaaagagtcaaatggctctc |
| | | ctcaagcgtattcaacaaggggctgaaggatgcccagaaggtaccccattgtatgggatctgatc |
| | | tggggcctcggtacacatgctttacatgtgtttagtcgaggttaaaaaaacgtctaggccccccga |
| | | accacggggacgtggttttcctttgaaaaacacgatgataatatggccacaatgcacagctcagc |
| | | actgctctgttgcctggtcctcctgactggggtgagggctgaggtgcagctggttgaaagtggag |
| | | gagggcttgttcaacctggtagatccttgagactttcttgtgctgcttctggcttcacctttgatgatta |
| | | tgcaatgcactgggtgaggcaggcacctggaaaggggctggagtgggtatcagccatcacatg |
| | | gaacagtggccatattgactatgctgatagtgtggaaggtagattcactatatccagagacaatgc |
| | | caaaaactctttatacctgcagatgaattcactaagggcagaggatactgctgtctattactgtgct |
| | | aaggtatcatatctcagcacagcatcctctctggactactggggacaagggacattggttactgtg |
| | | agctctgcctccaccaagggcccaagtgtcttccccctggcaccctcctccaagagcacctctg |
| | | ggggcacagcagccctgggctgcctggtcaaggactacttccctgaacctgtgactgtgtcttgg |
| | | aactcaggtgccctgaccagtggagtgcacaccttcccagctgtcctacagtcctcaggactcta |
| | | ctccctgagctctgtggtgacagtgccctccagcagcttgggcacccagacctacatctgcaatg |
| | | tgaatcacaagcccagcaacaccaaggtggacaagaaagttgagcccaaatcttgtgacaaaa |
| | | ctcacacatgcccaccttgcccagcacctgaactcctggggggaccatcagtcttcctcttcccc |
| | | ccaaaacccaaggacaccctcatgatctccagaacccctgaggtcacatgtgtggtggtggatg |
| | | tgagccatgaagaccctgaggtcaagttcaactggtatgtggatggtgtggaggtgcataatgcc |
| | | aagacaaagcccagggaggagcagtacaacagcacttacagagtggtcagtgtcctcacagtc |
| | | ctgcaccaggactggctgaatggcaaggagtacaagtgcaaggtctccaacaaagccctccca |
| | | gcccccattgagaaaaccatctccaaagccaaagggcagcccagggaaccacaggtgtacac |
| | | cctgcccccatccagagatgagctgaccaagaaccaggtctccctgacctgcctggtcaaagg |
| | | cttctatccctctgacattgctgtggagtgggagagcaatgggcagccagagaacaactacaag |
| | | accactcctcctgtgctggactctgatggctccttcttcctctactccaagctcacagtggacaaga |
| | | gcaggtggcagcaggggaatgtcttctcatgcagtgtgatgcatgaggctctgcacaaccacta |
| | | cactcagaagagcctctccctgtctcctggcaaatgacaattgaataaaagatctttattttcattag |
| | | atctgtgtgttggttttttgtgtggagctcgagaggaacccctagtgatggagttggccactccctc |
| | | tctgcgcgctcgctcgctcactgaggccgggcgaccaaaggtcgcccgacgcccgggctttgc |
| | | ccgggcggcctcagtgagcgagcgagcgcgcagctgcagatctg |
| Full: 4914 | EC | ctgcgcgctcgctcgctcactgaggccgcccgggcaaagcccgggcgtcgggcgacctttgg |
| CAG | #12 | tcgcccggcctcagtgagcgagcgagcgcgcagagagggagtggccaactccatcactagg |
| Promoter: | L- | ggttccttagtattaaacgcgtgtcgacattgattattgactagttattaatagtaatcaattacgggg |
| 146-1870 | IRES- | tcattagttcatagcccatatatggagttccgcgttacataacttacggtaaatggcccgcctggct |
| IL-2 leader: | H- | gaccgcccaacgacccccgcccattgacgtcaataatgacgtatgttcccatagtaacgccaata |
| 1898-1956 | 4xmiR142 | gggactttccattgacgtcaatgggtggactatttacggtaaactgcccacttggcagtacatcaa |
| LC: 1957- | | gtgtatcatatgccaagtacgccccctattgacgtcaatgacggtaaatggcccgcctggcattat |
| 2602 | | gcccagtacatgaccttatgggactttcctacttggcagtacatctacgtattagtcatcgctattac |
| IRES: 2603- | | catgggtcgaggtgagccccacgttctgcttcactctccccatctcccccccctccccaccccca |
| 3187 | | attttgtatttatttattttttaattattttgtgcagcgatgggggcggggggggggggggcgcgcg |
| IL-10 leader: | | ccaggcggggcggggcggggcgaggggcggggcggggcgaggcggagaggtgcggcg |
| 3188-3240 | | gcagccaatcagagcggcgcgctccgaaagtttccttttatggcgaggcggcggcggcggcg |
| HC: 3241- | | gccctataaaaagcgaagcgcgcggggggggagtcgctgcgcgctgccttcgccccgtgc |
| 4597 | | cccgctccgccgccgcctcgcgccgcccgccccggctctgactgaccgcgttactcccacagg |
| 4xmiR-142: | | tgagcgggcgggacggcccttctcctccgggctgtaattagcgcttggtttaatgacggctcgttt |
| 4606-4709 | | cttttctgtggctgcgtgaaagccttgaggggctccgggagggccctttgtgcggggggagcg |
| Synthetic | | gctcggggggtgcgtgcgtgtgtgtgtgcgtggggagcgccgcgtgcggctccgcgctgccc |
| poly(A): | | ggcggctgtgagcgctgcgggcgcggcgcggggctttgtgcgctccgcagtgtgcgcgaga |
| 4716-4764 | | ggagcgcggccgggggcggtgccacgcggtgcggggggggctgcgaggggaacaaagg |
| (SEQ ID | | ctgcgtgcggggtgtgtgcgtggggggggtgagcagggggtgtgggcgcggcggtcgggct |
| NO: 73 | | gtaacccccccctgcacccccctccccgagttgctgagcacggcccggcttcgggtgcgggg |
| (including | | tccgtgcggggcgtggcgcggggctcgccgtgccgggcggggggggcggcgggtgggg |
| ITRs)) | | gtgccgggcggggggggccgcctcgggccggggagggctcgggggaggggcgcggcg |
| Antibody | | gcccccggagcgccggcggctgtcgaggcgcggcgagccgcagccattgccttttatggtaat |
| expression | | cgtgcgagagggcgcagggacttcctttgtcccaaatctgtgcggagccgaaatctgggaggc |
| cassette (w/o | | gccgccgcaccccctctagcgggcgcggggcgaagcggtgcggcgccggcaggaaggaa |
| ITRs | | atgggcggggagggccttcgtgcgtcgccgcgccgccgtccccttctccctctccagcctcgg |
| (nucleotides | | ggctgtccgcggggggacggctgccttcgggggggacggggcagggcggggttcggcttct |
| 146-4764; | | ggcgtgtgaccggcggtctagactctgctaaccatgttcatgccttcttctctttcctacagctcctg |
| SEQ ID NO: | | ggcaacgtgctggttgttgtgctgtctcatcattttggcaaagaattcaagcttccagctagcgcca |
| 127) | | ccatgtacaggatgcaactcctgtcttgcattgcactaagtcttgcacttgtcacaaactcagacat |
| | | ccagatgacccaaagcccctcctctctgtcagccagtgtgggggatagagtcacaattacttgca |
| | | gagcttcccagggaataaggaactacttggcatggtatcagcaaaagcctgggaaagctccaaa |
| | | gttgcttatctatgctgccagcactctgcagtcaggtgttcctagtagattctcaggcagtggaagt |
| | | gggactgactttacattaactatttcctctctgcaacctgaggatgtggccacctattactgtcagag |
| | | gtataatagagcaccttacacatttggccaaggtaccaaagtagaaatcaagaggactgtggctg |
| | | caccatctgtcttcatcttccccccatctgatgagcagttgaaatctggaactgcctctgttgtgtgc |
| | | ctgctgaataacttctatcccagagaggccaaagtacagtggaaggtggataatgccctccaatc |
| | | aggcaactcccaggagagtgtcacagagcaggacagcaaggacagcacctacagcctcagc |
| | | agcaccctgaccctgagcaaagcagactatgagaaacacaaagtctatgcctgtgaagtcaccc |
| | | atcagggcctgagctccccagtcacaaagagcttcaacaggggagagtgttgagcccctctccc |
| | | tcccccccccctaacgttactggccgaagccgcttggaataaggccggtgtgcgtttgtctatatg |
| | | ttattttccaccatattgccgtcttttggcaatgtgagggcccggaaacctggccctgtcttcttgac |
| | | gagcattcctaggggtctttcccctctcgccaaaggaatgcaaggtctgttgaatgtcgtgaagga |
| | | agcagttcctctggaagcttcttgaagacaaacaacgtctgtagcgaccctttgcaggcagcgga |
| | | accccccacctggcgacaggtgcctctgcggccaaaagccacgtgtataagatacacctgcaa |
| | | aggcggcacaaccccagtgccacgttgtgagttggatagttgtggaaagagtcaaatggctctc |
| | | ctcaagcgtattcaacaaggggctgaaggatgcccagaaggtaccccattgtatgggatctgatc |
| | | tggggcctcggtacacatgctttacatgtgtttagtcgaggttaaaaaaacgtctaggccccccga |
| | | accacggggacgtggttttcctttgaaaaacacgatgataatatggccacaatgcacagctcagc |
| | | actgctctgttgcctggtcctcctgactggggtgagggctgaggtgcagctggttgaaagtggag |
| | | gagggcttgttcaacctggtagatccttgagactttcttgtgctgcttctggcttcacctttgatgatta |
| | | tgcaatgcactgggtgaggcaggcacctggaaaggggctggagtgggtatcagccatcacatg |
| | | gaacagtggccatattgactatgctgatagtgtggaaggtagattcactatatccagagacaatgc |
| | | caaaaactctttatacctgcagatgaattcactaagggcagaggatactgctgtctattactgtgct |
| | | aaggtatcatatctcagcacagcatcctctctggactactggggacaagggacattggttactgtg |
| | | agctctgcctccaccaagggcccaagtgtcttccccctggcaccctcctccaagagcacctctg |
| | | ggggcacagcagccctgggctgcctggtcaaggactacttccctgaacctgtgactgtgtcttgg |
| | | aactcaggtgccctgaccagtggagtgcacaccttcccagctgtcctacagtcctcaggactcta |
| | | ctccctgagctctgtggtgacagtgccctccagcagcttgggcacccagacctacatctgcaatg |
| | | tgaatcacaagcccagcaacaccaaggtggacaagaaagttgagcccaaatcttgtgacaaaa |
| | | ctcacacatgcccaccttgcccagcacctgaactcctggggggaccatcagtcttcctcttcccc |
| | | ccaaaacccaaggacaccctcatgatctccagaacccctgaggtcacatgtgtggtggtggatg |
| | | tgagccatgaagaccctgaggtcaagttcaactggtatgtggatggtgtggaggtgcataatgcc |
| | | aagacaaagcccagggaggagcagtacaacagcacttacagagtggtcagtgtcctcacagtc |
| | | ctgcaccaggactggctgaatggcaaggagtacaagtgcaaggtctccaacaaagccctccca |
| | | gcccccattgagaaaaccatctccaaagccaaagggcagcccagggaaccacaggtgtacac |
| | | cctgcccccatccagagatgagctgaccaagaaccaggtctccctgacctgcctggtcaaagg |
| | | cttctatccctctgacattgctgtggagtgggagagcaatgggcagccagagaacaactacaag |
| | | accactcctcctgtgctggactctgatggctccttcttcctctactccaagctcacagtggacaaga |
| | | gcaggtggcagcaggggaatgtcttctcatgcagtgtgatgcatgaggctctgcacaaccacta |
| | | cactcagaagagcctctccctgtctcctggcaaatgagtttaaactccataaagtaggaaacacta |
| | | cactattccataaagtaggaaacactacatcactccataaagtaggaaacactacaagtctccata |
| | | aagtaggaaacactacacaattgaataaaagatctttattttcattagatctgtgtgttggttttttgtgt |
| | | ggagctcgagaggaacccctagtgatggagttggccactccctctctgcgcgctcgctcgctca |
| | | ctgaggccgggcgaccaaaggtcgcccgacgcccgggctttgcccgggcggcctcagtgag |
| | | cgagcgagcgcgcagctgcagatctg |
| Full: 4170 | EC | ctgcgcgctcgctcgctcactgaggccgcccgggcaaagcccgggcgtcgggcgacctttgg |
| Synthetic | #13 | tcgcccggcctcagtgagcgagcgagcgcgcagagagggagtggccaactccatcactagg |
| poly(A): | DP- | ggttccttagtattaaacgcgtgtcgaccacacaaaaaaccaacacacagatctaatgaaaataa |
| 152-200 | HL- | agatcttttattcctaggtcatttgcccggagacagggagaggctcttctgcgtgtagtggttgtgc |
| HC: 207- | opposite | agagcctcatgcatcacggagcatgagaagacgttcccctgctgccacctgctcttgtccacggt |
| 1562 | | gagcttggagtagaggaagaaggagccgtcggagtccagcacgggaggcgtggtcttgtagtt |
| IL-10 leader: | | gttctccggctgcccattgctctcccactccacggcgatgtcgctgggatagaagcctttgacca |
| 1563-1616 | | ggcaggtcaggctgacttggttcttggtcagctcatcccgggatgggggcagggtgtacacctg |
| Intron: 1666- | | tggttctcggggctgccctttggctttggagattgttttctcgataggggctgggagggctttgttg |
| 1798 | | gagaccttgcacttgtactccttgccattcagccagtcctggtgcaggacggtgaggacgctgac |
| 5′ LTR: | | cacacggtacgtgctgttgtactgctcctcccgcggctttgtcttggcattatgcacctccacgccg |
| 1825-2093 | | tccacgtaccagttgaacttgacctcagggtcttcgtggctcacgtccaccaccacgcatgtgac |
| Promoter 1: | | ctcaggggtccgggagatcatgagggtgtccttgggttttggggggaagaggaagactgacgg |
| 2106-2712 | | tccccccaggagttcaggtgctgggcacggtgggcatgtgtgagttttgtcacaagatttgggct |
| (EF-1a | | caactttcttgtccaccttggtgttgctgggcttgtgattcacgttgcagatgtaggtctgggtgccc |
| promoter: | | aagctgctggagggcacggtcaccacgctgctgagggagtagagtcctgaggactgtaggac |
| 2106-2335 | | agccgggaaggtgtgcacgccgctggtcagggcgcctgagttccacgacaccgtcaccggttc |
| and CMV | | ggggaagtagtccttgaccaggcagcccagggccgctgtgcccccagaggtgctcttggagg |
| enhancer: | | agggtgccagggggaagaccgatgggcccttggtggaggcggagctcacagtaaccaatgtc |
| 2336-2712) | | ccttgtccccagtagtccagagaggatgcggtgctgagatatgacaccttagcacagtaatagac |
| Promoter 2: | | ggccgtatcctctgcacgtagtgaattcatctgcaggtataaagagtttttggcattgtctcgggata |
| 2713-3071 | | tagtgaagcgaccttccacgctatcagcatagtcaatatggcccgagttccatgtgatggctgata |
| (CMV | | cccactccagcccctttccaggcgcctgcctcacccagtgcattgcgtaatcgtcaaaggtgaag |
| promoter: | | ccagaagcggcgcaagaaagtctcaaggaccgaccgggttgaacgagccctccgccgctttc |
| 2713-2930 | | gaccagctgcacctcggccctcaccccagtcaggaggaccaggcaacagagcagtgctgagc |
| and SV40 | | tgtgcatggtggtttaaactagccttaagagctgtaattgaactgggagtggacacctgtggagag |
| intron: 2975- | | aaaggcaaagtggatgtcagtaagaccaataggtgcctatcagaaacgcaagagtcttctctgtc |
| 3071) | | tcgacaagcccagtttctattggtctccttaaacctgtcttgtaaccttgatacttacctgcccagtgc |
| IL-2 leader: | | ctcacgaccaacttcgatctgtaacggcgcagaacagaaaacgaaacaaagacgtagagttga |
| 3138-3197 | | gcaagcagggtcaggcaaagcgtggagagccggctgagtctaggtaggctccaagggagcg |
| LC: 3198- | | ccggacaaaggcccggtctcgacctgagctttaaacttacctagacggcggacgcagttcagg |
| 3842 | | aggcaccacaggcgggaggcggcagaacgcgactcaaccggcgtggatggcggcctcagg |
| BGH | | tagggcggcgggcgcgtgaaggagagatgcgagcccctcgaagcttcagctgtgttctggcg |
| poly(A): | | gcaaacccgttgcgaaaaagaacgttcacggcgactactgcacttatatacggttctcccccacc |
| 3849-4023 | | ctcgggaaaaaggcggagccagtacacgacatcactttcccagtttaccccgcgccaccttctct |
| (SEQ ID | | aggcaccggttcaattgccgacccctccccccaacttctcggggactgtgggcgatgtgcgctct |
| NO: 74 | | gcccactgacgggcaccggagcattgattattgactagttattaatagtaatcaattacggggtcat |
| (including | | tagttcatagcccatatatggagttccgcgttacataacttacggtaaatggcccgcctggctgac |
| ITRS)) | | cgcccaacgacccccgcccattgacgtcaataatgacgtatgttcccatagtaacgccaatagg |
| Antibody | | gactttccattgacgtcaatgggtggactatttacggtaaactgcccacttggcagtacatcaagtg |
| expression | | tatcatatgccaagtacgccccctattgacgtcaatgacggtaaatggcccgcctggcattatgcc |
| cassette (w/o | | cagtacatgaccttatgggactttcctacttggcagtacatctacgtattagtcatcgctattaccatg |
| ITRs | | gtgatgcggttttggcagtacatcaatgggcgtggatagcggtttgactcacggggatttccaagt |
| (nucleotides | | ctccaccccattgacgtcaatgggagtttgttttggcaccaaaatcaacgggactttccaaaatgtc |
| 152-4023; | | gtaacaactccgccccattgacgcaaatgggcggtaggcgtgtacggtgggaggtctatataag |
| SEQ ID NO: | | cagagctcgtttagtgaaccgtcagatcgcctggagaaggaactgaaaaaccagaaagttaact |
| 128) | | ggtaagtttagtctttttgtcttttatttcaggtcccggatccggtggtggtgcaaatcaaagaactgc |
| | | tcctcagtggatgttgcctttacttctaggcctgtacggaagtgttacttctgctctaaaagctgcgg |
| | | aattgtagcccggttaattaagccaccatgtacaggatgcaactcctgtcttgcattgcactaagtc |
| | | ttgcacttgtcacaaactcggacatccagatgacccaaagcccctcctctctgtcagccagtgtgg |
| | | gcgatcgggtcacaattacttgcagagcttcgcagggaataaggaactacctcgcatggtatcag |
| | | caaaagcctgggaaagcgccaaagttgcttatctacgccgccagcacgctgcagtccggtgttc |
| | | cgtctcgcttctctggcagtggaagcgggaccgactttacattaactatttcctctctgcaacccga |
| | | ggatgtggccacctattactgtcagcgatataatcgtgcaccttacacattcggccaaggtaccaa |
| | | agtagaaatcaagcgaactgtggctgcaccatctgtcttcatcttcccgccatctgatgagcagtt |
| | | gaaatctggaactgcctctgttgtgtgcctgctgaataacttctatcccagagaggccaaagtaca |
| | | gtggaaggtggataacgccctccaatcgggcaactcccaggagagtgtcacagagcaggaca |
| | | gcaaggacagcacctacagcctcagcagcaccctgacgctgagcaaagcagactacgagaaa |
| | | cacaaagtctacgcctgcgaagtcacccatcagggcctgagctcgcccgtcacaaagagcttca |
| | | acaggggagagtgttgagcatgcctgtgccttctagttgccagccatctgttgtttgcccctcccc |
| | | cgtgccttccttgaccctggaaggtgccactcccactgtcctttcctaataaaatgaggaaattgca |
| | | tcgcattgtctgagtaggtgtcattctattctggggggtggggggggcaggacagcaaggggg |
| | | actcgagaggaacccctagtgatggagttggccactccctctctgcgcgctcgctcgctcactga |
| | | ggccgggcgaccaaaggtcgcccgacgcccgggctttgcccgggcggcctcagtgagcgag |
| | | cgagcgcgcagctgcagatctg |
| Full: 4294 | EC | ctgcgcgctcgctcgctcactgaggccgcccgggcaaagcccgggcgtcgggcgacctttgg |
| CMVe/p: | #14 | tcgcccggcctcagtgagcgagcgagcgcgcagagagggagtggccaactccatcactagg |
| 149-746 | DP- | ggttccttagtattaaacgcgtgtcgacattgattattgactagttattaatagtaatcaattacgggg |
| IL-10 leader: | HL- | tcattagttcatagcccatatatggagttccgcgttacataacttacggtaaatggcccgcctggct |
| 954-1007 | same | gaccgcccaacgacccccgcccattgacgtcaataatgacgtatgttcccatagtaacgccaata |
| HC: 1008- | | gggactttccattgacgtcaatgggtggactatttacggtaaactgcccacttggcagtacatcaa |
| 2363 | | gtgtatcatatgccaagtacgccccctattgacgtcaatgacggtaaatggcccgcctggcattat |
| BGH poly | | gcccagtacatgaccttatgggactttcctacttggcagtacatctacgtattagtcatcgctattac |
| (A): 2370- | | catggtgatgcggttttggcagtacatcaatgggcgtggatagcggtttgactcacggggatttcc |
| 2549 | | aagtctccaccccattgacgtcaatgggagtttgttttggcaccaaaatcaacgggactttccaaa |
| Pause | | atgtcgtaacaactccgccccattgacgcaaatgggcggtaggcgtgtacggtgggaggtctat |
| element: | | ataagcagagctcgtttagtgaaccgtcagatcgcctggagaaggaactgaaaaaccagaaagt |
| 2559-2650 | | taactggtaagtttagtctttttgtcttttatttcaggtcccggatccggtggtggtgcaaatcaaaga |
| | | actgctcctcagtggatgttgcctttacttctaggcctgtacggaagtgttacttctgctctaaaagct |
| | | gcggaattgtagcccggttaattaagccaccatgcacagctcagcactgctctgttgcctggtcct |
| | | cctgactggggtgagggccgaggtgcagctggtcgaaagcggcggagggctcgttcaacccg |
| | | gtcggtccttgagactttcttgcgccgcttctggcttcacctttgacgattacgcaatgcactgggtg |
| | | aggcaggcgcctggaaaggggctggagtgggtatcagccatcacatggaactcgggccatatt |
| EF1α: 2669- | | gactatgctgatagcgtggaaggtcgcttcactatatcccgagacaatgccaaaaactctttatac |
| 2898 | | ctgcagatgaattcactacgtgcagaggatacggccgtctattactgtgctaaggtgtcatatctca |
| 5′ LTR: | | gcaccgcatcctctctggactactggggacaagggacattggttactgtgagctccgcctccacc |
| 2911-3179 | | aagggcccatcggtcttccccctggcaccctcctccaagagcacctctgggggcacagcggcc |
| IL-2 leader: | | ctgggctgcctggtcaaggactacttccccgaaccggtgacggtgtcgtggaactcaggcgcc |
| 3388-3447 | | ctgaccagcggcgtgcacaccttcccggctgtcctacagtcctcaggactctactccctcagcag |
| LC: 3448- | | cgtggtgaccgtgccctccagcagcttgggcacccagacctacatctgcaacgtgaatcacaag |
| 4092 | | cccagcaacaccaaggtggacaagaaagttgagcccaaatcttgtgacaaaactcacacatgc |
| Synthetic | | ccaccgtgcccagcacctgaactcctggggggaccgtcagtcttcctcttccccccaaaaccca |
| Poly(A): | | aggacaccctcatgatctcccggacccctgaggtcacatgcgtggtggtggacgtgagccacg |
| 4099-4147 | | aagaccctgaggtcaagttcaactggtacgtggacggcgtggaggtgcataatgccaagacaa |
| (SEQ ID | | agccgcgggaggagcagtacaacagcacgtaccgtgtggtcagcgtcctcaccgtcctgcacc |
| NO: 75 | | aggactggctgaatggcaaggagtacaagtgcaaggtctccaacaaagccctcccagccccta |
| (including | | tcgagaaaacaatctccaaagccaaagggcagccccgagaaccacaggtgtacaccctgccc |
| ITRs)) | | ccatcccgggatgagctgaccaagaaccaagtcagcctgacctgcctggtcaaaggcttctatc |
| Antibody | | ccagcgacatcgccgtggagtgggagagcaatgggcagccggagaacaactacaagaccac |
| expression | | gcctcccgtgctggactccgacggctccttcttcctctactccaagctcaccgtggacaagagca |
| cassette (w/o | | ggtggcagcaggggaacgtcttctcatgctccgtgatgcatgaggctctgcacaaccactacac |
| ITRs | | gcagaagagcctctccctgtctccgggcaaatgagcatgcctgtgccttctagttgccagccatct |
| (nucleotides | | gttgtttgcccctcccccgtgccttccttgaccctggaaggtgccactcccactgtcctttcctaata |
| 149-4147; | | aaatgaggaaattgcatcgcattgtctgagtaggtgtcattctattctggggggtggggggggc |
| SEQ ID NO: | | aggacagcaagggggaggattctcagaactaacatacgctctccatcaaaacaaaacgaaaca |
| 129) | | aaacaaactagcaaaataggctgtccccagtgcaagtgcaggtgccagaacatttctctatcgaa |
| | | ggatctgcgatcgctccggtgcccgtcagtgggcagagcgcacatcgcccacagtccccgag |
| | | aagttggggggaggggtcggcaattgaaccggtgcctagagaaggtggcgcggggtaaactg |
| | | ggaaagtgatgtcgtgtactggctccgcctttttcccgaggggggggagaaccgtatataagtg |
| | | cagtagtcgccgtgaacgttctttttcgcaacgggtttgccgccagaacacagctgaagcttcga |
| | | ggggctcgcatctctccttcacgcgcccgccgccctacctgaggccgccatccacgccggttga |
| | | gtcgcgttctgccgcctcccgcctgtggtgcctcctgaactgcgtccgccgtctaggtaagtttaa |
| | | agctcaggtcgagaccgggcctttgtccggcgctcccttggagcctacctagactcagccggct |
| | | ctccacgctttgcctgaccctgcttgctcaactctacgtctttgtttcgttttctgttctgcgccgttaca |
| | | gatcgaagttggtcgtgaggcactgggcaggtaagtatcaaggttacaagacaggtttaaggag |
| | | accaatagaaactgggcttgtcgagacagagaagactcttgcgtttctgataggcacctattggtc |
| | | ttactgacatccactttgcctttctctccacaggtgtccactcccagttcaattacagctcttaaggct |
| | | agtttaaaccaccatgtacaggatgcaactcctgtcttgcattgcactaagtcttgcacttgtcacaa |
| | | actcggacatccagatgacccaaagcccctcctctctgtcagccagtgtgggcgatcgggtcac |
| | | aattacttgcagagcttcgcagggaataaggaactacctcgcatggtatcagcaaaagcctggg |
| | | aaagcgccaaagttgcttatctacgccgccagcacgctgcagtccggtgttccgtctcgcttctct |
| | | ggcagtggaagcgggaccgactttacattaactatttcctctctgcaacccgaggatgtggccac |
| | | ctattactgtcagcgatataatcgtgcaccttacacattcggccaaggtaccaaagtagaaatcaa |
| | | gcgaactgtggctgcaccatctgtcttcatcttcccgccatctgatgagcagttgaaatctggaact |
| | | gcctctgttgtgtgcctgctgaataacttctatcccagagaggccaaagtacagtggaaggtggat |
| | | aacgccctccaatcgggcaactcccaggagagtgtcacagagcaggacagcaaggacagca |
| | | cctacagcctcagcagcaccctgacgctgagcaaagcagactacgagaaacacaaagtctacg |
| | | cctgcgaagtcacccatcagggcctgagctcgcccgtcacaaagagcttcaacaggggagagt |
| | | gttgacctaggaataaaagatctttattttcattagatctgtgtgttggttttttgtgtgctcgagagga |
| | | acccctagtgatggagttggccactccctctctgcgcgctcgctcgctcactgaggccgggcga |
| | | ccaaaggtcgcccgacgcccgggctttgcccgggcggcctcagtgagcgagcgagcgcgca |
| | | gctgcagatctg |
| Full: 4170 | EC | ctgcgcgctcgctcgctcactgaggccgcccgggcaaagcccgggcgtcgggcgacctttgg |
| Synthetic | #15 | tcgcccggcctcagtgagcgagcgagcgcgcagagagggagtggccaactccatcactagg |
| Poly(A): | DP- | ggttccttagtattaaacgcgtgtcgaccacacaaaaaaccaacacacagatctaatgaaaataa |
| 152-200 | LH- | agatcttttattcctaggtcaacactctcccctgttgaagctctttgtgacgggcgagctcaggccct |
| LC: 207-851 | opposite | gatgggtgacttcgcaggcgtagactttgtgtttctcgtagtctgctttgctcagcgtcagggtgct |
| IL-2 leader: | | gctgaggctgtaggtgctgtccttgctgtcctgctctgtgacactctcctgggagttgcccgattgg |
| 852-911 | | agggcgttatccaccttccactgtactttggcctctctgggatagaagttattcagcaggcacaca |
| Intron: 961- | | acagaggcagttccagatttcaactgctcatcagatggcgggaagatgaagacagatggtgcag |
| 1093 | | ccacagttcgcttgatttctactttggtaccttggccgaatgtgtaaggtgcacgattatatcgctga |
| 5′LTR: | | cagtaataggtggccacatcctcgggttgcagagaggaaatagttaatgtaaagtcggtcccgct |
| 1120-1388 | | tccactgccagagaagcgagacggaacaccggactgcagcgtgctggcggcgtagataagca |
| Promoter 1: | | actttggcgctttcccaggcttttgctgataccatgcgaggtagttccttattccctgcgaagctctg |
| 1401-2007 | | caagtaattgtgacccgatcgcccacactggctgacagagaggaggggctttgggtcatctgga |
| (EF-1α | | tgtccgagtttgtgacaagtgcaagacttagtgcaatgcaagacaggagttgcatcctgtacatgg |
| promoter: | | tggtttaaactagccttaagagctgtaattgaactgggagtggacacctgtggagagaaaggcaa |
| 1401-1630 | | agtggatgtcagtaagaccaataggtgcctatcagaaacgcaagagtcttctctgtctcgacaag |
| and CMV | | cccagtttctattggtctccttaaacctgtcttgtaaccttgatacttacctgcccagtgcctcacgac |
| enhancer: | | caacttcgatctgtaacggcgcagaacagaaaacgaaacaaagacgtagagttgagcaagcag |
| 1630-2007) | | ggtcaggcaaagcgtggagagccggctgagtctaggtaggctccaagggagcgccggacaa |
| Promoter 2: | | aggcccggtctcgacctgagctttaaacttacctagacggcggacgcagttcaggaggcacca |
| 2008-2366 | | caggcgggaggcggcagaacgcgactcaaccggcgtggatggcggcctcaggtagggcgg |
| (CMV | | cgggcgcgtgaaggagagatgcgagcccctcgaagcttcagctgtgttctggcggcaaaccc |
| promoter: | | gttgcgaaaaagaacgttcacggcgactactgcacttatatacggttctcccccaccctcgggaa |
| 2008-2225 | | aaaggcggagccagtacacgacatcactttcccagtttaccccgcgccaccttctctaggcacc |
| and SV40 | | ggttcaattgccgacccctccccccaacttctcggggactgtgggcgatgtgcgctctgcccact |
| intron: 2270- | | gacgggcaccggagcattgattattgactagttattaatagtaatcaattacggggtcattagttcat |
| 2366) | | agcccatatatggagttccgcgttacataacttacggtaaatggcccgcctggctgaccgcccaa |
| IL-10 leader: | | cgacccccgcccattgacgtcaataatgacgtatgttcccatagtaacgccaatagggactttcca |
| 2433-2486 | | ttgacgtcaatgggtggactatttacggtaaactgcccacttggcagtacatcaagtgtatcatatg |
| HC: 2487- | | ccaagtacgccccctattgacgtcaatgacggtaaatggcccgcctggcattatgcccagtacat |
| 3842 | | gaccttatgggactttcctacttggcagtacatctacgtattagtcatcgctattaccatggtgatgc |
| BGH poly | | ggttttggcagtacatcaatgggcgtggatagcggtttgactcacggggatttccaagtctccacc |
| (A): 3849- | | ccattgacgtcaatgggagtttgttttggcaccaaaatcaacgggactttccaaaatgtcgtaacaa |
| 4023 | | ctccgccccattgacgcaaatgggcggtaggcgtgtacggtgggaggtctatataagcagagct |
| (SEQ ID | | cgtttagtgaaccgtcagatcgcctggagaaggaactgaaaaaccagaaagttaactggtaagtt |
| NO: 76 | | tagtctttttgtcttttatttcaggtcccggatccggtggtggtgcaaatcaaagaactgctcctcagt |
| (including | | ggatgttgcctttacttctaggcctgtacggaagtgttacttctgctctaaaagctgcggaattgtag |
| ITRs)) | | cccggttaattaagccaccatgcacagctcagcactgctctgttgcctggtcctcctgactggggt |
| Antibody | | gagggccgaggtgcagctggtcgaaagcggcggagggctcgttcaacccggtcggtccttga |
| expression | | gactttcttgcgccgcttctggcttcacctttgacgattacgcaatgcactgggtgaggcaggcgc |
| cassette (w/o | | ctggaaaggggctggagtgggtatcagccatcacatggaactcgggccatattgactatgctgat |
| ITRs | | agcgtggaaggtcgcttcactatatcccgagacaatgccaaaaactctttatacctgcagatgaat |
| (nucleotides | | tcactacgtgcagaggatacggccgtctattactgtgctaaggtgtcatatctcagcaccgcatcc |
| 152-4023; | | tctctggactactggggacaagggacattggttactgtgagctccgcctccaccaagggcccatc |
| SEQ ID NO: | | ggtcttccccctggcaccctcctccaagagcacctctgggggcacagcggccctgggctgcct |
| 130) | | ggtcaaggactacttccccgaaccggtgacggtgtcgtggaactcaggcgccctgaccagcgg |
| | | cgtgcacaccttcccggctgtcctacagtcctcaggactctactccctcagcagcgtggtgaccg |
| | | tgccctccagcagcttgggcacccagacctacatctgcaacgtgaatcacaagcccagcaaca |
| | | ccaaggtggacaagaaagttgagcccaaatcttgtgacaaaactcacacatgcccaccgtgccc |
| | | agcacctgaactcctggggggaccgtcagtcttcctcttccccccaaaacccaaggacaccctc |
| | | atgatctcccggacccctgaggtcacatgcgtggtggtggacgtgagccacgaagaccctgag |
| | | gtcaagttcaactggtacgtggacggcgtggaggtgcataatgccaagacaaagccgcggga |
| | | ggagcagtacaacagcacgtaccgtgtggtcagcgtcctcaccgtcctgcaccaggactggct |
| | | gaatggcaaggagtacaagtgcaaggtctccaacaaagccctcccagcccctatcgagaaaac |
| | | aatctccaaagccaaagggcagccccgagaaccacaggtgtacaccctgcccccatcccggg |
| | | atgagctgaccaagaaccaagtcagcctgacctgcctggtcaaaggcttctatcccagcgacat |
| | | cgccgtggagtgggagagcaatgggcagccggagaacaactacaagaccacgcctcccgtg |
| | | ctggactccgacggctccttcttcctctactccaagctcaccgtggacaagagcaggtggcagc |
| | | aggggaacgtcttctcatgctccgtgatgcatgaggctctgcacaaccactacacgcagaagag |
| | | cctctccctgtctccgggcaaatgagcatgcctgtgccttctagttgccagccatctgttgtttgccc |
| | | ctcccccgtgccttccttgaccctggaaggtgccactcccactgtcctttcctaataaaatgagga |
| | | aattgcatcgcattgtctgagtaggtgtcattctattctggggggtggggts gggcaggacagca |
| | | agggggactcgagaggaacccctagtgatggagttggccactccctctctgcgcgctcgctcg |
| | | ctcactgaggccgggcgaccaaaggtcgcccgacgcccgggctttgcccgggcggcctcagt |
| | | gagcgagcgagcgcgcagctgcagatctg |
| Full: 4294 | EC | ctgcgcgctcgctcgctcactgaggccgcccgggcaaagcccgggcgtcgggcgacctttgg |
| CMVe/p: | #16 | tcgcccggcctcagtgagcgagcgagcgcgcagagagggagtggccaactccatcactagg |
| 149-746 | DP- | ggttccttagtattaaacgcgtgtcgacattgattattgactagttattaatagtaatcaattacgggg |
| IL-2 leader: | LH- | tcattagttcatagcccatatatggagttccgcgttacataacttacggtaaatggcccgcctggct |
| 954-1013 | Same | gaccgcccaacgacccccgcccattgacgtcaataatgacgtatgttcccatagtaacgccaata |
| LC: 1014- | | gggactttccattgacgtcaatgggtggactatttacggtaaactgcccacttggcagtacatcaa |
| 1658 | | gtgtatcatatgccaagtacgccccctattgacgtcaatgacggtaaatggcccgcctggcattat |
| BGH poly | | gcccagtacatgaccttatgggactttcctacttggcagtacatctacgtattagtcatcgctattac |
| (A): 1665- | | catggtgatgcggttttggcagtacatcaatgggcgtggatagcggtttgactcacggggatttcc |
| 1844 | | aagtctccaccccattgacgtcaatgggagtttgttttggcaccaaaatcaacgggactttccaaa |
| Pause | | atgtcgtaacaactccgccccattgacgcaaatgggcggtaggcgtgtacggtgggaggtctat |
| element: | | ataagcagagctcgtttagtgaaccgtcagatcgcctggagaaggaactgaaaaaccagaaagt |
| 1854-1945 | | taactggtaagtttagtctttttgtcttttatttcaggtcccggatccggtggtggtgcaaatcaaaga |
| EF1α: 1964- | | actgctcctcagtggatgttgcctttacttctaggcctgtacggaagtgttacttctgctctaaaagct |
| 2193 | | gcggaattgtagcccggttaattaagccaccatgtacaggatgcaactcctgtcttgcattgcact |
| 5′ LTR: | | aagtcttgcacttgtcacaaactcggacatccagatgacccaaagcccctcctctctgtcagcca |
| 2206-2474 | | gtgtgggcgatcgggtcacaattacttgcagagcttcgcagggaataaggaactacctcgcatg |
| IL-10 leader: | | gtatcagcaaaagcctgggaaagcgccaaagttgcttatctacgccgccagcacgctgcagtcc |
| 2683-1736 | | ggtgttccgtctcgcttctctggcagtggaagcgggaccgactttacattaactatttcctctctgca |
| HC: 1737- | | acccgaggatgtggccacctattactgtcagcgatataatcgtgcaccttacacattcggccaag |
| 4092 | | gtaccaaagtagaaatcaagcgaactgtggctgcaccatctgtcttcatcttcccgccatctgatg |
| Synthetic | | agcagttgaaatctggaactgcctctgttgtgtgcctgctgaataacttctatcccagagaggcca |
| poly(A): | | aagtacagtggaaggtggataacgccctccaatcgggcaactcccaggagagtgtcacagagc |
| 4099-4147 | | aggacagcaaggacagcacctacagcctcagcagcaccctgacgctgagcaaagcagactac |
| (SEQ ID | | gagaaacacaaagtctacgcctgcgaagtcacccatcagggcctgagctcgcccgtcacaaag |
| NO: 77 | | agcttcaacaggggagagtgttgagcatgcctgtgccttctagttgccagccatctgttgtttgccc |
| (including | | ctcccccgtgccttccttgaccctggaaggtgccactcccactgtcctttcctaataaaatgagga |
| ITRs)) | | aattgcatcgcattgtctgagtaggtgtcattctattctggggggtggggggggcaggacagca |
| Antibody | | agggggaggattctcagaactaacatacgctctccatcaaaacaaaacgaaacaaaacaaacta |
| expression | | gcaaaataggctgtccccagtgcaagtgcaggtgccagaacatttctctatcgaaggatctgcga |
| cassette (w/o | | tcgctccggtgcccgtcagtgggcagagcgcacatcgcccacagtccccgagaagttggggg |
| ITRs | | gaggggtcggcaattgaaccggtgcctagagaaggtggcgcggggtaaactgggaaagtgat |
| (nucleotides | | gtcgtgtactggctccgcctttttcccgagggtgggggagaaccgtatataagtgcagtagtcgc |
| 149-4147; | | cgtgaacgttctttttcgcaacgggtttgccgccagaacacagctgaagcttcgaggggctcgca |
| SEQ ID NO: | | tctctccttcacgcgcccgccgccctacctgaggccgccatccacgccggttgagtcgcgttctg |
| 131) | | ccgcctcccgcctgtggtgcctcctgaactgcgtccgccgtctaggtaagtttaaagctcaggtc |
| | | gagaccgggcctttgtccggcgctcccttggagcctacctagactcagccggctctccacgcttt |
| | | gcctgaccctgcttgctcaactctacgtctttgtttcgttttctgttctgcgccgttacagatcgaagtt |
| | | ggtcgtgaggcactgggcaggtaagtatcaaggttacaagacaggtttaaggagaccaataga |
| | | aactgggcttgtcgagacagagaagactcttgcgtttctgataggcacctattggtcttactgacat |
| | | ccactttgcctttctctccacaggtgtccactcccagttcaattacagctcttaaggctagtttaaacc |
| | | accatgcacagctcagcactgctctgttgcctggtcctcctgactggggtgagggccgaggtgc |
| | | agctggtcgaaagcggcggagggctcgttcaacccggtcggtccttgagactttcttgcgccgc |
| | | ttctggcttcacctttgacgattacgcaatgcactgggtgaggcaggcgcctggaaaggggctg |
| | | gagtgggtatcagccatcacatggaactcgggccatattgactatgctgatagcgtggaaggtcg |
| | | cttcactatatcccgagacaatgccaaaaactctttatacctgcagatgaattcactacgtgcagag |
| | | gatacggccgtctattactgtgctaaggtgtcatatctcagcaccgcatcctctctggactactggg |
| | | gacaagggacattggttactgtgagctccgcctccaccaagggcccatcggtcttccccctggc |
| | | accctcctccaagagcacctctgggggcacagcggccctgggctgcctggtcaaggactactt |
| | | ccccgaaccggtgacggtgtcgtggaactcaggcgccctgaccagcggcgtgcacaccttccc |
| | | ggctgtcctacagtcctcaggactctactccctcagcagcgtggtgaccgtgccctccagcagct |
| | | tgggcacccagacctacatctgcaacgtgaatcacaagcccagcaacaccaaggtggacaag |
| | | aaagttgagcccaaatcttgtgacaaaactcacacatgcccaccgtgcccagcacctgaactcct |
| | | ggggggaccgtcagtcttcctcttccccccaaaacccaaggacaccctcatgatctcccggacc |
| | | cctgaggtcacatgcgtggtggtggacgtgagccacgaagaccctgaggtcaagttcaactggt |
| | | acgtggacggcgtggaggtgcataatgccaagacaaagccgcgggaggagcagtacaacag |
| | | cacgtaccgtgtggtcagcgtcctcaccgtcctgcaccaggactggctgaatggcaaggagtac |
| | | aagtgcaaggtctccaacaaagccctcccagcccctatcgagaaaacaatctccaaagccaaa |
| | | gggcagccccgagaaccacaggtgtacaccctgcccccatcccgggatgagctgaccaagaa |
| | | ccaagtcagcctgacctgcctggtcaaaggcttctatcccagcgacatcgccgtggagtgggag |
| | | agcaatgggcagccggagaacaactacaagaccacgcctcccgtgctggactccgacggctc |
| | | cttcttcctctactccaagctcaccgtggacaagagcaggtggcagcaggggaacgtcttctcat |
| | | gctccgtgatgcatgaggctctgcacaaccactacacgcagaagagcctctccctgtctccggg |
| | | caaatgacctaggaataaaagatctttattttcattagatctgtgtgttggttttttgtgtgctcgagag |
| | | gaacccctagtgatggagttggccactccctctctgcgcgctcgctcgctcactgaggccgggc |
| | | gaccaaaggtcgcccgacgcccgggctttgcccgggggcctcagtgagcgagcgagcgcg |
| | | cagctgcagatctg |
| Full: 4147 | EC | ctgcgcgctcgctcgctcactgaggccgcccgggcaaagcccgggcgtcgggcgacctttgg |
| CMVe/p: | #17 | tcgcccggcctcagtgagcgagcgagcgcgcagagagggagtggccaactccatcactagg |
| 149-746 | Dual | ggttccttagtattaaacgcgtgtcgacattgattattgactagttattaatagtaatcaattacgggg |
| IL-2 signal | promoter- | tcattagttcatagcccatatatggagttccgcgttacataacttacggtaaatggcccgcctggct |
| sequence: | L-H | gaccgcccaacgacccccgcccattgacgtcaataatgacgtatgttcccatagtaacgccaata |
| 954-1013 | | gggactttccattgacgtcaatgggtggactatttacggtaaactgcccacttggcagtacatcaa |
| LC: 1014- | | gtgtatcatatgccaagtacgccccctattgacgtcaatgacggtaaatggcccgcctggcattat |
| 1658 | | gcccagtacatgaccttatgggactttcctacttggcagtacatctacgtattagtcatcgctattac |
| bGH polyA: | | catggtgatgcggttttggcagtacatcaatgggcgtggatagcggtttgactcacggggatttcc |
| 1665-1844 | | aagtctccaccccattgacgtcaatgggagtttgttttggcaccaaaatcaacgggactttccaaa |
| Pause | | atgtcgtaacaactccgccccattgacgcaaatgggcggtaggcgtgtacggtgggaggtctat |
| element: | | ataagcagagctcgtttagtgaaccgtcagatcgcctggagaaggaactgaaaaaccagaaagt |
| 1854-1945 | | taactggtaagtttagtctttttgtcttttatttcaggtcccggatccggtggtggtgcaaatcaaaga |
| EF1α | | actgctcctcagtggatgttgcctttacttctaggcctgtacggaagtgttacttctgctctaaaagct |
| promoter: | | gcggaattgtagcccggttaattaagccaccatgtacaggatgcaactcctgtcttgcattgcact |
| 1964-2193 | | aagtcttgcacttgtcacaaactcagacatccagatgacccaaagcccctcctctctgtcagccag |
| IL-10 signal | | tgtgggggatagagtcacaattacttgcagagcttcccagggaataaggaactacttggcatggt |
| sequence: | | atcagcaaaagcctgggaaagctccaaagttgcttatctatgctgccagcactctgcagtcaggt |
| 2683-2736 | | gttcctagtagattctcaggcagtggaagtgggactgactttacattaactatttcctctctgcaacc |
| HC: 2737- | | tgaggatgtggccacctattactgtcagaggtataatagagcaccttacacatttggccaaggtac |
| 4089 | | caaagtagaaatcaagaggactgtggctgcaccatctgtcttcatcttccccccatctgatgagca |
| polyA: 4099- | | gttgaaatctggaactgcctctgttgtgtgcctgctgaataacttctatcccagagaggccaaagta |
| 4147 | | cagtggaaggtggataatgccctccaatcaggcaactcccaggagagtgtcacagagcaggac |
| (SEQ ID | | agcaaggacagcacctacagcctcagcagcaccctgaccctgagcaaagcagactatgagaa |
| NO: 132 | | acacaaagtctatgcctgtgaagtcacccatcagggcctgagctccccagtcacaaagagcttc |
| (including | | aacaggggagagtgttgagcatgcctgtgccttctagttgccagccatctgttgtttgcccctccc |
| ITRs)) | | ccgtgccttccttgaccctggaaggtgccactcccactgtcctttcctaataaaatgaggaaattgc |
| Antibody | | atcgcattgtctgagtaggtgtcattctattctggggggggggtggggcaggacagcaagggg |
| expression | | gaggattctcagaactaacatacgctctccatcaaaacaaaacgaaacaaaacaaactagcaaa |
| cassette (w/o | | ataggctgtccccagtgcaagtgcaggtgccagaacatttctctatcgaaggatctgcgatcgct |
| ITRs | | ccggtgcccgtcagtgggcagagcgcacatcgcccacagtccccgagaagttggggggagg |
| (nucleotides | | ggtcggcaattgaaccggtgcctagagaaggtggcgcggggtaaactgggaaagtgatgtcgt |
| 149-4147; | | gtactggctccgcctttttcccgaggggggggagaaccgtatataagtgcagtagtcgccgtga |
| SEQ ID NO: | | acgttctttttcgcaacgggtttgccgccagaacacagctgaagcttcgaggggctcgcatctctc |
| 133) | | cttcacgcgcccgccgccctacctgaggccgccatccacgccggttgagtcgcgttctgccgcc |
| | | tcccgcctgtggtgcctcctgaactgcgtccgccgtctaggtaagtttaaagctcaggtcgagac |
| | | cgggcctttgtccggcgctcccttggagcctacctagactcagccggctctccacgctttgcctg |
| | | accctgcttgctcaactctacgtctttgtttcgttttctgttctgcgccgttacagatcgaagttggtcg |
| | | tgaggcactgggcaggtaagtatcaaggttacaagacaggtttaaggagaccaatagaaactgg |
| | | gcttgtcgagacagagaagactcttgcgtttctgataggcacctattggtcttactgacatccacttt |
| | | gcctttctctccacaggtgtccactcccagttcaattacagctcttaaggctagtttaaaccaccatg |
| | | cacagctcagcactgctctgttgcctggtcctcctgactggggtgagggctgaggtgcagctggt |
| | | tgaaagtggaggagggcttgttcaacctggtagatccttgagactttcttgtgctgcttctggcttca |
| | | cctttgatgattatgcaatgcactgggtgaggcaggcacctggaaaggggctggagtgggtatc |
| | | agccatcacatggaacagtggccatattgactatgctgatagtgtggaaggtagattcactatatc |
| | | cagagacaatgccaaaaactctttatacctgcagatgaattcactaagggcagaggatactgctg |
| | | tctattactgtgctaaggtatcatatctcagcacagcatcctctctggactactggggacaaggga |
| | | cattggttactgtgagctctgcctccaccaagggcccaagtgtcttccccctggcaccctcctcca |
| | | agagcacctctgggggcacagcagccctgggctgcctggtcaaggactacttccctgaacctgt |
| | | gactgtgtcttggaactcaggtgccctgaccagtggagtgcacaccttcccagctgtcctacagt |
| | | cctcaggactctactccctgagctctgtggtgacagtgccctccagcagcttgggcacccagac |
| | | ctacatctgcaatgtgaatcacaagcccagcaacaccaaggtggacaagaaagttgagcccaa |
| | | atcttgtgacaaaactcacacatgcccaccttgcccagcacctgaactcctggggggaccatca |
| | | gtcttcctcttccccccaaaacccaaggacaccctcatgatctccagaacccctgaggtcacatgt |
| | | gtggtggtggatgtgagccatgaagaccctgaggtcaagttcaactggtatgtggatggtgtgga |
| | | ggtgcataatgccaagacaaagcccagggaggagcagtacaacagcacttacagagtggtca |
| | | gtgtcctcacagtcctgcaccaggactggctgaatggcaaggagtacaagtgcaaggtctccaa |
| | | caaagccctcccagcccccattgagaaaaccatctccaaagccaaagggcagcccagggaac |
| | | cacaggtgtacaccctgcccccatccagagatgagctgaccaagaaccaggtctccctgacctg |
| | | cctggtcaaaggcttctatccctctgacattgctgtggagtgggagagcaatgggcagccagag |
| | | aacaactacaagaccactcctcctgtgctggactctgatggctccttcttcctctactccaagctca |
| | | cagtggacaagagcaggtggcagcaggggaatgtcttctcatgcagtgtgatgcatgaggctct |
| | | gcacaaccactacactcagaagagcctctccctgtctcctggcaaatgacctaggaataaaagat |
| | | ctttattttcattagatctgtgtgttggttttttgtgtg |
| Full: 4485 | EC | ctgcgcgctcgctcgctcactgaggccgcccgggcaaagcccgggcgtcgggcgacctttgg |
| CAG: 146- | #18 | tcgcccggcctcagtgagcgagcgagcgcgcagagagggagtggccaactccatcactagg |
| 1870 | ada- | ggttccttagtattaaacgcgtgtcgacattgattattgactagttattaatagtaatcaattacgggg |
| IL-10 leader: | limumab | tcattagttcatagcccatatatggagttccgcgttacataacttacggtaaatggcccgcctggct |
| 1898-1951 | | gaccgcccaacgacccccgcccattgacgtcaataatgacgtatgttcccatagtaacgccaata |
| HC: 1952- | H- | gggactttccattgacgtcaatgggtggactatttacggtaaactgcccacttggcagtacatcaa |
| 3304 | F2A-L | gtgtatcatatgccaagtacgccccctattgacgtcaatgacggtaaatggcccgcctggcattat |
| Furin: 3305- | | gcccagtacatgaccttatgggactttcctacttggcagtacatctacgtattagtcatcgctattac |
| 3316 | | catgggtcgaggtgagccccacgttctgcttcactctccccatctcccccccctccccaccccca |
| 2A: 3329- | | attttgtatttatttattttttaattattttgtgcagcgatgggggcggggggggggggggcgcgcg |
| 3400 | | ccaggcggggcggggcggggcgaggggcggggcggggcgaggcggagaggtgcggcg |
| IL-2 leader: | | gcagccaatcagagcggcgcgctccgaaagtttccttttatggcgaggcggcggcggcggcg |
| 3401-3460 | | gccctataaaaagcgaagcgcgcggcgggcgggagtcgctgcgcgctgccttcgccccgtgc |
| LC: 3461- | | cccgctccgccgccgcctcgcgccgcccgccccggctctgactgaccgcgttactcccacagg |
| 4105 | | tgagcgggcgggacggcccttctcctccgggctgtaattagcgcttggtttaatgacggctcgttt |
| bGH polyA: | | cttttctgtggctgcgtgaaagccttgaggggctccgggagggccctttgtgcggggggagcg |
| 4112-4335 | | gctcggggggtgcgtgcgtgtgtgtgtgcgtggggagcgccgcgtgcggctccgcgctgccc |
| (SEQ ID | | ggcggctgtgagcgctgcgggcgcggcgcggggctttgtgcgctccgcagtgtgcgcgaga |
| NO: 134 | | ggagcgcggccgggggcggtgccacgcggtgcggggggggctgcgaggggaacaaagg |
| (including | | ctgcgtgcggggtgtgtgcgtggggggggtgagcagggggtgtgggcgcggcggtcgggct |
| ITRs)) | | gtaacccccccctgcacccccctccccgagttgctgagcacggcccggcttcgggtgcggggc |
| Antibody | | tccgtgcggggcgtggcgcggggctcgccgtgccgggcggggggtggcggcgggtgggg |
| expression | | gtgccgggcggggcggggccgcctcgggccggggagggctcgggggaggggcgcggcg |
| cassette (w/o | | gcccccggagcgccggcggctgtcgaggcgcggcgagccgcagccattgccttttatggtaat |
| ITRs | | cgtgcgagagggcgcagggacttcctttgtcccaaatctgtgcggagccgaaatctgggaggc |
| (nucleotides | | gccgccgcaccccctctagcgggcgcggggcgaagcggtgcggcgccggcaggaaggaa |
| 149-4335; | | atgggggggagggccttcgtgcgtcgccgcgccgccgtccccttctccctctccagcctcgg |
| SEQ ID NO: | | ggctgtccgcggggggacggctgccttcgggggggacggggcagggcggggttcggcttct |
| 135) | | ggcgtgtgaccggcggtctagactctgctaaccatgttcatgccttcttctctttcctacagctcctg |
| | | ggcaacgtgctggttgttgtgctgtctcatcattttggcaaagaattcaagcttccagctagcgcca |
| | | ccatgcacagctcagcactgctctgttgcctggtcctcctgactggggtgagggccgaggtgca |
| | | gctggtcgaaagcggcggagggctcgttcaacccggtcggtccttgagactttcttgcgccgctt |
| | | ctggcttcacctttgacgattacgcaatgcactgggtgaggcaggcgcctggaaaggggctgga |
| | | gtgggtatcagccatcacatggaactcgggccatattgactatgctgatagcgtggaaggtcgct |
| | | tcactatatcccgagacaatgccaaaaactctttatacctgcagatgaattcactacgtgcagagg |
| | | atacggccgtctattactgtgctaaggtgtcatatctcagcaccgcatcctctctggactactgggg |
| | | acaagggacattggttactgtgagctccgcctccaccaagggcccatcggtcttccccctggca |
| | | ccctcctccaagagcacctctgggggcacagcggccctgggctgcctggtcaaggactacttc |
| | | cccgaaccggtgacggtgtcgtggaactcaggcgccctgaccagcggcgtgcacaccttccc |
| | | ggctgtcctacagtcctcaggactctactccctcagcagcgtggtgaccgtgccctccagcagct |
| | | tgggcacccagacctacatctgcaacgtgaatcacaagcccagcaacaccaaggtggacaag |
| | | aaagttgagcccaaatcttgtgacaaaactcacacatgcccaccgtgcccagcacctgaactcct |
| | | ggggggaccgtcagtcttcctcttccccccaaaacccaaggacaccctcatgatctcccggacc |
| | | cctgaggtcacatgcgtggtggtggacgtgagccacgaagaccctgaggtcaagttcaactggt |
| | | acgtggacggcgtggaggtgcataatgccaagacaaagccgcgggaggagcagtacaacag |
| | | cacgtaccgtgtggtcagcgtcctcaccgtcctgcaccaggactggctgaatggcaaggagtac |
| | | aagtgcaaggtctccaacaaagccctcccagcccctatcgagaaaacaatctccaaagccaaa |
| | | gggcagccccgagaaccacaggtgtacaccctgcccccatcccgggatgagctgaccaagaa |
| | | ccaagtcagcctgacctgcctggtcaaaggcttctatcccagcgacatcgccgtggagtgggag |
| | | agcaatgggcagccggagaacaactacaagaccacgcctcccgtgctggactccgacggctc |
| | | cttcttcctctactccaagctcaccgtggacaagagcaggtggcagcaggggaacgtcttctcat |
| | | gctccgtgatgcatgaggctctgcacaaccactacacgcagaagagcctctccctgtctccggg |
| | | taaaagaaagagaaggagtggctcaggagcccctgtgaaacagaccctgaactttgacctcttg |
| | | aagcttgctggggatgtggagtctaatcctggtccaatgtacaggatgcaactcctgtcttgcattg |
| | | cactaagtcttgcacttgtcacaaactcggacatccagatgacccaaagcccctcctctctgtcag |
| | | ccagtgtgggcgatcgggtcacaattacttgcagagcttcgcagggaataaggaactacctcgc |
| | | atggtatcagcaaaagcctgggaaagcgccaaagttgcttatctacgccgccagcacgctgcag |
| | | tccggtgttccgtctcgcttctctggcagtggaagcgggaccgactttacattaactatttcctctct |
| | | gcaacccgaggatgtggccacctattactgtcagcgatataatcgtgcaccttacacattcggcc |
| | | aaggtaccaaagtagaaatcaagcgaactgtggctgcaccatctgtcttcatcttcccgccatctg |
| | | atgagcagttgaaatctggaactgcctctgttgtgtgcctgctgaataacttctatcccagagaggc |
| | | caaagtacagtggaaggtggataacgccctccaatcgggcaactcccaggagagtgtcacaga |
| | | gcaggacagcaaggacagcacctacagcctcagcagcaccctgacgctgagcaaagcagac |
| | | tacgagaaacacaaagtctacgcctgcgaagtcacccatcagggcctgagctcgcccgtcaca |
| | | aagagcttcaacaggggagagtgttgacaattgctgtgccttctagttgccagccatctgttgtttg |
| | | cccctcccccgtgccttccttgaccctggaaggtgccactcccactgtcctttcctaataaaatgag |
| | | gaaattgcatcgcattgtctgagtaggtgtcattctattctggggggtggggggggcaggacag |
| | | caagggggaggattgggaagacaatagcaggcatgctggggatgcggtgggctctatggagc |
| | | tcgagaggaacccctagtgatggagttggccactccctctctgcgcgctcgctcgctcactgag |
| | | gccgggcgaccaaaggtcgcccgacgcccgggctttgcccgggcggcctcagtgagcgagc |
| | | gagcgcgcagctgcagatctg |
| Full: 4082 | EC | ctgcgcgctcgctcgctcactgaggccgcccgggcaaagcccgggcgtcgggcgacctttgg |
| CMVe/CAG: | #19 | tcgcccggcctcagtgagcgagcgagcgcgcagagagggagtggccaactccatcactagg |
| 149-1870 | ada- | ggttccttagtattaaacgcgtgtcgacattgattattgactagttattaatagtaatcaattacgggg |
| IL-2 signal | limumab | tcattagttcatagcccatatatggagttccgcgttacataacttacggtaaatggcccgcctggct |
| sequence: | H- | gaccgcccaacgacccccgcccattgacgtcaataatgacgtatgttcccatagtaacgccaata |
| 1898-1957 | IRES- | gggactttccattgacgtcaatgggtggactatttacggtaaactgcccacttggcagtacatcaa |
| LC: 1958- | L | gtgtatcatatgccaagtacgccccctattgacgtcaatgacggtaaatggcccgcctggcattat |
| 2602 | | gcccagtacatgaccttatgggactttcctacttggcagtacatctacgtattagtcatcgctattac |
| IRES: 2603- | | catgggtcgaggtgagccccacgttctgcttcactctccccatctcccccccctccccaccccca |
| 3187 | | attttgtatttatttattttttaattattttgtgcagcgatgggggcggggggggggggggggcgcgcg |
| IL-10 signal | | ccaggcggggcggggcggggcgagggcgggggcggggcgaggcggagaggtgcggcg |
| sequence: | | gcagccaatcagagcggcgcgctccgaaagtttccttttatggcgaggcggcggcggcggcg |
| 3188-3241 | | gccctataaaaagcgaagcgcgcggggggggagtcgctgcgcgctgccttcgccccgtgc |
| HC: 3242- | | cccgctccgccgccgcctcgcgccgcccgccccggctctgactgaccgcgttactcccacagg |
| 4594 | | tgagcgggcgggacggcccttctcctccgggctgtaattagcgcttggtttaatgacggctcgttt |
| polyA: 4604- | | cttttctgtggctgcgtgaaagccttgaggggctccgggagggccctttgtgcggggggagcg |
| 4652 | | gctcggggggtgcgtgcgtgtgtgtgtgcgtggggagcgccgcgtgcggctccgcgctgccc |
| (SEQ ID | | ggcggctgtgagcgctgcgggcgcggcgcggggctttgtgcgctccgcagtgtgcgcgaga |
| NO: 136 | | ggagcgcggccgggggcggtgccacgcggtgcggggggggctgcgaggggaacaaagg |
| (including | | ctgcgtgcggggtgtgtgcgtggggggggtgagcagggggtgtgggcgcggcggtcgggct |
| ITRs)) | | gtaacccccccctgcacccccctccccgagttgctgagcacggcccggcttcgggtgcggggc |
| Antibody | | tccgtgcggggcgtggcgcggggctcgccgtgccgggcggggggtggcggcgggtgggg |
| expression | | gtgccgggcggggggggccgcctcgggccggggagggctcgggggaggggcgcggcg |
| cassette (w/o | | gcccccggagcgccggcggctgtcgaggcgcggcgagccgcagccattgccttttatggtaat |
| ITRs | | cgtgcgagagggcgcagggacttcctttgtcccaaatctgtgcggagccgaaatctgggaggc |
| (nucleotides | | gccgccgcaccccctctagcgggcgcggggcgaagcggtgcggcgccggcaggaaggaa |
| 149-4652; | | atgggcggggagggccttcgtgcgtcgccgcgccgccgtccccttctccctctccagcctcgg |
| SEQ ID NO: | | ggctgtccgcggggggacggctgccttcgggggggacggggcagggcggggttcggcttct |
| 137) | | ggcgtgtgaccggcggtctagactctgctaaccatgttcatgccttcttctctttcctacagctcctg |
| | | ggcaacgtgctggttgttgtgctgtctcatcattttggcaaagaattcaagcttccagctagcgcca |
| | | ccatgtacaggatgcaactcctgtcttgcattgcactaagtcttgcacttgtcacaaactcggacat |
| | | ccagatgacccaaagcccctcctctctgtcagccagtgtgggcgatcgggtcacaattacttgca |
| | | gagcttcgcagggaataaggaactacctcgcatggtatcagcaaaagcctgggaaagcgccaa |
| | | agttgcttatctacgccgccagcacgctgcagtccggtgttccgtctcgcttctctggcagtggaa |
| | | gcgggaccgactttacattaactatttcctctctgcaacccgaggatgtggccacctattactgtca |
| | | gcgatataatcgtgcaccttacacattcggccaaggtaccaaagtagaaatcaagcgaactgtgg |
| | | ctgcaccatctgtcttcatcttcccgccatctgatgagcagttgaaatctggaactgcctctgttgtg |
| | | tgcctgctgaataacttctatcccagagaggccaaagtacagtggaaggtggataacgccctcc |
| | | aatcgggcaactcccaggagagtgtcacagagcaggacagcaaggacagcacctacagcctc |
| | | agcagcaccctgacgctgagcaaagcagactacgagaaacacaaagtctacgcctgcgaagt |
| | | cacccatcagggcctgagctcgcccgtcacaaagagcttcaacaggggagagtgttgagcccc |
| | | tctccctcccccccccctaacgttactggccgaagccgcttggaataaggccggtgtgcgtttgtc |
| | | tatatgttattttccaccatattgccgtcttttggcaatgtgagggcccggaaacctggccctgtcttc |
| | | ttgacgagcattcctaggggtctttcccctctcgccaaaggaatgcaaggtctgttgaatgtcgtg |
| | | aaggaagcagttcctctggaagcttcttgaagacaaacaacgtctgtagcgaccctttgcaggca |
| | | gcggaaccccccacctggcgacaggtgcctctgcggccaaaagccacgtgtataagatacacc |
| | | tgcaaaggcggcacaaccccagtgccacgttgtgagttggatagttgtggaaagagtcaaatgg |
| | | ctctcctcaagcgtattcaacaaggggctgaaggatgcccagaaggtaccccattgtatgggatc |
| | | tgatctggggcctcggtacacatgctttacatgtgtttagtcgaggttaaaaaaacgtctaggcccc |
| | | ccgaaccacggggacgtggttttcctttgaaaaacacgatgataatatggccacaatgcacagct |
| | | cagcactgctctgttgcctggtcctcctgactggggtgagggccgaggtgcagctggtcgaaag |
| | | cggcggagggctcgttcaacccggtcggtccttgagactttcttgcgccgcttctggcttcaccttt |
| | | gacgattacgcaatgcactgggtgaggcaggcgcctggaaaggggctggagtgggtatcagc |
| | | catcacatggaactcgggccatattgactatgctgatagcgtggaaggtcgcttcactatatcccg |
| | | agacaatgccaaaaactctttatacctgcagatgaattcactacgtgcagaggatacggccgtcta |
| | | ttactgtgctaaggtgtcatatctcagcaccgcatcctctctggactactggggacaagggacatt |
| | | ggttactgtgagctccgcctccaccaagggcccatcggtcttccccctggcaccctcctccaaga |
| | | gcacctctgggggcacagcggccctgggctgcctggtcaaggactacttccccgaaccggtga |
| | | cggtgtcgtggaactcaggcgccctgaccagcggcgtgcacaccttcccggctgtcctacagtc |
| | | ctcaggactctactccctcagcagcgtggtgaccgtgccctccagcagcttgggcacccagacc |
| | | tacatctgcaacgtgaatcacaagcccagcaacaccaaggtggacaagaaagttgagcccaaa |
| | | tcttgtgacaaaactcacacatgcccaccgtgcccagcacctgaactcctggggggaccgtcag |
| | | tcttcctcttccccccaaaacccaaggacaccctcatgatctcccggacccctgaggtcacatgc |
| | | gtggtggtggacgtgagccacgaagaccctgaggtcaagttcaactggtacgtggacggcgtg |
| | | gaggtgcataatgccaagacaaagccgcgggaggagcagtacaacagcacgtaccgtgtggt |
| | | cagcgtcctcaccgtcctgcaccaggactggctgaatggcaaggagtacaagtgcaaggtctc |
| | | caacaaagccctcccagcccctatcgagaaaacaatctccaaagccaaagggcagccccgag |
| | | aaccacaggtgtacaccctgcccccatcccgggatgagctgaccaagaaccaagtcagcctga |
| | | cctgcctggtcaaaggcttctatcccagcgacatcgccgtggagtgggagagcaatgggcagc |
| | | cggagaacaactacaagaccacgcctcccgtgctggactccgacggctccttcttcctctactcc |
| | | aagctcaccgtggacaagagcaggtggcagcaggggaacgtcttctcatgctccgtgatgcat |
| | | gaggctctgcacaaccactacacgcagaagagcctctccctgtctccgggcaaatgacaattga |
| | | ataaaagatctttattttcattagatctgtgtgttggttttttgtgtggagctcgagaggaacccctagt |
| | | gatggagttggccactccctctctgcgcgctcgctcgctcactgaggccgggcgaccaaaggtc |
| | | gcccgacgcccgggctttgcccgggcggcctcagtgagcgagcgagcgcgcagctgcagat |
| | | ctg |
| Full: 4294 | EC | ctgcgcgctcgctcgctcactgaggccgcccgggcaaagcccgggcgtcgggcgacctttgg |
| CMVe/p: | #20 | tcgcccggcctcagtgagcgagcgagcgcgcagagagggagtggccaactccatcactagg |
| 149-746 | ada- | ggttccttagtattaaacgcgtgtcgacattgattattgactagttattaatagtaatcaattacgggg |
| IL-2 signal | limumab | tcattagttcatagcccatatatggagttccgcgttacataacttacggtaaatggcccgcctggct |
| sequence: | dual | gaccgcccaacgacccccgcccattgacgtcaataatgacgtatgttcccatagtaacgccaata |
| 954-1013 | promoter | gggactttccattgacgtcaatgggtggactatttacggtaaactgcccacttggcagtacatcaa |
| LC: 1014- | | gtgtatcatatgccaagtacgccccctattgacgtcaatgacggtaaatggcccgcctggcattat |
| 1658 | | gcccagtacatgaccttatgggactttcctacttggcagtacatctacgtattagtcatcgctattac |
| bGH polyA: | | catggtgatgcggttttggcagtacatcaatgggcgtggatagcggtttgactcacggggatttcc |
| 1665-1844 | | aagtctccaccccattgacgtcaatgggagtttgttttggcaccaaaatcaacgggactttccaaa |
| pause site: | | atgtcgtaacaactccgccccattgacgcaaatgggcggtaggcgtgtacggtgggaggtctat |
| 1854-1945 | | ataagcagagctcgtttagtgaaccgtcagatcgcctggagaaggaactgaaaaaccagaaagt |
| EF1α: 1964- | | taactggtaagtttagtctttttgtcttttatttcaggtcccggatccggtggtggtgcaaatcaaaga |
| 2193 | | actgctcctcagtggatgttgcctttacttctaggcctgtacggaagtgttacttctgctctaaaagct |
| IL-10 signal | | gcggaattgtagcccggttaattaagccaccatgtacaggatgcaactcctgtcttgcattgcact |
| sequence: | | aagtcttgcacttgtcacaaactcggacatccagatgacccaaagcccctcctctctgtcagcca |
| 2683-2736 | | gtgtgggcgatcgggtcacaattacttgcagagcttcgcagggaataaggaactacctcgcatg |
| HC: 2737- | | gtatcagcaaaagcctgggaaagcgccaaagttgcttatctacgccgccagcacgctgcagtcc |
| 4089 | | ggtgttccgtctcgcttctctggcagtggaagcgggaccgactttacattaactatttcctctctgca |
| polyA: 4099- | | acccgaggatgtggccacctattactgtcagcgatataatcgtgcaccttacacattcggccaag |
| 4147 | | gtaccaaagtagaaatcaagcgaactgtggctgcaccatctgtcttcatcttcccgccatctgatg |
| (SEQ ID | | agcagttgaaatctggaactgcctctgttgtgtgcctgctgaataacttctatcccagagaggcca |
| NO: 138 | | aagtacagtggaaggtggataacgccctccaatcgggcaactcccaggagagtgtcacagagc |
| (including | | aggacagcaaggacagcacctacagcctcagcagcaccctgacgctgagcaaagcagactac |
| ITRs)) | | agcttcaacaggggagagtgttgagcatgcctgtgccttctagttgccagccatctgttgtttgccc |
| Antibody | | gagaaacacaaagtctacgcctgcgaagtcacccatcagggcctgagctcgcccgtcacaaag |
| expression | | ctcccccgtgccttccttgaccctggaaggtgccactcccactgtcctttcctaataaaatgagga |
| cassette (w/o | | aattgcatcgcattgtctgagtaggtgtcattctattctggggggtggggggggcaggacagca |
| ITRs | | agggggaggattctcagaactaacatacgctctccatcaaaacaaaacgaaacaaaacaaacta |
| (nucleotides | | gcaaaataggctgtccccagtgcaagtgcaggtgccagaacatttctctatcgaaggatctgcga |
| 149-4147; | | tcgctccggtgcccgtcagtgggcagagcgcacatcgcccacagtccccgagaagttggggg |
| SEQ ID NO: | | gaggggtcggcaattgaaccggtgcctagagaaggtggcgcggggtaaactgggaaagtgat |
| 139) | | gtcgtgtactggctccgcctttttcccgaggggggggagaaccgtatataagtgcagtagtcgc |
| | | tctctccttcacgcgcccgccgccctacctgaggccgccatccacgccggttgagtcgcgttctg |
| | | cgtgaacgttctttttcgcaacgggtttgccgccagaacacagctgaagcttcgaggggctcgca |
| | | ccgcctcccgcctgtggtgcctcctgaactgcgtccgccgtctaggtaagtttaaagctcaggtc |
| | | gagaccgggcctttgtccggcgctcccttggagcctacctagactcagccggctctccacgcttt |
| | | gcctgaccctgcttgctcaactctacgtctttgtttcgttttctgttctgcgccgttacagatcgaagtt |
| | | ggtcgtgaggcactgggcaggtaagtatcaaggttacaagacaggtttaaggagaccaataga |
| | | aactgggcttgtcgagacagagaagactcttgcgtttctgataggcacctattggtcttactgacat |
| | | ccactttgcctttctctccacaggtgtccactcccagttcaattacagctcttaaggctagtttaaacc |
| | | accatgcacagctcagcactgctctgttgcctggtcctcctgactggggtgagggccgaggtgc |
| | | ttctggcttcacctttgacgattacgcaatgcactgggtgaggcaggcgcctggaaaggggctg |
| | | agctggtcgaaagcggcggagggctcgttcaacccggtcggtccttgagactttcttgcgccgc |
| | | gagtgggtatcagccatcacatggaactcgggccatattgactatgctgatagcgtggaaggtcg |
| | | cttcactatatcccgagacaatgccaaaaactctttatacctgcagatgaattcactacgtgcagag |
| | | gatacggccgtctattactgtgctaaggtgtcatatctcagcaccgcatcctctctggactactggg |
| | | gacaagggacattggttactgtgagctccgcctccaccaagggcccatcggtcttccccctggc |
| | | ccccgaaccggtgacggtgtcgtggaactcaggcgccctgaccagcggcgtgcacaccttccc |
| | | accctcctccaagagcacctctgggggcacagcggccctgggctgcctggtcaaggactactt |
| | | ggctgtcctacagtcctcaggactctactccctcagcagcgtggtgaccgtgccctccagcagct |
| | | tgggcacccagacctacatctgcaacgtgaatcacaagcccagcaacaccaaggtggacaag |
| | | aaagttgagcccaaatcttgtgacaaaactcacacatgcccaccgtgcccagcacctgaactcct |
| | | ggggggaccgtcagtcttcctcttccccccaaaacccaaggacaccctcatgatctcccggacc |
| | | cctgaggtcacatgcgtggtggtggacgtgagccacgaagaccctgaggtcaagttcaactggt |
| | | acgtggacggcgtggaggtgcataatgccaagacaaagccgcgggaggagcagtacaacag |
| | | cacgtaccgtgtggtcagcgtcctcaccgtcctgcaccaggactggctgaatggcaaggagtac |
| | | aagtgcaaggtctccaacaaagccctcccagcccctatcgagaaaacaatctccaaagccaaa |
| | | gggcagccccgagaaccacaggtgtacaccctgcccccatcccgggatgagctgaccaagaa |
| | | ccaagtcagcctgacctgcctggtcaaaggcttctatcccagcgacatcgccgtggagtgggag |
| | | agcaatgggcagccggagaacaactacaagaccacgcctcccgtgctggactccgacggctc |
| | | cttcttcctctactccaagctcaccgtggacaagagcaggtggcagcaggggaacgtcttctcat |
| | | gctccgtgatgcatgaggctctgcacaaccactacacgcagaagagcctctccctgtctccggg |
| | | caaatgacctaggaataaaagatctttattttcattagatctgtgtgttggttttttgtgtgctcgagag |
| | | gaacccctagtgatggagttggccactccctctctgcgcgctcgctcgctcactgaggccgggc |
| | | gaccaaaggtcgcccgacgcccgggctttgcccgggcggcctcagtgagcgagcgagcgcg |
| | | cagctgcagatctg |
| Full: 4294 | EC | ctgcgcgctcgctcgctcactgaggccgcccgggcaaagcccgggcgtcgggcgacctttgg |
| CMVe/p: | #21 | tcgcccggcctcagtgagcgagcgagcgcgcagagagggagtggccaactccatcactagg |
| 149-746 | | ggttccttagtattaaacgcgtgtcgacattgattattgactagttattaatagtaatcaattacgggg |
| IL-2 signal | | tcattagttcatagcccatatatggagttccgcgttacataacttacggtaaatggcccgcctggct |
| sequence: | | gggactttccattgacgtcaatgggtggactatttacggtaaactgcccacttggcagtacatcaa |
| 954-1013 | | gaccgcccaacgacccccgcccattgacgtcaataatgacgtatgttcccatagtaacgccaata |
| LC: 1014- | | gtgtatcatatgccaagtacgccccctattgacgtcaatgacggtaaatggcccgcctggcattat |
| 1658 | | gcccagtacatgaccttatgggactttcctacttggcagtacatctacgtattagtcatcgctattac |
| bGH poly A: | | catggtgatgcggttttggcagtacatcaatgggcgtggatagcggtttgactcacggggatttcc |
| 1665-1776 | | aagtctccaccccattgacgtcaatgggagtttgttttggcaccaaaatcaacgggactttccaaa |
| pause | | atgtcgtaacaactccgccccattgacgcaaatgggggtaggcgtgtacggtgggaggtctat |
| element: | | ataagcagagctcgtttagtgaaccgtcagatcgcctggagaaggaactgaaaaaccagaaagt |
| 1854-1945 | | taactggtaagtttagtctttttgtcttttatttcaggtcccggatccggtggtggtgcaaatcaaaga |
| EF1α: 1964- | | actgctcctcagtggatgttgcctttacttctaggcctgtacggaagtgttacttctgctctaaaagct |
| 2193 | | gcggaattgtagcccggttaattaagccaccatgtacaggatgcaactcctgtcttgcattgcact |
| IL-10 signal | | aagtcttgcacttgtcacaaactcagacatccagatgacccaaagcccctcctctctgtcagccag |
| sequence: | | tgtgggggatcgcgtcacaattacttgcagagcttcccagggaataaggaactacctcgcgtggt |
| 2683-2736 | | atcagcaaaagcctgggaaagcgccaaagttgcttatctatgccgccagcactctgcagtcagg |
| HC: 2737- | | tgttcctagtagattctctggcagtggaagcgggactgactttacattaactatttcctctctgcaac |
| 4089 | | ccaaagtagaaatcaagcggactgtggctgcaccatctgtcttcatcttcccgccatctgatgagc |
| polyA: 4099- | | ctgaggatgtggccacctattactgtcagcggtataatcgcgcaccttacacatttggccaaggta |
| 4147 | | agttgaaatctggaactgcctctgttgtgtgcctgctgaataacttctatcccagagaggccaaagt |
| (SEQ ID | | acagcaaggacagcacctacagcctcagcagcaccctgaccctgagcaaagcagactatgag |
| NO: 140 | | acagtggaaggtggataatgccctccaaagtggcaactcccaggagagtgtcacagagcagg |
| (including | | aaacacaaagtctacgcgtgtgaagtcacccatcagggcctgagctccccagtcacaaagagct |
| ITRs)) | | tcaacaggggagagtgttgagcatgcctgtgccttctagttgccagccatctgttgtttgcccctcc |
| Antibody | | cccgtgccttccttgaccctggaaggtgccactcccactgtcctttcctaataaaatgaggaaattg |
| expression | | catcgcattgtctgagtaggtgtcattctattctggggggtggggggggcaggacagcaaggg |
| cassette (w/o | | ggaggattctcagaactaacatacgctctccatcaaaacaaaacgaaaaaaacaaactagcaa |
| ITRs | | aataggctgtccccagtgcaagtgcaggtgccagaacatttctctatcgaaggatctgcgatcgc |
| (nucleotides | | tccggtgcccgtcagtgggcagagcgcacatcgcccacagtccccgagaagttggggggagg |
| 149-4147; | | ggtcggcaattgaaccggtgcctagagaaggtggcgcggggtaaactgggaaagtgatgtcgt |
| SEQ ID NO: | | gtactggctccgcctttttcccgaggggggggagaaccgtatataagtgcagtagtcgccgtga |
| 141) | | acgttctttttcgcaacgggtttgccgccagaacacagctgaagcttcgaggggctcgcatctctc |
| | | cttcacgcgcccgccgccctacctgaggccgccatccacgccggttgagtcgcgttctgccgcc |
| | | tcccgcctgtggtgcctcctgaactgcgtccgccgtctaggtaagtttaaagctcaggtcgagac |
| | | cgggcctttgtccggcgctcccttggagcctacctagactcagccggctctccacgctttgcctg |
| | | accctgcttgctcaactctacgtctttgtttcgttttctgttctgcgccgttacagatcgaagttggtcg |
| | | tgaggcactgggcaggtaagtatcaaggttacaagacaggtttaaggagaccaatagaaactgg |
| | | gcttgtcgagacagagaagactcttgcgtttctgataggcacctattggtcttactgacatccacttt |
| | | cacagctcagcactgctctgttgcctggtcctcctgactggggtgagggctgaggtgcagctggt |
| | | gcctttctctccacaggtgtccactcccagttcaattacagctcttaaggctagtttaaaccaccatg |
| | | tgaaagcggcggagggcttgttcaacctggtagatccttgagactttcttgcgccgcttctggcttc |
| | | acctttgatgattatgcaatgcactgggtgaggcaggcgcctggaaaggggctggagtgggtat |
| | | cagccatcacatggaacagtggccatattgactatgctgatagtgtggaaggtagattcactatat |
| | | cccgcgacaatgccaaaaactctttatacctgcagatgaattcactacgcgcagaggatactgcg |
| | | gtctattactgtgctaaggtgtcatatctcagcaccgcatcctctctggactactggggacaaggg |
| | | acattggttactgtgagctccgcctccaccaagggcccaagtgtcttccccctggcaccctcctcc |
| | | aagagcacctctgggggcacagcggccctgggctgcctggtcaaggactacttccctgaacct |
| | | gtgacagtgtcttggaactcaggcgccctgaccagcggagtgcacaccttcccagctgtcctac |
| | | agtcctcaggactctactccctcagcagtgtggtgactgtgccctccagcagcttgggcacccag |
| | | acctacatctgcaatgtgaatcacaagcccagcaacaccaaggtggacaagaaagttgagccc |
| | | aaatcttgtgacaaaactcacacatgcccaccttgcccagcacctgaactcctggggggaccatc |
| | | agtcttcctcttccccccaaaacccaaggacaccctcatgatctcccgcacccctgaggtcacat |
| | | gtgtggtggtggatgtgagccatgaagaccctgaggtcaagttcaactggtatgtggatggtgtg |
| | | gaggtgcataatgccaagacaaagccgcgggaggagcagtacaacagcacatacagagtggt |
| | | ctctgtcctcactgtcctgcaccaggactggctgaatggcaaggagtacaagtgcaaggtctcca |
| | | acaaagccctcccagcccctattgagaaaacaatctccaaagccaaagggcagcccagggaa |
| | | ccacaggtgtacaccctgcccccatcccgcgatgagctgaccaagaaccaggtctccctgacct |
| | | gcctggtcaaaggcttctatcccagtgacattgctgtggagtgggagagcaatgggcagcctga |
| | | gaacaactacaagaccacacctccagtgctggactctgatggctccttcttcctctactccaagct |
| | | cactgtggacaagagcaggtggcagcaggggaatgtcttctcatgcagtgtgatgcatgaggct |
| | | ctgcacaaccactacacacagaagagcctctccctgtctcctggcaaatgacctaggaataaaa |
| | | gatctttattttcattagatctgtgtgttggttttttgtgtgctcgagaggaacccctagtgatggagtt |
| | | ggccactccctctctgcgcgctcgctcgctcactgaggccgggcgaccaaaggtcgcccgacg |
| | | cccgggctttgcccgggcggcctcagtgagcgagcgagcgcgcagctgcagatctg |
| Full: 4595 | EC | ctgcgcgctcgctcgctcactgaggccgcccgggcaaagcccgggcgtcgggcgacctttgg |
| CAG: 146- | #22 | tcgcccggcctcagtgagcgagcgagcgcgcagagagggagtggccaactccatcactagg |
| 1870 | | ggttccttagtattaaacgcgtgtcgacattgattattgactagttattaatagtaatcaattacgggg |
| IL-10 signal | | tcattagttcatagcccatatatggagttccgcgttacataacttacggtaaatggcccgcctggct |
| sequence: | | gaccgcccaacgacccccgcccattgacgtcaataatgacgtatgttcccatagtaacgccaata |
| 1898-1951 | | gggactttccattgacgtcaatgggtggactatttacggtaaactgcccacttggcagtacatcaa |
| Heavy chain: | | gtgtatcatatgccaagtacgccccctattgacgtcaatgacggtaaatggcccgcctggcattat |
| 1952-3300 | | gcccagtacatgaccttatgggactttcctacttggcagtacatctacgtattagtcatcgctattac |
| Furin: 3305- | | catgggtcgaggtgagccccacgttctgcttcactctccccatctcccccccctccccaccccca |
| 3328 | | attttgtatttatttattttttaattattttgtgcagcgatgggggcggggggggggggggcgcgcg |
| 2A: 3329- | | ccaggcggggcggggcggggcgaggggcggggcggggcgaggcggagaggtgcggcg |
| 3400 | | gcagccaatcagagcggcgcgctccgaaagtttccttttatggcgaggcggcggcggcggcg |
| IL-2 signal | | gccctataaaaagcgaagcgcgcggggggggagtcgctgcgcgctgccttcgccccgtgc |
| sequence: | | cccgctccgccgccgcctcgcgccgcccgccccggctctgactgaccgcgttactcccacagg |
| 3401-3460 | | tgagcgggcgggacggcccttctcctccgggctgtaattagcgcttggtttaatgacggctcgttt |
| Light chain: | | cttttctgtggctgcgtgaaagccttgaggggctccgggagggccctttgtgcggggggagcg |
| 3461-4105 | | gctcggggggtgcgtgcgtgtgtgtgtgcgtggggagcgccgcgtgcggctccgcgctgccc |
| (SEQ ID | | ggcggctgtgagcgctgcgggcgcggcgcggggctttgtgcgctccgcagtgtgcgcgaga |
| NO: 157) | | ggagcgcggccgggggcggtgccacgcggtgcggggggggctgcgaggggaacaaagg |
| Antibody | | ctgcgtgcggggtgtgtgcgtggggggggtgagcagggggtgtgggcgcggcggtcgggct |
| expression | | gtaacccccccctgcacccccctccccgagttgctgagcacggcccggcttcgggtgcggggc |
| cassette (w/o | | tccgtgcggggcgtggcgcggggctcgccgtgccgggcggggggtggcggcgggtgggg |
| ITRs | | gtgccgggcggggggggccgcctcgggccggggagggctcgggggaggggcgcggcg |
| (nucleotides | | gcccccggagcgccggcggctgtcgaggcgcggcgagccgcagccattgccttttatggtaat |
| 146-4105; | | cgtgcgagagggcgcagggacttcctttgtcccaaatctgtgcggagccgaaatctgggaggc |
| SEQ ID NO: | | gccgccgcaccccctctagcgggcgcggggcgaagcggtgcggcgccggcaggaaggaa |
| 158) | | atgggcggggagggccttcgtgcgtcgccgcgccgccgtccccttctccctctccagcctcgg |
| | | ggctgtccgcggggggacggctgccttcgggggggacggggcagggcggggttcggcttct |
| | | ggcgtgtgaccggcggtctagactctgctaaccatgttcatgccttcttctctttcctacagctcctg |
| | | ggcaacgtgctggttgttgtgctgtctcatcattttggcaaagaattcaagcttccagctagcgcca |
| | | ccatgcacagctcagcactgctctgttgcctggtcctcctgactggggtgagggccgaggtgca |
| | | gctggtcgaaagcggcggagggctcgttcaacccggtcggtccttgagactttcttgcgccgctt |
| | | ctggcttcacctttgacgattacgcaatgcactgggtgaggcaggcgcctggaaaggggctgga |
| | | gtgggtatcagccatcacatggaactcgggccatattgactatgctgatagcgtggaaggtcgct |
| | | tcactatatcccgagacaatgccaaaaactctttatacctgcagatgaattcactacgtgcagagg |
| | | atacggccgtctattactgtgctaaggtgtcatatctcagcaccgcatcctctctggactactgggg |
| | | acaagggacattggttactgtgagctccgcctccaccaagggcccatcggtcttccccctggca |
| | | ccctcctccaagagcacctctgggggcacagcggccctgggctgcctggtcaaggactacttc |
| | | cccgaaccggtgacggtgtgtggaactcaggcgccctgaccagcggcgtgcacaccttccc |
| | | ggctgtcctacagtcctcaggactctactccctcagcagcgtggtgaccgtgccctccagcagct |
| | | tgggcacccagacctacatctgcaacgtgaatcacaagcccagcaacaccaaggtggacaag |
| | | aaagttgagcccaaatcttgtgacaaaactcacacatgcccaccgtgcccagcacctgaactcct |
| | | ggggggaccgtcagtcttcctcttccccccaaaacccaaggacaccctcatgatctcccggacc |
| | | cctgaggtcacatgcgtggtggtggacgtgagccacgaagaccctgaggtcaagttcaactggt |
| | | acgtggacggcgtggaggtgcataatgccaagacaaagccgcgggaggagcagtacaacag |
| | | cacgtaccgtgtggtcagcgtcctcaccgtcctgcaccaggactggctgaatggcaaggagtac |
| | | aagtgcaaggtctccaacaaagccctcccagcccctatcgagaaaacaatctccaaagccaaa |
| | | gggcagccccgagaaccacaggtgtacaccctgcccccatcccgggatgagctgaccaagaa |
| | | ccaagtcagcctgacctgcctggtcaaaggcttctatcccagcgacatcgccgtggagtgggag |
| | | agcaatgggcagccggagaacaactacaagaccacgcctcccgtgctggactccgacggctc |
| | | cttcttcctctactccaagctcaccgtggacaagagcaggtggcagcaggggaacgtcttctcat |
| | | gctccgtgatgcatgaggctctgcacaaccactacacgcagaagagcctctccctgtctccggg |
| | | taaaagaaagagaaggagtggctcaggagcccctgtgaaacagaccctgaactttgacctcttg |
| | | aagcttgctggggatgtggagtctaatcctggtccaatgtacaggatgcaactcctgtcttgcattg |
| | | cactaagtcttgcacttgtcacaaactcggacatccagatgacccaaagcccctcctctctgtcag |
| | | ccagtgtgggcgatcgggtcacaattacttgcagagcttcgcagggaataaggaactacctcgc |
| | | atggtatcagcaaaagcctgggaaagcgccaaagttgcttatctacgccgccagcacgctgcag |
| | | tccggtgttccgtctcgcttctctggcagtggaagcgggaccgactttacattaactatttcctctct |
| | | gcaacccgaggatgtggccacctattactgtcagcgatataatcgtgcaccttacacattcggcc |
| | | aaggtaccaaagtagaaatcaagcgaactgtggctgcaccatctgtcttcatcttcccgccatctg |
| | | atgagcagttgaaatctggaactgcctctgttgtgtgcctgctgaataacttctatcccagagaggc |
| | | caaagtacagtggaaggtggataacgccctccaatcgggcaactcccaggagagtgtcacaga |
| | | gcaggacagcaaggacagcacctacagcctcagcagcaccctgacgctgagcaaagcagac |
| | | tacgagaaacacaaagtctacgcctgcgaagtcacccatcagggcctgagctcgcccgtcaca |
| | | aagagcttcaacaggggagagtgttgacctaggtccataaagtaggaaacactacactattccat |
| | | aaagtaggaaacactacatcactccataaagtaggaaacactacaagtctccataaagtaggaa |
| | | acactacacaattgctgtgccttctagttgccagccatctgttgtttgcccctcccccgtgccttcctt |
| | | gaccctggaaggtgccactcccactgtcctttcctaataaaatgaggaaattgcatcgcattgtctg |
| | | agtaggtgtcattctattctggggggtggggggggcaggacagcaagggggaggattggga |
| | | agacaatagcaggcatgctggggatgcggtgggctctatggagctcgagaggaacccctagtg |
| | | atggagttggccactccctctctgcgcgctcgctcgctcactgaggccgggcgaccaaaggtcg |
| | | cccgacgcccgggctttgcccgggcggcctcagtgagcgagcgagcgcgcagctgcagatct |
| | | g |
| Full: 4485 | EC | ctgcgcgctcgctcgctcactgaggccgcccgggcaaagcccgggcgtcgggcgacctttgg |
| CAG: 146- | #23 | tcgcccggcctcagtgagcgagcgagcgcgcagagagggagtggccaactccatcactagg |
| 1870 | | ggttccttagtattaaacgcgtgtcgacattgattattgactagttattaatagtaatcaattacgggg |
| IL-10 signal | | tcattagttcatagcccatatatggagttccgcgttacataacttacggtaaatggcccgcctggct |
| sequence: | | gaccgcccaacgacccccgcccattgacgtcaataatgacgtatgttcccatagtaacgccaata |
| 1898-1951 | | gggactttccattgacgtcaatgggtggactatttacggtaaactgcccacttggcagtacatcaa |
| Heavy chain: | | gtgtatcatatgccaagtacgccccctattgacgtcaatgacggtaaatggcccgcctggcattat |
| 1952-3304 | | gcccagtacatgaccttatgggactttcctacttggcagtacatctacgtattagtcatcgctattac |
| Furin: 3305- | | catgggtcgaggtgagccccacgttctgcttcactctccccatctcccccccctccccaccccca |
| 3328 | | attttgtatttatttattttttaattattttgtgcagcgatgggggcggggggggggggggcgcgcg |
| 2A: 3329- | | ccaggcggggcggggcggggcgaggggggggcggggcgaggcggagaggtgcggcg |
| 3400 | | gcagccaatcagagcggcgcgctccgaaagtttccttttatggcgaggcggcggcggcggcg |
| IL-2 signal | | gccctataaaaagcgaagcgcgcggggggggagtcgctgcgcgctgccttcgccccgtgc |
| sequence: | | cccgctccgccgccgcctcgcgccgcccgccccggctctgactgaccgcgttactcccacagg |
| 3401-3460 | | tgagcgggcgggacggcccttctcctccgggctgtaattagcgcttggtttaatgacggctcgttt |
| Light chain: | | cttttctgtggctgcgtgaaagccttgaggggctccgggagggccctttgtgcggggggagcg |
| 3461-4105 | | gctcggggggtgcgtgcgtgtgtgtgtgcgtggggagcgccgcgtgcggctccgcgctgccc |
| bGH polyA: | | ggcggctgtgagcgctgcgggcgcggcgcggggctttgtgcgctccgcagtgtgcgcgaga |
| 4112-4335 | | ggagcgcggccgggggcggtgccacgcggtgcggggggggctgcgaggggaacaaagg |
| (SEQ ID | | ctgcgtgcggggtgtgtgcgtggggggggtgagcagggggtgtgggcgcggcggtcgggct |
| NO: 153 | | gtaacccccccctgcacccccctccccgagttgctgagcacggcccggcttcgggtgcggggc |
| (including | | tccgtgcggggcgtggcgcggggctcgccgtgccgggcggggggtggcggcgggtgggg |
| ITRs)) | | gtgccgggcggggcggggccgcctcgggccggggagggctcgggggaggggcgcggcg |
| Antibody | | gcccccggagcgccggcggctgtcgaggcgcggcgagccgcagccattgccttttatggtaat |
| expression | | cgtgcgagagggcgcagggacttcctttgtcccaaatctgtgcggagccgaaatctgggaggc |
| cassette (w/o | | gccgccgcaccccctctagcgggcgcggggcgaagcggtgcggcgccggcaggaaggaa |
| ITRs | | atgggcggggagggccttcgtgcgtcgccgcgccgccgtccccttctccctctccagcctcgg |
| (nucleotides | | ggctgtccgcggggggacggctgccttcgggggggacggggcagggcggggttcggcttct |
| 146-4335; | | ggcgtgtgaccggcggtctagactctgctaaccatgttcatgccttcttctctttcctacagctcctg |
| SEQ ID NO: | | ggcaacgtgctggttgttgtgctgtctcatcattttggcaaagaattcaagcttccagctagcgcca |
| 154) | | ccatgcacagctcagcactgctctgttgcctggtcctcctgactggggtgagggctgaggtgca |
| | | gctggttgaaagcggcggagggcttgttcaacctggtagatccttgagactttcttgcgccgcttc |
| | | tggcttcacctttgatgattatgcaatgcactgggtgaggcaggcgcctggaaaggggctggagt |
| | | gggtatcagccatcacatggaacagtggccatattgactatgctgatagtgtggaaggtagattca |
| | | ctatatcccgcgacaatgccaaaaactctttatacctgcagatgaattcactacgcgcagaggata |
| | | ctgcggtctattactgtgctaaggtgtcatatctcagcaccgcatcctctctggactactggggaca |
| | | agggacattggttactgtgagctccgcctccaccaagggcccaagtgtcttccccctggcaccct |
| | | cctccaagagcacctctgggggcacagcggccctgggctgcctggtcaaggactacttccctg |
| | | aacctgtgacagtgtcttggaactcaggcgccctgaccagcggagtgcacaccttcccagctgt |
| | | cctacagtcctcaggactctactccctcagcagtgtggtgactgtgccctccagcagcttgggca |
| | | cccagacctacatctgcaatgtgaatcacaagcccagcaacaccaaggtggacaagaaagttg |
| | | agcccaaatcttgtgacaaaactcacacatgcccaccttgcccagcacctgaactcctgggggg |
| | | accatcagtcttcctcttccccccaaaacccaaggacaccctcatgatctcccgcacccctgagg |
| | | tcacatgtgtggtggtggatgtgagccatgaagaccctgaggtcaagttcaactggtatgtggat |
| | | ggtgtggaggtgcataatgccaagacaaagccgcgggaggagcagtacaacagcacatacag |
| | | agtggtctctgtcctcactgtcctgcaccaggactggctgaatggcaaggagtacaagtgcaag |
| | | gtctccaacaaagccctcccagcccctattgagaaaacaatctccaaagccaaagggcagccc |
| | | agggaaccacaggtgtacaccctgcccccatcccgcgatgagctgaccaagaaccaggtctcc |
| | | ctgacctgcctggtcaaaggcttctatcccagtgacattgctgtggagtgggagagcaatgggca |
| | | gcctgagaacaactacaagaccacacctccagtgctggactctgatggctccttcttcctctactc |
| | | caagctcactgtggacaagagcaggtggcagcaggggaatgtcttctcatgcagtgtgatgcat |
| | | gaggctctgcacaaccactacacacagaagagcctctccctgtctcctggtaaaagaaagagaa |
| | | ggagtggctcaggagcccctgtgaaacagaccctgaactttgacctcttgaagcttgctggggat |
| | | gtggagtctaatcctggtccaatgtacaggatgcaactcctgtcttgcattgcactaagtcttgcact |
| | | tgtcacaaactcagacatccagatgacccaaagcccctcctctctgtcagccagtgtgggggatc |
| | | gcgtcacaattacttgcagagcttcccagggaataaggaactacctcgcgtggtatcagcaaaa |
| | | gcctgggaaagcgccaaagttgcttatctatgccgccagcactctgcagtcaggtgttcctagta |
| | | gattctctggcagtggaagcgggactgactttacattaactatttcctctctgcaacctgaggatgt |
| | | ggccacctattactgtcagcggtataatcgcgcaccttacacatttggccaaggtaccaaagtag |
| | | aaatcaagcggactgtggctgcaccatctgtcttcatcttcccgccatctgatgagcagttgaaatc |
| | | tggaactgcctctgttgtgtgcctgctgaataacttctatcccagagaggccaaagtacagtggaa |
| | | ggtggataatgccctccaaagtggcaactcccaggagagtgtcacagagcaggacagcaagg |
| | | acagcacctacagcctcagcagcaccctgaccctgagcaaagcagactatgagaaacacaaa |
| | | gtctacgcgtgtgaagtcacccatcagggcctgagctccccagtcacaaagagcttcaacaggg |
| | | gagagtgttgacaattgctgtgccttctagttgccagccatctgttgtttgcccctcccccgtgcctt |
| | | ccttgaccctggaaggtgccactcccactgtcctttcctaataaaatgaggaaattgcatcgcattg |
| | | tctgagtaggtgtcattctattctggggggtggggtggggcaggacagcaagggggaggattg |
| | | ggaagacaatagcaggcatgctggggatgcggtgggctctatggagctcgagaggaacccct |
| | | agtgatggagttggccactccctctctgcgcgctcgctcgctcactgaggccgggcgaccaaag |
| | | gtcgcccgacgcccgggctttgcccgggcggcctcagtgagcgagcgagcgcgcagctgca |
| | | gatctg |
| Full: 4737 | EC | ctgcgcgctcgctcgctcactgaggccgcccgggcaaagcccgggcgtcgggcgacctttgg |
| CAG: 146- | #24 | tcgcccggcctcagtgagcgagcgagcgcgcagagagggagtggccaactccatcactagg |
| 1870 | | ggttccttagtattaaacgcgtgtcgacattgattattgactagttattaatagtaatcaattacgggg |
| IL10 signal | | tcattagttcatagcccatatatggagttccgcgttacataacttacggtaaatggcccgcctggct |
| sequence: | | gaccgcccaacgacccccgcccattgacgtcaataatgacgtatgttcccatagtaacgccaata |
| 1898-1951 | | gggactttccattgacgtcaatgggtggactatttacggtaaactgcccacttggcagtacatcaa |
| Heavy chain: | | gtgtatcatatgccaagtacgccccctattgacgtcaatgacggtaaatggcccgcctggcattat |
| 1952-3304 | | gcccagtacatgaccttatgggactttcctacttggcagtacatctacgtattagtcatcgctattac |
| Furin: 3305- | | catgggtcgaggtgagccccacgttctgcttcactctccccatctcccccccctccccaccccca |
| 3328 | | attttgtatttatttattttttaattattttgtgcagcgatgggggcggggggggggggggcgcgcg |
| 2A: 3329- | | ccaggcggggcggggcggggcgaggggcggggcggggcgaggcggagaggtgcggcg |
| 3400 | | gcagccaatcagagcggcgcgctccgaaagtttccttttatggcgaggcggcggcggcggcg |
| IL-2 signal | | gccctataaaaagcgaagcgcgcggcgggcgggagtcgctgcgcgctgccttcgccccgtgc |
| sequence: | | tgagcggggggacggcccttctcctccgggctgtaattagcgcttggtttaatgacggctcgttt |
| 3401-3460 | | cccgctccgccgccgcctcgcgccgcccgccccggctctgactgaccgcgttactcccacagg |
| Light chain: | | cttttctgtggctgcgtgaaagccttgaggggctccgggagggccctttgtgcggggggagcg |
| 3461-4105 | | gctcggggggtgcgtgcgtgtgtgtgtgcgtggggagcgccgcgtgcggctccgcgctgccc |
| HGH polyA: | | ggcggctgtgagcgctgcgggcgcggcgcggggctttgtgcgctccgcagtgtgcgcgaga |
| 4608-4737 | | ggagcgcggccgggggcggtgccacgcggtgcggggggggctgcgaggggaacaaagg |
| (SEQ ID | | gtaacccccccctgcacccccctccccgagttgctgagcacggcccggcttcgggtgcggggc |
| NO: 155 | | ctgcgtgcggggtgtgtgcgtggggggggtgagcagggggtgtgggcgcggcggtcgggct |
| (including | | tccgtgcggggcgtggcgcggggctcgccgtgccgggcggggggggcggcgggtgggg |
| ITRs)) | | gtgccgggcggggggggccgcctcgggccggggagggctcgggggaggggcgcggcg |
| Antibody | | gcccccggagcgccggggctgtcgaggcgcggcgagccgcagccattgccttttatggtaat |
| expression | | cgtgcgagagggcgcagggacttcctttgtcccaaatctgtgcggagccgaaatctgggaggc |
| cassette (w/o | | gccgccgcaccccctctagcgggcgcggggcgaagcggtgcggcgccggcaggaaggaa |
| ITRs | | atgggcggggagggccttcgtgcgtcgccgcgccgccgtccccttctccctctccagcctcgg |
| (nucleotides | | ggctgtccgcggggggacggctgccttcgggggggacggggcagggcggggttcggcttct |
| 146-4737; | | ggcgtgtgaccggcggtctagactctgctaaccatgttcatgccttcttctctttcctacagctcctg |
| SEQ ID NO: | | ggcaacgtgctggttgttgtgctgtctcatcattttggcaaagaattcaagcttccagctagcgcca |
| 156) | | ccatgcacagctcagcactgctctgttgcctggtcctcctgactggggtgagggctgaggtgca |
| | | gctggttgaaagcggcggagggcttgttcaacctggtagatccttgagactttcttgcgccgcttc |
| | | tggcttcacctttgatgattatgcaatgcactgggtgaggcaggcgcctggaaaggggctggagt |
| | | gggtatcagccatcacatggaacagtggccatattgactatgctgatagtgtggaaggtagattca |
| | | ctatatcccgcgacaatgccaaaaactctttatacctgcagatgaattcactacgcgcagaggata |
| | | ctgcggtctattactgtgctaaggtgtcatatctcagcaccgcatcctctctggactactggggaca |
| | | agggacattggttactgtgagctccgcctccaccaagggcccaagtgtcttccccctggcaccct |
| | | aacctgtgacagtgtcttggaactcaggcgccctgaccagcggagtgcacaccttcccagctgt |
| | | cctccaagagcacctctgggggcacagcggccctgggctgcctggtcaaggactacttccctg |
| | | cctacagtcctcaggactctactccctcagcagtgtggtgactgtgccctccagcagcttgggca |
| | | cccagacctacatctgcaatgtgaatcacaagcccagcaacaccaaggtggacaagaaagttg |
| | | agcccaaatcttgtgacaaaactcacacatgcccaccttgcccagcacctgaactcctgggggg |
| | | accatcagtcttcctcttccccccaaaacccaaggacaccctcatgatctcccgcacccctgagg |
| | | tcacatgtgtggtggtggatgtgagccatgaagaccctgaggtcaagttcaactggtatgtggat |
| | | ggtgtggaggtgcataatgccaagacaaagccgcgggaggagcagtacaacagcacatacag |
| | | agtggtctctgtcctcactgtcctgcaccaggactggctgaatggcaaggagtacaagtgcaag |
| | | agggaaccacaggtgtacaccctgcccccatcccgcgatgagctgaccaagaaccaggtctcc |
| | | gtctccaacaaagccctcccagcccctattgagaaaacaatctccaaagccaaagggcagccc |
| | | ctgacctgcctggtcaaaggcttctatcccagtgacattgctgtggagtgggagagcaatgggca |
| | | gcctgagaacaactacaagaccacacctccagtgctggactctgatggctccttcttcctctactc |
| | | caagctcactgtggacaagagcaggtggcagcaggggaatgtcttctcatgcagtgtgatgcat |
| | | gaggctctgcacaaccactacacacagaagagcctctccctgtctcctggtaaaagaaagagaa |
| | | ggagtggctcaggagcccctgtgaaacagaccctgaactttgacctcttgaagcttgctggggat |
| | | gtggagtctaatcctggtccaatgtacaggatgcaactcctgtcttgcattgcactaagtcttgcact |
| | | tgtcacaaactcagacatccagatgacccaaagcccctcctctctgtcagccagtgtgggggatc |
| | | gcgtcacaattacttgcagagcttcccagggaataaggaactacctcgcgtggtatcagcaaaa |
| | | gcctgggaaagcgccaaagttgcttatctatgccgccagcactctgcagtcaggtgttcctagta |
| | | gattctctggcagtggaagcgggactgactttacattaactatttcctctctgcaacctgaggatgt |
| | | ggccacctattactgtcagcggtataatcgcgcaccttacacatttggccaaggtaccaaagtag |
| | | aaatcaagcggactgtggctgcaccatctgtcttcatcttcccgccatctgatgagcagttgaaatc |
| | | tggaactgcctctgttgtgtgcctgctgaataacttctatcccagagaggccaaagtacagtggaa |
| | | ggtggataatgccctccaaagtggcaactcccaggagagtgtcacagagcaggacagcaagg |
| | | acagcacctacagcctcagcagcaccctgaccctgagcaaagcagactatgagaaacacaaa |
| | | gtctacgcgtgtgaagtcacccatcagggcctgagctccccagtcacaaagagcttcaacaggg |
| | | gagagtgttgacaattggatctacgggtggcatccctgtgacccctccccagtgcctctcctggc |
| | | cctggaagttgccactccagtgcccaccagccttgtcctaataaaattaagttgcatcattttgtctg |
| | | actaggtgtccttctataatattatggggtggaggggggtggtatggagcaaggggcaagttgg |
| | | gaagacaacctgtagggcctgcggggtctattgggaaccaagctggagtgcagtggcacaatc |
| | | ttggctcactgcaatctccgcctcctgggttcaagcgattctcctgcctcagcctcccgagttgttg |
| | | ggattccaggcatgcatgaccaggctcagctaatttttgtttttttggtagagacggggtttcaccat |
| | | attggccaggctggtctccaactcctaatctcaggtgatctacccaccttggcctcccaaattgctg |
| | | ggattacaggcgtgaaccactgctcccttccctgtccttctggagctcgagaggaacccctagtg |
| | | atggagttggccactccctctctgcgcgctcgctcgctcactgaggccgggcgaccaaaggtcg |
| | | cccgacgcccgggctttgcccgggggcctcagtgagcgagcgagcgcgcag |

### Example 2: Evaluation of Methods to Enable Multicistronic Transgene Expression in an AAV Vector System

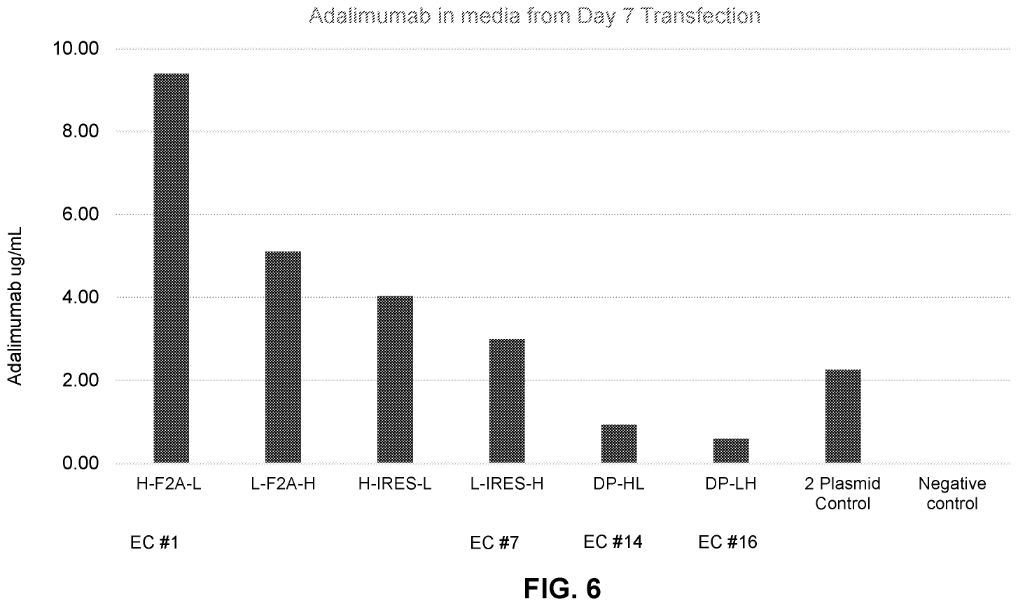

A multicistronic monoclonal antibody construct was prepared to compare expression for different configurations of heavy versus light chain and usage of linker (F2A or IRES) verses dual promoter system. The ORFs and expression cassettes encoding monoclonal antibodies are provided in Tables 16-17 (Expression Cassettes #1, 7, 14, and 16 and SEQ ID NOs: 62, 68, 75, and 77) and FIGS. 5A-5F. In vitro transfection of plasmids in HEK293 cells were evaluated and subsequently the plasmid constructs were evaluated via a hydrodynamic tail vein injection of a C57BL6 mouse.

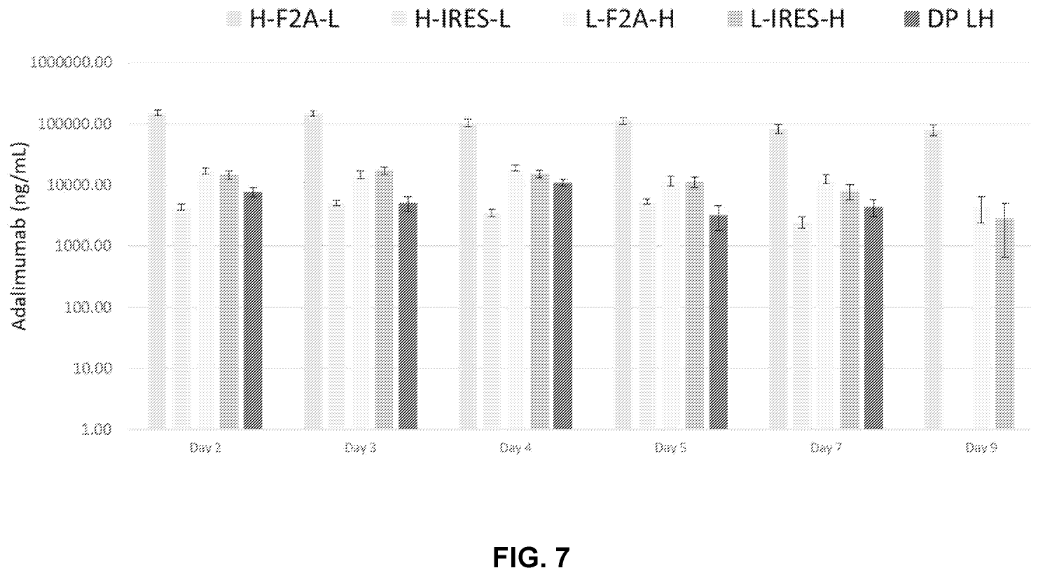

Based on in vitro transfection of HEK293 cells, the construct containing the F2A in a heavy chain then light chain configuration was the highest expressing plasmid. However, the western blots of the cell supernatant showed that the antibody secreted using the F2A were not completely processed. The band likely corresponded to an incomplete removal of the F2A peptide by furin. The IRES system produced approximately half the amount of antibody as compared to F2A when quantified by ELISA (FIG. 6); however, the IRES plasmids appear to produce an unequal ratio of heavy and light chains. While lowest concentrations were observed from the dual promoter system, it ultimately behaved similarly to the IRES with unequal ratios of heavy and light chain.

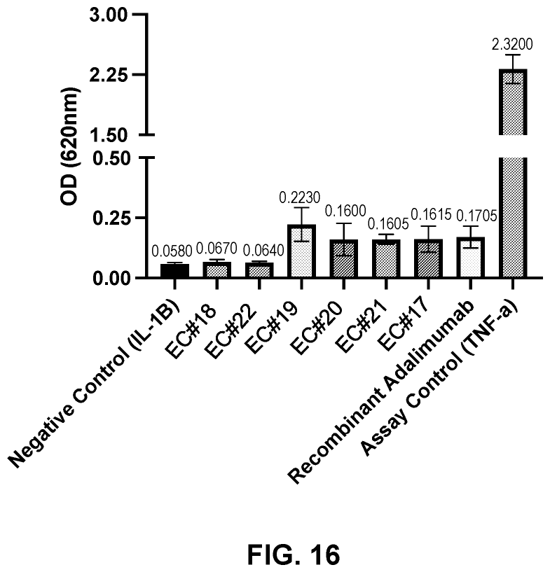

HEK293 cells were transduced at an MOI of 3E4 with AAV particles comprising the multicistronic monoclonal antibody constructs (including Expression Cassettes #1, 7, 14, and 16) and the expression of adalimumab was quantified. A comparison of the expression levels is shown in FIG. 15. Similar to the in vitro transfection results for these constructs, the IRES configuration expressed low levels of adalimumab.

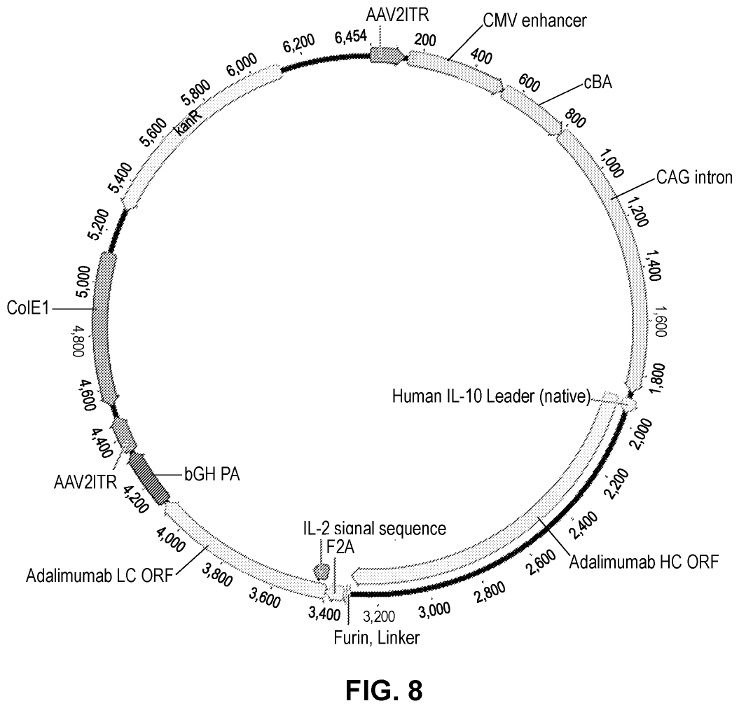

Subsequently, the constructs were further evaluated via hydrodynamic tail vein injection in mice. Mice were injected hydrodynamically at 0.1 mg/kg with the plasmids of interest and serum samples were analyzed on days 2, 3, 4, 5, 7, and 9. The pattern of expression levels in mice were generally similar to those observed in vitro with the F2A heavy followed by light chain resulting in the highest level of expression (FIG. 7). The F2A construct had the longest observed PK profile as well as substantially higher (˜10×) serum levels of antibody.

### Example 3: TNFa Neutralizing Capacity of Multicistronic Antibody Constructs

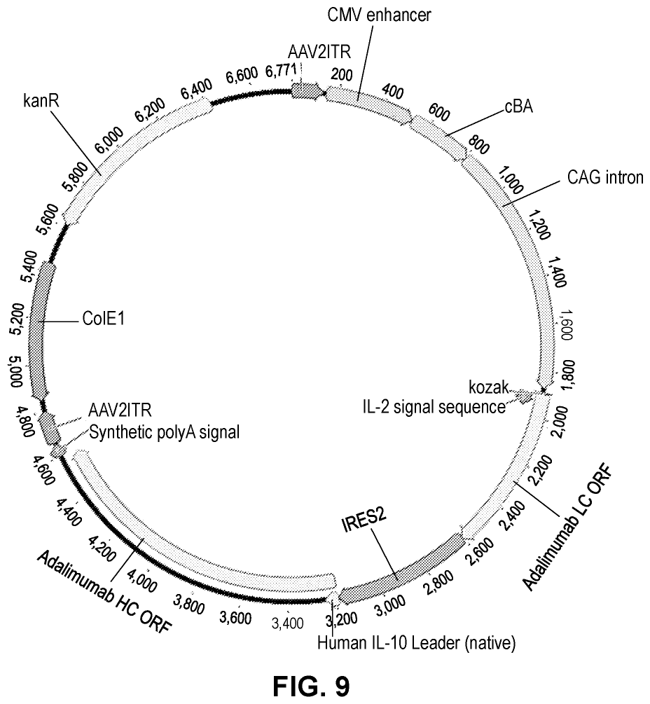

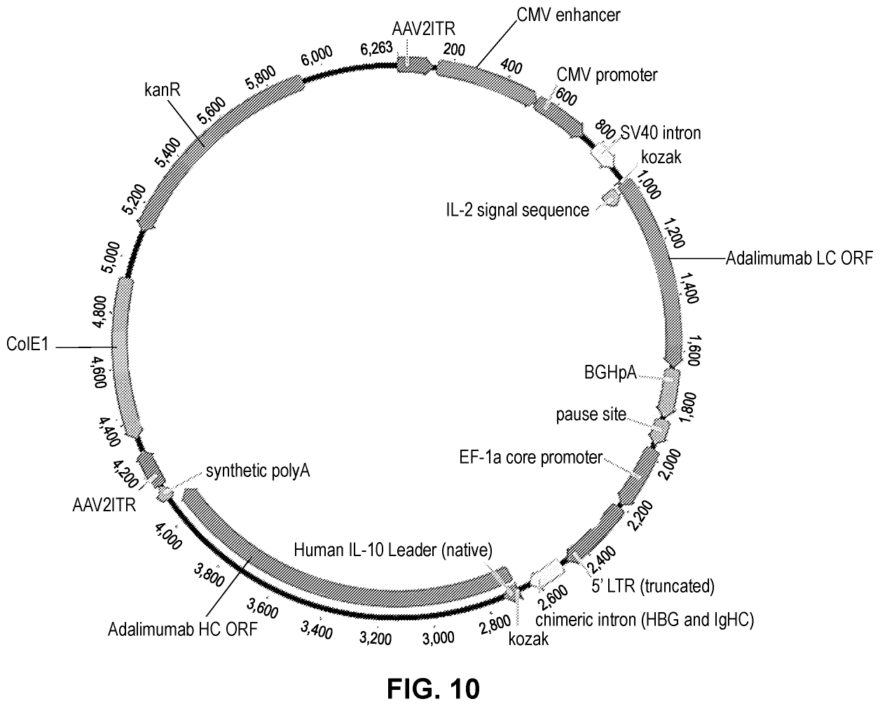

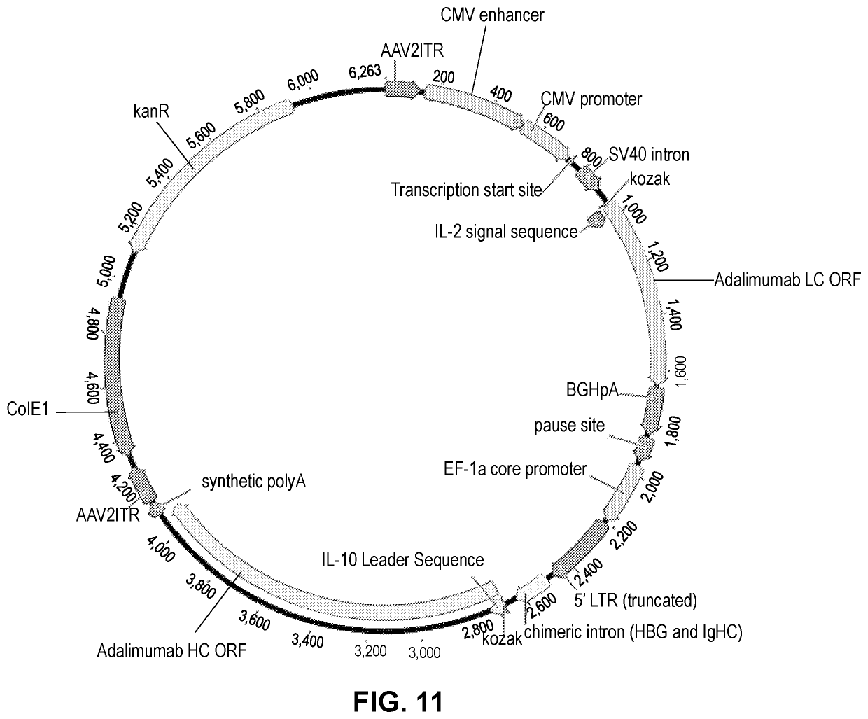

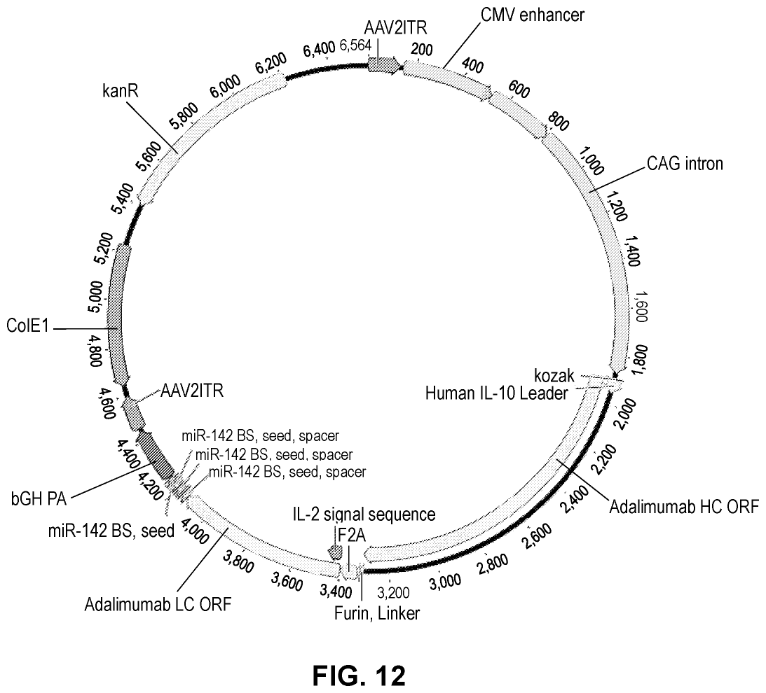

The TNFa neutralizing capacity of the multicistronic antibody constructs were evaluated. The constructs included the ORFs and expression cassettes encoding monoclonal antibodies as provided in Tables 16-17 (expression cassettes 17-21, SEQ ID NOs: 132-134, and FIGS. 8-11).

The TNFa neutralizing assay was carried out using HEK-Blue™ TNF-α cells. These cells are specifically designed for the detection of TNF-α by monitoring the activation of the AP-1/NF-κB pathway. These cells are derived from the human embryonic kidney 293 cell line by stable transfection with a SEAP (secreted embryonic alkaline phosphatase) reporter gene under the control of the IFN-β minimal promoter fused to five AP-1 and five NF-κB binding sites. Stimulation of HEK-Blue™ TNF-α cells with TNF-α triggers a signaling cascade leading to the activation of AP-1/NF-κB and the subsequent production of SEAR This is then assessed using QUANTI-Blue™ Solution, a SEAP detection reagent, and capturing the OD at 620 nm.

High OD corresponds to more SEAP production, which correlates to more of free TNF-alpha. On the contrary, low OD values correspond to low SEAP levels that correlate to less free TNF-alpha (as Adalimumab would neutralize it) to activate the AP-1/NF-κB pathway.

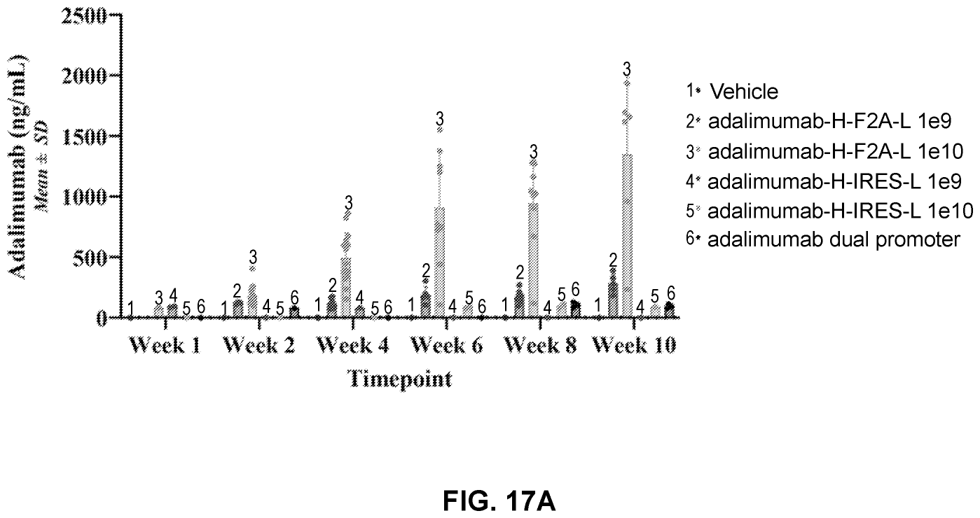

HEK-Blue™ TNF-α cells were transduced with AAV2 vectors encoding anti-TNFa monoclonal antibodies (AAV2 vectors including expression cassettes 17-21, SEQ ID NOs: 132-134, and FIGS. 8-11). 72 hrs after transduction, the supernatant was collected and the amount of adalimumab was quantified. Analysis shows that all antibody constructs were able to neutralize TNFa compared to the recombinant adalimumab control (FIG. 16). The F2A system (expression cassettes EC #18 and #22) had a higher neutralizing effect than the IRES system (expression cassette EC #19) and the Duel Pomoter systems (expression cassette EC #17 and #20). Unexpectedly, adalimumab expressed from the F2A system (expression cassettes EC #18 and #22) had a higher neutralizing effect than recombinant adalimumab by approximately 2× to 3×.

### Example 4: Evaluation of Anti-TNFa Antibody Expression in the Serum and Ocular Tissue of SCID Mice

The expression of an adalimumab was evaluated after administration of an AAV2 vector encoding adalimumab. The mice received a single 1E9 or 1E10 vg bilateral IVT injection (injection volume=0.5 μL) of an AAV2 vector including an expression cassette encoding adalimumab as described in Table 18. Serum samples and ocular tissues were collected on Weeks 2, 4, 8, and 12. Additional serum samples were collected on Weeks 1, 6, and 10. For both serum and ocular samples, adalimumab concentrations were quantified using a commercially available antibody ELISA kit (Abcam #ab237641).

TABLE 18
Design of Pharmacokinetic analysis of AAV vectors driving
expression of anti-TNFa antibodies in SCID mice

| Group | Number of | Animal | Treatment | Route and | Euthanasia/Tissue |
|---|---|---|---|---|---|
| # | Animals/Sex | Strain | (Day/Amount) | Volume (OU) | Collections |
| 1 | 12 (Female) | SCID | Vehicle Control | Intravitreal | Ocular Exams: |
| | | | in 1x PBS | injection 0.5 | Weeks 2, 4, 8, and 12 |
| 2 | 18 (Female) | | Adalimumab H- | μl | Serum (non-terminal: |
| | | | F2A-L (EC #18; | | 100 μL; terminal |
| | | | SEQ ID NO: 134) | | animals: 1 mL): |
| | | | 1E9 vg/eye in 1X | | Weeks 1, 2, 4, 6, 8, |
| | | | PBS | | 10, and 12 |
| 3 | 18 (Female) | | Adalimumab H- | | Ocular Tissues: |
| | | | F2A-L (EC #18; | | Weeks 2, 4, 8, and |
| | | | SEQ ID NO: 134) | | 12: |
| | | | 1E10 vg/eye in | | Group 1: n = 2 |
| | | | phosphate buffer | | animals/timepoint/gr |
| | | | (no salt) | | oup were euthanized |
| 4 | 18 (Female) | | Adalimumab H- | | and eyes collected for |
| | | | IRES-L (EC#19; | | ELISA analysis^{∞§} |
| | | | SEQ ID NO: 136) | | Groups 2-6: n = 3 |
| | | | 1E9 vg/eye in 1X | | animals/timepoint/gr |
| | | | PBS | | oup were euthanized |
| 5 | 18 (Female) | | Adalimumab H- | | and eyes collected for |
| | | | IRES-L (EC#19; | | ELISA analysis^{∞§} |
| | | | SEQ ID NO: 136) | | Histopathology: |
| | | | 1E10 vg/eye in | | Group 1: n = 2 |
| | | | phosphate buffer | | animals/timepoint/ |
| | | | (no salt) | | group were processed |
| 6 | 18 (Female) | | Adalimumab- | | for histopathology |
| | | | Dual Promoter | | Groups 2-6: n = 3 |
| | | | (EC#20; SEQ ID | | animals/timepoint/ |
| | | | NO: 138) | | group were processed |
| | | | 1E9 vg/eye in | | for histopathology |
| | | | phosphate buffer | | |
| | | | (no salt) | | |

Abbreviations: OU: both eyes; RoA: Route of Administration; N: number of animals; IVT: intravitreal; SC: subcutaneous.
^{∞}Each eye was homogenized, including the lens, for PK analysis via ELISA.
^{§}Both serum and ocular homogenate were evaluated for anti-TNFa antibody levels.

On Day 0 prior to injection, mice were given buprenorphine 0.01-0.05 mg/kg SC. A topical mydriatic (1.0% Tropicamide HCL, and 2.5% Phenylephrine HCL) was applied at least 15 minutes prior to the injection. Animals were be tranquilized for the intravitreal injections and one drop of 0.5% proparacaine HCL was applied to both eyes. Alternatively, mice were anesthetized with inhaled isoflurane. The injection was made superiorly using a 33 G needle and a Hamilton Syringe. After dispensing the syringe contents, the syringe needle was slowly withdrawn.

At the timepoints specified in the table above, non-terminal animals had about 100 μL of blood drawn. Briefly, animals were gently restrained and a 4 mm lancet (Fisher Scientific cat #NC9922361) was used to puncture the submandibular vein. Blood was collected into a 0.5 mL BD microtainer tube with serum separator. Blood was allowed to clot at room temperature for at least 20 minutes prior to serum processing. The samples were centrifuged at room temperature for 10 minutes at 4,000×g in a benchtop microfuge. Within 20 minutes of the blood collection time, the clear serum was transferred to a prelabelled polypropylene tube and will be stored frozen at −80° C. until analysis.

At the timepoints specified in the experimental design table, animals were euthanized via asphyxiation with carbon dioxide and death was confirmed by exsanguination.

Histopathology (weeks 4 and 12): Immediately following euthanasia, eyes including the optic nerve tail were collected into 10% neutral buffered formalin. The eyes were placed in 70% ethanol the following day. The eyes were processed to paraffin blocks for sectioning. Sagittal sections of each eye (5 μm thickness) were prepared for all animals. At least 3 slides containing a ribbon of approximately 5 sections were collected sequentially. The optic nerve was included in the sectioning. The slides were stained with hematoxylin and eosin (H&E) and examined using light microscopy. No abnormal findings were observed (data not shown).

Terminal Blood Collection and Euthanasia: To collect blood from terminal animals, a 25G needle was inserted into the heart and the animal was exsanguinated and euthanized, and blood was collected into a 1.5 mL RNAse/DNAse-free microfuge tube and allowed to clot at room temperature for at least 20 minutes prior to serum processing. The samples were centrifuged at room temperature for 10 minutes at 4,000×g in a benchtop microfuge. Within 30 minutes of the blood collection time, the clear serum was transferred to a prelabelled polypropylene tube and will be stored frozen at −80° C. until analysis.

PK Ocular Tissue Collection: Immediately following euthanasia, eyes were enucleated and trimmed of extraneous material. The ocular tissue were placed into a pre-weighed RNAse/DNAse-free Precellys 2 mL homogenization tubes and re-weighed. Tissues were stored at −80° C. until homogenized for anti-TNFa ELISAs.

Tissue Homogenization: Tissues were homogenized in 10× by weight volume (minimum volume: 100 μL) of phosphate-buffered saline containing protease inhibitor in 2 mL Precellys tubes under the following conditions: a Precellys Evolution homogenizer was used at 3×6,500 rpm for 30 seconds each with a 30 second delay. Following homogenization, tissues were spun for 5 minutes at 10,000 g and the supernatant was collected and transferred to a new tube and was stored frozen at −80° C. until analysis.

Adalimumab Tissue Concentration Analysis: Adalimumab antibody ELISA were used. A 9-point standard curve ranging from 1,000 ng/mL adalimumab to 7.8125 ng/mL adalimumab and a blank were run in duplicate in both assay buffer (AB; for ocular homogenate (OH) samples) and in 1:10 pooled serum (diluted in assay buffer; for comparison to serum samples). Two control samples at 500 ng/mL and at 30 ng/mL adalimumab were run in duplicate in assay buffer and 1:10 pooled serum. Serum samples were diluted 1:10 in assay buffer. Ocular homogenate samples were diluted 1:5 in assay buffer. Concentration of adalimumab in unknown samples were interpolated against the standard curve using a 4-parameter logistic (4PL) curve with 1/y₂ weighting. OH sample concentrations (ng/mL) were divided by the original stock concentration (mg/mL) and the ng adalimumab per mg tissue was plotted.

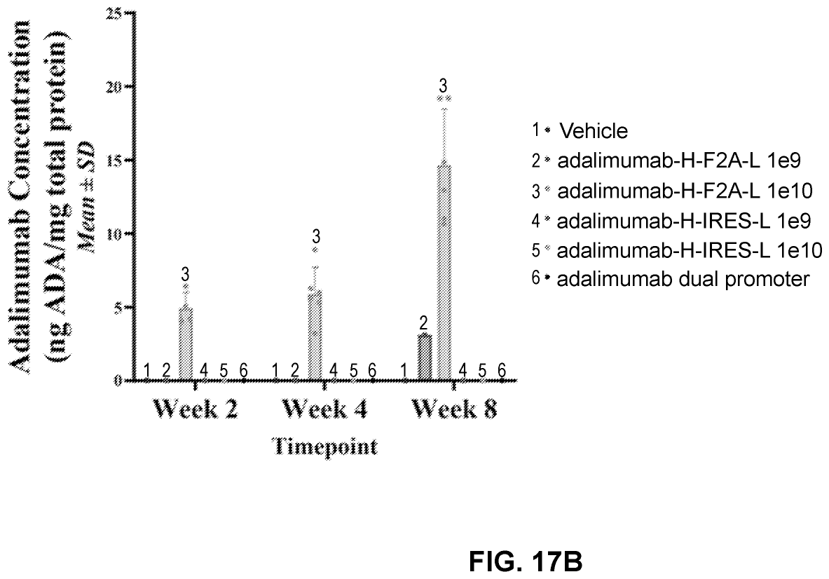

Serum levels of adalimumab were quantified 1, 2, 4, 6, 8, and 10 weeks after administration of AAV2 vectors encoding adalimumab (FIG. 17A). Administration of adalimumab H-F2A-L constructs resulted in higher levels of serum adalimumab compared to adalimumab H-IRES-L or adalimumab dual promoter constructs at both doses. Similarly, ocular expression of adalimumab was higher from H-F2A-L constructs compared to adalimumab H-IRES-L or adalimumab dual promoter constructs (FIG. 17B).

### Example 5: Estimated IVT Human Dosing Range of AAV2-Adalimumab

Similar to the brain, the eye is known to be isolated from systemic circulation by the blood-aqueous barrier and the blood-retina barrier. The two barriers together tightly restrict the movement of therapeutic proteins and other molecules between the systemic and ocular compartments making it challenging to treat many ocular diseases with systemic therapies. Published literature indicates that only about 1% of the total dose of therapeutic protein (e.g., ranibizumab) reaches systemic circulation after intravitreal injection in humans and higher species (e.g., non-human primates); however, ocular concentrations are not reported for mice (see, e.g., Bakri et al., Ophthalmology, 114(5):855-9 (2007); Caruso et al., Mol Pharm 17(2):695-709 (2020); Xu et al., Invest Ophthalmol Vis Sci 54(3):1616-24 (2013)). Therefore, recombinant adalimumab (Humira) was utilized to establish ocular pharmacokinetic profiles after intravitreal (IVT) injection in severe combined immunodeficiency (SCID) mice. The pharmacokinetic profile of recombinant adalimumab was compared to the PK of an IVT administered AAV2 vector encoding adalimumab according to the methods provided herein.

For studies with recombinant adalimumab (Humira), mice received a 3.2 and 1.6 ug dose on days 1 and 7, respectively, as a 0.5 μL bilateral IVT injection. Serum samples and ocular tissues were collected on Days 1, 3, 7 (prior to the 2^{nd} dose), 8, 10, 14 and 21 post injection. For studies using the AAV vector, mice received a single 1E9 or 1E10 vg bilateral IVT injection (0.5 μL) of an AAV2 vector encoding adalimumab (expression cassettes #18, 19, or 20). Serum samples and ocular tissues were collected on Weeks 2, 4, 8, and 12. Additional serum samples were collected on Weeks 1, 6, and 10. For both serum and ocular samples, adalimumab concentrations were quantified using a commercially available antibody ELISA kit (cat #ab237641).

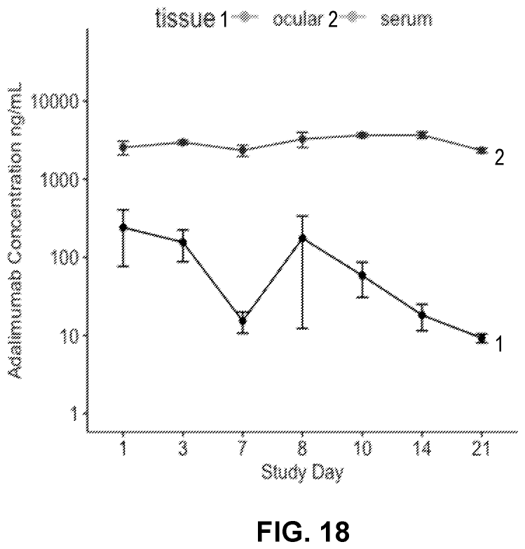

In mice that received recombinant adalimumab (Humira) IVT injection, the distribution of adalimumab from the ocular compartment to serum was rapid. After initial rapid distribution a small concentration remained detectable in the eye throughout the duration of the study (FIG. 18) which was likely due to redistribution of adalimumab from the systemic circulation back to the ocular compartment. Contrary to what is found in humans after IVT injection of an antibody, mice had substantially higher serum concentrations within 24 hours (FIG. 18), with the systemic clearance remaining similar to what was reported for adalimumab in mice. These results indicate an interspecies difference in the distribution of the antibody between the compartments. In mice that received IVT injection of AAV2-adalimumab, ocular concentrations of adalimumab elevated over 8 weeks and plateaued by 12 weeks (FIG. 17B). Similar to what was observed after injection of recombinant adalimumab (Humira), adalimumab concentrations were higher in the serum as compared to what was observed in the eye (FIGS. 17A-17B).

While the movement of large therapeutic proteins is known to be generally restricted between central and peripheral compartments, the results disclosed herein suggest that in mice, adalimumab moves rapidly from the eye and into systemic circulation with most (>80%) of the antibody in the serum 24 hours after injection. For reference, in humans, large therapeutic proteins are reported to have an ocular distribution half-life of 6 to 9 days. Because of this apparent inter-species disparity in the kinetics of exit from the ocular compartment, an estimation of human exposure using mouse ocular data alone is likely insufficient. Therefore, to calculate an estimated equivalent human exposure, a composite of mouse serum and ocular concentrations at week 4 was utilized after a 1E9 or 1E10 vg intravitreal dose of AAV2-adalimumab in mice (FIG. 17B). The composite data was adjusted assuming a 1% of adalimumab will distribute to the systemic compartment after a unilateral IVT injection in a human subject.

$$Equivalent Human Ocular Exposure = \frac{Mouse Serum Conc . + Mouse Ocular Conc .}{2} \times 0.99$$

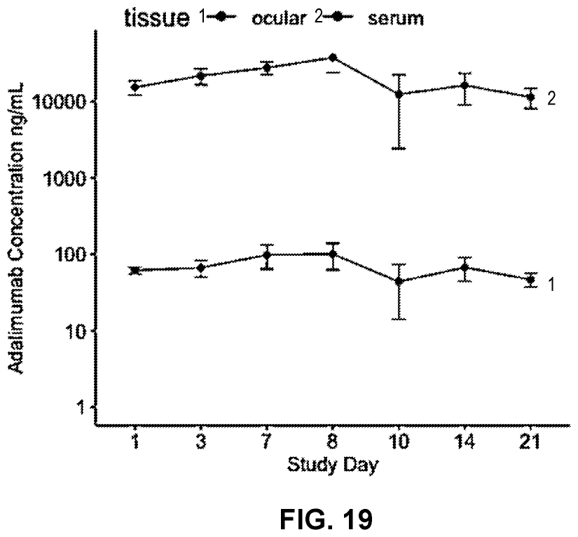

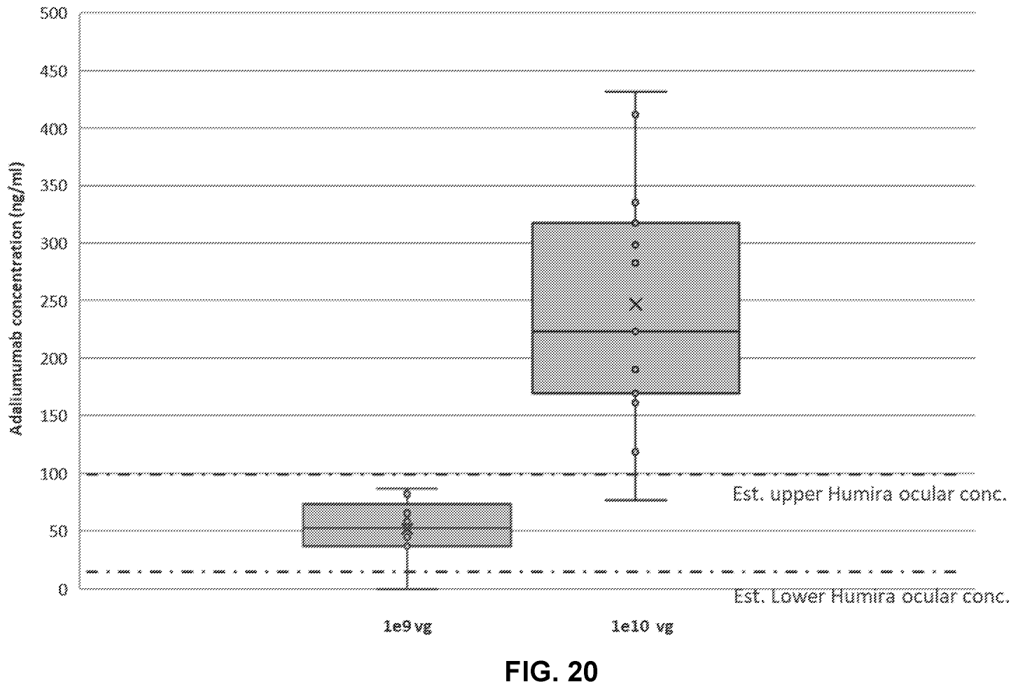

Based on this calculation estimate, the 1E9 vg IVT dose of AAV2-adalimumab produces an ocular adalimumab exposure within the upper and lower bounds of the serum adalimumab exposure after subcutaneous recombinant adalimumab (Humira) injection (subcutaneous profiles shown in (FIG. 19)), and the 1E10 vg dose exceeds those bounds (FIG. 20). Upper and lower bounds were based on 0.1 and 1% of adalimumab steady state concentrations (about 10 μg/ml) in serum after 40 mg subcutaneous administration as reported in the recombinant adalimumab (Humira) label. The 1% ocular concentration is also consistent with data, which indicates that <1% adalimumab is found in the eye as compared to systemic concentration (FIG. 19) in SCID mice that received 32 μg SC adalimumab on day 1 and 16 μg SC adalimumab on day 7.

To identify an estimated human dose for AAV2-adalimumab, composite serum and mouse concentrations at 1E9 vg dose were utilized.
Based on allometric scaling of the intravitreal volume between mouse (about 4 μL) and human (about 4 mLs) a 1000 fold scaling factor projected a potential efficacious dose of about 1E12 vg in humans.
In addition, based on AAV2 serotype, intravitreal route of administration and expressing a secreted large therapeutic protein a projected a potential efficacious dose of 1E9 vg in humans.

In consideration of the data disclose herein and the factors above, an estimated efficacious ocular dose of AAV-adalimumab in humans was calculated to be between about 1E9 and about 3E12 vg.

What is claimed is:

1. A recombinant adeno-associated virus (rAAV) particle comprising a capsid and a vector genome, the vector genome comprising an inverted terminal repeat (ITR) and an antibody expression cassette, wherein the antibody expression cassette comprises (a) a promoter, (b) a first leader sequence operably linked to a nucleic acid sequence encoding a heavy chain variable region (VH) of an anti-tumor necrosis factor alpha (anti-TNFalpha) antibody or an antigen-binding fragment thereof, (c) a linker sequence comprising a proteolytic cleavage site, and (d) a second leader sequence operably linked to a nucleic acid sequence encoding a light chain variable region (VL) of an anti-TNFalpha antibody or an antigen-binding fragment thereof, optionally, wherein the AAV capsid serotype is AAV2 or a modified version thereof.

2. The rAAV particle of claim 1, wherein the nucleic acid sequence encoding the VH of the anti-TNFalpha antibody or an antigen-binding fragment thereof comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 7, 8, 9, 102, 143, or 147; and the nucleic acid sequence encoding the VL of the anti-TNFalpha antibody or an antigen-binding fragment thereof comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 11, 12, 13, 103, 145, or 149.

3. A vector comprising an antibody expression cassette comprising:
(a) a promoter;
(b) a first leader sequence;
(c) a nucleic acid sequence encoding a heavy chain variable region (VH) of an anti-tumor necrosis factor alpha (anti-TNFalpha) antibody or an antigen-binding fragment thereof comprising a nucleotide sequence at least 85%, 86%, 87%, 88%, 89%, 90%, 91%, 92%, 93%, 94%, 95%, 96%, 97%, 98%, 99%, or 100% identical to SEQ ID NO: 7, 8, 9, 102, 143, or 147;
(d) a linker sequence;
(e) a second leader sequence;
(f) a nucleic acid sequence encoding a light chain variable region (VL) of an anti-TNFalpha antibody or an antigen-binding fragment thereof comprising a nucleotide sequence at least 85%, 86%, 87%, 88%, 89%, 90%, 91%, 92%, 93%, 94%, 95%, 96%, 97%, 98%, 99%, or 100% identical to SEQ ID NO: 11, 12, 13, 103, 145, or 149.

4. The rAAV particle or the vector of any one of claims 1-3, wherein the nucleic acid sequence encoding the VH comprises a nucleotide sequence at least 90%, 91%, 92%, 93%, 94%, 95%, 96%, 97%, 98%, 99%, or 100% identical to SEQ ID NO: 7, 8, 9, 102, 143, or 147.

5. The rAAV particle or the vector of any one of claims 1-4, wherein the nucleic acid sequence encoding the VL comprises a nucleotide sequence at least 90%, 91%, 92%, 93%, 94%, 95%, 96%, 97%, 98%, 99%, or 100% identical to SEQ ID NO: 11, 12, 13, 103, 145, or 149.

6. The rAAV particle or the vector of any one of claims 1-5, wherein the nucleic acid sequence encoding the having chain (HC) comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 17, 18, 19, 110, 142, 146, 150, or 151.

7. The rAAV particle or the vector of any one of claims 1-6, wherein the nucleic acid sequence encoding the light chain (LC) comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 21, 22, 23, 111, 144, or 148.

8. The rAAV particle or the vector of any one of claims 1-7, wherein the linker sequence comprises a proteolytic cleavage site comprising a furin cleavage site, a 2A cleavage site, or a combination thereof.

9. The rAAV particle or the vector of claim 9, wherein the proteolytic cleavage site comprises a furin cleavage site and a 2A cleavage site.

10. The rAAV particle or the vector of claim 8 or 9, wherein the furin cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 26.

11. The rAAV particle or the vector of any one of claims 8-10, wherein the 2A cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 28.

12. The rAAV particle or the vector of any one of claims 1-11, wherein the first leader sequence is an IL-10 leader sequence.

13. The rAAV particle or the vector of any one of claims 1-12, wherein the second leader sequence is an IL-2 leader sequence.

14. The rAAV particle or the vector of claim 12 or 13, wherein the IL-10 leader sequence has a nucleotide sequence at least 85%, 86%, 87%, 88%, 89%, 90%, 91%, 92%, 93%, 94%, 95%, 96%, 97%, 98%, 99%, or 100% identical to SEQ ID NO: 30 or 112.

15. The rAAV particle or the vector of claim 13 or 14, wherein the IL-2 leader sequence has a nucleotide sequence at least 85%, 86%, 87%, 88%, 89%, 90%, 91%, 92%, 93%, 94%, 95%, 96%, 97%, 98%, 99%, or 100% identical to any of SEQ ID NO: 29 or 113.

16. The rAAV particle or the vector of any one of any one of claims 1-15, wherein the promoter is a CAG promoter, a CBA promoter, a CMV promoter, an EF1α promoter, a CMV promoter with a CMV enhancer, a CMV promoter with a SV40 intron, an EF1α with a CMV enhancer, or tissue specific promoter.

17. The rAAV particle or the vector of any one of claims 1-16, wherein the promoter is a CAG promoter.

18. The rAAV particle or the vector of any one of claims 1-17, wherein the promoter comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 38.

19. The rAAV particle or the vector of any one of claims 1-18, wherein the anti-TNFalpha antibody is a monoclonal antibody.

20. The rAAV particle or the vector of any one of claims 1-19, wherein the anti-TNFalpha antibody is adalimumab.

21. The rAAV particle or the vector of any one of claims 1-20, wherein the antibody expression cassette comprises a poly(A) sequence.

22. The rAAV particle or the vector of claim 21, wherein the poly(A) sequence is selected from a bGHpA, a hGHpA, a SV40pA, hGHpA, or a synthetic pA.

23. The rAAV particle or the vector of claim 21 or 22, wherein the poly(A) sequence comprises a bGHpA.

24. The rAAV particle or the vector of any one of claims 21-23, wherein the poly(A) comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 39, 40, or 114.

25. The rAAV particle or the vector of any one of claims 1-24, wherein the antibody expression cassette comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 42-51 or any combination thereof.

26. The rAAV particle or the vector of any one of claims 1-25, wherein the antibody expression cassette comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 52-61 or any combination thereof.

27. The rAAV particle or the vector of any one of claims 1-26, wherein the antibody expression cassette comprises an open reading frame (ORF) comprising the nucleotide sequence at least 85%, 86%, 87%, 88%, 89%, 90%, 91%, 92%, 93%, 94%, 95%, 96%, 97%, 98%, 99%, or 100% identical to SEQ ID NO: 49 and the nucleotide sequence at least 85%, 86%, 87%, 88%, 89%, 90%, 91%, 92%, 93%, 94%, 95%, 96%, 97%, 98%, 99%, or 100% identical to SEQ ID NO: 56.

28. The AAV particle or the vector of any one of claims 1-27, wherein the antibody expression cassette comprises a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 62-77 115-141, or 153-158.

29. The AAV particle or the vector of any one of claims 1-27, wherein the antibody expression cassette comprises a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 64 or 115.

30. The vector of any one of claims 3-29 which comprises an inverted terminal repeat (ITR).

31. The rAAV or the vector of any one of claims 1-30, wherein the AAV ITR comprises a pair of ITRs flanking the antibody expression cassette.

32. The rAAV particle or the vector of claim 31, wherein the ITRs are AAV2 serotype.

33. The vector of any one of claims 3-32, wherein the vector is packaged in an AAV capsid.

34. The rAAV or the vector of any one of claims 1-33, wherein the AAV capsid serotype is selected from the group consisting of AAV1, AAV2, AAV3, AAV4, AAV5, AAV6, AAV7, AAV8, AAVrh8, AAVrh9, AAV9, AAVrh10, AAVshH10, AAV10, AAV11, AAV12, or a modified version thereof.

35. The rAAV or the vector of claim 34, wherein the AAV capsid serotype is AAV2 or a modified version thereof, optionally, AAV2 Quad Y-F, AAV2 Quad Y-F+T491V, or AAV2.7m8.

36. A host cell comprising the rAAV particle or the vector of any one of claims 1-35.

37. A composition comprising the rAAV particle or the vector of any one of claims 1-35 and a carrier.

38. The composition of claim 37, wherein the carrier is water or saline.

39. A method of expressing an anti-TNFalpha antibody or antigen-binding fragment thereof in a cell, comprising administering to the cell the rAAV particle, the vector, or the composition of any one of the previous claims, thereby expressing the anti-TNFalpha antibody or antigen-binding fragment thereof in the cell.

40. The method of claim 39, wherein the administration is in vitro.

41. The method of claim 39, wherein the administration is in vivo.

42. A method of expressing an anti-TNFalpha antibody or antigen-binding fragment thereof in a subject in need thereof, comprising administering to the subject the rAAV particle, the vector, or the composition of any one of the previous claims, thereby expressing the anti-TNFalpha antibody or antigen-binding fragment thereof in the subject.

43. A method of neutralizing TNFalpha in a subject comprising administering to the subject the rAAV particle, the vector, or the composition of any one of the previous claims, wherein the anti-TNFalpha antibody or antigen-binding fragment thereof expressed in the subject is capable of neutralizing TNFalpha.

44. The method of claim 43, wherein the TNFalpha neutralization is increased compared to TNFalpha neutralization in a subject administered recombinant adalimumab.

45. The method of any one of claims 42-44, wherein the subject suffers from an immune disease or disorder, an autoimmune disease or disorder, or an ocular disease or disorder.

46. A method of treating an immune disease or disorder, an autoimmune disease or disorder, or an ocular disease or disorder in a subject in need thereof comprising administering to the subject an effective amount of the rAAV particle, the vector, or the composition of any one of the previous claims, thereby expressing the anti-TNFalpha antibody or antigen-binding fragment thereof in the subject and treating the immune disease or disorder, an autoimmune disease or disorder, or an ocular disease or disorder.

47. The method of claim 45 or 46, wherein the subject suffers from an ocular disease or disorder.

48. A method of treating an ocular disease or disorder in a subject in need thereof comprising intravitreally administering to the subject an effective amount of a recombinant adeno-associated virus (rAAV) particle comprising a capsid and a vector genome, the vector genome comprising an inverted terminal repeat (ITR) and an antibody expression cassette, wherein the antibody expression cassette comprises (a) a promoter, (b) a nucleic acid sequence encoding a heavy chain variable region (VH) of an anti-tumor necrosis factor alpha (anti-TNFalpha) antibody or an antigen-binding fragment thereof, (c) a linker sequence, and (d) a nucleic acid sequence encoding a light chain variable region (VL) of an anti-TNFalpha antibody or an antigen-binding fragment thereof, optionally, wherein the AAV capsid serotype is AAV2 or a modified version thereof, thereby expressing the anti-TNFalpha antibody or antigen-binding fragment thereof in the subject and treating the ocular disease or disorder.

49. The method of any one of claims 45-48, wherein the ocular disease or disorder is uveitis.

50. The method of claim 49, wherein the uveitis is non-infectious uveitis, optionally selected from intermediate uveitis, posterior uveitis, and panuveitis.

51. The method of any of claims 45-49, wherein the ocular disease or disorder is a corneal disease, optionally selected from peripheral ulcerative keratitis, corneal hemangiogenesis, and noninfectious corneal melting.

52. The method of any one of claims 41-51, wherein the administration is suitable for delivery of the rAAV particle or the vector to one or both eyes.

53. The method of any one of claims 41-52, wherein the administration is by injection.

54. The method of any one of claims 41-53, wherein the administration is intravitreal.

55. The method of any one of claims 41-54, wherein the administration is a single dose.

56. The method of any one of claims 41-55, wherein the anti-TNFalpha antibody steady state concentration in the ocular fluid of the eye of the subject after administration is 10 ng/mL to 1000 ng/mL (1 μg/mL), 10 ng/mL to 500 ng/mL, or 10 ng/mL to 100 ng/mL.

57. The method of any one of claims 41-56, wherein the administration comprises a dose within the range of 1E9 vector genomes (vg) to 3E12 vg; 1E9 vg to 1E12 vg; 1E9 vg to 1E11 vg; or 1E9 vg to 3E10 vg.

58. The method of any one of claims 41-57, wherein the administration comprises a dose within the range of 1E9 vg to 1E12 vg.

59. The method of any one of claims 41-58, wherein the administration comprises a dose within the range of 1E9 vg to 1E11 vg.

60. A vector comprising an antibody expression cassette comprising:
(a) a promoter;
(b) a nucleic acid sequence encoding a heavy chain variable region (VH);
(c) a linker sequence comprising a proteolytic cleavage site;
(d) a nucleic acid sequence encoding a light chain variable region (VL).

61. The vector of claim 60, wherein the antibody expression cassette comprises (i) the promoter; (ii) a nucleic acid sequence encoding a heavy chain comprising the VH; (iii) the linker sequence comprising a furin and/or a 2A site; and (iv) a nucleic acid sequence encoding a light chain comprising the VL, which are in 5′-3′ orientation.

62. The vector of claim 60 or 61, wherein the promoter is selected from the group consisting of CAG, CBA, CMV, EF1α, CMV promoter with a CMV enhancer, CMV promoter with a SV40 intron, EF1α with a CMV enhancer, or tissue specific promoter.

63. The vector of any one of claims 60-61, wherein the promoter has a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38.

64. The vector of any one of claims 60-63, wherein the furin site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 26.

65. The vector of any one of claims 60-64, wherein the 2A site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 28.

66. The vector of any one of claims 60-65, wherein polynucleotide also comprises a leader sequence operably linked to the nucleic acid sequence encoding the heavy chain and/or the nucleic acid sequence encoding the light chain.

67. The vector of any one of claims 60-66, wherein the polynucleotide comprises a poly(A) sequence.

68. The vector of any one of claims 60-67 which comprises an inverted terminal repeat (ITR).

69. The vector of claim 68, wherein the AAV ITR comprises a pair of ITRs flanking the antibody expression cassette.

70. The vector of claim 69, wherein the ITRs are AAV2 serotype.

71. A recombinant AAV (rAAV) particle, comprising an AAV capsid and the vector of any one of claims 60-70.

72. The rAAV particle of claim 71, wherein AAV serotype is selected from the group consisting of AAV1, AAV2, AAV3, AAV4, AAV5, AAV6, AAV7, AAV8, AAVrh8, AAVrh9, AAV9, AAVrh10, AAV10, AAVshH10, AAV11, AAV12, or a modified version thereof, optionally, AAV2 Quad Y-F, AAV2 Quad Y-F+T491V, or AAV2.7m8.

73. A host cell comprising the vector or the rAAV particle of claim 71 or 72.

74. The rAAV particle of any one of claims 71-73 which is suitable for a single dose administration.

75. A method of expressing an antibody or antigen-binding fragment thereof in a cell or a subject, comprising administering to the cell or the subject a vector of any one of claims 60-70 or a rAAV particle of any one of claims 71-74, thereby expressing the antibody or antigen-binding fragment thereof in the subject.

76. A method of obtaining an effective steady state concentration of an anti-TNFalpha antibody in the eye of a subject in need thereof comprising intravitrial, intrastromal or transconjunctival administration of an single dose of an rAAV particle or vector comprising an antibody expression cassette encoding the anti-TNFalpha antibody to the subject, wherein the subject suffers from an ocular disease or disorder.

77. The method of claim 76, wherein the single dose comprises a pharmaceutical composition comprising 1E9 vg to 3E12 vg, 1E9 vg to 1E12 vg, 1E9 vg to 1E11 vg, or 1E9 vg to 3E10 vg.

78. The method of claim 76 or 77, wherein the single dose of the pharmaceutical composition is administered to an eye at a volume of 25 μL to 100 μL, 25 μL to 75 μL, 25 μL to 70 μL, 25 μL to 65 μL, 25 μL to 60 μL, 40 μL to 60 μL 25 μL to 55 μL, or 25 μL to 50 μL.

79. The method of any one of claims 76-78, wherein the administration is by injection.

80. The method of any one of claims 76-79, wherein the administration is intravitreal

81. The method of any one of claims 76-79, wherein the ocular disease or disorder is uveitis.

82. The method of claim 81, wherein the uveitis is non-infectious uveitis, optionally selected from intermediate uveitis, posterior uveitis, and panuveitis.

83. The method of any one of claims 76-79, wherein the administration is intrastromal or transconjunctival.

84. The method of any one of claims 76-79 or 83, wherein the ocular disease or disorder is a corneal disease, optionally selected from peripheral ulcerative keratitis, corneal hemangiogenesis, and noninfectious corneal melting.

85. The method of any one of claims 76-84, wherein the anti-TNFalpha antibody steady state concentration in the ocular fluid of the eye of the subject after administration is 10 ng/mL to 1000 ng/mL (1 μg/mL).

86. The method of any one of claims 76-85, wherein the anti-TNFalpha antibody steady state concentration in the ocular fluid of the eye of the subject after administration is 10 ng/mL to 500 ng/mL; 10 ng/mL to 250 ng/mL; 10 ng/mL to 100 ng/mL; or 20 ng/mL to 80 ng/mL.

87. The method of any one of claims 76-86, wherein the anti-TNFalpha antibody steady state concentration in the serum of the subject after administration is less than 1% of the total anti-TNFalpha antibody concentration after administration (to one or both eyes).

88. The method of any one of claims 76-87, wherein the anti-TNFalpha antibody steady state concentration in the serum of the subject after administration is less than 20 ng/mL.

89. The method of any one of claims 76-88, wherein the rAAV particle or the vector of any one of claims 1-35, 60-74 or the composition of claim 37 or 38.

90. The method of any one of claims 41-59 and 75-89, wherein the anti-TNFalpha antibody steady state concentration in the ocular fluid of the eye of the subject after administration is at least 10 ng/mL, at least 20 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 250 ng/mL, at least 300 ng/mL, at least 400 ng/mL, at least 500 ng/mL, at least 600 ng/mL, at least 700 ng/mL, at least 800 ng/mL, at least 900 ng/mL, or at least 1 μg/mL.

91. The method of any one of claims 41-59 and 75-90, wherein the anti-TNFalpha antibody steady state concentration in the ocular fluid of the eye of the subject after administration is between 10 ng/mL to 1000 ng/mL (1 μg/mL), 10 ng/mL to 500 ng/mL, or 10 ng/mL to 100 ng/mL.

92. The method of claim 90 or 92, wherein the ocular fluid is aqueous or viterious humor.

93. The method of any one of claims 41-59 and 75-92, wherein the anti-TNFalpha antibody steady state concentration in the serum of the subject after administration is less than 1% of the total anti-TNFalpha antibody concentration after administration to the subject.

94. The method of any one of claims 41-59 and 75-93, wherein the anti-TNFalpha antibody steady state concentration in the serum of the subject after administration is less than 20 ng/mL, less than 15 ng/mL, less than 10 ng/mL, less than 5 ng/mL, less than 1 ng/mL, or less than 0.5 ng/mL.

95. The method of any one of claims 41-59 and 75-94, wherein the anti-TNFalpha antibody steady state concentration in the serum of the subject after administration is 0.1 ng/mL to 20 ng/mL, 0.5 ng/mL to 20 ng/mL, 0.5 ng/mL to 10 ng/mL, or 0.5 ng/mL to 5 ng/mL.
